SEQUENCE LISTING (102 sequences)

SEQ ID NO: 1 - protein, 238 aa, Aequorea victoria
MSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 2 - protein, 248 aa, Artificial Sequence
  Variant of green fluorescent protein (GFP) of Aequorea victoria
MGHHHHHHGGASKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSALSKDPNEKRDHMVLLEFVTAAGITHGMDELYK

SEQ ID NO: 3 - protein, 248 aa, Artificial Sequence
  Variant of green fluorescent protein (GFP) of Aequorea victoria
MGHHHHHHGGASKGEELFTGVVPILVELDGDVNGHEFSVRGEGEGDATEGELTLKFICTTGELPVPWPTLVTTLTYGVQCFSRYPDHMKQHDFFKSAMPEGYVQERTISFKDDGTYKTRAEVKFEGDTLVNRIELKGIDFKEDGNILGHKLEYNFNSHDVYITADKQENGIKAEFEIRHNVEDGSVQLADHYQQNTPIGDGPVLLPDDHYLSTESALSKDPNEDRDHMVLLEFVTAAGIDHGMDELYK

SEQ ID NO: 4 - protein, 248 aa, Artificial Sequence
  Variant of green fluorescent protein (GFP) of Aequorea victoria
MGHHHHHHGGASKGEELFDGEVPILVELDGDVNGHEFSVRGEGEGDATEGELTLKFICTTGELPVPWPTLVTTLTYGVQCFSRYPDHMDQHDFFKSAMPEGYVQERTISFKDDGTYKTRAEVKFEGDTLVNRIELKGIDFKEDGNILGHKLEYNFNSHDVYITADKQENGIKAEFEIRHNVEDGSVQLADHYQQNTPIGDGPVLLPDDHYLSTESALSKDPNEDRDHMVLLEFVTAAGIDHGMDELYK

SEQ ID NO: 5 - protein, 248 aa, Artificial Sequence
  Variant of green fluorescent protein (GFP) of Aequorea victoria
MGHHHHHHGGASKGEELFDGVVPILVELDGDVNGHEFSVRGEGEGDATEGELTLKFICTTGELPVPWPTLVTTLTYGVQCFSDYPDHMDQHDFFKSAMPEGYVQERTISFKDDGTYKTRAEVKFEGDTLVNRIELKGIDFKEDGNILGHKLEYNFNSHDVYITADKQENGIKAEFEIRHNVEDGSVQLADHYQQNTPIGDGPVLLPDDHYLSTESALSKDPNEDRDHMVLLEFVTAAGIDHGMDELYK

SEQ ID NO: 6 - protein, 248 aa, Artificial Sequence
  Variant of green fluorescent protein (GFP) of Aequorea victoria
MGHHHHHHGGASKGERLFTGVVPILVELDGDVNGHKFSVRGEGEGDATRGKLTLKFICTTGKLPVPWPTLVTTLTYGVQCFSRYPKHMKRHDFFKSAMPEGYVQERTISFKKDGTYKTRAEVKFEGRTLVNRIELKGRDFKEKGNILGHKLEYNFNSHNVYITADKRKNGIKANFKIRHNVKDGSVQLADHYQQNTPIGRGPVLLPRNHYLSTRSALSKDPKEKRDHMVLLEFVTAAGITHGMDELYK

SEQ ID NO: 7 - protein, 248 aa, Artificial Sequence
  Variant of green fluorescent protein (GFP) of Aequorea victoria
MGHHHHHHGGASKGERLFRGKVPILVELKGDVNGHKFSVRGKGKGDATRGKLTLKFICTTGKLPVPWPTLVTTLTYGVQCFSRYPKHMKRHDFFKSAMPKGYVQERTISFKKDGKYKTRAEVKFEGRTLVNRIKLKGRDFKEKGNILGHKLRYNFNSHKVYITADKRKNGIKAKFKIRHNVKDGSVQLADHYQQNTPIGRGPVLLPRNHYLSTRSKLSKDPKEKRDHMVLLEFVTAAGIKHGRDERYK

SEQ ID NO: 8 - protein, 248 aa, Artificial Sequence
  Variant of green fluorescent protein (GFP) of Aequorea victoria
MGHHHHHHGGRSKGKRLFRGKVPILVELKGDVNGHKFSVRGKGKGDATRGKLTLKFICTTGKLPVPWPTLVTTLTYGVQCFSRYPKHMKRHDFFKSAMPKGYVQERTISFKKDGKYKTRAEVKFEGRTLVNRIKLKGRDFKEKGNILGHKLRYNFNSHKVYITADKRKNGIKAKFKIRHNVKDGSVQLADHYQQNTPIGRGPVLLPRKHYLSTRSKLSKDPKEKRDHMVLLEFVTAAGIKHGRKERYK

SEQ ID NO: 9 - protein, 248 aa, Artificial Sequence
  Variant of green fluorescent protein (GFP) of Aequorea victoria
MGHHHHHHGGRSKGKRLFRGKVPILVKLKGDVNGHKFSVRGKGKGDATRGKLTLKFICTTGKLPVPWPTLVTTLTYGVQCFSRYPKHMKRHDFFKSAMPKGYVQERTISFKKDGKYKTRAEVKFKGRTLVNRIKLKGRDFKEKGNILGHKLRYNFNSHKVYITADKRKNGIKAKFKIRHNVKDGSVQLAKHYQQNTPIGRGPVLLPRKHYLSTRSKLSKDPKEKRDHMVLLEFVTAAGIKHGRKERYK

SEQ ID NO: 10 - protein, 248 aa, Artificial Sequence
  Variant of green fluorescent protein (GFP) of Aequorea victoria
MGHHHHHHGGRSKGKRLFRGKVPILVKLKGDVNGHKFSVRGKGKGDATRGKLTLKFICTTGKLPVPWPTLVTTLTYGVQCFSRYPKHMKRHDFFKSAMPKGYVQERTISFKKDGKYKTRAEVKFKGRTLVNRIKLKGRDFKEKGNILGHKLRYNFNSHKVYITADKRKNGIKAKFKIRHNVKDGSVQLAKHYQQNTPIGRGPVLLPRKHYLSTRSKLSKDPKEKRDHMVLKEFVTAAGIKHGRKERYK

SEQ ID NO: 11 - protein, 280 aa, Artificial Sequence
  Variant of green fluorescent protein (GFP) of Aequorea victoria
MGHHHHHHGGASKGERLFRGKVPILVELKGDVNGHKFSVRGKGKGDATRGKLTLKFICTTGKLPVPWPTLVTTLTYGVQCFSRYPKHMKRHDFFKSAMPKGYVQERTISFKKDGKYKTRAEVKFEGRTLVNRIKLKGRDFKEKGNILGHKLRYNFNSHKVYITADKRKNGIKAKFKIRHNVKDGSVQLADHYQQNTPIGRGPVLLPRNHYLSTRSKLSKDPKEKRDHMVLLEFVTAAGIKHGRDERYKGSAGSAAGSGEFGLFGAIAGFIENGWEGMIDG

SEQ ID NO: 12 - protein, 26 aa, Artificial Sequence
  Variant of green fluorescent protein (GFP) of Aequorea victoria
GIGAVLKVLTTGLPALISWIKRKRQQ

SEQ ID NO: 13 - protein, 26 aa, Artificial Sequence
  melittin peptide variant
CIGAVLKVLTTGLPALISWIKRKRQQ

SEQ ID NO: 14 - protein, 16 aa, Artificial Sequence
  penetratin peptide
RQIKIWFQNRRMKWKK

SEQ ID NO: 15 - protein, 30 aa, Artificial Sequence
  bovine PrP (1-30) peptide
MVKSKIGSWILVLFVAMWSDVGLCKKRPKP

SEQ ID NO: 16 - protein, 24 aa, Artificial Sequence
  MPG delta NLS peptide
GALFLGWLGAAGSTMGAPKSKRKV

SEQ ID NO: 17 - protein, 21 aa, Artificial Sequence
  TP-10 peptide
AGYLLGKINLKALAALAKKIL

SEQ ID NO: 18 - protein, 23 aa, Artificial Sequence
  EB1 peptide
LIRLWSHLIHIWFQNRRLKWKKK

SEQ ID NO: 19 - protein, 253 aa, Homo sapiens
STTEVVMENVTAFWEEGFGELFEKAKGTPVLKDINFKIERGQLLAVAGSTGAGKTSLLMMIMGELEPSEGKIKHSGRISFCSQNSWIMPGTIKENIIGVSYDEYRYRSVIKACQLEEDISKFAEKDNIVLITLSGGQRARISLARAVYKDADLYLLDSPFGYLDVLTEKEIFESCVCKLMANKTRILVTSKMEHLKKADKILILHEGSSYFYGTFSELQNLRPDFSSKLMSFDQFSAERRNSILTETLHRFSL

SEQ ID NO: 20 - protein, 213 aa, Homo sapiens
DPKFESKAALLAARGPEELLCFTERLEDLVCFWEEAASAGVGPGQYSFSYQLEDEPWKLCRLHQAPTARGAVRFWCSLPTADTSSFVPLELRVTAASGAPRYHRVIHINEVVLLDAPVGLVARLADESGHVVLRWLPPPETPMTSHIRYEVDVSAGQGAGSVQRVEILEGRTECVLSNLRGRTRYTFAVRARMAEPSFGGFWSEWSEPVSLLT

SEQ ID NO: 21 - protein, 299 aa, Homo sapiens
CGHISVSAPIVHLGDPITASCIIKQNCSHLDPEPQILWRLGAELQPGGRQQRLSDGTQESIITLPHLNHTQAFLSCSLNWGNSLQILDQVELRAGYPPAIPHNLSCLMNLTTSSLICQWEPGPETHLPTSFTLKSFKSRGNCQTQGDSILDCVPKDGQSHCSIPRKHLLLYQNMGIWVQAENALGTSMSPQLCLDPMDVVKLEPPMLRTMDPQAGCLQLSWEPWQPGLHINQKCELRHKPQRGEASWALVGPLPLEALQYELCGLLPATAYTLQIRCIRWPLPGHWSDWSPSLELRTTE

SEQ ID NO: 22 - protein, 191 aa, Homo sapiens
EPKFTKCRSPERETFSCHWTDEGPIQLFYTRRNEWKECPDYVSAGENSCYFNSSFTSIAIPYCIKLTSNGGTVDEKCFSVDEIVQPDPPIALNWTLLNVSLTGIHADIQVRWEAPRNADIQKGWMVLEYELQYKEVNETKWKMMDPILTTSVPVYSLKVDKEYEVRVRSKQRNSGNYGEFSEVLYVTLPQM

SEQ ID NO: 23 - protein, 310 aa, Homo sapiens
CKEREEKIILVSSANEIDVRPCPLNPNEHKGTITWYKDDSKTPVSTEQASRIHQHKEKLWFVPAKVEDSGHYYCVVRNSSYCLRIKISAKFVENEPNLCYNAQAIFKQKLPVAGDGGLVCPYMEFFKNENNELPKLQWYKDCKPLLLDNIHFSGVKDRLIVMNVAEKHRGNYTCHASYTYLGKQYPITRVIEFITLEENKPTRPVIVSPANETMEVDLGSQIQLICNVTGQLSDIAYWKWNGSVIDEDDPVLGEDYYSVENPANKRRSTLITVLNISEIESRFYKHPFTCFAKNTHGIDAAYIQLIYPVT

SEQ ID NO: 24 - protein, 160 aa, Artificial Sequence
  Variant of green fluorescent protein (GFP) of Aequorea victoria
SVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIEN

SEQ ID NO: 25 - protein, 158 aa, Homo sapiens
MDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLP

SEQ ID NO: 26 - protein, 146 aa, Homo sapiens
GDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 27 - protein, 146 aa, Homo sapiens; identical to SEQ ID NO: 26

SEQ ID NO: 28 - protein, 299 aa, Homo sapiens
IARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 29 - protein, 195 aa, Homo sapiens
SVPSQKTYQGSYGFRLGFLHSGTAKSVTCTYSPALNKLFCQLAKTCPVQLWVDSTPPPGTRVRAMAIYKQSQHMTEVVRRCPHHERCSDSDGLAPPQHLIRVEGNLRAEYLDDRNTFRHSVVVPCEPPEVGSDCTTIHYNYMCYSSCMGGMNRRPILTIITLEDSSGNLLGRDSFEVRVCACPGRDRRTEEENLR

SEQ ID NO: 30 - protein, 196 aa, Homo sapiens
SSVPSQKTYQGSYGFRLGFLHSGTAKSVTCTYSPALNKLFCQLAKTCPVQLWVDSTPPPGTRVRAMAIYKQSQHMTEVVRRCPHHERCSDSDGLAPPQHLIRVEGNLRAEYLDDRNTFRHSVVVPCEPPEVGSDCTTIHYNYMCYSSCMGGMNRRPILTIITLEDSSGNLLGRDSFEVRVCACPGRDRRTEEENLR

SEQ ID NO: 31 - protein, 314 aa, Homo sapiens
MDGPYLQILEQPKQRGFRFRYVCEGPSHGGLPGASSEKNKKSYPQVKICNYVGPAKVIVQLVTNGKNIHLHAHSLVGKHCEDGICTVTAGPKDMVVGFANLGILHVTKKKVFETLEARMTEACIRGYNPGLLVHPDLAYLQAEGGGDRQLGDREKELIRQAALQQTKEMDLSVVRLMFTAFLPDSTGSFTRRLEPVVSDAIYDSKAPNASNLKIVRMDRTAGCVTGGEEIYLLCDKVQKDDIQIRFYEEEENGGVWEGFGDFSPTDVHRQFAIVFKTPKYKDINITKPASVFVQLRRKSDLETSEPKPFLYYPE

SEQ ID NO: 32 - protein, 9 aa, Homo sapiens
TTCCGLRQY

SEQ ID NO: 33 - protein, 303 aa, Homo sapiens
IKGGLFADIASHPWQAAIFAKHHRRGGERFLCGGILISSCWILSAAHCFQQQQQEEEEERRRRRFFFFFPPPPPPHHLTVILGRTYRVVPGEEEQKFEVEKYIVHKEFDDDTYDNDIALLQLKSSSSSDDDDDSSSSSSSSSSRRRRRCAQESSVVRTVCLPPADLQLPDWTECELSGYGKHEALSPFYSERLKEAHVRLYPSSRCTTTSSSQQQHLLNRTVTDNMLCAGDTTTRRRSSSNNNLHDACQGDSGGPLVCLNDGRMTLVGIISWGLGCGGQQKDVPGVYTKVTNYLDWIRDNMRP

SEQ ID NO: 34 - protein, 255 aa, Homo sapiens
VVGGEDAKPGQFPWQVVLNGKVDAFCGGSIVNEKWIVTAAHCVEETTGVKITVVAGEHNIEETEHTEQKRNVIRIIPHHNYNNNAAAAAAINKYNHDIALLELDEPLVLNSYVTPICIADKEYTTTNNNIIIFLKFGSGYVSGWGRVFHKGRSALVLQYLRVPLVDRATCLRSTKFTIYNNMFCAGGFFHEGGGRRDSCQGDSGGPHVTEVEGTSFLTGIISWGEECAAMMKGKYGIYTKVSRYVNWIKEKTKLT

SEQ ID NO: 35 - protein, 57 aa, Homo sapiens
MTCNIKNGRCEQFCKNSADNKVVCSCTEGYRLAENQKSCEPAVPFPCGRVSVSQTSK

SEQ ID NO: 36 - protein, 496 aa, Homo sapiens
EFARPCIPKSFGYSSVVCVCNATYCDSFDPPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 37 - protein, 390 aa, Homo sapiens
LDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTM

SEQ ID NO: 38 - protein, 479 aa, Homo sapiens
RPPNIVLIFADDLGYGDLGCYGHPSSTTPNLDQLAAGGLRFTDFYVPVSLPSRAALLTGRLPVRMGMYPGVLVPSSRGGLPLEEVTVAEVLAARGYLTGMAGKWHLGVGPEGAFLPPHQGFHRFLGIPYSHDQGPCQNLTCFPPATPCDGGCDQGLVPIPLLANLSVEAQPPWLPGLEARYMAFAHDLMADAQRQDRPFFLYYASHHTHYPQFSGQSFAERSGRGPFGDSLMELDAAVGTLMTAIGDLGLLEETLVIFTADNGPETMRMSRGGCSGLLRCGKGTTYEGGVREPALAFWPGHIAPGVTHELASSLDLLPTLAALAGAPLPNVTLDGFDLSPLLLGTGKSPRQSLFFYPSYPDEVRGVFAVRTGKYKAHFFTQGSAHSDTTADPACHASSSLTAHEPPLLYDLSKDPGENYNLLGATPEVLQALKQLQLLKAQLDAAVTFGPSQVARGEDPALQICCHPGCTPRPACCHCP

SEQ ID NO: 39 - protein, 474 aa, Homo sapiens
SRPPHLVFLLADDLGWNDVGFHGSRIRTPHLDALAAGGVLLDNYYTQPLTPSRSQLLTGRYQIRTGLQHQIIWPCQPSCVPLDEKLLPQLLKEAGYTTHMVGKWHLGMYRKECLPTRRGFDTYFGYLLGSEDYYSHERCTLIDALNVTRCALDFRDGEEVATGYKNMYSTNIFTKRAIALITNHPPEKPLFLYLALQSVHEPLQVPEEYLKPYDFIQDKNRHHYAGMVSLMDEAVGNVTAALKSSGLWNNTVFIFSTDNGGQTLAGGNNWPLRGRKWSLWEGGVRGVGFVASPLLKQKGVKNRELIHISDWLPTLVKLARGHTNGTKPLDGFDVWKTISEGSPSPRIELLHNIDPNFVDSSPCSAFNTSVHAAIRHGNWKLLTGYPGCGYWFPPPSQYNVSEIPSSDPPTKTLWLFDIDRDPEERHDLSREYPHIVTKLLSRLQFYHKHSVPVYFPAQDPRCDPKATGVWGPWM

SEQ ID NO: 40 - protein, 469 aa, Homo sapiens
FQYDVSSAAQPGCSVLDEAFQRYRDLLFGTLEKNVLVVSVVTPGCNQLPTLESVENYTLTINDDQCLLLSETVWGALRGLETFSQLVWKSAEGTFFINKTEIEDFPRFPHRGLLLDTSRHYLPLSSILDTLDVMAYNKLNVFHWHLVDDPSFPYESFTFPELMRKGSYNPVTHIYTAQDVKEVIEYARLRGIRVLAEFDTPGHTLSWGPGIPGLLTPCYSGSEPSGTFGPVNPSLNNTYEFMSTFFLEVSSVFPDFYLHLGGDEVDFTCWKSNPEIQDFMRKKGFGEDFKQLESFYIQTLLDIVSSYGKGYVVWQEVFDNKVKIQPDTIIQVWREDIPVNYMKELELVTKAGFRALLSAPWYLNRISYGPDWKDFYVVEPLAFEGTPEQKALVIGGEACMWGEYVDNTNLVPRLWPRAGAVAERLWSNKLTSDLTFAYERLSHFRCELLRRGVQAQPLNVGFCEQEFEQ

SEQ ID NO: 41 - protein, 492 aa, Homo sapiens
LWPWPQNFQTSDQRYVLYPNNFQFQYDVSSAAQPGCSVLDEAFQRYRDLLFGTLEKNVLVVSVVTPGCNQLPTLESVENYTLTINDDQCLLLSETVWGALRGLETFSQLVWKSAEGTFFINKTEIEDFPRFPHRGLLLDTSRHYLPLSSILDTLDVMAYNKLNVFHWHLVDDPSFPYESFTFPELMRKGSYNPVTHIYTAQDVKEVIEYARLRGIRVLAEFDTPGHTLSWGPGIPGLLTPCYSGSEPSGTFGPVNPSLNNTYEFMSTFFLEVSSVFPDFYLHLGGDEVDFTCWKSNPEIQDFMRKKGFGEDFKQLESFYIQTLLDIVSSYGKGYVVWQEVFDNKVKIQPDTIIQVWREDIPVNYMKELELVTKAGFRALLSAPWYLNRISYGPDWKDFYVVEPLAFEGTPEQKALVIGGEACMWGEYVDNTNLVPRLWPRAGAVAERLWSNKLTSDLTFAYERLSHFRCELLRRGVQAQPLNVGFCEQEFEQ

SEQ ID NO: 42 - protein, 480 aa, Homo sapiens
PALWPLPLSVKMTPNLLHLAPENFYISHSPNSTAGPSCTLLEEAFRRYHGYIFGTQVQQLLVSITLQSECDAFPNISSDESYTLLVKEPVAVLKANRVWGALRGLETFSQLVYQDSYGTFTINESTIIDSPRFSHRGILIDTSRHYLPVKIILKTLDAMAFNKFNVLHWHIVDDQSFPYQSITFPELSNKGSYSLSHVYTPNDVRMVIEYARLRGIRVLPEFDTPGHTLSWGKGQKDLLTPCYSDSFGPINPTLNTTYSFLTTFFKEISEVFPDQFIHLGGDEVEFKCWESNPKIQDFMRQKGFGTDFKKLESFYIQKVLDIIATINKGSIVWQEVFDDKAKLAPGTIVEVWKDSAYPEELSRVTASGFPVILSAPWYLDLISYGQDWRKYYKVEPLDFGGTQKQKQLFIGGEACLWGEYVDATNLTPRLWPRASAVGERLWSSKDVRDMDDAYDRLTRHRCRMVERGIAAQPLYAGYCN

SEQ ID NO: 43 - protein, 481 aa, Homo sapiens
PALWPLPLSVKMTPNLLHLAPENFYISHSPNSTAGPSCTLLEEAFRRYHGYIFGTQVQQLLVSITLQSECDAFPNISSDESYTLLVKEPVAVLKANRVWGALRGLETFSQLVYQDSYGTFTINESTIIDSPRFSHRGILIDTSRHYLPVKIILKTLDAMAFNKFNVLHWHIVDDQSFPYQSITFPELSNKGSYSLSHVYTPNDVRMVIEYARLRGIRVLPEFDTPGHTLSWGKGQKDLLTPCYSLDSFGPINPTLNTTYSFLTTFFKEISEVFPDQFIHLGGDEVEFKCWESNPKIQDFMRQKGFGTDFKKLESFYIQKVLDIIATINKGSIVWQEVFDDKAKLAPGTIVEVWKDSAYPEELSRVTASGFPVILSAPWYLDLISYGQDWRKYYKVEPLDFGGTQKQKQLFIGGEACLWGEYVDATNLTPRLWPRASAVGERLWSSKDVRDMDDAYDRLTRHRCRMVERGIAAQPLYAGYCN

SEQ ID NO: 44 - protein, 492 aa, Homo sapiens; identical to SEQ ID NO: 41

SEQ ID NO: 45 - protein, 307 aa, Homo sapiens
VPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCTQYIRHGSKPMYTPEPDICHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVL

SEQ ID NO: 46 - protein, 452 aa, Homo sapiens
APDQDEIQRLPGLAKQPSFRQYSGYLKSSGSKHLHYWFVESQKDPENSPVVLWLNGGPGCSSLDGLLTEHGPFLVQPDGVTLEYNPYSWNLIANVLYLESPAGVGFSYSDDKFYATNDTEVAQSNFEALQDFFRLFPEYKNNKLFLTGESYAGIYIPTLAVLVMQDPSMNLQGLAVGNGLSSYEQNDNSLVYFAYYHGLLGNRLWSSLQTHCCSQNKCNFYDNKDLECVTNLQEVARIVGNSGLNIYNLYAPCAGGVPSHFRYEKDTVVVQDLGNIFTRLPLKRMWHQALLRSGDKVRMDPPCTNTTAASTYLNNPYVRKALNIPEQLPQWDMCNFLVNLQYRRLYRSMNSQYLKLLSSQKYQILLYNGDVDMACNFMGDEWFVDSLNQKMEVQRRPWLVKYGDSGEQIAGFVKEFSHIAFLTIKGAGHMVPTDKPLAAFTMFSRFLNKQPY

SEQ ID NO: 47 - protein, 359 aa, Homo sapiens
DETTALVCDNGSGLVKAGFAGDDAPRAVFPSIVGRPRDSYVGDEAQSKRGILTLKYPIEGIITNWDDMEKIWHHTFYNELRVAPEEHPTLLTEAPLNPKANREKMTQIMFETFNVPAMYVAIQAVLSLYASGRTTGIVLDSGDGVTHNVPIYEGYALPHAIMRLDLAGRDLTDYLMKILTERGYSFVTTAEREIVRDIKEKLCYVALDFENEMATAASSSSLEKSYELPDGQVITIGNERFRCPETLFQPSFIGMESAGIHETTYNSIMKCDIDIRKDLYANNVMSGGTTMYPGIADRMQKEITALAPSTMKIKIIAPPERKYSVWIGGSILASLSTFQQMWITKQEYDEAGPSIVHRK

SEQ ID NO: 48 - protein, 432 aa, Artificial Sequence
  Variant of green fluorescent protein (GFP) of Aequorea victoria
PREIITLQLGQCGNQIGFEFWKQLCAEHGISPEAIVEEFATEGTDRKDVFFYQADDEHYIPRAVLLDLEPRVIHSILNSPYAKLYNPENIYLSEHGGGAGNNWASGFSQGEKIHEDIFDIIDREADGSDSLEGFVLCHSIAGGTGSGLGSYLLERLNDRYPKKLVQTYSVFPNQDEMSDVVVQPYNSLLTLKRLTQNADCLVVLDNTALNRIATDRLHIQNPSFSQINQLVSTIMSASTTTLRYPGYMNNDLIGLIASLIPTPRLHFLMTGYTPLTSVRKTTVLDVMRRLLQPKNVMVSTGRDTNHCYIAILNIIQGEVDPTQVHKSLQRIRERKLANFIPWGPASIQVALSRKSPYRVSGLMMANHTSISSLFERTCRQYDKLRKREAFLEQFRKEDMFKDNFDEMDTSREIVQQLIDEYHAATRPDYISW

SEQ ID NO: 49 - protein, 424 aa, Homo sapiens
REIITLQLGQCGNQIGFEFWKQLCAEHGISPEAIVEEFATEGTDRKDVFFYQADDEHYIPRAVLLDLEPRVIHSILNSPYAKLYNPENIYLSEHGAGNNWASGFSQGEKIHEDIFDIIDREADGSDSLEGFVLCHSIAGGTGSGLGSYLLERLNDRYPKKLVQTYSVFPNQDEMSDVVVQPYNSLLTLKRLTQNADCLVVLDNTALNRIATDRLHIQNPSFSQINQLVSTIMSASTTTLRYPGYMNNDLIGLIASLIPTPRLHFLMTGYTPLTKTTVLDVMRRLLQPKNVMVSTTNHCYIAILNIIQGEVDPTQVHKSLQRIRERLANFIPWGPASIQVALSRKSPYLPRVSGLMMANHTSISSLFERTCRQYDKLRKREAFLEQFRKEDMFKDNFDEMDTSREIVQQLIDEYHAATRPDYISW

SEQ ID NO: 50 - protein, 129 aa, Homo sapiens
GSSPLLKSAEREKEMASMKEEFTRLKEALEKSEARRKELEEKMVSLLQEKNDLQLQVQAEQDNLADAEERCDQLIKNKIQLEAKVKEMNKRLEDEEEMNAELTAKKRKLEDECSELKRDIDDLELTLAK

SEQ ID NO: 51 - protein, 125 aa, Homo sapiens
SPLLKSAEREKEMASMKEEFTRLKEALEKSEARRKELEEKMVSLLQEKNDLQLQVQAEQDNLADAEERCDQLIKNKIQLEAKVKEMNKRLEDEEEMNAELTAKKRKLEDECSELKRDIDDLELTL

SEQ ID NO: 52 - protein, 127 aa, Homo sapiens
SSPLLKSAEREKEMASMKEEFTRLKEALEKSEARRKELEEKMVSLLQEKNDLQLQVQAEQDNLADAEERCDQLIKNKIQLEAKVKEMNKRLEDEEEMNAELTAKKRKLEDECSELKRDIDDLELTLA

SEQ ID NO: 53 - protein, 127 aa, Homo sapiens
SPLLKSAEREKEMASMKEEFTRLKEALEKSEARRKELEEKMVSLLQEKNDLQLQVQAEQDNLADAEERCDQLIKNKIQLEAKVKEMNKRLEDEEEMNAELTAKKRKLEDECSELKRDIDDLELTLAK

SEQ ID NO: 54 - protein, 121 aa, Homo sapiens
EKLCATYKLCHPEELVLLGHSLGIPWAPLLAGCLSQLHSGLFLYQGLLQALEGISPELGPTLDTLQLDVADFATTIWQQMEELGMMPAFASAFQRRAGGVLVASHLQSFLEVSYRVLRHLA

SEQ ID NO: 55 - protein, 105 aa, Homo sapiens
EHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIP

SEQ ID NO: 56 - protein, 165 aa, Homo sapiens
CDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 57 - protein, 166 aa, Homo sapiens
MSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 58 - protein, 242 aa, Homo sapiens
MQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIDELIQVMAELGANVSGEFVKEAENLKKYFNDNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAA

SEQ ID NO: 59 - protein, 122 aa, Homo sapiens
STKKTQLQLEHLLLDLQMILNGINNYKNPKLTRMLTFKFYMPKKATELKHLQCLEEELKPLEEVLNLAQNFHLRPRDLISNINVIVLELKGFMCEYADETATIVEFLNRWITFCQSIISTLT

SEQ ID NO: 60 - protein, 123 aa, Homo sapiens
ALWQFNGMIKCKIPSSEPLLDFNNYGCYCGLGGSGTPVDDLDRCCQTHDNCYKQAKKLDSCKVLVDNPYTNNYSYSCSNNEITCSSENNACEAFICNCDRNAAICFSKVPYNKEHKNLDAANC

SEQ ID NO: 61 - protein, 152 aa, Homo sapiens
APVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVS

SEQ ID NO: 62 - protein, 150 aa, Homo sapiens
PHRRDLCSRSIWLARKIRSDLTALTESYVKHQGLWSELTEAERLQENLQAYRTFHVLLARLLEDQQVHFTPTEGDFHQAIHTLLLQVAAFAYQIEELMILLEYKIPRNEADGMLFEKKLWGLKVLQELSQWTVRSIHDLRFISSHQTGIP

SEQ ID NO: 63 - protein, 126 aa, Homo sapiens
HRRDLCSRSIWLARKIRSDLTALTESYVKHQGLELTEAERLQENLQAYRTFHVLLARLLEDQQEGDFHQAIHTLLLQVAAFAYQIEELMILLEYKIPRNKKLWGLKVLQELSQWTVRSIHDLRFIS

SEQ ID NO: 64 - protein, 148 aa, Homo sapiens
DKPVAHVVANPQAEGQLQWSNRRANALLANGVELRDNQLVVPIEGLFLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 65 - protein, 144 aa, Homo sapiens
KPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 66 - protein, 141 aa, Homo sapiens
TTGCAEHCSLNEKITVPDTKVNFYAWKRMEVGQQAVEVWQGLALLSEAVLRGQALLVKSSQPWEPLQLHVDKAVSGLRSLTTLLRALGAQKEAISNSDAASAAPLRTITADTFRKLFRVYSNFLRGKLKLYTGEACRTGDR

SEQ ID NO: 67 - protein, 21 aa, Homo sapiens
GIVEQCCTSICSLYQLENYCN

SEQ ID NO: 68 - protein, 29 aa, Homo sapiens
FVNQHLCGSHLVEALYLVCGERGFFYTPK

SEQ ID NO: 69 - protein, 166 aa, Homo sapiens
FPTIPLSRLADNAWLRADRLNQLAFDTYQEFEEAYIPKEQIHSFWWNPQTSLCPSESIPTPSNKEETQQKSNLELLRISLLLIQSWLEPVQFLRSVFANSLVYGASDSNVYDLLKDLEEGIQTLMGRLEALLKNYGLLYCFNKDMSKVSTYLRTVQCRSVEGSCGF

SEQ ID NO: 70 - protein, 242 aa, Homo sapiens
CHHRICHCSNRVFLCQESKVTEIPSDLPRNAIELRFVLTKLRVIQKGAFSGFGDLEKIEISQNDVLEVIEADVFSNLPKLHEIRIEKANNLLYINPEAFQNLPNLQYLLISNTGIKHLPDVHKIHSLQKVLLDIQDNINIHTIERNSFVGLSFESVILWLNKNGIQEIHNCAFNGTQLDELNLSDNNNLEELPNDVFHGASGPVILDISRTRIHSLPSYGLENLKKLRARSTYNLKKLPTLE

SEQ ID NO: 71 - protein, 130 aa, Homo sapiens
IQKVQDDTKTLIKTIVTRINDILDFIPGLHPILTLSKMDQTLAVYQQILTSMPSRNVIQISNDLENLRDLLHVLAFSKSCHLPEASGLETLDSLGGVLEASGYSTEVVALSRLQGSLQDMLWQLDLSPGC

SEQ ID NO: 72 - protein, 62 aa, Homo sapiens
PETLCGAELVDALQFVCGDRGFYFNKPTGYGSSSRRAPQTGIVDECCFRSCDLRRLEMYCAP

SEQ ID NO: 73 - protein, 125 aa, Homo sapiens
MYRSAFSVGLETRVTVPNVPIRFTKIFYNQQNHYDGSTGKFYCNIPGLYYFSYHITVYMKDVKVSLFKKDKAVLFTYDQYQENVDQASGSVLLHLEVGDQVWLQVYYADNVNDSTFTGFLLYHDT

SEQ ID NO: 74 - protein, 111 aa, Homo sapiens
MYRSAFSVGLPNVPIRFTKIFYNQQNHYDGSTGKFYCNIPGLYYFSYHITVYMKDVKVSLFKKDKVLFTYDQYQEKVDQASGSVLLHLEVGDQVWLQVYDSTFTGFLLYHD

SEQ ID NO: 75 - protein, 102 aa, Homo sapiens
MYRSAFSVGLETRVTVPIRFTKIFYNQQNHYDGSTGKFYCNIPGLYYFSYHITVDVKVSLFKKDKAVLFTQASGSVLLHLEVGDQVWLQNDSTFTGFLLYHD

SEQ ID NO: 76 - protein, 736 aa, Homo sapiens
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDLWIGLLGCHNSDFRNRGMTALLKVSSCDGLKNTGDYYEDSYED

SEQ ID NO: 77 - protein, 644 aa, Homo sapiens
RSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYSSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 78 - protein, 578 aa, Homo sapiens
SEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAA

SEQ ID NO: 79 - protein, 578 aa, Homo sapiens; identical to SEQ ID NO: 78

SEQ ID NO: 80 - protein, 375 aa, Homo sapiens
HPTFNKITPNLAEFAFSLYRQLAHQSNSTNIFFSPVSIAAAFAMLSLGAKGDTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 81 - protein, 147 aa, Homo sapiens
SEQASNVLSPADKTNVKAAWGKVGAHAGEYGAEALERMFLSFPTTKTYFPHFDLSHGSAQVKGHGKKVADALTNAVAHVDDMPNALSALSDLHAHKLRVDPVNFKLLSHCLLVTLAAHLPAEFTPAVHASLDKFLASVSTVLTSKYR

SEQ ID NO: 82 - protein, 146 aa, Homo sapiens
VHLTPEEKSAVTALWGKVNVDEVGGEALGRLLVVYPWTQRFFESFGDLSTPDAVMGNPKVKAHGKKVLGAFSDGLAHLDNLKGTFATLSELHCDKLHVDPENFRLLGNVLVCVLAHHFGKEFTPPVQAAYQKVVAGVANALAHKYH

SEQ ID NO: 83 - protein, 248 aa, Artificial Sequence; identical to SEQ ID NO: 5
  Variant of green fluorescent protein (GFP) of Aequorea victoria

SEQ ID NO: 84 - protein, 248 aa, Artificial Sequence; identical to SEQ ID NO: 6
  Variant of green fluorescent protein (GFP) of Aequorea victoria

SEQ ID NO: 85 - protein, 248 aa, Artificial Sequence
  Variant of green fluorescent protein (GFP) of Aequorea victoria
MGHHHHHHGGASKGERLFTGVVPILVELDGDVNGHKFSVRGKGKGDATRGKLTLKFICTTGKLPVPWPTLVTTLTYGVQCFSRYPKHMKRHDFFKSAMPKGYVQERTISFKKDGTYKTRAEVKFEGRTLVNRIKLKGRDFKEKGNILGHKLRYNFNSHNVYITADKRKNGIKANFKIRHNVKDGSVQLADHYQQNTPIGRGPVLLPRNHYLSTRSALSKDPKEKRDHMVLLEFVTAAGITHGMDELYK

SEQ ID NO: 86 - protein, 248 aa, Artificial Sequence; identical to SEQ ID NO: 7
  Variant of green fluorescent protein (GFP) of Aequorea victoria

SEQ ID NO: 87 - protein, 280 aa, Artificial Sequence; identical to SEQ ID NO: 11
  Variant of green fluorescent protein (GFP) of Aequorea victoria

SEQ ID NO: 88 - DNA, 24 bp, artificial sequence
  synthetic oligonucleotide (Forward GAPDH)
caactcactcaagattgtcagcaa

SEQ ID NO: 89 - DNA, 20 bp, Artificial sequence
  synthetic oligonucleotide (Reverse GAPDH)
gggatggactgtggtcatga

SEQ ID NO: 90 - DNA, 26 bp, Artificial sequence
  synthetic oligonucleotide (Forward beta-actin)
atagcacagcctggatagcaacgtac

SEQ ID NO: 91 - DNA, 25 bp, Artificial sequence
  synthetic oligonucleotide (Reverse beta-actin)
caccttctacaatgagctgcgtgtg

SEQ ID NO: 92 - RNA, 20 nt, Artificial sequence
  synthetic oligonucleotide (siRNA)
gcaugccauuaccuggccau

SEQ ID NO: 93 - protein, 248 aa, Artificial Sequence; identical to SEQ ID NO: 7
  Variant of green fluorescent protein (GFP) of Aequorea victoria

SEQ ID NO: 94 - protein, 280 aa, Artificial Sequence; identical to SEQ ID NO: 11
  Variant of green fluorescent protein (GFP) of Aequorea victoria

SEQ ID NO: 95 - protein, 238 aa, Artificial Sequence; identical to SEQ ID NO: 1
  Variant of green fluorescent protein (GFP) of Aequorea victoria

SEQ ID NO: 96 - protein, 248 aa, Artificial Sequence; identical to SEQ ID NO: 85
  Variant of green fluorescent protein (GFP) of Aequorea victoria

SEQ ID NO: 97 - protein, 4 aa, Artificial sequence
  linker cleavable by lysosomal thiol proteinases
AGVF

SEQ ID NO: 98 - protein, 4 aa, Artificial sequence
  linker cleavable by lysosomal cysteine proteinases
GFLG

SEQ ID NO: 99 - protein, 4 aa, Artificial sequence
  linker cleavable by cathepsin B
ALAL

SEQ ID NO: 100 - protein, 4 aa, Artificial sequence
  linker cleavable by cathepsin B
ALAL

SEQ ID NO: 101 - protein, 5 aa, Artificial sequence
  linker cleavable by cathepsin D
ALALA

SEQ ID NO: 102 - protein, 28 aa, Artificial sequence
  exemplary linker cleavable by cathepsin B
GGSGGSGGSGGSALALGGSGGSGGSGGS

(19) United States
(12) Patent Application Publication
(10) Pub. No.: US 2011/0112040 A1
(43) Pub. Date: May 12, 2011

(54) SUPERCHARGED PROTEINS FOR CELL PENETRATION

(75) Inventors: David R. Liu, Lexington, MA (US); Brian R. Mcnaughton, Cambridge, MA (US); James Joseph Cronican, Somerville, MA (US); David B. Thompson, Cambridge, MA (US)

(73) Assignee: President and Fellows of Harvard College, Cambridge, MA (US)

(21) Appl. No.: 12/989,829

(22) PCT Filed: Apr. 28, 2009
(86) PCT No.: PCT/US09/41984

§ 371 Date: Jan. 10, 2011

Related U.S. Application Data
(60) Provisional application No. 61/048,370, filed on Apr. 28, 2008.
(60) Provisional application No. 61/105,287, filed on Oct. 14, 2008.

(51) Int. Cl. A61K 38/14; C12N 5/00; C12Q 1/02; C07K 14/00; C12N 15/63

(52) U.S. Cl. 514/20.9; 435/375; 435/29; 530/395; 530/396; 435/320.1

(57) ABSTRACT

Compositions, systems and related methods for delivering a supercharged protein or a complex of a supercharged protein and therapeutic agent (e g, nucleic acid, peptide, small molecule) to cells are disclosed. Superpositively charged proteins may be associated with nucleic acids (which typically have a net negative charge) via electrostatic interactions. The systems and methods may involve altering the primary sequence of a protein in order to “supercharge” the protein (e g, to generate a superpositively-charged protein). The compositions may be used to treat proliferative diseases, infectious diseases, cardiovascular diseases, inborn errors in metabolism, genetic diseases, etc.

### RELATED APPLICATIONS

The present invention claims priority under 35 U.S.C. §119(e) to U.S. provisional patent applications: U.S. Ser. No. 61/048,370, filed Apr. 28, 2008; and U.S. Ser. No. 61/105,287, filed Oct. 14, 2008, each of which is incorporated herein by reference.

### GOVERNMENT SUPPORT

This invention was made with U.S. Government support under contract number R01 GM 065400 awarded by the National Institutes of Health/NIGMS. The U.S. Government has certain rights in the invention.

### BACKGROUND OF THE INVENTION

The effectiveness of an agent intended for use as a therapeutic, diagnostic, or other application is often highly dependent on its ability to penetrate cellular membranes or tissue to induce a desired change in biological activity. Although many therapeutic drugs, diagnostic or other product candidates, whether protein, nucleic acid, organic small molecule, or inorganic small molecule, show promising biological activity in vitro, many fail to reach or penetrate target cells to achieve the desired effect, often due to physiochemical properties that result in inadequate biodistribution in vivo.

In particular, nucleic acids have great potential as effective therapeutic agents and as research tools. The generality and sequence-specificity of siRNA-mediated gene regulation has raised the possibility of using siRNAs as gene-specific therapeutic agents (Bumcrot et al., 2006, Nat. Chem. Biol., 2:711-19; incorporated herein by reference). The suppression of gene expression by short interfering RNA (siRNA) has also emerged as a valuable tool for studying gene and protein function (Dorsett et al., 2004, Nat. Rev. Drug Discov., 3:318-29; Dykxhoorn et al., 2003, Nat. Rev. Mol. Cell. Biol., 4:457-67; Elbashir et al., 2001, Nature, 411:494-98; each of which is incorporated herein by reference). However, the delivery of nucleic acids such as siRNAs to cells has been found to be unpredictable and is typically inefficient. One obstacle to effective delivery of nucleic acids to cells is inducing cells to take up the nucleic acid. Much work has been done to identify agents that can aid in the delivery of nucleic acids to cells. Commercially available cationic lipid reagents are typically used to transfect siRNA in cell culture. The effectiveness of cationic lipid-based siRNA delivery, however, varies greatly by cell type. Also, a number of cell lines including some primary neuron, T-cell, fibroblast, and epithelial cell lines have demonstrated resistance to common cationic lipid transfection techniques (Carlotti et al., 2004, Mol. Ther., 9:209-17; Ma et al., 2002, Neuroscience, 112:1-5; McManus et al., 2002, J. Immunol., 169:5754-60; Strait et al., 2007, Am. J. Physiol. Renal Physiol., 293:F601-06; each of which is incorporated herein by reference). Alternative transfection approaches including electroporation (Jantsch et al., 2008, J. Immunol. Methods, 337:71-77; incorporated herein by reference) and virus-mediated siRNA delivery (Brummelkamp et al., 2002, Cancer Cell, 2:243-47; Stewart et al., 2003, RNA, 9:493-501; each of which is incorporated herein by reference) have also been used; however, these methods can be cytotoxic or perturb cellular function in unpredictable ways and have limited value for the delivery of nucleic acids (e.g., siRNA) as therapeutic agents in a subject.

Recent efforts to address the challenges of nucleic acid delivery have resulted in a variety of new nucleic acid delivery platforms. These methods include lipidoids (Akinc et al., 2008, Nat. Biotechnol., 26:561-69; incorporated herein by reference), cationic polymers (Segura and Hubbell, 2007, Bioconjug. Chem., 18:736-45; incorporated herein by reference), inorganic nanoparticles (Sokolova and Epple, Angew Chem. Int. Ed. Engl., 47:1382-95; incorporated herein by reference), carbon nanotubes (Liu et al., 2007, Angew Chem. Int. Ed. Engl., 46:2023-27; incorporated herein by reference), cell-penetrating peptides (Deshayes et al., 2005, Cell Mol. Life. Sci., 62:1839-49; and Meade and Dowdy, 2008, Adv. Drug Deliv. Rev., 60: 530-36; both of which are incorporated herein by reference), and chemically modified siRNA (Krutzfeldt et al., 2005, Nature 438: 685-89; incorporated herein by reference). Each of these delivery systems offers benefits for particular applications; in most cases, however, questions regarding cytotoxicity, ease of preparation, stability, or generality remain. Easily prepared reagents capable of effectively delivering nucleic acids (e.g., siRNA) to a variety of cell lines without significant cytotoxicity therefore remain of considerable interest.

Given the current interest in RNAi therapies and other nucleic acid-based therapies, there remains a need in the art for reagents and systems that can be used to deliver nucleic acids as well as other agents (e.g. peptides, proteins, small molecules) to a wide variety of cell types predictably and efficiently.

### SUMMARY OF THE INVENTION

The present invention provides novel systems, compositions, preparations, and related methods for delivering nucleic acids and other agents (e.g., peptides, proteins, small molecules) into cells using a protein that has been modified to result in an increase or decrease in the overall surface charge on the protein, referred to henceforth as “supercharging.” Thus, supercharging can be used to promote the entry into a cell in vivo or in vitro of a supercharged protein, or agent(s) associated with the supercharged protein that together form a complex. Such systems and methods may comprise the use of proteins that have been engineered to be supercharged and include all such modifications, including but not limited to, those involving changes in amino acid sequence as well as the attachment of charged moieties to the protein. Examples of engineered supercharged proteins are described in international PCT patent application, PCT/US07/70254, filed Jun. 1, 2007, published as WO 2007/143574 on Dec. 13, 2007; and in U.S. provisional patent applications, U.S. Ser. No. 60/810,364, filed Jun. 2, 2006, and U.S. Ser. No. 60/836,607, filed Aug. 9, 2006; each of which is entitled “Protein Surface Remodeling,” and each of which is incorporated herein by reference. Further examples of supercharged proteins useful in drug delivery are also described herein. The present invention also contemplates the use of naturally occurring supercharged proteins to enhance cell penetration of associated agents that together form a complex or to enhance the cell penetration of the naturally occurring supercharged protein itself. Typically, the supercharged protein, engineered or naturally occurring, is positively charged. In certain embodiments, superpositively charged proteins may be associated with nucleic acids (which typically have a net negative charge) via electrostatic interactions, thereby aiding in the delivery of the nucleic acid to a cell. Superpositively charged proteins may also be associated covalently or non-covalently with the nucleic acid to be delivered in other ways. Other agents such as peptides or small molecules may also be delivered to cells using supercharged proteins that are covalently bound or otherwise associated (e.g., electrostatic interactions) with the agent to be delivered. In certain embodiments, the supercharged protein is fused with a second protein sequence. For example, in certain embodiments, the agent to be delivered and the superpositively charged protein are expressed together in a single polypeptide chain as a fusion protein. In certain embodiments, the fusion protein has a linker, e.g., a cleavable linker between the supercharged protein and the other protein component. In certain embodiments, the agent to be delivered and the supercharged protein, e.g., a superpositively charged protein, are associated with each other via a cleavable linker (e.g., a linker cleavable by a protease or esterase, disulfide bond). The supercharged protein, e.g., a superpositively charged protein, useful in the present invention is typically non-antigenic, biodegradable, and/or biocompatible. In certain embodiments, the superpositively charged protein does not have biological activity or any deleterious biological activity. In certain embodiments the supercharged protein has a mutation or other alteration (e.g., a post-translational modification such as a cleavage or other covalent modification) which decreases or abolishes a biological activity exhibited by the protein prior to supercharging. This may be of particular interest when the supercharged protein is of interest not because of its own biological activity but for use in delivering an agent to a cell. Without wishing to be bound by a particular theory, anionic cell-surface proteoglycans are thought to serve as a receptor for the actin-dependent endocytosis of the superpositively charged protein bound to its payload. The inventive supercharged proteins or delivery system using supercharged, e.g., superpositively charged proteins, may include the use of other pharmaceutically acceptable excipients such as polymers, lipids, carbohydrates, small molecules, targeting moieties, endosomolytic agents, proteins, peptides, etc. For example, a supercharged protein or complex of a supercharged protein, e.g., a superpositively charged protein, and agent to be delivered may be contained within or be associated with a microparticle, nanoparticle, picoparticle, micelle, liposome, or other drug delivery system. In other embodiments, only the agent to be delivered and the supercharged protein are used to deliver the agent to a cell. In certain embodiments, the supercharged protein is chosen to deliver itself or an associated agent to a particular cell or tissue type. In certain embodiments, the supercharged, e.g., superpositively charged, protein or agent to be delivered and the supercharged protein are combined with an agent that disrupts endosomolytic vesicles or enhances the degradation of endosomes (e.g., chloroquine, pyrene butyric acid, fusogenic peptides, polyethyleneimine, hemagglutinin 2 (HA2) peptide, melittin peptide). Thus, escape of the agent to be delivered from the endosome into the cytosol is enhanced.

In some embodiments, the inventive systems and methods involve altering the primary sequence of a protein in order to “supercharge” the protein. In other embodiments, the inventive systems and methods involve the attachment of charged moieties to the protein in order to “supercharge” the protein. That is, the overall net charge on the modified protein is increased (either more positive charge or more negative charge) compared to the unmodified protein. In certain embodiments, the protein is supercharged, e.g., superpositively charged, to enable the delivery of nucleic acids or other agents to a cell. Any protein may be “supercharged”. Typically, the protein is non-immunogenic and either naturally or upon supercharging has the ability to transfect or deliver itself or an associated agent into a cell. In certain embodiments, the activity of the supercharged protein is approximately or substantially the same as the protein without modification. In other embodiments, the activity of the supercharged protein is substantially decreased as compared to the protein without modification. Such activity may not be relevant to the delivery of itself or an associated agent, e.g., nucleic acids, to cells as described herein. In some embodiments, supercharging a protein results in increasing the protein's resistance to aggregation, solubility, ability to refold, and/or general stability under a wide range of conditions as well as increasing the protein's ability to deliver itself or an associated agent, e.g., nucleic acids, to a cell. In certain embodiments, the supercharged protein helps to target itself or an associated agent to be delivered to a particular cell type, tissue, or organ. In certain embodiments, supercharging a protein includes the steps of: (a) identifying surface residues of a protein of interest; (b) optionally, identifying the particular surface residues that are not highly conserved among other proteins related to the protein of interest (i.e., determining which amino acids are not essential for the activity or function of the protein); (c) determining the hydrophilicity of the identified surface residues; and (d) replacing at least one or more of the identified charged or polar, solvent-exposed residues with an amino acid that is charged at physiological pH. See published international PCT patent application, PCT/US07/70254, filed Jun. 1, 2007, published as WO 2007/143574 on Dec. 13, 2007; and U.S. Provisional patent applications, U.S. Ser. No. 60/810,364, filed Jun. 2, 2006, and U.S. Ser. No. 60/836,607, filed Aug. 9, 2006; each of which is entitled “Protein Surface Remodeling”; and each of which is incorporated herein by reference. Exemplary methods of preparing supercharged proteins and exemplary protein sequences illustrating the use of method are described herein. In certain embodiments, to make a positively charged “supercharged” protein, the residues identified for modification are mutated either to lysine (Lys) or arginine (Arg) residues (i.e., amino acids that are positively charged at physiological pH). In certain embodiments, to make a negatively charged “supercharged” protein, the residues identified for modification are mutated either to aspartate (Asp) or glutamate (Glu) residues (i.e., amino acids that are negatively charged at physiological pH). Each of the above steps may be carried out using any technique, computer software, algorithm, methodology, paradigm, etc. known in the art. After the modified protein is created, it may be tested for its activity and/or the desired property being sought (e.g., the ability to delivery a nucleic acid or other agent into a cell). In certain embodiments, the supercharged protein is less susceptible to aggregation. In certain embodiments, a positively charged “supercharged” protein (e.g., superpositively charged green fluorescent protein (GFP) such +36 GFP) is useful in delivering a nucleic acid (e.g., an siRNA agent) to a cell (e.g., a mammalian cell, a human cell). In certain embodiments, the inventive system allows for the delivery of nucleic acids into cells normally resistant to transfection (e.g., neuronal cells, T-cells, fibroblasts, and epithelial cells). In certain embodiments, rather than engineering a supercharged protein, a naturally occurring supercharged protein is identified and used in the inventive drug delivery system. Examples of naturally occurring supercharged proteins include, but are not limited to, cyclon (ID No.: Q9H6F5), PNRC1 (ID No.: Q12796), RNPS1 (ID No.: Q15287), SURF6 (ID No.: O75683), AR6P (ID No.: Q66PJ3), NKAP (ID No.: Q8N5F7), EBP2 (ID No.: Q99848), LSM11 (ID No.: P83369), RL4 (ID No.: P36578), KRR1 (ID No.: Q13601), RY-1 (ID No.: Q8WVK2), BriX (ID No.: Q8TDN6), MNDA (ID No.: P41218), H1b (ID No.: P16401), cyclin (ID No.: Q9UK58), MDK (ID No.: P21741), Midkine (ID No.: P21741), PROK (ID No.: Q9HC23), FGFS (ID No.: P12034), SFRS (ID No.: Q8N9Q2), AKIP (ID No.: Q9NWT8), CDK (ID No.: Q8N726), beta-defensin (ID No.: P81534), Defensin 3 (ID No.: P81534); PAVAC (ID No.: P18509), PACAP (ID No.: P18509), eotaxin-3 (ID No.: Q9Y258), histone H2A (ID No.: Q7L7L0), HMGB1 (ID No.: P09429), C-Jun (ID No.: P05412), TERF 1 (ID No.: P54274), N-DEK (ID No.: P35659), PIAS 1 (ID No.: O75925), Ku70 (ID No.: P12956), HBEGF (ID No.: Q99075), and HGF (ID No.: P14210).

In certain embodiments, once a supercharged protein has been obtained, systems and methods in accordance with the invention involve associating one or more nucleic acids or other agents with the supercharged protein and contacting the resulting complex with a cell under suitable conditions for the cell to take up the payload. The nucleic acid may be a DNA, RNA, and/or hybrid or derivative thereof. In certain embodiments, the nucleic acid is an RNAi agent, RNAi-inducing agent, short interfering RNA (siRNA), short hairpin RNA (shRNA), micro RNA (miRNA), antisense RNA, ribozyme, catalytic DNA, RNA that induces triple helix formation, aptamer, vector, plasmid, viral genome, artificial chromosome, etc. In some embodiments, the nucleic acid is single-stranded. In other embodiments, the nucleic acid is double-stranded. In some embodiments, a nucleic acid may comprise one or more detectable labels (e.g., fluorescent tags and/or radioactive atoms). In certain embodiments, the nucleic acid is modified or derivatized (e.g., to be less susceptible to degradation, to improve transfection efficiency). In certain embodiments, the modification of the nucleic acid prevents the degradation of the nucleic acid. In certain embodiments, the modification of the nucleic acid aids in the delivery of the nucleic acid to a cell. Other agents that may be delivered using a supercharged protein include small molecules, peptides, and proteins. The resulting complex may then be combined or associated with other pharmaceutically acceptable excipient(s) to form a composition suitable for delivering the agent to a cell, tissue, organ, or subject.

Supercharged proteins may be associated with nucleic acids (or other agents) via non-covalent interactions to form a complex. Although covalent association of the supercharged protein with a nucleic acid is possible, it is typically not necessary to achieve delivery of the nucleic acid. In some embodiments, supercharged proteins are associated with nucleic acids via electrostatic interactions. Supercharged proteins may be associated with nucleic acids through other non-covalent interactions or covalent interactions. The supercharged proteins may have a net positive charge of at least +5, +10, +15, +20, +25, +30, +35, +40, or +50. In some embodiments, superpositively charged proteins are associated with nucleic acids that have an overall net negative charge. The resulting complex may have a net negative or positive charge. In certain embodiments, the complex has a net positive charge. For example, +36 GFP may be associated with a negatively charged siRNA.

Supercharged proteins may be associated with other agents besides nucleic acids via non-covalent or covalent interactions. For example, a negatively charged protein may be associated with a superpositively charged protein through electrostatic interactions. For agents that are not charged or do not have sufficient charge, the agent may be covalently associated with the supercharged protein to effect delivery of the agent to a cell. For example, a peptide therapeutic may be fused to the supercharged protein in order to deliver the peptide therapeutic to a cell. In certain embodiments, the supercharged protein and the peptide may be joined via a cleavable linker. To give but another example, a small molecule may be conjugated to a supercharged protein for delivery to a cell. The agent may also be associated with the supercharged protein through non-covalent interactions (e.g., ligand-receptor interaction, dipole-dipole interaction, etc.).

The present invention provides complexes comprising supercharged proteins and one or more molecules of the agent to be delivered. In some embodiments, such complexes comprise multiple agent molecules per supercharged protein molecule. In some embodiments, such complexes comprise 1, 2, 3, 4, 5, 6, 7, 8, 9, 10, 20, or more agent (e.g., nucleic acids) molecules per supercharged protein molecule. In certain particular embodiments, a complex comprises approximately 1-2 nucleic acid molecules (e.g., siRNA) to approximately 1 supercharged protein molecule. In other embodiments, such complexes comprise multiple protein molecules per agent molecule. In some embodiments, such complexes comprise 1, 2, 3, 4, 5, 6, 7, 8, 9, 10, 20, or more protein molecules per agent molecule. In certain embodiments, such complexes comprise approximately one agent molecule and approximately one superpositively charged protein molecule. In certain embodiments, the overall net charge on the agent/supercharged protein complex is negative. In certain embodiments, the overall net charge on the agent/supercharged protein complex is positive. In certain embodiments, the overall net charge on the agent/supercharged protein complex is neutral. In certain particular embodiments, the overall net charge on the nucleic acid/supercharged protein complex is positive.

In another aspect, the present invention provides pharmaceutical compositions comprising: a) one or more supercharged proteins; b) one or more complexes of supercharged protein and an agent to be delivered; or c) one or more of a) or one or more of b), in accordance with the invention and at least one pharmaceutically acceptable excipient. The amount of the complex in the composition may be the amount useful to induce a desired biological response in the cell, for example, increase or decrease the expression of a particular gene in the cell. In certain embodiments, the complex is associated with a targeting moiety (e.g., small molecule, protein, peptide, carbohydrate, etc.) used to direct the delivery of the agent to a particular cell, type of cell, tissue, or organ.

In some embodiments, a supercharged protein or complexes comprising supercharged proteins, engineered or naturally occurring, and one or more nucleic acids (and/or pharmaceutical compositions thereof) are useful as therapeutic agents. In some embodiments, a nucleic acid and/or supercharged protein may be therapeutically active. In certain embodiments, the nucleic acid is therapeutically active. For example, some conditions (e.g., cancer, inflammatory diseases) are associated with the expression of certain mRNAs and/or proteins. Supercharged proteins associated with RNAi agents targeting an expressed mRNA may be useful for treating such conditions. Alternatively, some conditions are associated with underexpression of certain mRNAs and/or proteins (e.g., cancer, inborn errors in metabolism). Supercharged proteins associated with vectors that drive expression of the deficient mRNA and/or protein may be useful for treating such conditions.

The present invention also provides kits useful for producing the inventive supercharged protein or supercharged protein/agent complexes or compositions thereof, and/or using such complexes to transfect or deliver the supercharged protein or an agent into a cell. The inventive kits may also include instructions for administering or using the inventive supercharged proteins or complexes, or a pharmaceutical composition thereof. For example, the kit may include instructions for prescribing the pharmaceutical composition to a subject. The kit may include enough materials for multiple unit doses of the agent. The kit may be designed for therapeutic or research purposes. The kit may optionally include the agent (e.g. siRNA, peptide, drug) to be delivered, or the agent may be provided by the end user.

The present invention also provides a method of introducing a supercharged protein or an agent associated with a supercharged protein, or both, into a cell. The inventive method comprises contacting the supercharged protein, or a supercharged protein and an agent associated with the supercharged protein with the cell, e.g., under conditions sufficient to allow penetration of said supercharged protein, or an agent associated with a supercharged protein, into the cell, thereby introducing a supercharged protein, or an agent associated with a supercharged protein, or both, into a cell. In certain embodiments, sufficient supercharged protein or agent enters the cell to allow for one or more of detection of: the supercharged protein or agent in the cell; a change in a biological property of the cell, e.g., growth rate, pattern of gene expression, or viability, of the cell; or detection of a biological effect of the supercharged protein or agent. In certain embodiments, the contact is performed in vitro. In certain embodiments, the contact is performed in vivo, e.g., in the body of a subject, e.g., a human or other animal. In one in vivo embodiment, sufficient supercharged protein, agent, or both is present in the cell to provide a detectable effect in the subject, e.g., a therapeutic effect. In one in vivo embodiment, sufficient supercharged protein, agent, or both is present in the cell to allow imaging of one or more penetrated cells or tissues. In certain embodiments, the observed or detectable effect arises from cell penetration.

The present invention also provides a method of evaluating a supercharged protein for cell penetration comprising: optionally, selecting a supercharged protein; providing said supercharged protein; and contacting said supercharged protein with a cell and determining if the supercharged protein penetrates the cell, thereby providing an evaluation of a supercharged protein for cell penetration.

The present invention also provides a method of evaluating a supercharged protein for cell penetration comprising: selecting a protein to be supercharged; obtaining a set of one or a plurality of residues to be varied to produce a supercharged protein, wherein the set was generated by a method described herein (obtaining includes generating the set or receiving the identity of one or more members of the set from another party); providing (e.g., by making or receiving it from another party) a supercharged protein having said set of varied residues; and contacting said supercharged protein with a cell and determining if the supercharged protein penetrates the cell, thereby of evaluating a supercharged protein for cell penetration. The method can allow for a party to develop supercharged proteins or to collaborate with others to do so.

### DEFINITIONS

Agent to be delivered: As used herein, the phrase “agent to be delivered” refers to any substance that can be delivered to a subject, organ, tissue, cell, subcellular locale, and/or extracellular matrix locale. In some embodiments, the agent to be delivered is a biologically active agent, i.e., it has activity in a biological system and/or organism. For instance, a substance that, when administered to an organism, has a biological effect on that organism, is considered to be biologically active. In particular embodiments, where an agent to be delivered is a biologically active agent, a portion of that agent that shares at least one biological activity of the agent as a whole is typically referred to as a “biologically active” portion. In some embodiments, an agent to be delivered is a therapeutic agent. As used herein, the term “therapeutic agent” refers to any agent that, when administered to a subject, has a beneficial effect. The term “therapeutic agent” refers to any agent that, when administered to a subject, has a therapeutic, diagnostic, and/or prophylactic effect and/or elicits a desired biological and/or pharmacological effect. As used herein, the term “therapeutic agent” may be a nucleic acid that is delivered to a cell by via its association with a supercharged protein. In certain embodiments, the agent to be delivered is a nucleic acid. In certain embodiments, the agent to be delivered is DNA. In certain embodiments, the agent to be delivered is RNA. In certain embodiments, the agent to be delivered is a peptide or protein. In certain embodiments, the agent to be delivered is a small molecule. In some embodiments, the agent to be delivered is useful as an in vivo or in vitro imaging agent. In some of these embodiments, it is, and in others it is not, biologically active.

Animal: As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically-engineered animal, or a clone.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. A supercharged protein is typically associated with a nucleic acid by a mechanism that involves non-covalent binding (e.g., electrostatic interactions). In certain embodiments, a positively charged, supercharged protein is associated with a nucleic acid through electrostatic interactions to form a complex. In some embodiments, a sufficient number of weaker interactions can provide sufficient stability for moieties to remain physically associated under a variety of different conditions. In certain embodiments, the agent to be delivered is covalently bound to the supercharged protein.

Biocompatible: As used herein, the term “biocompatible” refers to substances that are not toxic to cells. In some embodiments, a substance is considered to be “biocompatible” if its addition to cells in vivo does not induce inflammation and/or other adverse effects in vivo. In some embodiments, a substance is considered to be “biocompatible” if its addition to cells in vitro or in vivo results in less than or equal to about 50%, about 45%, about 40%, about 35%, about 30%, about 25%, about 20%, about 15%, about 10%, about 5%, or less than about 5% cell death.

Biodegradable: As used herein, the term “biodegradable” refers to substances that are degraded under physiological conditions. In some embodiments, a biodegradable substance is a substance that is broken down by cellular machinery. In some embodiments, a biodegradable substance is a substance that is broken down by chemical processes.

Biologically active: As used herein, the phrase “biologically active” refers to a characteristic of any substance that has activity in a biological system and/or organism. For instance, a substance that, when administered to an organism, has a biological effect on that organism, is considered to be biologically active. In particular embodiments, where a nucleic acid is biologically active, a portion of that nucleic acid that shares at least one biological activity of the whole nucleic acid is typically referred to as a “biologically active” portion.

Carbohydrate: The term “carbohydrate” refers to a sugar or polymer of sugars. The terms “saccharide,” “polysaccharide,” “carbohydrate,” and “oligosaccharide” may be used interchangeably. Most carbohydrates are aldehydes or ketones with many hydroxyl groups, usually one on each carbon atom of the molecule. Carbohydrates generally have the molecular formula CₙH₂ₙOₙ. A carbohydrate may be a monosaccharide, a disaccharide, trisaccharide, oligosaccharide, or polysaccharide. The most basic carbohydrate is a monosaccharide, such as glucose, sucrose, galactose, mannose, ribose, arabinose, xylose, and fructose. Disaccharides are two joined monosaccharides. Exemplary disaccharides include sucrose, maltose, cellobiose, and lactose. Typically, an oligosaccharide includes between three and six monosaccharide units (e.g., raffinose, stachyose), and polysaccharides include six or more monosaccharide units. Exemplary polysaccharides include starch, glycogen, and cellulose. Carbohydrates may contain modified saccharide units such as 2′-deoxyribose wherein a hydroxyl group is removed, 2′-fluororibose wherein a hydroxyl group is replace with a fluorine, or N-acetylglucosamine, a nitrogen-containing form of glucose (e.g., 2′-fluororibose, deoxyribose, and hexose). Carbohydrates may exist in many different forms, for example, conformers, cyclic forms, acyclic forms, stereoisomers, tautomers, anomers, and isomers.

Characteristic portion: As used herein, the term a “characteristic portion” of a substance, in the broadest sense, is one that shares some degree of sequence and/or structural identity and/or at least one functional characteristic with the relevant intact substance. For example, a “characteristic portion” of a protein or polypeptide is one that contains a continuous stretch of amino acids, or a collection of continuous stretches of amino acids, that together are characteristic of a protein or polypeptide. In some embodiments, each such continuous stretch generally will contain at least 2, at least 5, at least 10, at least 15, at least 20, at least 50, or more amino acids. A “characteristic portion” of a nucleic acid is one that contains a continuous stretch of nucleotides, or a collection of continuous stretches of nucleotides, that together are characteristic of a nucleic acid. In some embodiments, each such continuous stretch generally will contain at least 2, at least 5, at least 10, at least 15, at least 20, at least 50, or more nucleotides. In some embodiments, a characteristic portion is biologically active.

Conserved: As used herein, the term “conserved” refers to nucleotides or amino acid residues of a polynucleotide sequence or amino acid sequence, respectively, that are those that occur unaltered in the same position of two or more related sequences being compared. Nucleotides or amino acids that are relatively conserved are those that are conserved amongst more related sequences than nucleotides or amino acids appearing elsewhere in the sequences. In some embodiments, two or more sequences are said to be “completely conserved” if they are 100% identical to one another. In some embodiments, two or more sequences are said to be “highly conserved” if they are at least 70% identical, at least 80% identical, at least 90% identical, or at least 95% identical to one another. In some embodiments, two or more sequences are said to be “highly conserved” if they are about 70% identical, about 80% identical, about 90% identical, about 95%, about 98%, or about 99% identical to one another. In some embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In some embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Functional: As used herein, a “functional” biological molecule is a biological molecule in a form in which it exhibits a property and/or activity by which it is characterized.

Fusion protein: As used herein, a “fusion protein” includes a first protein moiety, e.g., a supercharged protein, having a peptide linkage with a second protein moiety. In certain embodiments, the fusion protein is encoded by a single fusion gene.

Gene: As used herein, the term “gene” has its meaning as understood in the art. It will be appreciated by those of ordinary skill in the art that the term “gene” may include gene regulatory sequences (e.g., promoters, enhancers, etc.) and/or intron sequences. It will further be appreciated that definitions of gene include references to nucleic acids that do not encode proteins but rather encode functional RNA molecules such as RNAi agents, ribozymes, tRNAs, etc. For the purpose of clarity we note that, as used in the present application, the term “gene” generally refers to a portion of a nucleic acid that encodes a protein; the term may optionally encompass regulatory sequences, as will be clear from context to those of ordinary skill in the art. This definition is not intended to exclude application of the term “gene” to non-protein-coding expression units but rather to clarify that, in most cases, the term as used in this document refers to a protein-coding nucleic acid.

Gene product or expression product: As used herein, the term “gene product” or “expression product” generally refers to an RNA transcribed from the gene (pre- and/or post-processing) or a polypeptide (pre- and/or post-modification) encoded by an RNA transcribed from the gene.

Green fluorescent protein: As used herein, the term “green fluorescent protein” (GFP) refers to a protein originally isolated from the jellyfish Aequorea victoria that fluoresces green when exposed to blue light or a derivative of such a protein (e.g., a supercharged version of the protein). The amino acid sequence of wild type GFP is as follows:

(SEQ ID NO: XX)

MSKGEELFTG VVPILVELDG DVNGHKFSVS GEGEGDATYG
KLTLKFICTT GKLPVPWPTL VTTFSYGVQC FSRYPDHMKQ
HDFFKSAMPE GYVQERTIFF KDDGNYKTRA EVKFEGDTLV
NRIELKGIDF KEDGNILGHK LEYNYNSHNV YIMADKQKNG
IKVNFKIRHN IEDGSVQLAD HYQQNTPIGD GPVLLPDNHY
LSTQSALSKD PNEKRDHMVL LEFVTAAGIT HGMDELYK.

Proteins that are at least 70%, at least 75%, at least 80%, at least 85%, at least 90%, at least 95%, at least 98%, or at least 99% homologous are also considered to be green fluorescent proteins. In certain embodiments, the green fluorescent protein is supercharged. In certain embodiments, the green fluorescent protein is superpositively charged (e.g., +15 GFP, +25 GFP, and +36 GFP as described herein). In certain embodiments, the GFP may be modified to include a polyhistidine tag for ease in purification of the protein. In certain embodiments, the GFP may be fused with another protein or peptide (e.g., hemagglutinin 2 (HA2) peptide). In certain embodiments, the GFP may be further modified biologically or chemically (e.g., post-translational modifications, proteolysis, etc.).

Homology: As used herein, the term “homology” refers to the overall relatedness between polymeric molecules, e.g. between nucleic acid molecules (e.g. DNA molecules and/or RNA molecules) and/or between polypeptide molecules. In some embodiments, polymeric molecules are considered to be “homologous” to one another if their sequences are at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% identical. In some embodiments, polymeric molecules are considered to be “homologous” to one another if their sequences are at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% similar. The term “homologous” necessarily refers to a comparison between at least two sequences (nucleotides sequences or amino acid sequences). In accordance with the invention, two nucleotide sequences are considered to be homologous if the polypeptides they encode are at least about 50% identical, at least about 60% identical, at least about 70% identical, at least about 80% identical, or at least about 90% identical for at least one stretch of at least about 20 amino acids. In some embodiments, homologous nucleotide sequences are characterized by the ability to encode a stretch of at least 4-5 uniquely specified amino acids. Both the identity and the approximate spacing of these amino acids relative to one another must be considered for nucleotide sequences to be considered homologous. For nucleotide sequences less than 60 nucleotides in length, homology is determined by the ability to encode a stretch of at least 4-5 uniquely specified amino acids. In accordance with the invention, two protein sequences are considered to be homologous if the proteins are at least about 50% identical, at least about 60% identical, at least about 70% identical, at least about 80% identical, or at least about 90% identical for at least one stretch of at least about 20 amino acids.

Hydrophilic: As used herein, a “hydrophilic” substance is a substance that may be soluble in polar dispersion media. In some embodiments, a hydrophilic substance can transiently bond with polar dispersion media. In some embodiments, a hydrophilic substance transiently bonds with polar dispersion media through hydrogen bonding. In some embodiments, the polar dispersion medium is water. In some embodiments, a hydrophilic substance may be ionic. In some embodiments, a hydrophilic substance may be non-ionic. In some embodiments, a substance is hydrophilic relative to another substance because it is more soluble in water, polar dispersion media, or hydrophilic dispersion media than is the other substance. In some embodiments, a substance is hydrophilic relative to another substance because it is less soluble in oil, non-polar dispersion media, or hydrophobic dispersion media than is the other substance.

Hydrophobic: As used herein, a “hydrophobic” substance is a substance that may be soluble in non-polar dispersion media. In some embodiments, a hydrophobic substance is repelled from polar dispersion media. In some embodiments, the polar dispersion medium is water. In some embodiments, hydrophobic substances are non-polar. In some embodiments, a substance is hydrophobic relative to another substance because it is more soluble in oil, non-polar dispersion media, or hydrophobic dispersion media than is the other substance. In some embodiments, a substance is hydrophobic relative to another substance because it is less soluble in water, polar dispersion media, or hydrophilic dispersion media than is the other substance.

Identity: As used herein, the term “identity” refers to the overall relatedness between polymeric molecules, e.g., between nucleic acid molecules (e.g. DNA molecules and/or RNA molecules) and/or between polypeptide molecules. Calculation of the percent identity of two nucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Atschul, S. F. et al., J. Molec. Biol., 215, 403 (1990)).

Inhibit expression of a gene: As used herein, the phrase “inhibit expression of a gene” means to cause a reduction in the amount of an expression product of the gene. The expression product can be an RNA transcribed from the gene (e.g., an mRNA) or a polypeptide translated from an mRNA transcribed from the gene. Typically a reduction in the level of an mRNA results in a reduction in the level of a polypeptide translated therefrom. The level of expression may be determined using standard techniques for measuring mRNA or protein.

In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g., animal, plant, or microbe).

Isolated: As used herein, the term “isolated” refers to a substance or entity that has been (1) separated from at least some of the components with which it was associated when initially produced (whether in nature or in an experimental setting), and/or (2) produced, prepared, and/or manufactured by the hand of man. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

microRNA (miRNA): As used herein, the term “microRNA” or “miRNA” refers to an RNAi agent that is approximately 21 nucleotides (nt)-23 nt in length. miRNAs can range between 18 nt-26 nt in length. Typically, miRNAs are single-stranded. However, in some embodiments, miRNAs may be at least partially double-stranded. In certain embodiments, miRNAs may comprise an RNA duplex (referred to herein as a “duplex region”) and may optionally further comprises one to three single-stranded overhangs. In some embodiments, an RNAi agent comprises a duplex region ranging from 15 bp to 29 bp in length and optionally further comprising one or two single-stranded overhangs. An miRNA may be formed from two RNA molecules that hybridize together, or may alternatively be generated from a single RNA molecule that includes a self-hybridizing portion. In general, free 5′ ends of miRNA molecules have phosphate groups, and free 3′ ends have hydroxyl groups. The duplex portion of an miRNA usually, but does not necessarily, comprise one or more bulges consisting of one or more unpaired nucleotides. One strand of an miRNA includes a portion that hybridizes with a target RNA. In certain embodiments, one strand of the miRNA is not precisely complementary with a region of the target RNA, meaning that the miRNA hybridizes to the target RNA with one or more mismatches. In some embodiments, one strand of the miRNA is precisely complementary with a region of the target RNA, meaning that the miRNA hybridizes to the target RNA with no mismatches. Typically, miRNAs are thought to mediate inhibition of gene expression by inhibiting translation of target transcripts. However, in some embodiments, miRNAs may mediate inhibition of gene expression by causing degradation of target transcripts.

Nucleic acid: As used herein, the term “nucleic acid,” in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising individual nucleic acid residues. As used herein, the terms “oligonucleotide” and “polynucleotide” can be used interchangeably to refer to a polymer of nucleotides (e.g., a string of at least two nucleotides). In some embodiments, “nucleic acid” encompasses RNA as well as single and/or double-stranded DNA and/or cDNA. Furthermore, the terms “nucleic acid,” “DNA,” “RNA,” and/or similar terms include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the present invention. The term “nucleotide sequence encoding an amino acid sequence” includes all nucleotide sequences that are degenerate versions of each other and/or encode the same amino acid sequence. Nucleotide sequences that encode proteins and/or RNA may include introns. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, backbone modifications, etc. A nucleic acid sequence is presented in the 5′ to 3′ direction unless otherwise indicated. The term “nucleic acid segment” is used herein to refer to a nucleic acid sequence that is a portion of a longer nucleic acid sequence. In many embodiments, a nucleic acid segment comprises at least 3, at least 4, at least 5, at least 6, at least 7, at least 8, at least 9, at least 10, or more residues. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages). In some embodiments, the present invention is specifically directed to “unmodified nucleic acids,” meaning nucleic acids (e.g. polynucleotides and residues, including nucleotides and/or nucleosides) that have not been chemically modified in order to facilitate or achieve delivery.

Polymer: As used herein, the term “polymer” refers to any substance comprising at least two repeating structural units (i.e., “monomers”) which are associated with one another. In some embodiments, monomers are covalently associated with one another. In some embodiments, monomers are non-covalently associated with one another. Polymers may be homopolymers or copolymers comprising two or more monomers. In terms of sequence, copolymers may be random, block, graft, or comprise a combination of random, block, and/or graft sequences. In some embodiments, block copolymers are diblock copolymers. In some embodiments, block copolymers are triblock copolymers. In some embodiments, polymers can be linear or branched polymers. In some embodiments, polymers in accordance with the invention comprise blends, mixtures, and/or adducts of any of the polymers described herein. Typically, polymers in accordance with the present invention are organic polymers. In some embodiments, polymers are hydrophilic. In some embodiments, polymers are hydrophobic. In some embodiments, polymers modified with one or more moieties and/or functional groups.

Protein: As used herein, the term “protein” refers to a polypeptide (i.e., a string of at least two amino acids linked to one another by peptide bonds). Proteins may include moieties other than amino acids (e.g., may be glycoproteins) and/or may be otherwise processed or modified. Those of ordinary skill in the art will appreciate that a “protein” can be a complete polypeptide chain as produced by a cell (with or without a signal sequence), or can be a functional portion thereof. Those of ordinary skill will further appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., addition of a chemical entity such as a carbohydrate group, a phosphate group, a farnesyl group, an isofarnesyl group, a fatty acid group, an amide group, a terminal acetyl group, a linker for conjugation, functionalization, or other modification (e.g., alpha amidation), etc. In a preferred embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide. In certain embodiments, the modifications of the peptide lead to a more biologically active peptide. In some embodiments, polypeptides may comprise natural amino acids, non-natural amino acids, synthetic amino acids, amino acid analogs, and combinations thereof. The term “peptide” is typically used to refer to a polypeptide having a length of less than about 100 amino acids.

RNA interference (RNAi): As used herein, the term “RNA interference” or “RNAi” refers to sequence-specific inhibition of gene expression and/or reduction in target RNA levels mediated by an RNA, which RNA comprises a portion that is substantially complementary to a target RNA. Typically, at least part of the substantially complementary portion is within the double stranded region of the RNA. In some embodiments, RNAi can occur via selective intracellular degradation of RNA. In some embodiments, RNAi can occur by translational repression.

RNAi agent: As used herein, the term “RNAi agent” or “RNAi” refers to an RNA, optionally including one or more nucleotide analogs or modifications, having a structure characteristic of molecules that can mediate inhibition of gene expression through an RNAi mechanism. In some embodiments, RNAi agents mediate inhibition of gene expression by causing degradation of target transcripts. In some embodiments, RNAi agents mediate inhibition of gene expression by inhibiting translation of target transcripts. Generally, an RNAi agent includes a portion that is substantially complementary to a target RNA. In some embodiments, RNAi agents are at least partly double-stranded. In some embodiments, RNAi agents are single-stranded. In some embodiments, exemplary RNAi agents can include siRNA, shRNA, and/or miRNA. In some embodiments, RNAi agents may be composed entirely of natural RNA nucleotides (i.e., adenine, guanine, cytosine, and uracil). In some embodiments, RNAi agents may include one or more non-natural RNA nucleotides (e.g., nucleotide analogs, DNA nucleotides, etc.). Inclusion of non-natural RNA nucleic acid residues may be used to make the RNAi agent more resistant to cellular degradation than RNA. In some embodiments, the term “RNAi agent” may refer to any RNA, RNA derivative, and/or nucleic acid encoding an RNA that induces an RNAi effect (e.g., degradation of target RNA and/or inhibition of translation). In some embodiments, an RNAi agent may comprise a blunt-ended (i.e., without overhangs) dsRNA that can act as a Dicer substrate. For example, such an RNAi agent may comprise a blunt-ended dsRNA which is ≧25 base pairs length, which may optionally be chemically modified to abrogate an immune response.

RNAi-inducing agent: As used herein, the term “RNAi-inducing agent” encompasses any entity that delivers, regulates, and/or modifies the activity of an RNAi agent. In some embodiments, RNAi-inducing agents may include vectors (other than naturally occurring molecules not modified by the hand of man) whose presence within a cell results in RNAi and leads to reduced expression of a transcript to which the RNAi-inducing agent is targeted. In some embodiments, RNAi-inducing agents are RNAi-inducing vectors. In some embodiments, RNAi-inducing agents are compositions comprising RNAi agents and one or more pharmaceutically acceptable excipients and/or carriers. In some embodiments, an RNAi-inducing agent is an “RNAi-inducing vector,” which refers to a vector whose presence within a cell results in production of one or more RNAs that self-hybridize or hybridize to each other to form an RNAi agent (e.g. siRNA, shRNA, and/or miRNA). In various embodiments, this term encompasses plasmids, e.g., DNA vectors (whose sequence may comprise sequence elements derived from a virus), or viruses (other than naturally occurring viruses or plasmids that have not been modified by the hand of man), whose presence within a cell results in production of one or more RNAs that self-hybridize or hybridize to each other to form an RNAi agent. In general, the vector comprises a nucleic acid operably linked to expression signal(s) so that one or more RNAs that hybridize or self-hybridize to form an RNAi agent are transcribed when the vector is present within a cell. Thus the vector provides a template for intracellular synthesis of the RNA or RNAs or precursors thereof. For purposes of inducing RNAi, presence of a viral genome in a cell (e.g., following fusion of the viral envelope with the cell membrane) is considered sufficient to constitute presence of the virus within the cell. In addition, for purposes of inducing RNAi, a vector is considered to be present within a cell if it is introduced into the cell, enters the cell, or is inherited from a parental cell, regardless of whether it is subsequently modified or processed within the cell. An RNAi-inducing vector is considered to be targeted to a transcript if presence of the vector within a cell results in production of one or more RNAs that hybridize to each other or self-hybridize to form an RNAi agent that is targeted to the transcript, i.e., if presence of the vector within a cell results in production of one or more RNAi agents targeted to the transcript.

Short, interfering RNA (siRNA): As used herein, the term “short, interfering RNA” or “siRNA” refers to an RNAi agent comprising an RNA duplex (referred to herein as a “duplex region”) that is approximately 19 base pairs (bp) in length and optionally further comprises one to three single-stranded overhangs. In some embodiments, an RNAi agent comprises a duplex region ranging from 15 bp to 29 bp in length and optionally further comprising one or two single-stranded overhangs. An siRNA may be formed from two RNA molecules that hybridize together, or may alternatively be generated from a single RNA molecule that includes a self-hybridizing portion. In general, free 5′ ends of siRNA molecules have phosphate groups, and free 3′ ends have hydroxyl groups. The duplex portion of an siRNA may, but typically does not, comprise one or more bulges consisting of one or more unpaired nucleotides. One strand of an siRNA includes a portion that hybridizes with a target transcript. In certain embodiments, one strand of the siRNA is precisely complementary with a region of the target transcript, meaning that the siRNA hybridizes to the target transcript without a single mismatch. In some embodiments, one or more mismatches between the siRNA and the targeted portion of the target transcript may exist. In some embodiments in which perfect complementarity is not achieved, any mismatches are generally located at or near the siRNA termini. In some embodiments, siRNAs mediate inhibition of gene expression by causing degradation of target transcripts.

Short hairpin RNA (shRNA): As used herein, the term “short hairpin RNA” or “shRNA” refers to an RNAi agent comprising an RNA having at least two complementary portions hybridized or capable of hybridizing to form a double-stranded (duplex) structure sufficiently long to mediate RNAi (typically at least approximately 19 bp in length), and at least one single-stranded portion, typically ranging between approximately 1 nucleotide (nt) and approximately 10 nt in length that forms a loop. In some embodiments, an shRNA comprises a duplex portion ranging from 15 bp to 29 bp in length and at least one single-stranded portion, typically ranging between approximately 1 nt and approximately 10 nt in length that forms a loop. The duplex portion may, but typically does not, comprise one or more bulges consisting of one or more unpaired nucleotides. In some embodiments, siRNAs mediate inhibition of gene expression by causing degradation of target transcripts. shRNAs are thought to be processed into siRNAs by the conserved cellular RNAi machinery. Thus shRNAs may be precursors of siRNAs. Regardless, siRNAs in general are capable of inhibiting expression of a target RNA, similar to siRNAs.

Small molecule: In general, a “small molecule” refers to a substantially non-peptidic, non-oligomeric organic compound either prepared in the laboratory or found in nature. Small molecules, as used herein, can refer to compounds that are “natural product-like,” however, the term “small molecule” is not limited to “natural product-like” compounds. Rather, a small molecule is typically characterized in that it contains several carbon-carbon bonds, and has a molecular weight of less than 1500 g/mol, less than 1250 g/mol, less than 1000 g/mol, less than 750 g/mol, less than 500 g/mol, or less than 250 g/mol, although this characterization is not intended to be limiting for the purposes of the present invention. In certain other embodiments, natural-product-like small molecules are utilized.

Similarity: As used herein, the term “similarity” refers to the overall relatedness between polymeric molecules, e.g. between nucleic acid molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Stable: As used herein, the term “stable” as applied to a protein refers to any aspect of protein stability. The stable modified protein as compared to the original unmodified protein possesses any one or more of the following characteristics: more soluble, more resistant to aggregation, more resistant to denaturation, more resistant to unfolding, more resistant to improper or undesired folding, greater ability to renature, increased thermal stability, increased stability in a variety of environments (e.g., pH, salt concentration, presence of detergents, presence of denaturing agents, etc.), and increased stability in non-aqueous environments. In certain embodiments, the stable modified protein exhibits at least two of the above characteristics. In certain embodiments, the stable modified protein exhibits at least three of the above characteristics. Such characteristics may allow the active protein to be produced at higher levels. For example, the modified protein can be overexpressed at a higher level without aggregation than the unmodified version of the protein. Such characteristics may also allow the protein to be used as a therapeutic agent or a research tool.

Subject: As used herein, the term “subject” or “patient” refers to any organism to which a composition in accordance with the invention may be administered, e.g., for experimental, diagnostic, prophylactic, and/or therapeutic purposes. Typical subjects include animals (e.g., mammals such as mice, rats, rabbits, non-human primates, and humans) and/or plants.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of a disease, disorder, and/or condition.

Supercharge: As used herein, the term “supercharge” refers to any modification of a protein that results in the increase or decrease of the overall net charge of the protein. Modifications include, but are not limited to, alterations in amino acid sequence or addition of charged moieties (e.g., carboxylic acid groups, phosphate groups, sulfate groups, amino groups). Supercharging also refers to the association of an agent with a charged protein, naturally occurring or modified, to form a complex with increased or decreased charge relative to the agent alone.

Supercharged complex: As defined herein, a “supercharged complex” refers to the combination of one or more agents associated with a supercharged protein, engineered or naturally occurring, that collectively has an increased or decreased charge relative to the agent alone.

Susceptible to: An individual who is “susceptible to” a disease, disorder, and/or condition has not been diagnosed with and/or may not exhibit symptoms of the disease, disorder, and/or condition. In some embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Targeting agent or targeting moiety: As used herein, the term “targeting agent” or “targeting moiety” refers to any substance that binds to a component associated with a cell, tissue, and/or organ. Such a component is referred to as a “target” or a “marker.” A targeting agent or targeting moiety may be a polypeptide, glycoprotein, nucleic acid, small molecule, carbohydrate, lipid, etc. In some embodiments, a targeting agent or targeting moiety is an antibody or characteristic portion thereof. In some embodiments, a targeting agent or targeting moiety is a receptor or characteristic portion thereof. In some embodiments, a targeting agent or targeting moiety is a ligand or characteristic portion thereof. In some embodiments, a targeting agent or targeting moiety is a nucleic acid targeting agent (e.g. an aptamer) that binds to a cell type specific marker. In some embodiments, a targeting agent or targeting moiety is an organic small molecule. In some embodiments, a targeting agent or targeting moiety is an inorganic small molecule.

Target gene: As used herein, the term “target gene” refers to any gene whose expression is altered by an RNAi or other agent.

Target transcript: As used herein, the term “target transcript” refers to any mRNA transcribed from a target gene.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to a disease, disorder, and/or condition, to treat, improve symptoms of, diagnose, prevent, and/or delay the onset of the disease, disorder, and/or condition.

Treating: As used herein, the term “treating” refers to partially or completely alleviating, ameliorating, improving, relieving, delaying onset of, inhibiting progression of, reducing severity of, and/or reducing incidence of one or more symptoms or features of a particular disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. In some embodiments, treatment comprises delivery of a supercharged protein associated with a therapeutically active nucleic acid to a subject in need thereof.

Unmodified: As used herein, “unmodified” refers to the protein or agent prior to being supercharged or associated in a complex with a supercharged protein, engineered or naturally occurring.

Vector: As used herein, “vector” refers to a nucleic acid molecule which can transport another nucleic acid to which it has been linked. In some embodiment, vectors can achieve extra-chromosomal replication and/or expression of nucleic acids to which they are linked in a host cell such as a eukaryotic and/or prokaryotic cell. Vectors capable of directing the expression of operatively linked genes are referred to herein as “expression vectors.”

### BRIEF DESCRIPTION OF THE DRAWING

FIG. 1. Supercharged green fluorescent proteins (GFPs). (A) Protein sequences of GFP variants, with fluorophore-forming residues highlighted green, negatively charged residues highlighted red, and positively charged residues highlighted blue. (B-D) Electrostatic surface potentials of sfGFP (B), GFP(+36) (C), and GFP(−30) (D), colored from −25 kT/e (red) to +25 kT/e (blue).

FIG. 2. Intramolecular properties of GFP variants. (A) Staining and UV fluorescence of purified GFP variants. Each lane and tube contains 0.2 μg of protein. (B) Circular dichroism spectra of GFP variants. (C) Thermodynamic stability of GFP variants, measured by guanidinium-induced unfolding.

FIG. 3. Intermolecular properties of supercharged proteins. (A) UV-illuminated samples of purified GFP variants (“native”), those samples heated 1 minute at 100° C. (“boiled”), and those samples subsequently cooled for 2 hours at 25° C. (“cooled”). (B) Aggregation of GFP variants was induced with 40% TFE at 25° C. and monitored by right-angle light scattering. (C) Supercharged GFPs adhere reversibly to oppositely charged macromolecules. Sample 1: 6 μg of GFP(+36) in 30 μl of 25 mM Tris pH 7.0 and 100 mM NaCl. Sample 2: 6 μg of GFP(−30) added to sample 1. Sample 3: 30 μg of salmon sperm DNA added to sample 1. Sample 4: 20 μg of E. coli tRNA added to sample 1. Sample 5: Addition of 1 M NaCl to sample 4. Samples 6-8: identical to samples 1, 2, and 4, respectively, except using sfGFP instead of GFP(+36). All samples were spun briefly in a microcentrifuge and visualized under UV light.

FIG. 4. (A) Excitation and (B) emission spectra of GFP variants. Each sample contained an equal amount of protein as quantitated by chromophore absorbance at 490 nm.

FIG. 5. Supercharged Surfaces Dominate Intermolecular Interactions. Supercharged GFPs adhere non-specifically and reversibly with oppositely charged macromolecules (“protein Velcro”). Such interactions can result in the formation of precipitates. Unlike aggregates of denatured proteins, these precipitates contain folded, fluorescent GFP and dissolve in 1 M salt. Shown here are: +36 GFP alone; +36 GFP mixed with −30 GFP; +36 GFP mixed with tRNA; +36 GFP mixed with tRNA in 1 M NaCl; sf GFP (−7); and sfGFP mixed with −30 GFP.

FIG. 6. Superpositive GFP Binds siRNA. GFP-siRNA complex does not co-migrate with siRNA in an agarose gel −+36 GFP was incubated with siRNA, and the resulting complexes were subjected to agarose gel electrophoresis. Various +36 GFP:siRNA ratios were tested in this assay: 0:1, 1:1, 1:2, 1:3, 1:4, 1:5, and 1:10. +36 GFP was shown to form a stable complex with siRNA in a ˜1:3 stoichiometry. Non-superpositive proteins were shown not to bind siRNA. A 50:1 ratio of sfGFP:siRNA was tested, but, even at such high levels of excess, sfGFP did not associate with siRNA.

FIG. 7. Superpositive GFP Penetrates Cells. HeLa cells were incubated with GFP (either sf GFP (−7), −30 GFP, or +36 GFP), washed, fixed, and stained. +36 GFP, but not sfGFP or −30 GFP, potently penetrated HeLa cells. Left: DAPI staining of DNA to mark cells. Middle: GFP staining to mark where cellular uptake of GFP occurred. Right: movie showing +36 GFP localization as it occurs.

FIG. 8. Superpositive GFP Delivers siRNA into Human Cells. +36 GFP was shown to potently deliver siRNA into HeLa cells. Left: Lipofectamine 2000 and Cy3-siRNA; right: +36 GFP and Cy3-siRNA. +36 GFP was shown to potently deliver siRNA into HeLa cells. Hoescht channel, blue, was used to visualize DNA, thereby marking the position of cells; Cy3 channel, red, was used to visualize Cy3-tagged siRNA; GFP channel, green, was used to visualize GFP; yellow indicates sites of co-localization between siRNA and GFP.

FIG. 9. Delivery of siRNA into Cell Lines Resistant to Traditional Transfection: murine 3T3-L₁ pre-adipocyte cells (“3T3L cells”). 3T3L cells were treated with either: lipofectamine 2000 and Cy3-siRNA (left); or +36 GFP and Cy3-siRNA (right). 3T3L cells were poorly transfected by Lipofectamine but were efficiently transfected by +36 GFP. Hoescht channel, blue, was used to visualize DNA, thereby marking the position of cells; Cy3 channel, red, was used to visualize Cy3-tagged siRNA; GFP channel, green, was used to visualize GFP. Yellow indicates sites of co-localization between siRNA and GFP.

FIG. 10. Delivery of siRNA into Cell Lines Resistant to Traditional Transfection: rat IMCD cells. Rat IMCD cells were treated with either Lipofectamine 2000 and Cy3-siRNA (left); or +36 GFP and Cy3-siRNA (right). Rat IMCD cells were poorly transfected by Lipofectamine but were efficiently transfected by +36 GFP. Hoescht channel, blue, was used to visualize DNA, thereby marking the position of cells; Cy3 channel, red, was used to visualize Cy3-tagged siRNA; GFP channel, green, was used to visualize GFP. Yellow indicates sites of co-localization between siRNA and GFP.

FIG. 11. Delivery of siRNA into Cell Lines Resistant to Traditional Transfection: human ST14A neurons. Human ST14A neurons were treated with either Lipofectamine 2000 and Cy3-siRNA (left); or +36 GFP and Cy3-siRNA (right). Human ST14A neurons were poorly transfected by Lipofectamine but were efficiently transfected by +36 GFP. DAPI channel, blue, was used to visualize DNA, thereby marking the position of cells; Cy3 channel, red, was used to visualize Cy3-tagged siRNA; GFP channel, green, was used to visualize GFP. Yellow indicates sites of co-localization between siRNA and GFP.

FIG. 12. Flow Cytometry Analysis of siRNA Transfection. LEFT: Lipofectamine. Each column corresponds to experiments performed with different transfection methods: lipofectamine (blue); and 20 nM+36 GFP (red). Each chart corresponds to experiments performed with different cell types: IMCD cells, PC12 cells, HeLa cells, 3T3L cells, and Jurkat cells. The X-axis represents measurements obtained from the Cy3 channel, which is a readout of siRNA fluorescence. The Y-axis represents cell count in flow cytometry experiments. Flow cytometry data indicate that cells were more efficiently transfected with siRNA using +36 GFP than Lipofectamine.

FIG. 13. siRNA Delivered with +36 GFP Can Induce Gene Knockdown. 50 nM GAPDH siRNA was transfected into five different cell types (HeLa, IMCD, 3T3L, PC12, and Jurkat cell lines) using either ˜2 μM lipofectamine 2000 (black bars) or 20 nM +36 GFP (green bars). The Y-axis represents GAPDH protein levels as a fraction of tubulin protein levels.

FIG. 14. Mechanistic Probes of Cell Penetration. HeLa cells were treated with one of a variety of probes for 30 minutes and were then treated with 5 nM +36 GFP. Samples included: (A) no probe; (B) 4° C. preincubation (inhibits energy-dependent processes); (C) 100 mM sucrose (inhibits clathrin-mediated endocytosis), left, and 25 μg/ml nystatin (disrupts caveolar function), right; (D) 25 μM cytochalisin B (inhibits macropinocytosis), left, and 5 μM monensin (inhibits endosome receptor recycling), right.

FIG. 15. Factors Contributing to Cell-Penetrating Activity. Charge magnitude was shown to contribute to cell-penetrating activity. In particular, +15 GFP or Lys₂₀₋₅₀ was shown not to penetrate cells. Left: 20 mM +15 GFP and 50 nM siRNA-Cy3. Middle: 20 nM +36 GFP. Right: 60 nM Lys₂₀₋₅₀ and 50 nM siRNA-Cy3. Hoescht channel, blue, was used to visualize DNA, thereby marking the position of cells; GFP channel, green, was used to visualize GFP.

FIG. 16. Supercharged GFP variants and their ability to penetrate cells. (A) Calculated electrostatic surface potential of GFP variants, colored from −25 kT/e (dark red) to +25 kT/e (dark blue). (B) Flow cytometry analysis showing amounts of internalized GFP in HeLa cells independently treated with 200 nM of each GFP variant and washed three times with PBS containing heparin to remove cell surface-bound GFP. (C) Flow cytometry analysis showing amounts of internalized +36 GFP (green) in HeLa, IMCD, 3T3-L, PC12, and Jurkat cells compared to background fluorescence in untreated cells (black).

FIG. 17. (A) Internalization of +36 GFP in HeLa cells after co-incubation for 1 hour at 37 C. (B) Inhibition of +36 GFP cell penetration in HeLa cells incubated at 4° C. for 1 hour. Cells were only partially washed to enable +36 GFP to remain partially bound to the cell surface. (C) and (D) +36 GFP internalization under the conditions in (A) but in the presence of caveolin-dependent endocytosis inhibitors filipin and nystatin, respectively. (E) +36 GFP internalization under the conditions in (A) but in the presence of the clathrin-dependent endocytosis inhibitor chlorpromazine. (F) Cellular localization of Alexa Fluor 647-labeled transferrin (red) and +36 GFP (green) 20 minutes after endocytosis. (G) Inhibition of +36 GFP internalization in HeLa cells in the presence of the actin polymerization inhibitor cytochalasin D. (H) Inhibition of +36 GFP internalization in HeLa cells treated with 80 mM sodium chlorate. (I) Internalization of +36 GFP in CHO cells incubated at 37° C. for 1 hour. (J) Lack of +36 GFP internalization in PDG-CHO cells. In (I) and (J) cell nuclei were stained with DAPI (blue).

FIG. 18. (A) Gel-shift assay showing unbound siRNA (33) stained by ethidium bromide to determine superpositive GFP:siRNA binding stoichiometry. 10 pmoles of siRNA was mixed with various molar ratios of each GFP for 10 minutes at 25° C., then analyzed by non-denaturing PAGE. The rightmost lane in each row shows a 100:1 mixture of sfGFP and siRNA. (B) Flow cytometry analysis showing levels of internalized siRNA in HeLa cells treated with a mixture of 50 nM Cy3-siRNA and 200 nM of +15, +25, or +36 GFP, followed by three heparin washes to remove non-internalized protein (see FIG. 22). Data from HeLa cells treated with siRNA but no transfection reagent is shown in black. (C) Flow cytometry analysis showing levels of Cy3-labeled siRNA delivered into HeLa, IMCD, 3T3-L, PC12, and Jurkat cells after incubation with a mixture of 50 nM Cy3-siRNA and either 200 nM +36 GFP (green) or ˜2 μM Lipofectamine 2000 (blue) in comparison to cells treated with siRNA without transfection reagent (black). Cells were washed before flow cytometry as described above. (D) Fluorescence microscopy images of stably adherent cell lines (HeLa, IMCD, and 3T3-L) 24 hours after a 4-hour treatment with 200 nM +36 GFP and 50 nM Cy3-siRNA. Each image is an overlay of three channels: blue (DAPI stain), red (Cy3-siRNA), and green (+36 GFP); yellow indicates the colocalization of red and green. Magnification for all three images was 40×.

FIG. 19. Suppression of GAPDH mRNA and protein levels resulting from siRNA delivery. (A) GAPDH mRNA level suppression in HeLa cells 48, 72, or 96 hours after treatment with 50 nM siRNA and ˜2 μM Lipofectamine 2000, or with 50 nM siRNA and 200 nM +36 GFP, as measured by RT-QPCR. Suppression levels shown are normalized to β-actin mRNA levels; 0% suppression is defined as the mRNA level in cells treated with ˜2 μM Lipofectamine 2000 and 50 nM scrambled negative control siRNA. (B) GAPDH protein level suppression in HeLa cells 48, 72, and 96 hours after treatment with siRNA and ˜2 μM Lipofectamine 2000, or with siRNA and 200 nM +36 GFP. (C) GAPDH protein level suppression in HeLa, IMCD, 3T3-L, PC12, and Jurkat cells 96 hours after treatment with 50 nM siRNA and ˜2 μM Lipofectamine 2000, 200 nM +36 GFP, or 200 nM +36 GFP-HA2. For (B) and (C), suppression levels shown are measured by Western blot and are normalized to β-tubulin protein levels; 0% suppression is defined as the protein level in cells treated with ˜2 μM Lipofectamine 2000 and a scrambled negative control siRNA. Values and error bars represent the mean and the standard deviation of three independent experiments in (A) and (B) and five independent experiments in (C).

FIG. 20. The siRNA transfection activities of a variety of cationic synthetic peptides compared with that of +15 and +36 GFP. Flow cytometry was used to measure the levels of internalized Cy3-siRNA in HeLa cells treated for 4 hours with a mixture of 50 nM Cy3-siRNA and either 200 nM or 2 μM of the peptide or protein shown.

FIG. 21. Plasmid DNA transfection into HeLa, IMCD, 3T3-L, PC 12, and Jurkat cells by Lipofectamine 2000, +36 GFP, or +36 GFP-HA2. Cells were treated with 800 ng pSV-β-galactosidase plasmid and 200 nM or 2 μM of +36 GFP or +36 GFP-HA2 for 4 hours. After 24 hours, β-galactosidase activity was measured using the β-Fluor kit (Novagen). Values and error bars represent the mean and standard deviation of three independent experiments.

FIG. 22. The effectiveness of the washing protocol used to remove cell surface-bound supercharged GFP. HeLa cells were treated with 200 nM +36 GFP at 4° C. (to block cell uptake of GFP, see the main text) for 1 hour. Cells were then washed three times (1 minute for each wash) with 4° C. PBS or with 4° C. 20 U/mL heparin sulfate in PBS, then analyzed by flow cytometry. Cells washed with PBS show significant GFP fluorescence presumably arising from cell-surface bound GFP. In contrast, cells washed with 20 U/mL heparin in PBS exhibit GFP fluorescence levels equivalent to untreated cells.

FIG. 23. Concentration dependence of +36 GFP cell penetration in HeLa cells. HeLa cells were treated with +36 GFP in serum-free media for 4 hours. Cells were trypsinized and replated in 10% FBS in DMEM on glass slides coated with Matrigel (BD Biosciences). After 24 hours at 37° C., cells were fixed with 4% formaldehyde in PBS, stained with DAPI, and imaged using a Leica DMRB inverted microscope. Magnification for all images is 20×.

FIG. 24. Fluorescence microscopy reveals no internalized Cy3-siRNA in IMCD and 3T3-L cells using Fugene 6 (Roche) transfection agent. Cells were treated with Fugene 6 in serum-free media for 4 hours following the manufacturer's protocol. Cells were trypsinized and pelleted. The trypsin-containing media was removed by aspiration and the cells were resuspended in 10% FBS in DMEM then plated on glass slides precoated with Matrigel™. Cells were allowed to adhere for 24 hours, fixed with 4% formaldehyde in PBS, stained with DAPI, and imaged using a Leica DMRB inverted microscope. Magnification for all images is 20×. No Cy3 fluorescence was observed (compare with FIG. 18D).

FIG. 25. (A) MTT cytotoxicity assay for five mammalian cell lines treated with 50 nM siRNA and ˜2 μM Lipofectamine 2000, +36 GFP, or +36 GFP-HA2. Data were taken 24 hours after treatment. Values and error bars reflect the mean and the standard deviation of three independent experiments. Cells treated with +36 GFP or +36 GFP-HA2 but without the MTT reagent did not exhibit significant absorbance under these conditions. (B) MTT cytotoxicity assay of HeLa cells treated with 50 nM siRNA and either 200 nM or 2 μM cationic polymer. Treatment with chloroquine or pyrene butyric acid proved cytotoxic (lanes 9 and 10, respectively).

FIG. 26. Gel-shift assay showing unbound linearized pSV-β-galactosidase plasmid DNA (Promega) to determine +36 GFP:plasmid DNA binding stoichiometry. In each lane 22 fmol of pSV-β-galactosidase linearized by EcoRI digestion was combined with various molar ratios of +36 GFP and incubated at 25° C. for 10 minutes. Samples were analyzed by electrophoresis at 140 V for 50 minutes on a 1% agarose gel containing ethidium bromide.

FIG. 27. SDS-PAGE analysis of purified GFP variants used in this work. The proteins were visualized by staining with Coomassie Blue. The migration points of molecular weight markers are listed on the left. Note that supercharged GFP migrates during SDS-PAGE in a manner that is partially dependent on theoretical net charge magnitude, rather than solely on actual molecular weight.

FIG. 28. Fluorescence spectra of all GFP analogs used in this study (10 nM each protein, excitation at 488 nm).

FIG. 29. (A) Representative Western blot data 4 days after treatment with ˜2 μM Lipofectamine 2000 and 50 nM negative control siRNA. (B) Representative Western blot data 4 days after treatment with 200 nM +36 GFP and 50 nM negative control siRNA. (C) Representative Western blot data showing GAPDH and β-tubulin levels 48, 72, and 96 hours after treatment with 50 nM GAPDH siRNA and either ˜2 μM Lipofectamine 2000 or 200 nM +36 GFP. (D) Representative Western blot data 4 days after treatment with ˜2 μM Lipofectamine 2000 and 50 nM GAPDH siRNA. (E) Representative Western blot data 4 days after treatment with 200 nM +36 GFP and 50 nM GAPDH siRNA. (F) Representative Western blot data 4 days after treatment with 200 nM +36 GFP-HA2 and 50 nM GAPDH siRNA. (G) Representative western blot data from HeLa cells four days after treatment with ˜2 μM Lipofectamine 2000 and 50 nM negative control siRNA, ˜2 μM Lipofectamine 2000 and 50 nM β-actin targeting siRNA, 200 nM +36 GFP and 50 nM β-actin targeting siRNA, or 200 nM +36 GFP and 50 nM negative control siRNA.

FIG. 30. Fluorescence microscopy reveals no internalized Cy3-siRNA or GFP in HeLa cells treated at either 4° C., or in HeLa cells pretreated with cytochalisin D (10 μg/mL). Image is of cells 1 hour after treatment with a solution containing 200 nM +36 GFP and 50 nM siRNA. Images were taken on an inverted spinning disk confocal microscope equipped with a filter to detect GFP emission. To facilitate visualization, cells were washed twice (one minute each) with 20 U/mL heparin in PBS to remove most (but not all) surface bound GFP-siRNA.

FIG. 31. (A) Dynamic Light Scattering (DLS) data showing the hydrodynamic radius (Hr) of particles formed from mixing 20 μM +36 GFP and 5 μM of a double-stranded RNA 20-mer. (B) Fluorescence microscopy image of the above sample. The image shown is an overlay of brightfield and GFP channel images; note that the larger features are actually smaller particles associated together as the sample dried. Scale bar=10 μm.

FIG. 32. (A) Digestion of +36 GFP and bovine serum albumin by proteinase K. 100 pmol of +36 GFP or bovine serum albumin (BSA) was treated with 0.6 units of proteinase K at 37° C. Samples were mixed with SDS protein loading buffer, heated to 90° C. for 10 minutes, and analyzed by SDS-PAGE on a 4-12% acrylamide gel staining with Coomassie Blue. (B) Stability of +36 GFP and BSA in murine serum. 100 pmol of each protein in PBS was mixed with 5 μL of murine serum to a total volume of 10 μL and incubated at 37° C. Samples were mixed with SDS protein loading buffer and heated to 90° C. for 10 minutes. The resulting mixture was analyzed by SDS-PAGE on a 4-12% acrylamide gel and the +36 GFP and BSA protein bands were revealed by Western blot. The bottom image is 5 μL of sample of +36 GFP-siRNA complexes (discussed in C) and analyzed for GFP by Western blot. (C) Stability of siRNA complexed with +36 GFP in murine serum. siRNA (10 pmol) was mixed with sfGFP (40 pmol) or +36 GFP (40 pmol), and incubated in 4 μL of PBS for 10 minutes at 25° C. The resulting solution was added to four volumes of mouse serum (20 μL total) and incubated at 37° C. for the indicated times, precipitated with ethanol, and analyzed by gel electrophoresis on a 15% acrylamide gel. (D) Stability of plasmid DNA complexed with +36 GFP or sfGFP in murine serum. Plasmid DNA (0.026 pmol) was mixed with 12.8 pmol of either +36 GFP or sfGFP in 4 μL of PBS for 10 minutes. To this solution was added 16 μL of mouse serum (20 μL total). Samples were incubated at 37° C. for the indicated times. DNA was isolated by extraction with phenol-chloroform and precipitation with ethanol, then analyzed by gel electrophoresis on a 1% agarose gel.

FIG. 33. Internalization of mCherry using (1) mCherry-TAT; (2) mCherry-Arg₉; and (3) mCherry-ALAL-+36 GFP in HeLa, PC12, and IMCD cell lines.

FIG. 34. Fluorescence microscopy images of HeLa, PC12, and IMCD cells four hours after treatment with 50 nM mCherry-ALAL-+36 GFP. Each image is an overlay of three channels: blue (DAPI stain for DNA), red (mCherry), and green (+36 GFP). Yellow indicates colocalization of red and green.

FIG. 35. Human proteins deliver siRNA to HeLa cells. (A) Human proteins were mixed at increasing mass ratios with siRNA and assayed for unbound siRNA by PAGE and ethidium bromide staining Decreasing band intensities demonstrate siRNA binding by human proteins. (B) Human proteins were mixed with Cy3-labelled siRNA and applied to HeLa cells for four hours. Cells were then washed and assayed for Cy3 fluorescence by flow cytometry. A shift of the peak to the right demonstrates siRNA internalization. (C) HeLa cells were transfected with siRNA using human proteins, incubated for three days, and assayed for degradation of a targeted mRNA. Targeted GAPDH mRNA levels were compared relative to β-actin mRNA levels. “Control” indicates use of a non-targeting siRNA. Lipofectamine 2000 was used as positive control.

### DETAILED DESCRIPTION OF CERTAIN EMBODIMENTS OF THE INVENTION

The present invention provides compositions, preparations, systems, and related methods for enhancing delivery of a protein or other agent to cells by supercharging the protein itself or by associating the protein or other agent (e.g., peptides, proteins, small molecules) with a supercharged protein. Such systems and methods generally comprise the use of supercharged proteins. In some embodiments, the supercharged protein itself is delivered to the interior of a cell, e.g., to cause a biological effect on the cell into which it penetrates for therapeutic benefit. Superchaged proteins can also be used to deliver other agents. For example, superpositively charged proteins may be associated with agents having a negative charge, e.g., nucleic acids (which typically have a net negative charge) or negatively charged peptides or proteins via electrostatic interactions to form complexes. Supernegatively charged proteins may be associated with agents having a positive charge. Agents to be delivered may also be associated with the supercharged protein through covalent linkages or other non-covalent interactions. In some embodiments, such compositions, preparations, systems, and methods involve altering the primary sequence of a protein in order to “supercharge” the protein (e.g., to generate a superpositively-charged protein). In certain embodiments, the inventive system uses a naturally occurring protein to form a complex. In certain embodiments, the inventive complex comprises a supercharged protein and one or more agents to be delivered (e.g., nucleic acid, protein, peptide, small molecule). In one example of cellular uptake, supercharged proteins have been found to be endocytosed by cells. The supercharged protein, or the supercharged protein mixed with an agent to be delivered to form a protein/agent complex, is effectively transfected into the cell. Mechanistic studies indicate the endocytosis of these complexes involves sulfated cell surface proteoglycans but does not involve clathrin or caveolin. In some embodiments, supercharged protein or complexes comprising supercharged proteins and one or more agents to be delivered are useful as therapeutic agents, diagnostic agents, or research tools. In some embodiments, an agent and/or supercharged protein may be therapeutically active. In some embodiments, a supercharged protein or complex is used to modulate the expression of a gene in a cell. In some embodiments, a supercharged protein or complex is used to modulate a biological pathway (e.g., a signaling pathway, a metabolic pathway) in a cell. In some embodiments, a supercharged protein or complex is used to inhibit the activity of an enzyme in a cell. In some embodiments, inventive supercharged proteins or complexes and/or pharmaceutical compositions thereof are administered to a subject in need thereof. In some embodiments, inventive supercharged proteins or complexes and/or compositions thereof are contacted with a cell under conditions effective to transfect the agent into a cell (e.g., human cells, mammalian cells, T-cells, neurons, stem cells, progenitor cells, blood cells, fibroblasts, epithelial cells, etc.). In some embodiments, delivery of a supercharged protein or complex to cells involves administering a supercharged protein or a complex comprising supercharged proteins associated with therapeutic agents to a subject in need thereof.

### Supercharged Proteins

Supercharged proteins can be produced by changing non-conserved amino acids on the surface of a protein to more polar or charged amino acid residues. The amino acid residues to be modified may be hydrophobic, hydrophilic, charged, or a combination thereof. Supercharged proteins can also be produced by the attachment of charged moieties to the protein in order to supercharge the protein. Supercharged proteins frequently are resistant to aggregation, have an increased ability to refold, resist improper folding, have improved solubility, and are generally more stable under a wide range of conditions, including denaturing conditions such as heat or the presence of a detergent.

Any protein may be modified using the inventive system to produce a supercharged protein. Natural as well as unnatural proteins (e.g., engineered proteins) may be modified. Example of proteins that may be modified include receptors, membrane bound proteins, transmembrane proteins, enzymes, transcription factors, extracellular proteins, therapeutic proteins, cytokines, messenger proteins, DNA-binding proteins, RNA-binding proteins, proteins involved in signal transduction, structural proteins, cytoplasmic proteins, nuclear proteins, hydrophobic proteins, hydrophilic proteins, etc. A protein to be modified may be derived from any species of plant, animal, and/or microorganism. In certain embodiments, the protein is a mammalian protein. In certain embodiments, the protein is a human protein. In certain embodiments, the protein is derived from an organism typically used in research. For example, the protein to be modified may be from a primate (e.g., ape, monkey), rodent (e.g., rabbit, hamster, gerbil), pig, dog, cat, fish (e.g., Danio rerio), nematode (e.g., C. elegans), yeast (e.g., Saccharomyces cervisiae), or bacteria (e.g., E. coli). In certain embodiments, the protein is non-immunogenic. In certain embodiments, the protein is non-antigenic. In certain embodiments, the protein does not have inherent biological activity or has been modified to have no biological activity. In certain embodiments, the protein is chosen based on its targeting ability. In certain embodiments, the protein is green fluorescent protein.

In some embodiments, the protein to be modified is one whose structure has been characterized, for example, by NMR or X-ray crystallography. In some embodiments, the protein to be modified is one whose structure has been correlated and/or related to biochemical activity (e.g., enzymatic activity, protein-protein interactions, etc.). In some embodiments, such information provides guidance for selection of amino acid residues to be modified or not modified (e.g., so that biological function is maintained or so that biological activity can be reduced or eliminated). In certain embodiments, the inherent biological activity of the protein is reduced or eliminated to reduce the risk of deleterious and/or undesired effects.

In some embodiments, the protein to be modified is one that is useful in the delivery of a nucleic acid or other agent to a cell. In some embodiments, the protein to be modified is an imaging, labeling, diagnostic, prophylactic, or therapeutic agent. In some embodiments, the protein to be modified is one that is useful for delivering an agent, e.g., a nucleic acid, to a particular cell. In some embodiments, the protein to be modified is one that has desired biological activity. In some embodiments, the protein to be modified is one that has desired targeting activity. In some embodiments, non-conserved surface residues of a protein of interest are identified and at least some of them replaced with a residue that is hydrophilic, polar, and/or charged at physiological pH. In some embodiments, non-conserved surface residues of a protein of interest are identified and at least some of them replaced with a residue that is positively charged at physiological pH.

The surface residues of the protein to be modified are identified using any method(s) known in the art. In certain embodiments, surface residues are identified by computer modeling of the protein. In certain embodiments, the three-dimensional structure of the protein is known and/or determined, and surface residues are identified by visualizing the structure of the protein. In some embodiments, surface residues are predicted using computer software. In certain particular embodiments, an Average Neighbor Atoms per Sidechain Atom (AvNAPSA) value is used to predict surface exposure. AvNAPSA is an automated measure of surface exposure which has been implemented as a computer program. A low AvNAPSA value indicates a surface exposed residue, whereas a high value indicates a residue in the interior of the protein. In certain embodiments, the software is used to predict the secondary structure and/or tertiary structure of a protein, and surface residues are identified based on this prediction. In some embodiments, the prediction of surface residues is based on hydrophobicity and hydrophilicity of the residues and their clustering in the primary sequence of the protein. Besides in silico methods, surface residues of the protein may also be identified using various biochemical techniques, for example, protease cleavage, surface modification, etc.

Optionally, of the surface residues, it is then determined which are conserved or important to the functioning of the protein. The step of determining which residues are conserved is optional when it is not necessary to preserve the underlying biological activity of the protein. Identification of conserved residues can be determined using any method known in the art. In certain embodiments, conserved residues are identified by aligning the primary sequence of the protein of interest with related proteins. These related proteins may be from the same family of proteins. For example, if the protein is an immunoglobulin, other immunoglobulin sequences may be used. Related proteins may also be the same protein from a different species. For example, conserved residues may be identified by aligning the sequences of the same protein from different species. To give but another example, proteins of similar function or biological activity may be aligned. Preferably, 2, 3, 4, 5, 6, 7, 8, 9, or different sequences are used to determine the conserved amino acids in the protein. In certain embodiments, a residue is considered conserved if over 50%, over 60%, over 70%, over 75%, over 80%, over 90%, or over 95% of the sequences have the same amino acid in a particular position. In other embodiments, the residue is considered conserved if over 50%, over 60%, over 70%, over 75%, over 80%, over 90%, or over 95% of the sequences have the same or a similar (e.g., valine, leucine, and isoleucine; glycine and alanine; glutamine and asparagine; or aspartate and glutamate) amino acid in a particular position. Many software packages are available for aligning and comparing protein sequences as described herein. As would be appreciated by one of skill in the art, either the conserved residues may be determined first or the surface residues may be determined first. The order does not matter. In certain embodiments, a computer software package may determine surface residues and conserved residues simultaneously. Important residues in the protein may also be identified by mutagenesis of the protein. For example, alanine scanning of the protein can be used to determine the important amino acid residues in the protein. In some embodiments, site-directed mutagenesis may be used. In certain embodiments, conserving the original biological activity of the protein is not important, and therefore, the steps of identifying the conserved residues and preserving them in the supercharged protein are not performed.

Each of the surface residues is identified as hydrophobic or hydrophilic. In certain embodiments, residues are assigned a hydrophobicity score. For example, each surface residue may be assigned an octanol/water logP value. Other hydrophobicity parameters may also be used. Such scales for amino acids have been discussed in: Janin, 1979, Nature, 277:491; Wolfenden et al., 1981, Biochemistry, 20:849; Kyte et al., 1982, J. Mol. Biol., 157:105; Rose et al., 1985, Science, 229:834; Cornette et al., 1987, J. Mol. Biol., 195:659; Charton and Charton, 1982, J. Theor. Biol., 99:629; each of which is incorporated by reference. Any of these hydrophobicity parameters may be used in the inventive method to determine which residues to modify. In certain embodiments, hydrophilic or charged residues are identified for modification.

At least one identified surface residue is then chosen for modification. In certain embodiments, hydrophobic residue(s) are chosen for modification. In other embodiments, hydrophilic and/or charged residue(s) are chosen for modification. In certain embodiments, more than one residue is chosen for modification. In certain embodiments, 1, 2, 3, 4, 5, 6, 7, 8, 9, or 10 of the identified residues are chosen for modification. In certain embodiments, over 10, over 15, over 20, or over 25 residues are chosen for modification. As would be appreciated by one of skill in the art, the larger the protein, the more residues that will need to be modified. Also, the more hydrophobic or susceptible to aggregation or precipitation the protein is, the more residues may need to be modified. In certain embodiments, multiple variants of a protein, each with different modifications, are produced and tested to determine the best variant in terms of delivery of a nucleic acid to a cell, stability, biocompatibility, and/or biological activity.

In certain embodiments, residues chosen for modification are mutated into more hydrophilic residues (including charged residues). Typically, residues are mutated into more hydrophilic natural amino acids. In certain embodiments, residues are mutated into amino acids that are charged at physiological pH. For example, a residue may be changed to an arginine, aspartate, glutamate, histidine, or lysine. In certain embodiments, all the residues to be modified are changed into the same different residue. For example, all the chosen residues are changed to a lysine residue. In other embodiments, the chosen residues are changed into different residues; however, all the final residues may be either positively charged or negatively charged at physiological pH. In certain embodiments, to create a negatively charged protein, all the residues to be mutated are converted to glutamate and/or aspartate residues. In certain embodiments, to create a positively charged protein, all the residues to be mutated are converted to lysine residues. For example, all the chosen residues for modification are asparagine, glutamine, lysine, and/or arginine, and these residues are mutated into aspartate or glutamate residues. To give but another example, all the chosen residues for modification are aspartate, glutamate, asparagine, and/or glutamine, and these residues are mutated into lysine. This approach allows for modifying the net charge on the protein to the greatest extent.

In some embodiments, a protein may be modified to keep the net charge on the modified protein the same as on the unmodified protein. In some embodiments, a protein may be modified to decrease the overall net charge on the protein while increasing the total number of charged residues on the surface. In certain embodiments, the theoretical net charge is increased by at least +1, at least +2, at least +3, at least +4, at least +5, at least +10, at least +15, at least +20, at least +25, at least +30, at least +35, or at least +40. In certain embodiments, the theoretical net charge is decreased by at least −1, at least −2, at least −3, at least −4, at least −5, at least −10, at least −15, at least −20, at least −25, at least −30, at least −35, or at least −40. In certain embodiments, the chosen amino acids are changed into non-ionic, polar residues (e.g., cysteine, serine, threonine, tyrosine, glutamine, asparagine).

In certain embodiments, the amino acid residues mutated to charged amino acids residues are separated from each other by at least 1, at least 2, at least 3, at least 4, at least 5, at least 6, at least 7, at least 8, at least 9, at least 10, at least 15, at least 20, or at least 25 amino acid residues. In certain embodiments, the amino acid residues mutated to positively charged amino acids residues (e.g., lysine) are separated from each other by at least 1, at least 2, at least 3, at least 4, at least 5, at least 6, at least 7, at least 8, at least 9, at least 10, at least 15, at least 20, or at least 25 amino acid residues. Typically, these intervening sequence are based on the primary amino acid of the protein being supercharged. In certain embodiments, only two charged amino acids are allowed to be in a row in a supercharged protein. In certain embodiments, only three or fewer charged amino acids are allowed to be in a row in a supercharged protein. In certain embodiments, only four or fewer charged amino acids are allowed to be in a row in a supercharged protein. In certain embodiments, only five or fewer charged amino acids are allowed to be in a row in a supercharged protein.

In certain embodiments, a surface exposed loop, helix, turn, or other secondary structure may contain only 1, 2, 3, 4, 5, 6, 7, 8, 9, or 10 charged residues. Distributing the charged residues over the protein typically is thought to allow for more stable proteins. In certain embodiments, only 1, 2, 3, 4, or 5 residues per 15-20 amino acids of the primary sequence are mutated to charged amino acids (e.g., lysine). In certain embodiments, on average only 1, 2, 3, 4, or 5 residues per 10 amino acids of the primary sequence are mutated to charged amino acids (e.g., lysine). In certain embodiments, on average only 1, 2, 3, 4, or 5 residues per 15 amino acids of the primary sequence are mutated to charged amino acids (e.g., lysine). In certain embodiments, on average only 1, 2, 3, 4, or 5 residues per 20 amino acids of the primary sequence are mutated to charged amino acids (e.g., lysine). In certain embodiments, on average only 1, 2, 3, 4, or 5 residues per 25 amino acids of the primary sequence are mutated to charged amino acids (e.g., lysine). In certain embodiments, on average only 1, 2, 3, 4, or 5 residues per 30 amino acids of the primary sequence are mutated to charged amino acids (e.g., lysine).

In certain embodiments, at least 50%, at least 60%, at least 70%, at least 80%, or at least 90% of the mutated charged amino acid residues of the supercharged protein are solvent exposed. In certain embodiments, at least 50%, at least 60%, at least 70%, at least 80%, or at least 90% of the mutated charged amino acids residues of the supercharged protein are on the surface of the protein. In certain embodiments, less than 5%, less than 10%, less than 20%, less than 30%, less than 40%, less than 50% of the mutated charged amino acid residues are not solvent exposed. In certain embodiments, less than 5%, less than 10%, less than 20%, less than 30%, less than 40%, less than 50% of the mutated charged amino acid residues are internal amino acid residues.

In some embodiments, amino acids are selected for modification using one or more predetermined criteria. For example, to generate a superpositively charged protein, AvNAPSA values may be used to identify aspartic acid, glutamic acid, asparagine, and/or glutamine residues with AvNAPSA values below a certain threshold value, and one or more (e.g., all) of these residues may be changed to lysines. In some embodiments, to generate a superpositively charged protein, AvNAPSA is used to identify aspartic acid, glutamic acid, asparagine, and/or glutamine residues with AvNAPSA below a certain threshold value, and one or more (e.g., all) of these are changed to arginines. In some embodiments, to generate a supernegative protein, AvNAPSA is used to identify asparagine, glutamine, lysine, and/or arginine residues with AvNAPSA values below a certain threshold value, and one or more (e.g., all) of these are changed to aspartic acid residues. In some embodiments, to generate a supernegatively charged protein, AvNAPSA is used to identify asparagine, glutamine, lysine, and/or arginine residues with AvNAPSA values below a certain threshold value, and one or more (e.g., all) of these are changed to glutamic acid residues. In some embodiments, the certain threshold value is 40 or below. In some embodiments, the certain threshold value is 35 or below. In some embodiments, the certain threshold value is 30 or below. In some embodiments, the certain threshold value is 25 or below. In some embodiments, the certain threshold value is 20 or below. In some embodiments, the certain threshold value is 19 or below, 18 or below, 17 or below, 16 or below, 15 or below, 14 or below, 13 or below, 12 or below, 11 or below, 10 or below, 9 or below, 8 or below, 7 or below, 6 or below, 5 or below, 4 or below, 3 or below, 2 or below, or 1 or below. In some embodiments, the certain threshold value is 0.

In some embodiments, solvent-exposed residues are identified by the number of neighbors. In general, residues that have more neighbors are less solvent-exposed than residues that have fewer neighbors. In some embodiments, solvent-exposed residues are identified by half sphere exposure, which accounts for the direction of the amino acid side chain (Hamelryck, 2005, Proteins, 59:8-48; incorporated herein by reference). In some embodiments, solvent-exposed residues are identified by computing the solvent exposed surface area, accessible surface area, and/or solvent excluded surface of each residue. See, e.g., Lee et al., J. Mol. Biol. 55(3):379-400, 1971; Richmond, J. Mol. Biol. 178:63-89, 1984; each of which is incorporated herein by reference.

The desired modifications or mutations in the protein may be accomplished using any techniques known in the art. Recombinant DNA techniques for introducing such changes in a protein sequence are well known in the art. In certain embodiments, the modifications are made by site-directed mutagenesis of the polynucleotide encoding the protein. Other techniques for introducing mutations are discussed in Molecular Cloning: A Laboratory Manual, 2nd Ed., ed. by Sambrook, Fritsch, and Maniatis (Cold Spring Harbor Laboratory Press: 1989); the treatise, Methods in Enzymology (Academic Press, Inc., N.Y.); Ausubel et al. Current Protocols in Molecular Biology (John Wiley & Sons, Inc., New York, 1999); each of which is incorporated herein by reference. The modified protein is expressed and tested. In certain embodiments, a series of variants is prepared, and each variant is tested to determine its biological activity and its stability. The variant chosen for subsequent use may be the most stable one, the most active one, or the one with the greatest overall combination of activity and stability. After a first set of variants is prepared an additional set of variants may be prepared based on what is learned from the first set. Variants are typically created and overexpressed using recombinant techniques known in the art.

Supercharged proteins may be further modified. Proteins including supercharged proteins can be modified using techniques known to those of skill in the art. For example, supercharged proteins may be modified chemically or biologically. One or more amino acids may be added, deleted, or changed from the primary sequence. For example, a polyhistidine tag or other tag may be added to the supercharged protein to aid in the purification of the protein. Other peptides or proteins may be added onto the supercharged protein to alter the biological, biochemical, and/or biophysical properties of the protein. For example, an endosomolytic peptide may be added to the primary sequence of the supercharged protein, or a targeting peptide may be added to the primary sequence of the supercharged protein. Other modifications of the supercharged protein include, but are not limited to, post-translational modifications (e.g., glycosylation, phosphorylation, acylation, lipidation, farnesylation, acetylation, proteolysis, etc.). In certain embodiments, the supercharged protein may be modified to reduce its immunogenicity. In certain embodiments, the supercharged protein may be modified to enhance its ability to delivery a nucleic acid to a cell. In certain embodiments, the supercharged protein may be conjugated to a polymer. For example, the protein may be PEGylated by conjugating the protein to a polyethylene glycol (PEG) polymer. One of skill in the art can envision a multitude of ways of modifying the supercharged protein without departing from the scope of the present invention. Methods described herein allow supercharging proteins by imposing changes in the protein sequence of the protein to be supercharged. Other methods can be used to produce supercharged proteins without modification of the protein sequence. For example, moeties that alter charge can be attached to proteins (e.g., by chemical or enzymatic reactions) to provide surface charge to achieve supercharging. In certain embodiments, the method of modifying proteins described in Shaw et al., Protein Science 17:1446, 2008 is used to supercharge a protein.

The international PCT patent application (PCT/US07/70254, filed Jun. 1, 2007, published as WO 2007/143574 on Dec. 13, 2007, entitled “Protein Surface Remodeling”; incorporated herein by reference) and U.S. Provisional patent applications (U.S. Ser. No. 60/810,364, filed Jun. 2, 2006, and U.S. Ser. No. 60/836,607, filed Aug. 9, 2006; both of which are entitled “Protein Surface Remodeling”; and both of which are incorporated herein by reference) describe the design and creation of variants of several different proteins. These variants have been shown to be more stable and to retain their fluorescence. For example, a green fluorescent protein (GFP) from Aequorea victoria is described in GenBank Accession Number P42212, incorporated herein by reference. The amino acid sequence of this wild type GFP is as follows:

(SEQ ID NO: 1)

MSKGEELFTGVVPILVELDGDVNGHKFSVSGEGEGDATYGKLTLKFI
CTTGKLPVPWPTLVTTFSYGVQCFSRYPDHMKQHDFFKSAMPEGYV
QERTIFFKDDGNYKTRAEVKFEGDTLVNRIELKGIDFKEDGNILGHKL
EYNYNSHNVYIMADKQKNGIKVNFKIRHNIEDGSVQLADHYQQNTPI
GDGPVLLPDNHYLSTQSALSKDPNEKRDHMVLLEFVTAAGITHGMDE
LYK

Wild type GFP has a theoretical net charge of −7. Variants with a theoretical net charge of −29, −30, −25, +15, +25, +36, +48, and +49 have been created. Even after heating the +36 GFP to 95° C., 100% of the variant protein is soluble and the protein retains ≧70% of its fluorescence. +15, +25, and +36 GFP have been found to be particularly useful in transfecting nucleic acids into cells. In particular, +36 GFP has been found to be highly cell permeable and capable of efficiently delivering nucleic acids into a variety of mammalian cells, including cell lines resistant to transfection using other transfection methods. Therefore, GFP or other proteins with a net charge of at least +25, at least +30, at least +35, or at least +40 are thought to be particularly useful in transfecting nucleic acids into a cell.

The amino acid sequences of the variants of GFP that have been created include:

GFP-NEG7
(SEQ ID NO: 2)
MGHHHHHHGGASKGEELFTGVVPILVELDGDVNGHKFSVRGEGEGDATNGKLTLK
FICTTGKLPVPWPTLVTTLTYGVQCFSRYPDHMKQHDFFKSAMPEGYVQERTISFKD
DGTYKTRAEVKFEGDTLVNRIELKGIDFKEDGNILGHKLEYNFNSHNVYITADKQKN
GIKANFKIRHNVEDGSVQLADHYQQNTPIGDGPVLLPDNHYLSTQSALSKDPNEKRD
HMVLLEFVTAAGITHGMDELYK
GFP-NEG25
(SEQ ID NO: 3)
MGHHHHHHGGASKGEELFTGVVPILVELDGDVNGHEFSVRGEGEGDATEGELTLKF
ICTTGELPVPWPTLVTTLTYGVQCFSRYPDHMKQHDFFKSAMPEGYVQERTISFKDD
GTYKTRAEVKFEGDTLVNRIELKGIDFKEDGNILGHKLEYNFNSHDVYITADKQENGI
KAEFEIRHNVEDGSVQLADHYQQNTPIGDGPVLLPDDHYLSTESALSKDPNEDRDHM
VLLEFVTAAGIDHGMDELYK
GFP-NEG29
(SEQ ID NO: 4)
MGHHHHHHGGASKGEELFDGEVPILVELDGDVNGHEFSVRGEGEGDATEGELTLKF
ICTTGELPVPWPTLVTTLTYGVQCFSRYPDHMDQHDFFKSAMPEGYVQERTISFKDD
GTYKTRAEVKFEGDTLVNRIELKGIDFKEDGNILGHKLEYNFNSHDVYITADKQENGI
KAEFEIRHNVEDGSVQLADHYQQNTPIGDGPVLLPDDHYLSTESALSKDPNEDRDHM
VLLEFVTAAGIDHGMDELYK
GFP-NEG30
(SEQ ID NO: 5)
MGHHHHHHGGASKGEELFDGVVPILVELDGDVNGHEFSVRGEGEGDATEGELTLKF
ICTTGELPVPWPTLVTTLTYGVQCFSDYPDHMDQHDFFKSAMPEGYVQERTISFKDD
GTYKTRAEVKFEGDTLVNRIELKGIDFKEDGNILGHKLEYNFNSHDVYITADKQENGI
KAEFEIRHNVEDGSVQLADHYQQNTPIGDGPVLLPDDHYLSTESALSKDPNEDRDHM
VLLEFVTAAGIDHGMDELYK
GFP-POS15
(SEQ ID NO: 6)
MGHHHHHHGGASKGERLFTGVVPILVELDGDVNGHKFSVRGEGEGDATRGKLTLK
FICTTGKLPVPWPTLVTTLTYGVQCFSRYPKHMKRHDFFKSAMPEGYVQERTISFKK
DGTYKTRAEVKFEGRTLVNRIELKGRDFKEKGNILGHKLEYNFNSHNVYITADKRKN
GIKANFKIRHNVKDGSVQLADHYQQNTPIGRGPVLLPRNHYLSTRSALSKDPKEKRD
HMVLLEFVTAAGITHGMDELYK
GFP-POS25
(SEQ ID NO: XX
MGHHHHHHGGASKGERLFTGVVPILVELDGDVNGHKFSVRGKGKGDATRGKLTLK
FICTTGKLPVPWPTLVTTLTYGVQCFSRYPKHMKRHDFFKSAMPKGYVQERTISFKK
DGTYKTRAEVKFEGRTLVNRIKLKGRDFKEKGNILGHKLRYNFNSHNVYITADKRK
NGIKANFKIRHNVKDGSVQLADHYQQNTPIGRGPVLLPRNHYLSTRSALSKDPKEKR
DHMVLLEFVTAAGITHGMDELYK
GFP-POS36
(SEQ ID NO: 7)
MGHHHHHHGGASKGERLFRGKVPILVELKGDVNGHKFSVRGKGKGDATRGKLTLK
FICTTGKLPVPWPTLVTTLTYGVQCFSRYPKHMKRHDFFKSAMPKGYVQERTISFKK
DGKYKTRAEVKFEGRTLVNRIKLKGRDFKEKGNILGHKLRYNFNSHKVYITADKRK
NGIKAKFKIRHNVKDGSVQLADHYQQNTPIGRGPVLLPRNHYLSTRSKLSKDPKEKR
DHMVLLEFVTAAGIKHGRDERYK
GFP-POS42
(SEQ ID NO: 8)
MGHHHHHHGGRSKGKRLFRGKVPILVELKGDVNGHKFSVRGKGKGDATRGKLTLK
FICTTGKLPVPWPTLVTTLTYGVQCFSRYPKHMKRHDFFKSAMPKGYVQERTISFKK
DGKYKTRAEVKFEGRTLVNRIKLKGRDFKEKGNILGHKLRYNFNSHKVYITADKRK
NGIKAKFKIRHNVKDGSVQLADHYQQNTPIGRGPVLLPRKHYLSTRSKLSKDPKEKR
DHMVLLEFVTAAGIKHGRKERYK
GFP-POS48
(SEQ ID NO: 9)
MGHHHHHHGGRSKGKRLFRGKVPILVKLKGDVNGHKFSVRGKGKGDATRGKLTLK
FICTTGKLPVPWPTLVTTLTYGVQCFSRYPKHMKRHDFFKSAMPKGYVQERTISFKK
DGKYKTRAEVKFKGRTLVNRIKLKGRDFKEKGNILGHKLRYNFNSHKVYITADKRK
NGIKAKFKIRHNVKDGSVQLAKHYQQNTPIGRGPVLLPRKHYLSTRSKLSKDPKEKR
DHMVLLEFVTAAGIKHGRKERYK
GFP-POS49
(SEQ ID NO: 10)
MGHHHHHHGGRSKGKRLFRGKVPILVKLKGDVNGHKFSVRGKGKGDATRGKLTLK
FICTTGKLPVPWPTLVTTLTYGVQCFSRYPKHMKRHDFFKSAMPKGYVQERTISFKK
DGKYKTRAEVKFKGRTLVNRIKLKGRDFKEKGNILGHKLRYNFNSHKVYITADKRK
NGIKAKFKIRHNVKDGSVQLAKHYQQNTPIGRGPVLLPRKHYLSTRSKLSKDPKEKR
DHMVLKEFVTAAGIKHGRKERYK

In order to promote the escape of the supercharged protein, or delivered agent, e.g., nucleic acid, from the endosomes, a supercharged protein may be fused to or associated with a protein, peptide, or other entity known to enhance endosome degradation or lysis of the endosome. In certain embodiments, the peptide is hemagglutinin 2 (HA2) peptide which is know to enhance endosome degradation. In certain particular embodiments, HA2 peptide is fused to supercharged GFP (e.g., +36 GFP). In certain particular embodiments, the fused protein is of the sequence:

+36 GFP-HA2

(SEQ ID NO: XX)

MGHHHHHHGGASKGERLFRGKVPILVELKGDVNGHKFSVRGKGKGDATRG
KLTLKFICTTGKLPVPWPTLVTTLTYGVQCFSRYPKHMKRHDFFKSAMPK
GYVQERTISFKKDGKYKTRAEVKFEGRTLVNRIKLKGRDFKEKGNILGHK
LRYNFNSHKVYITADKRKNGIKAKFKIRHNVKDGSVQLADHYQQNTPIGR
GPVLLPRNHYLSTRSKLSKDPKEKRDHMVLLEFVTAAGIKHGRDERYKG
SAGSAAGSGEFGLFGAIAGFIENGWEGMIDG

In certain embodiments, the endosomolytic peptide is melittin peptide (GIGAVLKVLTTGLPALISWIKRKRQQ, SEQ ID NO: XX) (Meyer et al. JACS 130(11):3272-3273, 2008; which is incorporated herein by reference). In certain embodiments, the melittin peptide is modified by one, two, three, four, or five amino acid substitutions, deletions, and/or additions. In certain embodiments, the melittin peptide is of the sequence: CIGAVLKVLTTGLPALISWIKRKRQQ (SEQ ID NO: XX). In certain particular embodiments, the melittin peptide is fued to supercharged GFP (e.g., +36 GFP).

In certain embodiments, the endosomolytic peptide is penetratin peptide (RQIKIWFQNRRMKWKK-amide, SEQ ID NO: XX), bovine PrP (1-30) peptide (MVKSKIGSWILVLFVAMWSDVGLCKKRPKP-amide, SEQ ID NO: XX), MPGΔ^{NLS} peptide (which lacks a functional nuclear localization sequence because of a K->S substitution) (GALFLGWLGAAGSTMGAPKSKRKV, SEQ ID NO: XX), TP-10 peptide (AGYLLGKINLKALAALAKKIL-amide, SEQ ID NO: XX), and/or EB1 peptide (LIRLWSHLIHIWFQNRRLKWKKK-amide, SEQ ID NO: XX) (Lundberg et al. 2007, FASEB J. 21:2664; incorporated herein by reference). In certain embodiments, the penetratin, PrP (1-30), MPG, TP-10, and/or EB1 peptide is modified by one, two, three, four, or five amino acid substitutions, deletions, and/or additions. In certain particular embodiments, the PrP (1-30), MPG, TP-10, and/or EB1 peptide is fued to supercharged GFP (e.g., +36 GFP).

Other peptides or proteins may also be fused to the supercharged protein. For example, a targeting peptide may be fused to the supercharged protein in order to selectively deliver the supercharged protein, or associated agent, e.g., nucleic acid, to a particular cell type. Peptides or proteins that enhance the transfection of the nucleic acid may also be used. In certain embodiments, the peptide fused to the supercharged protein is a peptide hormone. In certain embodiments, the peptide fused to the supercharged protein is a peptide ligand.

As would be appreciated by one of skill in the art, homologous proteins are also considered to be within the scope of this invention. For example, any protein that includes a stretch of about 20, about 30, about 40, about 50, or about 100 amino acids which are about 40%, about 50%, about 60%, about 70%, about 80%, about 90%, about 95%, or about 100% identical to any of the above sequences can be utilized in accordance with the invention. Alternatively or additionally, addition and deletion variants can be utilized in accordance with the invention. In certain embodiments, any GFP with a mutated residue as shown in any of the above sequences can be utilized in accordance with the invention. In certain embodiments, a protein sequence to be utilized in accordance with the invention includes 2, 3, 4, 5, 6, 7, 8, 9, 10, or more mutations as shown in any of the sequences above.

Other proteins that may be supercharged and used, e.g., in the delivery of agents, e.g., nucleic acids, include other GFP-style fluorescent proteins. In certain embodiments, the supercharged protein is a supercharged version of blue fluorescent protein. In certain embodiments, the supercharged protein is a supercharged version of cyan fluorescent protein. In certain embodiments, the supercharged protein is a supercharged version of yellow fluorescent protein. Exemplary fluorescent proteins include, but are not limited to, enhanced green fluorescent protein (EGFP), AcGFP, TurboGFP, Emerald, Azami Green, ZsGreen, EBFP, Sapphire, T-Sapphire, ECFP, mCFP, Cerulean, CyPet, AmCyan1, Midori-Ishi Cyan, mTFP1 (Teal), enhanced yellow fluorescent protein (EYFP), Topaz, Venus, mCitrine, YPet, PhiYFP, ZsYellow1, mBanana, Kusabira Orange, mOrange, dTomato, dTomato-Tandem, DsRed, DsRed2, DsRed-Express (T1), DsRed-Monomer, mTangerine, mStrawberry, AsRed2, mRFP1, JRed, mCherry, HcRed1, mRaspberry, HcRed1, HcRed-Tandem, mPlum, and AQ143.

Yet other proteins that may be supercharged and used, e.g., in the delivery of an agent, e.g., nucleic acids, include histone components or histone-like proteins. In certain embodiments, the histone component is histone linker H1. In certain embodiments, the histone component is core histone H2A. In certain embodiments, the histone component is core histone H2B. In certain embodiments, the histone component is core histone H3. In certain embodiments, the histone component is core histone H4. In certain embodiments, the protein is the archael histone-linke protein, HPhA. In certain embodiments, the protein is the bacterial histone-like protein, TmHU.

Other proteins that may be supercharged and used, e.g., in the delivery of an agent, e.g., nucleic acids, include high-mobility-group proteins (HMGs). In certain embodiments, the protein is HMG1. In certain embodiments, the protein is HMG17. In certain embodiments, the protein is HMG1-2.

Other proteins that may be supercharged and used, e.g., in the delivery of an agent, e.g., nucleic acids, include anti-cancer agents, such as anti-apoptotic agents, cell cycle regulators, etc.

Other proteins that may be supercharged and used, e.g., in the delivery of an agent, e.g., nucleic acids, are enzymes, including, but not limited to, amylases, pectinases, hydrolases, proteases, glucose isomerase, lipases, phytases, etc. In some embodiments, proteins that may be supercharged and used, e.g., in the delivery of an agent, e.g., nucleic acids, are lysosomal enzymes, including, but not limited to, alglucerase, imiglucerase, agalsidase beta, α-1-iduronidase, acid α-glucosidase, iduronate-2-sulfatase, N-acetylgalactosamine-4-sulfatase, etc. (Wang et al., 2008, NBT, 26:901-08; incorporated herein by reference).

Other proteins that may be supercharged and used, e.g., in the delivery of an agent, e.g., nucleic acids, are presented in Table 1. Some of the proteins listed in Table 1 include a listing of residues that may be modified in order to supercharge those proteins. The identity of the residues was identified computationally by downloading a PDB file of the protein of interest. The residues of the pdb file were sorted by ascending avNapsa values, and the first 15 ASP, GLU, ASN or GLN residues were proposed for mutation to LYS.

PDB files, by convention, number amino acids by their order in the wild type protein. The PDB file, however, may not contain the full length wildtype protein. The input protein sequence is the sequence of the amino acids that are included in the PDB. The proposed mutations provide the number of the amino acid in the full length wildtype protein and also the number in the input protein sequence. The proposed mutations are provided in the following format: Wildtype residue_Chain:Residue Number in Wildtype Protein Chain (Residue Number in Input Chain)_Proposed Residue. Wildtype residue refers to the identity of the amino acid in the wild type protein. Chain refers to the designation of the peptide chain of the specified mutation. Residue number in wildtype protein refers to the number of the amino acid in the designated protein chain of the specified mutation in the full length wild type protein. Residue number in input chain refers to the number of the amino acid in the designated protein chain that was included in the analyzed PDB.

TABLE 1
Exemplary Proteins that can be Supercharged

| | | 15 Possible Exemplary Mutations to Generate |
|---|---|---|
| | | Positively Supercharged Protein |
| PROTEIN TYPE | | Wildtype residue_Chain: Residue Number in |
| Protein Subtype | | Wildtype Protein Chain (Residue Number in Input |
| Protein (PDB #) | Input Protein Sequence | Chain)_Proposed Residue |
| MEMBRANE | | |
| PROTEINS | | |
| Cystic fibrosis | Chain A: | ASP_A: 513(102)_LYS, GLU_A: 514(103)_LYS, |
| transmembrane | STTEVVMENVTAFWEEGFGELFE | GLU_A: 656(238)_LYS, GLU_A: 474(64)_LYS, |
| conductance | KAKGTPVLKDINFKIERGQLLAVA | GLU_A: 528(117)_LYS, GLU_A: 535(124)_LYS, |
| regulator (CFTR) | GSTGAGKTSLLMMIMGELEPSEG | ASN_A: 635(220)_LYS, ASN_A: 494(84)_LYS, |
| (2bbs) | KIKHSGRISFCSQNSWIMPGTIKEN | ASP_A: 579(164)_LYS, ASP_A: 639(224)_LYS, |
| | IIGVSYDEYRYRSVIKACQLEEDIS | GLN_A: 652(234)_LYS, GLU_A: 402(15)_LYS, |
| | KFAEKDNIVLITLSGGQRARISLAR | ASP_A: 565(150)_LYS, GLU_A: 664(246)_LYS, |
| | AVYKDADLYLLDSPFGYLDVLTE | GLU_A: 403(16)_LYS, |
| | KEIFESCVCKLMANKTRILVTSKM | |
| | EHLKKADKILILHEGSSYFYGTFSE | |
| | LQNLRPDFSSKLMSFDQFSAERRN | |
| | SILTETLHRFSL (SEQ ID NO: XX) | |
| RECEPTORS | | |
| Cytokine Receptors | | |
| Type I | | |
| EPO receptor (1eer) | Chain B: | ASP_B: 8(1)_LYS, ASP_B: 133(126)_LYS, |
| | DPKFESKAALLAARGPEELLCFTE | ASP_B: 61(54)_LYS, GLU_B: 134(127)_LYS, |
| | RLEDLVCFWEEAASAGVGPGQYS | GLU_B: 147(140)_LYS, ASN_B: 185(178)_LYS, |
| | FSYQLEDEPWKLCRLHQAPTARG | GLU_B: 12(5)_LYS, GLU_B: 62(55)_LYS, |
| | AVRFWCSLPTADTSSFVPLELRVT | GLU_B: 24(17)_LYS, GLN_B: 164(157)_LYS, |
| | AASGAPRYHRVIHINEVVLLDAPV | GLN_B: 170(163)_LYS, GLU_B: 60(53)_LYS, |
| | GLVARLADESGHVVLRWLPPPET | GLU_B: 25(18)_LYS, GLN_B: 52(45)_LYS, |
| | PMTSHIRYEVDVSAGQGAGSVQR | GLU_B: 173(166)_LYS |
| | VEILEGRTECVLSNLRGRTRYTFA | |
| | VRARMAEPSFGGFWSEWSEPVSL | |
| | LT (SEQ ID NO: XX) | |
| GM-CSF receptor | | |
| G-CSF receptor | Chain B: | ASN_B: 84(82)_LYS, ASP_B: 57(55)_LYS, |
| (2d9q) | CGHISVSAPIVHLGDPITASCIIKQN | ASP_B: 213(211)_LYS, ASP_B: 158(156)_LYS, |
| | CSHLDPEPQILWRLGAELQPGGRQ | GLN_B: 222(213)_LYS, GLU_B: 253(244)_LYS, |
| | QRLSDGTQESIITLPHLNHTQAFLS | ASP_B: 149(147)_LYS, GLN_B: 234(225)_LYS, |
| | CSLNWGNSLQILDQVELRAGYPP | GLN_B: 160(158)_LYS, GLU_B: 270(261)_LYS, |
| | AIPHNLSCLMNLTTSSLICQWEPG | GLU_B: 45(43)_LYS, GLN_B: 145(143)_LYS, |
| | PETHLPTSFTLKSFKSRGNCQTQG | GLU_B: 308(299)_LYS, ASN_B: 28(26)_LYS, |
| | DSILDCVPKDGQSHCSIPRKHLLL | GLU_B: 93(91)_LYS |
| | YQNMGIWVQAENALGTSMSPQL | |
| | CLDPMDVVKLEPPMLRTMDPQA | |
| | GCLQLSWEPWQPGLHINQKCELR | |
| | HKPQRGEASWALVGPLPLEALQY | |
| | ELCGLLPATAYTLQIRCIRWPLPG | |
| | HWSDWSPSLELRTTE (SEQ ID NO: | |
| | XX) | |
| Growth hormone | Chain B: | ASN_B: 72(33)_LYS, GLN_B: 166(121)_LYS, |
| receptor (1axi) | EPKFTKCRSPERETFSCHWTDEGP | GLU_B: 183(138)_LYS, ASP_B: 190(145)_LYS, |
| | IQLFYTRRNEWKECPDYVSAGEN | GLU_B: 79(34)_LYS, GLU_B: 32(1)_LYS, |
| | SCYFNSSFTSIAIPYCIKLTSNGGT | ASP_B: 52(21)_LYS, GLU_B: 61(22)_LYS, |
| | VDEKCFSVDEIVQPDPPIALNWTL | ASN_B: 182(137)_LYS, ASN_B: 114(69)_LYS, |
| | LNVSLTGIHADIQVRWEAPRNADI | ASN_B: 218(173)_LYS, GLU_B: 91(46)_LYS, |
| | QKGWMVLEYELQYKEVNETKW | ASN_B: 162(117)_LYS, ASN_B: 97(52)_LYS, |
| | KMMDPILTTSVPVYSLKVDKEYE | ASN_B: 143(98)_LYS |
| | VRVRSKQRNSGNYGEFSEVLYVT | |
| | LPQM (SEQ ID NO: XX) | |
| Type II | | |
| Interferon receptors | | |
| Immunoglobulin | | |
| superfamily receptors | | |
| IL-1 receptor | Chain B: | ASN_B: 30(25)_LYS, ASN_B: 32(27)_LYS, |
| | CKEREEKIILVSSANEIDVRPCPLN | ASN_B: 102(97)_LYS, ASN_B: 135(130)_LYS, |
| | PNEHKGTITWYKDDSKTPVSTEQ | ASP_B: 253(248)_LYS, ASP_B: 254(249)_LYS, |
| | ASRIHQHKEKLWFVPAKVEDSGH | ASP_B: 153(148)_LYS, GLU_B: 252(247)_LYS, |
| | YYCVVRNSSYCLRIKISAKFVENE | GLU_B: 8(3)_LYS, ASP_B: 44(39)_LYS, |
| | PNLCYNAQAIFKQKLPVAGDGGL | GLU_B: 72(67)_LYS, ASN_B: 136(131)_LYS, |
| | VCPYMEFFKNENNELPKLQWYK | GLU_B: 137(132)_LYS, ASN_B: 204(199)_LYS, |
| | DCKPLLLDNIHFSGVKDRLIVMNV | ASN_B: 269(264)_LYS |
| | AEKHRGNYTCHASYTYLGKQYPI | |
| | TRVIEFITLEENKPTRPVIVSPANET | |
| | MEVDLGSQIQLICNVTGQLSDIAY | |
| | WKWNGSVIDEDDPVLGEDYYSV | |
| | ENPANKRRSTLITVLNISEIESRFY | |
| | KHPFTCFAKNTHGIDAAYIQLIYP | |
| | VT (SEQ ID NO: XX) | |
| C-kit receptor | | |
| TNF receptor family | | |
| TNF alpha receptor | Chain A: | GLU_A: 171(159)_LYS, ASN_A: 172(160)_LYS, |
| (CD120) (1ext) | SVCPQGKYIHPQNNSICCTKCHKG | GLN_B: 24(14)_LYS, GLN_A: 24(12)_LYS, |
| | TYLYNDCPGPGQDTDCRECESGS | GLU_A: 109(97)_LYS, ASN_A: 25(13)_LYS, |
| | FTASENHLRHCLSCSKCRKEMGQ | GLN_A: 169(157)_LYS, ASN_B: 25(15)_LYS, |
| | VEISSCTVDRDTVCGCRKNQYRH | GLU_B: 109(99)_LYS, ASN_A: 110(98)_LYS, |
| | YWSENLFQCFNCSLCLNGTVHLS | GLN_B: 48(38)_LYS, GLN_A: 17(5)_LYS, |
| | CQEKQNTVCTCHAGFFLRENECV | ASN_A: 26(14)_LYS, GLN_A: 48(36)_LYS, |
| | SCSNCKKSLECTKLCLPQIEN | GLN_B: 17(7)_LYS |
| | Chain B: | |
| | MDSVCPQGKYIHPQNNSICCTKC | |
| | HKGTYLYNDCPGPGQDTDCRECE | |
| | SGSFTASENHLRHCLSCSKCRKE | |
| | MGQVEISSCTVDRDTVCGCRKNQ | |
| | YRHYWSENLFQCFNCSLCLNGTV | |
| | HLSCQEKQNTVCTCHAGFFLREN | |
| | ECVSCSNCKKSLECTKLCLP (SEQ | |
| | ID NO: XX) | |
| Lymphotoxin β | Chain A: | ASN_A: 313(1)_LYS, ASP_A: 487(175)_LYS, |
| receptor (1rf3) | NTGLLESQLSRHDQMLSVHDIRL | ASN_A: 453(141)_LYS, GLU_A: 463(151)_LYS, |
| | ADMDLRFQVLETASYNGVLIWKI | ASP_A: 500(188)_LYS, GLU_A: 318(6)_LYS, |
| | RDYKRRKQEAVMGKTLSLYSQPF | GLN_A: 320(8)_LYS, ASP_A: 325(13)_LYS, |
| | YTGYFGYKMCARVYLNGDGMG | GLU_A: 346(34)_LYS, GLU_A: 417(105)_LYS, |
| | KGTHLSLFFVIMRGEYDALLPWPF | ASN_A: 481(169)_LYS, ASP_A: 503(191)_LYS, |
| | KQKVTLMLMDQGSSRRHLGDAF | GLN_A: 326(14)_LYS, ASP_A: 337(25)_LYS, |
| | KPDPNSSSFKKPTGEMNIASGCPV | ASP_A: 339(27)_LYS |
| | FVAQTVLENGTYIKDDTIFIKVIVD | |
| | TSDLPDP (SEQ ID NO: XX) | |
| CD40L (1aly) | Chain A: | ASP_A: 117(2)_LYS, GLN_A: 118(3)_LYS, |
| | GDQNPQIAAHVISEASSKTTSVLQ | ASN_A: 119(4)_LYS, ASN_A: 151(36)_LYS, |
| | WAEKGYYTMSNNLVTLENGKQL | ASN_A: 157(42)_LYS, GLN_A: 166(51)_LYS, |
| | TVKRQGLYYIYAQVTFCSNREASS | GLN_A: 186(71)_LYS, GLU_A: 202(87)_LYS, |
| | QAPFIASLCLKSPGRFERILLRAAN | GLU_A: 230(115)_LYS, GLN_A: 121(6)_LYS, |
| | THSSAKPCGQQSIHLGGVFELQPG | ASN_A: 150(35)_LYS, GLU_A: 156(41)_LYS, |
| | ASVFVNVTDPSQVSHGTGFTSFGL | ASN_A: 210(95)_LYS, GLN_A: 220(105)_LYS, |
| | LKL (SEQ ID NO: XX) | GLU_A: 182(67)_LYS |
| Chemokine receptors | | |
| IL-8 receptor | | |
| CCR1 | | |
| CXCR4 | | |
| TGF beta receptors | | |
| TGF beta receptors 1, | Chain A: | ASN_A: 344(144)_LYS, ASN_A: 456(252)_LYS, |
| 2, 3 (1vjy) | IARTIVLQESIGKGRFGEVWRGKW | ASN_A: 270(70)_LYS, GLN_A: 324(124)_LYS, |
| | RGEEVAVKIFSSREERSWFREAEI | GLN_A: 448(244)_LYS, GLU_A: 227(27)_LYS, |
| | YQTVMLRHENILGFIAADNKDNG | ASP_A: 366(166)_LYS, ASP_A: 430(226)_LYS, |
| | TWTQLWLVSDYHEHGSLFDYLN | ASP_A: 435(231)_LYS, GLN_A: 498(294)_LYS, |
| | RYTVTVEGMIKLALSTASGLAHL | GLN_A: 208(8)_LYS, ASP_A: 269(69)_LYS, |
| | HMEIVGTQGKPAIAHRDLKSKNIL | GLU_A: 447(243)_LYS, ASN_A: 453(249)_LYS, |
| | VKKNGTCCIADLGLAVRHDSATD | GLN_A: 494(290)_LYS |
| | TIDIRVGTKRYMAPEVLDDSINMK | |
| | HFESFKRADIYAMGLVFWEIARR | |
| | CSIGGIHEDYQLPYYDLVPSDPSV | |
| | EEMRKVVCEQKLRPNIPNRWQSC | |
| | EALRVMAKIMRECWYANGAARL | |
| | TALRIKKTLSQLSQQEGIKM (SEQ | |
| | ID NO: XX) | |
| TRANSCRIPTION | | |
| FACTORS | | |
| p53 (2vuk) | Chain A: | ASN_A: 210(115)_LYS, ASN_A: 288(193)_LYS, |
| | SVPSQKTYQGSYGFRLGFLHSGTA | GLN_B: 167(73)_LYS, ASN_B: 210(116)_LYS, |
| | KSVTCTYSPALNKLFCQLAKTCPV | ASN_B: 288(194)_LYS, GLU_A: 287(192)_LYS, |
| | QLWVDSTPPPGTRVRAMAIYKQS | GLU_B: 287(193)_LYS, ASP_A: 208(113)_LYS, |
| | QHMTEVVRRCPHHERCSDSDGLA | GLU_A: 224(129)_LYS, ASP_B: 208(114)_LYS, |
| | PPQHLIRVEGNLRAEYLDDRNTFR | GLU_B: 224(130)_LYS, ASP_A: 148(53)_LYS, |
| | HSVVVPCEPPEVGSDCTTIHYNY | ASP_A: 186(91)_LYS, ASP_B: 148(54)_LYS, |
| | MCYSSCMGGMNRRPILTIITLEDS | ASN_A: 131(36)_LYS |
| | SGNLLGRDSFEVRVCACPGRDRR | |
| | TEEENLR (SEQ ID NO: XX) | |
| | Chain B: | |
| | SSVPSQKTYQGSYGFRLGFLHSGT | |
| | AKSVTCTYSPALNKLFCQLAKTCP | |
| | VQLWVDSTPPPGTRVRAMAIYKQ | |
| | SQHMTEVVRRCPHHERCSDSDGL | |
| | APPQHLIRVEGNLRAEYLDDRNTF | |
| | RHSVVVPCEPPEVGSDCTTIHYNY | |
| | MCYSSCMGGMNRRPILTIITLEDS | |
| | SGNLLGRDSFEVRVCACPGRDRR | |
| | TEEENLR (SEQ ID NO: XX) | |
| NF-kappaB (2o61) | Chain B: | ASP_B: 38(2)_LYS, ASN_B: 75(39)_LYS, |
| | MDGPYLQILEQPKQRGFRFRYVC | ASN_B: 288(252)_LYS, GLU_B: 287(251)_LYS, |
| | EGPSHGGLPGASSEKNKKSYPQV | ASP_B: 188(152)_LYS, GLU_B: 286(250)_LYS, |
| | KICNYVGPAKVIVQLVTNGKNIHL | ASP_B: 318(282)_LYS, GLU_B: 60(24)_LYS, |
| | HAHSLVGKHCEDGICTVTAGPKD | GLU_B: 73(37)_LYS, GLN_B: 185(149)_LYS, |
| | MVVGFANLGILHVTKKKVFETLE | ASP_B: 220(184)_LYS, ASP_B: 336(300)_LYS, |
| | ARMTEACIRGYNPGLLVHPDLAY | ASP_B: 172(136)_LYS, GLU_B: 179(143)_LYS, |
| | LQAEGGGDRQLGDREKELIRQAA | GLU_B: 192(156)_LYS |
| | LQQTKEMDLSVVRLMFTAFLPDS | |
| | TGSFTRRLEPVVSDAIYDSKAPNA | |
| | SNLKIVRMDRTAGCVTGGEEIYLL | |
| | CDKVQKDDIQIRFYEEEENGGVW | |
| | EGFGDFSPTDVHRQFAIVFKTPKY | |
| | KDINITKPASVFVQLRRKSDLETSE | |
| | PKPFLYYPE (SEQ ID NO: XX) | |
| Additional exemplary | | |
| transcript. factors can | | |
| be found in Table 2 | | |
| ENZYMES | | |
| Misc enzymes | | |
| Tissue plasminogen | Chain A: TTCCGLRQY (SEQ ID NO: | ASP_B: 110(102)_LYS, GLN_B: 60(47)_LYS, |
| activator (1rtf) | XX) | GLU_B: 60(48)_LYS, ASP_B: 110(102)_LYS, |
| | Chain B: | ASP_B: 204(204)_LYS, ASP_B: 97(88)_LYS, |
| | IKGGLFADIASHPWQAAIFAKHHR | ASP_B: 127(122)_LYS, ASN_B: 186(186)_LYS, |
| | RGGERFLCGGILISSCWILSAAHCF | GLN_B: 60(47)_LYS, GLU_B: 60(48)_LYS, |
| | QQQQQEEEEERRRRRFFFFFPPPPP | ASN_B: 173(170)_LYS, ASP_B: 240(240)_LYS, |
| | PHHLTVILGRTYRVVPGEEEQKFE | GLN_B: 60(47)_LYS, GLU_B: 60(48)_LYS, |
| | VEKYIVHKEFDDDTYDNDIALLQ | GLU_B: 78(69)_LYS |
| | LKSSSSSDDDDDSSSSSSSSSSRRR | |
| | RRCAQESSVVRTVCLPPADLQLPD | |
| | WTECELSGYGKHEALSPFYSERL | |
| | KEAHVRLYPSSRCTTTSSSQQQHL | |
| | LNRTVTDNMLCAGDTTTRRRSSS | |
| | NNNLHDACQGDSGGPLVCLNDG | |
| | RMTLVGIISWGLGCGGQQKDVPG | |
| | VYTKVTNYLDWIRDNMRP (SEQ | |
| | ID NO: XX) | |
| Factor IX | Chain A: | ASN_A: 95(80)_LYS, ASP_B: 104(19)_LYS, |
| | VVGGEDAKPGQFPWQVVLNGKV | GLU_A: 60(44)_LYS, GLU_A: 204(194)_LYS, |
| | DAFCGGSIVNEKWIVTAAHCVEE | GLU_A: 240(230)_LYS, GLU_B: 119(34)_LYS, |
| | TTGVKITVVAGEHNIEETEHTEQK | ASN_B: 120(35)_LYS, GLU_A: 74(59)_LYS, |
| | RNVIRIIPHHNYNNNAAAAAAINK | GLU_A: 75(60)_LYS, ASN_A: 93(78)_LYS, |
| | YNHDIALLELDEPLVLNSYVTPICI | ASN_A: 97(84)_LYS, GLU_A: 127(114)_LYS, |
| | ADKEYTTTNNNIIIFLKFGSGYVSG | GLU_A: 186(175)_LYS, ASN_B: 105(20)_LYS, |
| | WGRVFHKGRSALVLQYLRVPLV | GLU_A: 60(44)_LYS |
| | DRATCLRSTKFTIYNNMFCAGGFF | |
| | HEGGGRRDSCQGDSGGPHVTEVE | |
| | GTSFLTGIISWGEECAAMMKGKY | |
| | GIYTKVSRYVNWIKEKTKLT (SEQ | |
| | ID NO: XX) | |
| | Chain B: | |
| | MTCNIKNGRCEQFCKNSADNKVV | |
| | CSCTEGYRLAENQKSCEPAVPFPC | |
| | GRVSVSQTSK (SEQ ID NO: XX) | |
| deoxyribonuclease I | | |
| (rhDNase) | | |
| Enzyme Replacement | | |
| glucocerebrosidase | Chain A: | GLU_A: −1(1)_LYS, GLU_A: 72(71)_LYS, |
| | EFARPCIPKSFGYSSVVCVCNATY | GLN_A: 497(496)_LYS, ASP_A: 27(29)_LYS, |
| | CDSFDPPALGTFSRYESTRSGRRM | ASN_A: 59(58)_LYS, GLN_A: 73(72)_LYS, |
| | ELSMGPIQANHTGTGLLLTLQPEQ | GLN_A: 143(142)_LYS, GLU_A: 151(150)_LYS, |
| | KFQKVKGFGGAMTDAAALNILAL | GLU_A: 222(221)_LYS, ASN_A: 270(269)_LYS, |
| | SPPAQNLLLKSYFSEEGIGYNIIRV | GLN_A: 440(439)_LYS, ASP_A: 453(452)_LYS, |
| | PMASCDFSIRTYTYADTPDDFQLH | ASN_A: 333(332)_LYS, ASN_A: 275(274)_LYS, |
| | NFSLPEEDTKLKIPLIHRALQLAQR | ASN_A: 442(441)_LYS |
| | PVSLLASPWTSPTWLKTNGAVNG | |
| | KGSLKGQPGDIYHQTWARYFVKF | |
| | LDAYAEHKLQFWAVTAENEPSAG | |
| | LLSGYPFQCLGFTPEHQRDFIARD | |
| | LGPTLANSTHHNVRLLMLDDQRL | |
| | LLPHWAKVVLTDPEAAKYVHGIA | |
| | VHWYLDFLAPAKATLGETHRLFP | |
| | NTMLFASEACVGSKFWEQSVRLG | |
| | SWDRGMQYSHSIITNLLYHVVGW | |
| | TDWNLALNPEGGPNWVRNFVDS | |
| | PIIVDITKDTFYKQPMFYHLGHFS | |
| | KFIPEGSQRVGLVASQKNDLDAV | |
| | ALMHPDGSAVVVVLNRSSKDVPL | |
| | TIKDPAVGFLETISPGYSIHTYLWH | |
| | RQ (SEQ ID NO: XX) | |
| alpha galactosidase A | Chain A: | GLU_A: 103(72)_LYS, GLN_A: 57(26)_LYS, |
| | LDNGLARTPTMGWLHWERFMCN | GLU_A: 58(27)_LYS, GLU_A: 178(147)_LYS, |
| | LDCQEEPDSCISEKLFMEMAELM | ASP_A: 101(70)_LYS, ASP_A: 175(144)_LYS, |
| | VSEGWKDAGYEYLCIDDCWMAP | GLN_A: 212(181)_LYS, GLN_A: 306(275)_LYS, |
| | QRDSEGRLQADPQRFPHGIRQLA | GLN_A: 333(302)_LYS, ASP_A: 335(304)_LYS, |
| | NYVHSKGLKLGIYADVGNKTCAG | GLU_A: 59(28)_LYS, GLN_A: 111(80)_LYS, |
| | FPGSFGYYDIDAQTFADWGVDLL | ASN_A: 215(184)_LYS, GLU_A: 251(220)_LYS, |
| | KFDGCYCDSLENLADGYKHMSL | GLU_A: 358(327)_LYS |
| | ALNRTGRSIVYSCEWPLYMWPFQ | |
| | KPNYTEIRQYCNHWRNFADIDDS | |
| | WKSIKSILDWTSFNQERIVDVAGP | |
| | GGWNDPDMLVIGNFGLSWNQQV | |
| | TQMALWAIMAAPLFMSNDLRHIS | |
| | PQAKALLQDKDVIAINQDPLGKQ | |
| | GYQLRQGDNFEVWERPLSGLAW | |
| | AVAMINRQEIGGPRSYTIAVASLG | |
| | KGVACNPACFITQLLPVKRKLGFY | |
| | EWTSRLRSHINPTGTVLLQLENTM | |
| | (SEQ ID NO: XX) | |
| arylsulfatase-A | Chain A: | ASN_A: 350(331)_LYS, GLU_A: 103(84)_LYS, |
| (iduronidase, α-L-) | RPPNIVLIFADDLGYGDLGCYGHP | GLU_A: 451(428)_LYS, GLN_A: 215(196)_LYS, |
| | SSTTPNLDQLAAGGLRFTDFYVPV | ASP_A: 216(197)_LYS, GLU_A: 424(405)_LYS, |
| | SLPSRAALLTGRLPVRMGMYPGV | ASP_A: 267(248)_LYS, GLU_A: 131(112)_LYS, |
| | LVPSSRGGLPLEEVTVAEVLAARG | ASP_A: 411(392)_LYS, GLN_A: 454(431)_LYS, |
| | YLTGMAGKWHLGVGPEGAFLPP | GLN_A: 465(442)_LYS, GLN_A: 51(33)_LYS, |
| | HQGFHRFLGIPYSHDQGPCQNLTC | ASN_A: 158(139)_LYS, ASP_A: 207(188)_LYS, |
| | FPPATPCDGGCDQGLVPIPLLANL | GLN_A: 371(352)_LYS |
| | SVEAQPPWLPGLEARYMAFAHDL | |
| | MADAQRQDRPFFLYYASHHTHYP | |
| | QFSGQSFAERSGRGPFGDSLMELD | |
| | AAVGTLMTAIGDLGLLEETLVIFT | |
| | ADNGPETMRMSRGGCSGLLRCG | |
| | KGTTYEGGVREPALAFWPGHIAP | |
| | GVTHELASSLDLLPTLAALAGAPL | |
| | PNVTLDGFDLSPLLLGTGKSPRQS | |
| | LFFYPSYPDEVRGVFAVRTGKYK | |
| | AHFFTQGSAHSDTTADPACHASSS | |
| | LTAHEPPLLYDLSKDPGENYNLLG | |
| | ATPEVLQALKQLQLLKAQLDAAV | |
| | TFGPSQVARGEDPALQICCHPGCT | |
| | PRPACCHCP (SEQ ID NO: XX) | |
| arylsulfatase B (N- | Chain A: | GLU_A: 229(187)_LYS, ASN_A: 188(146)_LYS, |
| acetylgalactos-amine- | SRPPHLVFLLADDLGWNDVGFHG | GLU_A: 249(207)_LYS, GLU_A: 250(208)_LYS, |
| 4-sulfatase) (1fsu) | SRIRTPHLDALAAGGVLLDNYYT | ASN_A: 366(324)_LYS, GLN_A: 456(397)_LYS, |
| | QPLTPSRSQLLTGRYQIRTGLQHQI | ASN_A: 458(399)_LYS, ASP_A: 125(83)_LYS, |
| | IWPCQPSCVPLDEKLLPQLLKEAG | ASN_A: 225(183)_LYS, ASP_A: 256(214)_LYS, |
| | YTTHMVGKWHLGMYRKECLPTR | GLU_A: 490(431)_LYS, GLU_A: 201(159)_LYS, |
| | RGFDTYFGYLLGSEDYYSHERCT | ASN_A: 208(166)_LYS, GLN_A: 259(217)_LYS, |
| | LIDALNVTRCALDFRDGEEVATG | ASN_A: 398(356)_LYS |
| | YKNMYSTNIFTKRAIALITNHPPE | |
| | KPLFLYLALQSVHEPLQVPEEYLK | |
| | PYDFIQDKNRHHYAGMVSLMDE | |
| | AVGNVTAALKSSGLWNNTVFIFS | |
| | TDNGGQTLAGGNNWPLRGRKWS | |
| | LWEGGVRGVGFVASPLLKQKGV | |
| | KNRELIHISDWLPTLVKLARGHTN | |
| | GTKPLDGFDVWKTISEGSPSPRIEL | |
| | LHNIDPNFVDSSPCSAFNTSVHAAI | |
| | RHGNWKLLTGYPGCGYWFPPPSQ | |
| | YNVSEIPSSDPPTKTLWLFDIDRDP | |
| | EERHDLSREYPHIVTKLLSRLQFY | |
| | HKHSVPVYFPAQDPRCDPKATGV | |
| | WGPWM (SEQ ID NO: XX) | |
| galactosylcera- | | |
| midase | | |
| beta-galactosidase | | |
| beta-hexosaminidase | Chain A: | GLN_A: 528(492)_LYS, GLU_A: 151(115)_LYS, |
| A (2gjx) | LWPWPQNFQTSDQRYVLYPNNFQ | ASP_A: 123(87)_LYS, GLU_A: 523(487)_LYS, |
| | FQYDVSSAAQPGCSVLDEAFQRY | GLU_A: 527(491)_LYS, GLU_A: 111(75)_LYS, |
| | RDLLFGTLEKNVLVVSVVTPGCN | GLN_A: 237(201)_LYS, ASP_A: 34(12)_LYS, |
| | QLPTLESVENYTLTINDDQCLLLS | ASN_A: 43(21)_LYS, ASN_A: 42(20)_LYS, |
| | ETVWGALRGLETFSQLVWKSAEG | GLN_A: 106(70)_LYS, ASN_A: 295(259)_LYS, |
| | TFFINKTEIEDFPRFPHRGLLLDTS | GLU_A: 447(411)_LYS, ASP_A: 492(456)_LYS, |
| | RHYLPLSSILDTLDVMAYNKLNV | ASN_A: 518(482)_LYS |
| | FHWHLVDDPSFPYESFTFPELMRK | |
| | GSYNPVTHIYTAQDVKEVIEYARL | |
| | RGIRVLAEFDTPGHTLSWGPGIPG | |
| | LLTPCYSGSEPSGTFGPVNPSLNN | |
| | TYEFMSTFFLEVSSVFPDFYLHLG | |
| | GDEVDFTCWKSNPEIQDFMRKKG | |
| | FGEDFKQLESFYIQTLLDIVSSYGK | |
| | GYVVWQEVFDNKVKIQPDTIIQV | |
| | WREDIPVNYMKELELVTKAGFRA | |
| | LLSAPWYLNRISYGPDWKDFYVV | |
| | EPLAFEGTPEQKALVIGGEACMW | |
| | GEYVDNTNLVPRLWPRAGAVAE | |
| | RLWSNKLTSDLTFAYERLSHFRCE | |
| | LLRRGVQAQPLNVGFCEQEFEQ | |
| | (SEQ ID NO: XX) | |
| Hexosaminidase A | Chain A: | ASP_B: 317(245)_LYS, ASP_A: 123(87)_LYS, |
| and B (2gjx) | LWPWPQNFQTSDQRYVLYPNNFQ | ASP_B: 518(446)_LYS, ASP_C: 317(246)_LYS, |
| | FQYDVSSAAQPGCSVLDEAFQRY | GLN_C: 475(404)_LYS, GLU_A: 111(75)_LYS, |
| | RDLLFGTLEKNVLVVSVVTPGCN | GLN_B: 475(403)_LYS, ASP_C: 518(447)_LYS, |
| | QLPTLESVENYTLTINDDQCLLLS | GLU_D: 111(75)_LYS, GLN_D: 528(492)_LYS, |
| | ETVWGALRGLETFSQLVWKSAEG | ASP_A: 34(12)_LYS, GLN_A: 528(492)_LYS, |
| | TFFINKTEIEDFPRFPHRGLLLDTS | ASN_B: 327(255)_LYS, GLN_B: 373(301)_LYS, |
| | RHYLPLSSILDTLDVMAYNKLNV | ASP_B: 523(451)_LYS |
| | FHWHLVDDPSFPYESFTFPELMRK | |
| | GSYNPVTHIYTAQDVKEVIEYARL | |
| | RGIRVLAEFDTPGHTLSWGPGIPG | |
| | LLTPCYSGSEPSGTFGPVNPSLNN | |
| | TYEFMSTFFLEVSSVFPDFYLHLG | |
| | GDEVDFTCWKSNPEIQDFMRKKG | |
| | FGEDFKQLESFYIQTLLDIVSSYGK | |
| | GYVVWQEVFDNKVKIQPDTIIQV | |
| | WREDIPVNYMKELELVTKAGFRA | |
| | LLSAPWYLNRISYGPDWKDFYVV | |
| | EPLAFEGTPEQKALVIGGEACMW | |
| | GEYVDNTNLVPRLWPRAGAVAE | |
| | RLWSNKLTSDLTFAYERLSHFRCE | |
| | LLRRGVQAQPLNVGFCEQEFEQ | |
| | (SEQ ID NO: XX) | |
| | Chain B: | |
| | PALWPLPLSVKMTPNLLHLAPENF | |
| | YISHSPNSTAGPSCTLLEEAFRRYH | |
| | GYIFGTQVQQLLVSITLQSECDAF | |
| | PNISSDESYTLLVKEPVAVLKANR | |
| | VWGALRGLETFSQLVYQDSYGTF | |
| | TINESTIIDSPRFSHRGILIDTSRHY | |
| | LPVKIILKTLDAMAFNKFNVLHW | |
| | HIVDDQSFPYQSITFPELSNKGSYS | |
| | LSHVYTPNDVRMVIEYARLRGIR | |
| | VLPEFDTPGHTLSWGKGQKDLLT | |
| | PCYSDSFGPINPTLNTTYSFLTTFF | |
| | KEISEVFPDQFIHLGGDEVEFKCW | |
| | ESNPKIQDFMRQKGFGTDFKKLES | |
| | FYIQKVLDIIATINKGSIVWQEVFD | |
| | DKAKLAPGTIVEVWKDSAYPEEL | |
| | SRVTASGFPVILSAPWYLDLISYG | |
| | QDWRKYYKVEPLDFGGTQKQKQ | |
| | LFIGGEACLWGEYVDATNLTPRL | |
| | WPRASAVGERLWSSKDVRDMDD | |
| | AYDRLTRHRCRMVERGIAAQPLY | |
| | AGYCN (SEQ ID NO: XX) | |
| | Chain C: | |
| | PALWPLPLSVKMTPNLLHLAPENF | |
| | YISHSPNSTAGPSCTLLEEAFRRYH | |
| | GYIFGTQVQQLLVSITLQSECDAF | |
| | PNISSDESYTLLVKEPVAVLKANR | |
| | VWGALRGLETFSQLVYQDSYGTF | |
| | TINESTIIDSPRFSHRGILIDTSRHY | |
| | LPVKIILKTLDAMAFNKFNVLHW | |
| | HIVDDQSFPYQSITFPELSNKGSYS | |
| | LSHVYTPNDVRMVIEYARLRGIR | |
| | VLPEFDTPGHTLSWGKGQKDLLT | |
| | PCYSLDSFGPINPTLNTTYSFLTTF | |
| | FKEISEVFPDQFIHLGGDEVEFKC | |
| | WESNPKIQDFMRQKGFGTDFKKL | |
| | ESFYIQKVLDIIATINKGSIVWQEV | |
| | FDDKAKLAPGTIVEVWKDSAYPE | |
| | ELSRVTASGFPVILSAPWYLDLISY | |
| | GQDWRKYYKVEPLDFGGTQKQK | |
| | QLFIGGEACLWGEYVDATNLTPR | |
| | LWPRASAVGERLWSSKDVRDMD | |
| | DAYDRLTRHRCRMVERGIAAQPL | |
| | YAGYCN (SEQ ID NO: XX) | |
| | Chain D: | |
| | LWPWPQNFQTSDQRYVLYPNNFQ | |
| | FQYDVSSAAQPGCSVLDEAFQRY | |
| | RDLLFGTLEKNVLVVSVVTPGCN | |
| | QLPTLESVENYTLTINDDQCLLLS | |
| | ETVWGALRGLETFSQLVWKSAEG | |
| | TFFINKTEIEDFPRFPHRGLLLDTS | |
| | RHYLPLSSILDTLDVMAYNKLNV | |
| | FHWHLVDDPSFPYESFTFPELMRK | |
| | GSYNPVTHIYTAQDVKEVIEYARL | |
| | RGIRVLAEFDTPGHTLSWGPGIPG | |
| | LLTPCYSGSEPSGTFGPVNPSLNN | |
| | TYEFMSTFFLEVSSVFPDFYLHLG | |
| | GDEVDFTCWKSNPEIQDFMRKKG | |
| | FGEDFKQLESFYIQTLLDIVSSYGK | |
| | GYVVWQEVFDNKVKIQPDTIIQV | |
| | WREDIPVNYMKELELVTKAGFRA | |
| | LLSAPWYLNRISYGPDWKDFYVV | |
| | EPLAFEGTPEQKALVIGGEACMW | |
| | GEYVDNTNLVPRLWPRAGAVAE | |
| | RLWSNKLTSDLTFAYERLSHFRCE | |
| | LLRRGVQAQPLNVGFCEQEFEQ | |
| | (SEQ ID NO: XX) | |
| SMPD1 gene product | | |
| NPC1 and NPC2 | | |
| (transmembrane | | |
| proteins) | | |
| ASAH1 (N- | | |
| acylsphingosine | | |
| amidohydrolase (acid | | |
| ceramidase) 1) | | |
| alpha-glucosidase | | |
| phenylalanine | Chain A: | ASP_A: 338(221)_LYS, GLU_A: 360(243)_LYS, |
| hydroxylase (PAH) | VPWFPRTIQELDRFANQILSYGAE | ASN_A: 376(259)_LYS, GLU_A: 381(264)_LYS, |
| (1j8u) | LDADHPGFKDPVYRARRKQFADI | GLN_A: 172(55)_LYS, GLU_A: 316(199)_LYS, |
| | AYNYRHGQPIPRVEYMEEEKKTW | ASN_A: 133(16)_LYS, ASP_A: 151(34)_LYS, |
| | GTVFKTLKSLYKTHACYEYNHIFP | ASN_A: 167(50)_LYS, GLU_A: 178(61)_LYS, |
| | LLEKYCGFHEDNIPQLEDVSQFLQ | ASP_A: 145(28)_LYS, GLU_A: 181(64)_LYS, |
| | TCTGFRLRPVAGLLSSRDFLGGLA | GLN_A: 134(17)_LYS, ASP_A: 143(26)_LYS, |
| | FRVFHCTQYIRHGSKPMYTPEPDI | GLU_A: 182(65)_LYS |
| | CHELLGHVPLFSDRSFAQFSQEIG | |
| | LASLGAPDEYIEKLATIYWFTVEF | |
| | GLCKQGDSIKAYGAGLLSSFGELQ | |
| | YCLSEKPKLLPLELEKTAIQNYTV | |
| | TEFQPLYYVAESFNDAKEKVRNF | |
| | AATIPRPFSVRYDPYTQRIEVL | |
| | (SEQ ID NO: XX) | |
| Cathepsin A | Chain A: | GLN_A: 215(215)_LYS, ASN_A: 216(216)_LYS, |
| | APDQDEIQRLPGLAKQPSFRQYSG | GLN_A: 327(327)_LYS, ASP_A: 404(404)_LYS, |
| | YLKSSGSKHLHYWFVESQKDPEN | ASP_A: 3(3)_LYS, ASP_A: 111(111)_LYS, |
| | SPVVLWLNGGPGCSSLDGLLTEH | GLN_A: 394(394)_LYS, GLN_A: 450(450)_LYS, |
| | GPFLVQPDGVTLEYNPYSWNLIA | ASP_A: 110(110)_LYS, GLN_A: 165(165)_LYS, |
| | NVLYLESPAGVGFSYSDDKFYAT | ASP_A: 266(266)_LYS, GLN_A: 288(288)_LYS, |
| | NDTEVAQSNFEALQDFFRLFPEYK | GLU_A: 326(326)_LYS, ASN_A: 388(388)_LYS, |
| | NNKLFLTGESYAGIYIPTLAVLVM | ASN_A: 448(448)_LYS |
| | QDPSMNLQGLAVGNGLSSYEQND | |
| | NSLVYFAYYHGLLGNRLWSSLQT | |
| | HCCSQNKCNFYDNKDLECVTNLQ | |
| | EVARIVGNSGLNIYNLYAPCAGG | |
| | VPSHFRYEKDTVVVQDLGNIFTRL | |
| | PLKRMWHQALLRSGDKVRMDPP | |
| | CTNTTAASTYLNNPYVRKALNIPE | |
| | QLPQWDMCNFLVNLQYRRLYRS | |
| | MNSQYLKLLSSQKYQILLYNGDV | |
| | DMACNFMGDEWFVDSLNQKME | |
| | VQRRPWLVKYGDSGEQIAGFVKE | |
| | FSHIAFLTIKGAGHMVPTDKPLAA | |
| | FTMFSRFLNKQPY (SEQ ID NO: | |
| | XX) | |
| STRUCTURAL | | |
| PROTEINS | | |
| Collagen | | |
| Elastin | | |
| Actin (1lot) | Chain B: | |
| | DETTALVCDNGSGLVKAGFAGDD | ASP_B: 3(1)_LYS, GLU_B: 4(2)_LYS, |
| | APRAVFPSIVGRPRDSYVGDEAQS | ASP_B: 244(230)_LYS, ASP_B: 51(38)_LYS, |
| | KRGILTLKYPIEGIITNWDDMEKI | ASP_B: 288(274)_LYS, GLN_B: 246(232)_LYS, |
| | WHHTFYNELRVAPEEHPTLLTEA | GLU_B: 167(153)_LYS, ASP_B: 286(272)_LYS, |
| | PLNPKANREKMTQIMFETENVPA | GLN_B: 354(340)_LYS, ASP_B: 80(66)_LYS, |
| | MYVAIQAVLSLYASGRTTGIVLDS | ASP_B: 222(208)_LYS, GLU_B: 224(210)_LYS, |
| | GDGVTHNVPIYEGYALPHAIMRL | GLU_B: 270(256)_LYS, GLU_B: 364(350)_LYS, |
| | DLAGRDLTDYLMKILTERGYSFV | GLU_B: 195(181)_LYS |
| | TTAEREIVRDIKEKLCYVALDFEN | |
| | EMATAASSSSLEKSYELPDGQVITI | |
| | GNERFRCPETLFQPSFIGMESAGIH | |
| | ETTYNSIMKCDIDIRKDLYANNV | |
| | MSGGTTMYPGIADRMQKEITALA | |
| | PSTMKIKIIAPPERKYSVWIGGSIL | |
| | ASLSTFQQMWITKQEYDEAGPSIV | |
| | HRK (SEQ ID NO: XX) | |
| Tubilin (3cb2) | Chain A: | ASP_A: 310(303)_LYS, GLU_A: 43(42)_LYS, |
| | PREIITLQLGQCGNQIGFEFWKQL | ASP_A: 56(55)_LYS, ASP_A: 57(56)_LYS, |
| | CAEHGISPEAIVEEFATEGTDRKD | GLU_A: 39(38)_LYS, GLU_A: 177(176)_LYS, |
| | VFFYQADDEHYIPRAVLLDLEPRV | ASP_A: 180(179)_LYS, GLU_B: 95(93)_LYS, |
| | IHSILNSPYAKLYNPENIYLSEHGG | ASP_B: 57(55)_LYS, ASP_B: 130(126)_LYS, |
| | GAGNNWASGESQGEKIHEDIFDII | ASP_B: 176(172)_LYS, ASN_A: 79(78)_LYS, |
| | DREADGSDSLEGFVLCHSIAGGTG | ASP_A: 127(126)_LYS, ASP_A: 130(129)_LYS, |
| | SGLGSYLLERLNDRYPKKLVQTY | ASP_A: 216(215)_LYS |
| | SVFPNQDEMSDVVVQPYNSLLTL | |
| | KRLTQNADCLVVLDNTALNRIAT | |
| | DRLHIQNPSFSQINQLVSTIMSAST | |
| | TTLRYPGYMNNDLIGLIASLIPTPR | |
| | LHFLMTGYTPLTSVRKTTVLDVM | |
| | RRLLQPKNVMVSTGRDTNHCYIA | |
| | ILNIIQGEVDPTQVHKSLQRIRERK | |
| | LANFIPWGPASIQVALSRKSPYRV | |
| | SGLMMANHTSISSLFERTCRQYD | |
| | KLRKREAFLEQFRKEDMFKDNFD | |
| | EMDTSREIVQQLIDEYHAATRPDY | |
| | ISW (SEQ ID NO: XX) | |
| | Chain B: | |
| | REIITLQLGQCGNQIGFEFWKQLC | |
| | AEHGISPEAIVEEFATEGTDRKDV | |
| | FFYQADDEHYIPRAVLLDLEPRVI | |
| | HSILNSPYAKLYNPENIYLSEHGA | |
| | GNNWASGFSQGEKIHEDIFDIIDRE | |
| | ADGSDSLEGFVLCHSIAGGTGSGL | |
| | GSYLLERLNDRYPKKLVQTYSVF | |
| | PNQDEMSDVVVQPYNSLLTLKRL | |
| | TQNADCLVVLDNTALNRIATDRL | |
| | HIQNPSFSQINQLVSTIMSASTTTL | |
| | RYPGYMNNDLIGLIASLIPTPRLHF | |
| | LMTGYTPLTKTTVLDVMRRLLQP | |
| | KNVMVSTTNHCYIAILNIIQGEVD | |
| | PTQVHKSLQRIRERLANFIPWGPA | |
| | SIQVALSRKSPYLPRVSGLMMAN | |
| | HTSISSLFERTCRQYDKLRKREAF | |
| | LEQFRKEDMFKDNFDEMDTSREI | |
| | VQQLIDEYHAATRPDYISW (SEQ | |
| | ID NO: XX) | |
| Keratin | | |
| Myosin (2fxo) | Chain A: | GLU_A: 844(10)_LYS, GLU_A: 854(20)_LYS, |
| | GSSPLLKSAEREKEMASMKEEFTR | GLU_B: 854(18)_LYS, GLN_B: 882(46)_LYS, |
| | LKEALEKSEARRKELEEKMVSLL | ASP_B: 956(120)_LYS, GLN_D: 882(46)_LYS, |
| | QEKNDLQLQVQAEQDNLADAEE | GLU_A: 848(14)_LYS, GLU_A: 875(41)_LYS, |
| | RCDQLIKNKIQLEAKVKEMNKRL | GLN_A: 882(48)_LYS, GLN_A: 914(80)_LYS, |
| | EDEEEMNAELTAKKRKLEDECSE | GLU_A: 921(87)_LYS, ASP_A: 956(122)_LYS, |
| | LKRDIDDLELTLAK (SEQ ID NO: | GLU_B: 848(12)_LYS, GLU_B: 864(28)_LYS, |
| | XX) | GLU_B: 875(39)_LYS |
| | Chain B: | |
| | SPLLKSAEREKEMASMKEEFTRL | |
| | KEALEKSEARRKELEEKMVSLLQ | |
| | EKNDLQLQVQAEQDNLADAEER | |
| | CDQLIKNKIQLEAKVKEMNKRLE | |
| | DEEEMNAELTAKKRKLEDECSEL | |
| | KRDIDDLELTL (SEQ ID NO: XX) | |
| | Chain C: | |
| | SSPLLKSAEREKEMASMKEEFTRL | |
| | KEALEKSEARRKELEEKMVSLLQ | |
| | EKNDLQLQVQAEQDNLADAEER | |
| | CDQLIKNKIQLEAKVKEMNKRLE | |
| | DEEEMNAELTAKKRKLEDECSEL | |
| | KRDIDDLELTLA (SEQ ID NO: | |
| | XX) | |
| | Chain D: | |
| | SPLLKSAEREKEMASMKEEFTRL | |
| | KEALEKSEARRKELEEKMVSLLQ | |
| | EKNDLQLQVQAEQDNLADAEER | |
| | CDQLIKNKIQLEAKVKEMNKRLE | |
| | DEEEMNAELTAKKRKLEDECSEL | |
| | KRDIDDLELTLAK (SEQ ID NO: | |
| | XX) | |
| EXTRACELLUL. | | |
| PROTEINS | | |
| Cytokines | | |
| Colony Stimulating | | |
| Factors | | |
| G-CSF | Chain A: | GLU_A: 123(106)_LYS, GLU_A: 122(105)_LYS, |
| | LPQSFLLKCLEQVRKIQGDGAALQ | GLN_A: 11(3)_LYS, GLU_A: 45(37)_LYS, |
| | EKLCATYKLCHPEELVLLGHSLGI | GLU_A: 46(38)_LYS, GLU_A: 98(81)_LYS, |
| | PWAPLLAGCLSQLHSGLFLYQGL | GLU_A: 19(11)_LYS, GLN_A: 119(102)_LYS, |
| | LQALEGISPELGPTLDTLQLDVAD | ASP_A: 112(95)_LYS, GLN_A: 77(60)_LYS, |
| | FATTIWQQMEELGMMPAFASAFQ | GLU_A: 33(25)_LYS, GLN_A: 90(73)_LYS, |
| | RRAGGVLVASHLQSFLEVSYRVL | GLU_A: 93(76)_LYS, ASP_A: 104(87)_LYS, |
| | RHLA (SEQ ID NO: XX) | GLU_A: 162(135)_LYS |
| GM-CSF | Chain B: | GLN_B: 50(37)_LYS, GLU_B: 14(1)_LYS, |
| | EHVNAIQEARRLLNLSRDTAAEM | GLU_B: 51(38)_LYS, GLN_B: 86(73)_LYS, |
| | NETVEVISEMFDLQEPTCLQTRLE | ASN_B: 27(14)_LYS, ASP_B: 48(35)_LYS, |
| | LYKQGLRGSLTKLKGPLTMMASH | ASN_B: 17(4)_LYS, ASP_B: 31(18)_LYS, |
| | YKQHCPPTPETSCATQIITFESFKE | GLU_B: 93(80)_LYS, GLN_B: 99(86)_LYS, |
| | NLKDFLLVIP (SEQ ID NO: XX) | GLU_B: 21(8)_LYS, ASN_B: 37(24)_LYS, |
| | | GLU_B: 45(32)_LYS, GLN_B: 64(51)_LYS, |
| | | GLU_B: 108(95)_LYS |
| Interferons | | |
| Interferon alfa-2 | Chain B: | LU_B: 165(165)_LYS, GLN_B: 5(5)_LYS, |
| | CDLPQTHSLGSRRTLMLLAQMRK | GLU_B: 107(107)_LYS, GLN_B: 46(46)_LYS, |
| | ISLFSCLKDRHDFGFPQEEFGNQF | GLN_B: 101(101)_LYS, ASN_B: 45(45)_LYS, |
| | QKAETIPVLHEMIQQIFNLFSTKDS | ASN_B: 65(65)_LYS, GLU_B: 132(132)_LYS, |
| | SAAWDETLLDKFYTELYQQLNDL | GLU_B: 159(159)_LYS, GLU_B: 41(41)_LYS, |
| | EACVIQGVGVTETPLMKEDSILAV | ASP_B: 82(82)_LYS, ASP_B: 2(2)_LYS, |
| | RKYFQRITLYLKEKKYSPCAWEV | GLN_B: 20(20)_LYS, ASP_B: 35(35)_LYS, |
| | VRAEIMRSFSLSTNLQESLRSKE | ASP_B: 71(71)_LYS |
| | (SEQ ID NO: XX) | |
| Interferon beta-1 | Chain A: | ASP_A: 110(110)_LYS, GLU_A: 29(29)_LYS, |
| | MSYNLLGFLQRSSNFQCQKLLWQ | ASN_A: 37(37)_LYS, GLU_A: 42(42)_LYS, |
| | LNGRLEYCLKDRMNFDIPEEIKQL | GLU_A: 109(109)_LYS, GLN_A: 46(46)_LYS, |
| | QQFQKEDAALTIYEMLQNIFAIFR | GLN_A: 48(48)_LYS, GLN_A: 49(49)_LYS, |
| | QDSSSTGWNETIVENLLANVYHQI | GLU_A: 103(103)_LYS, GLU_A: 107(107)_LYS, |
| | NHLKTVLEEKLEKEDFTRGKLMS | ASP_A: 39(39)_LYS, GLN_A: 51(51)_LYS, |
| | SLHLKRYYGRILHYLKAKEYSHC | GLU_A: 104(104)_LYS, ASN_A: 166(166)_LYS, |
| | AWTIVRVEILRNFYFINRLTGYLR | GLN_A: 23(23)_LYS |
| | N (SEQ ID NO: XX) | |
| Interferon gamma-1b | Chain A: | ASN_A: 225(143)_LYS, ASP_A: 224(142)_LYS, |
| | MQDPYVKEAENLKKYFNAGHSD | GLN_A: 1(2)_LYS, ASP_A: 2(3)_LYS, |
| | VADNGTLFLGILKNWKEESDRKI | GLN_A: 64(65)_LYS, GLU_A: 238(156)_LYS, |
| | MQSQIVSFYFKLFKNFKDDQSIQK | GLN_A: 264(182)_LYS, ASP_A: 24(25)_LYS, |
| | SVETIKEDMNVKFFNSNKKKRDD | ASN_A: 25(26)_LYS, ASP_A: 102(103)_LYS, |
| | FEKLTNYSVTDLNVQRKAIDELIQ | ASN_A: 297(215)_LYS, ASP_A: 302(220)_LYS, |
| | VMAELGANVSGEFVKEAENLKK | GLU_A: 38(39)_LYS, ASN_A: 59(60)_LYS, |
| | YFNDNGTLFLGILKNWKEESDRKI | ASP_A: 63(64)_LYS |
| | MQSQIVSFYFKLFKNFKDDQSIQK | |
| | SVETIKEDMNVKFFNSNKKKRDD | |
| | FEKLTNYSVTDLNVQRKAIHELIQ | |
| | VMAELSPAA (SEQ ID NO: XX) | |
| Interleukins | | |
| IL-2 (1M47) | Chain A: | ASN_A: 77(70)_LYS, ASN_A: 33(28)_LYS, |
| | STKKTQLQLEHLLLDLQMILNGIN | ASP_A: 109(98)_LYS, GLN_A: 74(69)_LYS, |
| | NYKNPKLTRMLTFKFYMPKKATE | ASP_A: 84(77)_LYS, GLU_A: 95(88)_LYS, |
| | LKHLQCLEEELKPLEEVLNLAQNF | GLU_A: 110(99)_LYS, ASN_A: 26(21)_LYS, |
| | HLRPRDLISNINVIVLELKGFMCE | ASN_A: 29(24)_LYS, ASN_A: 30(25)_LYS, |
| | YADETATIVEFLNRWITFCQSIIST | GLU_A: 52(47)_LYS, GLU_A: 68(63)_LYS, |
| | LT (SEQ ID NO: XX) | ASN_A: 71(66)_LYS, GLU_A: 61(56)_LYS, |
| | | GLU_A: 62(57)_LYS |
| IL-1 receptor | Chain A: | ASN_A: 79(79)_LYS, GLU_A: 114(114)_LYS, |
| antagonist (1irb) | ALWQFNGMIKCKIPSSEPLLDFNN | ASP_A: 59(59)_LYS, GLU_A: 87(87)_LYS, |
| | YGCYCGLGGSGTPVDDLDRCCQT | ASP_A: 21(21)_LYS, ASN_A: 50(50)_LYS, |
| | HDNCYKQAKKLDSCKVLVDNPY | ASP_A: 66(66)_LYS, GLU_A: 81(81)_LYS, |
| | TNNYSYSCSNNEITCSSENNACEA | ASP_A: 119(119)_LYS, ASN_A: 122(122)_LYS, |
| | FICNCDRNAAICFSKVPYNKEHKN | ASN_A: 80(80)_LYS, ASN_A: 89(89)_LYS, |
| | LDAANC (SEQ ID NO: XX) | ASN_A: 112(112)_LYS, GLU_A: 17(17)_LYS, |
| | | GLN_A: 54(54)_LYS |
| IL-1 (2nvh) | Chain A: | GLN_A: 34(34)_LYS, ASN_A: 53(53)_LYS, |
| | APVRSLNCTLRDSQQKSLVMSGP | ASP_A: 75(75)_LYS, ASP_A: 76(76)_LYS, |
| | YELKALHLQGQDMEQQVVFSMS | ASN_A: 107(107)_LYS, ASN_A: 89(89)_LYS, |
| | FVQGEESNDKIPVALGLKEKNLYL | ASN_A: 108(108)_LYS, ASP_A: 35(35)_LYS, |
| | SCVLKDDKPTLQLESVDPKNYPK | ASP_A: 86(86)_LYS, GLU_A: 50(50)_LYS, |
| | KKMEKRFVFNKIEINNKLEFESAQ | GLN_A: 141(141)_LYS, GLN_A: 32(32)_LYS, |
| | FPNWYISTSQAENMPVFLGGTKG | GLU_A: 37(37)_LYS, ASP_A: 54(54)_LYS, |
| | GQDITDFTMQFVS (SEQ ID NO: | GLU_A: 64(64)_LYS |
| | XX) | |
| Ciliary neurotrophic | Chain 1: | GLU_4: 66(34)_LYS, GLU_1: 66(37)_LYS, |
| factor (CNTF) (1cnt) | PHRRDLCSRSIWLARKIRSDLTAL | GLU_1: 153(116)_LYS, ASN_4: 137(99)_LYS, |
| | TESYVKHQGLWSELTEAERLQEN | ASP_1: 104(75)_LYS, GLU_1: 131(102)_LYS, |
| | LQAYRTFHVLLARLLEDQQVHFT | GLU_1: 138(109)_LYS, GLU_4: 71(39)_LYS, |
| | PTEGDFHQAIHTLLLQVAAFAYQI | ASP_1: 140(111)_LYS, GLU_1: 164(127)_LYS, |
| | EELMILLEYKIPRNEADGMLFEKK | GLN_1: 167(130)_LYS, GLU_4: 131(93)_LYS, |
| | LWGLKVLQELSQWTVRSIHDLRFI | ASP_1: 15(5)_LYS, GLU_1: 36(26)_LYS, |
| | SSHQTGIP (SEQ ID NO: XX) | ASN_1: 137(108)_LYS |
| | Chain 4: | |
| | HRRDLCSRSIWLARKIRSDLTALT | |
| | ESYVKHQGLELTEAERLQENLQA | |
| | YRTFHVLLARLLEDQQEGDFHQA | |
| | IHTLLLQVAAFAYQIEELMILLEY | |
| | KIPRNKKLWGLKVLQELSQWTVR | |
| | SIHDLRFIS (SEQ ID NO: XX) | |
| TNFs | | |
| TNF-alpha (4tsv) | Chain A: | ASP_A: 10(1)_LYS, GLU_A: 107(98)_LYS, |
| | DKPVAHVVANPQAEGQLQWSNR | GLN_A: 21(12)_LYS, GLN_A: 102(93)_LYS, |
| | RANALLANGVELRDNQLVVPIEG | GLU_A: 146(137)_LYS, ASN_A: 34(25)_LYS, |
| | LFLIYSQVLFKGQGCPSTHVLLTH | GLU_A: 23(14)_LYS, ASP_A: 45(36)_LYS, |
| | TISRIAVSYQTKVNLLSAIKSPCQR | GLN_A: 88(79)_LYS, GLN_A: 125(116)_LYS, |
| | ETPEGAEAKPWYEPIYLGGVFQLE | ASN_A: 39(30)_LYS, GLN_A: 67(58)_LYS, |
| | KGDRLSAEINRPDYLDFAESGQV | GLU_A: 110(101)_LYS, GLU_A: 53(44)_LYS, |
| | YFGIIAL (SEQ ID NO: XX) | ASN_A: 92(83)_LYS |
| TNF-beta | Chain A: | GLN_A: 107(80)_LYS, ASP_A: 50(23)_LYS, |
| (lymphotoxin) (1tnr) | KPAAHLIGDPSKQNSLLWRANTD | ASN_A: 62(35)_LYS, GLU_A: 127(100)_LYS, |
| | RAFLQDGFSLSNNSLLVPTSGIYF | GLN_A: 140(113)_LYS, ASN_A: 41(14)_LYS, |
| | VYSQVVFSGKAYSPKATSSPLYLA | ASP_A: 56(29)_LYS, ASN_A: 48(21)_LYS, |
| | HEVQLFSSQYPFHVPLLSSQKMV | GLN_A: 55(28)_LYS, GLN_A: 118(91)_LYS, |
| | YPGLQEPWLHSMYHGAAFQLTQ | GLN_A: 40(13)_LYS, GLN_A: 143(116)_LYS, |
| | GDQLSTHTDGIPHLVLSPSTVFFG | GLN_A: 126(99)_LYS, ASP_A: 152(125)_LYS, |
| | AFAL (SEQ ID NO: XX) | ASN_A: 63(36)_LYS |
| Peptide Hormones | | |
| Erythropoietin | Chain A: | ASP_A: 165(165)_LYS, GLU_A: 89(89)_LYS, |
| | APPRLICDSRVLERYLLEAKEAEKI | GLU_A: 31(31)_LYS, ASP_A: 123(123)_LYS, |
| | TTGCAEHCSLNEKITVPDTKVNFY | ASN_A: 47(47)_LYS, GLU_A: 55(55)_LYS, |
| | AWKRMEVGQQAVEVWQGLALL | GLN_A: 86(86)_LYS, ASN_A: 36(36)_LYS, |
| | SEAVLRGQALLVKSSQPWEPLQL | GLU_A: 37(37)_LYS, GLU_A: 159(159)_LYS, |
| | HVDKAVSGLRSLTTLLRALGAQK | ASP_A: 8(8)_LYS, GLN_A: 92(92)_LYS, |
| | EAISNSDAASAAPLRTITADTFRKL | ASP_A: 96(96)_LYS, GLU_A: 13(13)_LYS, |
| | FRVYSNFLRGKLKLYTGEACRTG | GLU_A: 21(21)_LYS |
| | DR (SEQ ID NO: XX) | |
| Insulin | Chain A: | ASN_B: 3(3)_LYS, GLU_B: 13(13)_LYS, |
| | GIVEQCCTSICSLYQLENYCN | GLU_B: 21(21)_LYS, GLU_A: 4(4)_LYS, |
| | (SEQ ID NO: XX) | GLN_A: 5(5)_LYS, ASN_A: 21(21)_LYS, |
| | Chain B: | GLN_A: 15(15)_LYS, ASN_A: 18(18)_LYS, |
| | FVNQHLCGSHLVEALYLVCGERG | GLN_B: 4(4)_LYS, GLU_A: 17(17)_LYS |
| | FFYTPK (SEQ ID NO: XX) | |
| Growth hormone | Chain A: | GLU_A: 129(129)_LYS, GLU_A: 39(39)_LYS, |
| (GH) (Somatotropin) | FPTIPLSRLADNAWLRADRLNQLA | ASN_A: 47(47)_LYS, ASN_A: 63(63)_LYS, |
| (1huw) | FDTYQEFEEAYIPKEQIHSFWWNP | GLU_A: 65(65)_LYS, GLU_A: 66(66)_LYS, |
| | QTSLCPSESIPTPSNKEETQQKSNL | GLU_A: 88(88)_LYS, GLN_A: 40(40)_LYS, |
| | ELLRISLLLIQSWLEPVQFLRSVFA | GLN_A: 69(69)_LYS, ASP_A: 107(107)_LYS, |
| | NSLVYGASDSNVYDLLKDLEEGI | ASP_A: 112(112)_LYS, GLU_A: 33(33)_LYS, |
| | QTLMGRLEALLKNYGLLYCFNKD | GLN_A: 91(91)_LYS, ASN_A: 99(99)_LYS, |
| | MSKVSTYLRTVQCRSVEGSCGF | ASP_A: 116(116)_LYS |
| | (SEQ ID NO: XX) | |
| Follicle-stimulating | Chain C: | ASP_C: 43(26)_LYS, ASN_C: 27(10)_LYS, |
| hormone (FSH) | CHHRICHCSNRVFLCQESKVTEIPS | ASN_C: 47(30)_LYS, ASN_C: 112(95)_LYS, |
| | DLPRNAIELRFVLTKLRVIQKGAF | ASN_C: 251(234)_LYS, GLU_C: 259(242)_LYS, |
| | SGFGDLEKIEISQNDVLEVIEADVF | GLU_C: 34(17)_LYS, GLU_C: 239(222)_LYS, |
| | SNLPKLHEIRIEKANNLLYINPEAF | ASN_C: 240(223)_LYS, GLU_C: 39(22)_LYS, |
| | QNLPNLQYLLISNTGIKHLPDVHK | ASP_C: 71(54)_LYS, ASN_C: 205(188)_LYS, |
| | IHSLQKVLLDIQDNINIHTIERNSF | GLU_C: 207(190)_LYS, ASN_C: 211(194)_LYS, |
| | VGLSFESVILWLNKNGIQEIHNCA | GLU_C: 76(59)_LYS |
| | FNGTQLDELNLSDNNNLEELPND | |
| | VFHGASGPVILDISRTRIHSLPSYG | |
| | LENLKKLRARSTYNLKKLPTLE | |
| | (SEQ ID NO: XX) | |
| Gonadotropin- | | |
| releasing hormone | | |
| (GnRH) | | |
| Thyrotropin-releasing | | |
| hormone (TRH) | | |
| somatostatin (growth- | | |
| hormone-inhibiting | | |
| hormone | | |
| Leptin (1ax8) | Chain A: | GLN_A: 4(2)_LYS, ASP_A: 23(21)_LYS, |
| | IQKVQDDTKTLIKTIVTRINDILDFI | ASP_A: 40(24)_LYS, GLU_A: 105(89)_LYS, |
| | PGLHPILTLSKMDQTLAVYQQILT | ASP_A: 108(92)_LYS, GLU_A: 100(84)_LYS, |
| | SMPSRNVIQISNDLENLRDLLHVL | ASP_A: 8(6)_LYS, ASN_A: 22(20)_LYS, |
| | AFSKSCHLPEASGLETLDSLGGVL | ASP_A: 141(125)_LYS, ASN_A: 78(62)_LYS, |
| | EASGYSTEVVALSRLQGSLQDML | ASP_A: 9(7)_LYS, GLN_A: 75(59)_LYS, |
| | WQLDLSPGC (SEQ ID NO: XX) | ASP_A: 85(69)_LYS, ASN_A: 72(56)_LYS, |
| | | GLU_A: 81(65)_LYS |
| Growth-hormone- | | |
| releasing hormone | | |
| (GHRH) | | |
| Insulin-like growth | Chain I: | GLU _I: 3(2)_LYS, ASP_I: 20(19)_LYS, |
| factor (or | PETLCGAELVDALQFVCGDRGFY | GLU _I: 9(8)_LYS, ASP_I: 12(11)_LYS, |
| somatomedin) (1wqj) | FNKPTGYGSSSRRAPQTGIVDECC | ASN_I: 26(25)_LYS, GLN_I: 40(39)_LYS, |
| | FRSCDLRRLEMYCAP (SEQ ID NO: | ASP_I: 53(52)_LYS, ASP_I: 45(44)_LYS, |
| | XX) | GLU_I: 58(57)_LYS, GLN_I: 15(14)_LYS, |
| | | GLU_I: 46(45)_LYS |
| Antimullerian | | |
| hormone (or | | |
| mullerian inhibiting | | |
| factor or hormone) | | |
| Adiponectin (1c28) | Chain A: | ASP_C: 173(55)_LYS, GLN_B: 191(72)_LYS, |
| | MYRSAFSVGLETRVTVPNVPIRFT | GLU_A: 194(82)_LYS, ASP_A: 182(70)_LYS, |
| | KIFYNQQNHYDGSTGKFYCNIPGL | GLN_B: 193(74)_LYS, GLN_A: 143(31)_LYS, |
| | YYFSYHITVYMKDVKVSLFKKDK | ASN_B: 130(12)_LYS, GLN_B: 143(25)_LYS, |
| | AVLFTYDQYQENVDQASGSVLLH | ASP_B: 182(64)_LYS, ASP_B: 190(71)_LYS, |
| | LEVGDQVWLQVYYADNVNDSTF | GLN_C: 143(28)_LYS, ASP_C: 182(64)_LYS, |
| | TGFLLYHDT (SEQ ID NO: XX) | ASP_B: 173(55)_LYS, ASP_B: 245(111)_LYS, |
| | Chain B: | ASN_A: 144(32)_LYS |
| | MYRSAFSVGLPNVPIRFTKIFYNQ | |
| | QNHYDGSTGKFYCNIPGLYYFSY | |
| | HITVYMKDVKVSLFKKDKVLFTY | |
| | DQYQEKVDQASGSVLLHLEVGD | |
| | QVWLQVYDSTFTGFLLYHD (SEQ | |
| | ID NO: XX) | |
| | Chain C: | |
| | MYRSAFSVGLETRVTVPIRFTKIF | |
| | YNQQNHYDGSTGKFYCNIPGLYY | |
| | FSYHITVDVKVSLFKKDKAVLFTQ | |
| | ASGSVLLHLEVGDQVWLQNDSTF | |
| | TGFLLYHD (SEQ ID NO: XX) | |
| Adrenocorticotropic | | |
| hormone (or | | |
| corticotropin) | | |
| Angiotensinogen and | | |
| angiotensin | | |
| Antidiuretic hormone | | |
| (or vasopressin, | | |
| arginine vasopressin) | | |
| Atrial-natriuretic | | |
| peptide (or | | |
| atriopeptin) | | |
| B-type natriuretic | | |
| peptide (BNP) | | |
| Calcitonin | | |
| Cholecystokinin | | |
| Corticotropin- | | |
| releasing hormone | | |
| Gastrin | | |
| Luteinizing hormone | | |
| (LH) | | |
| Coagulation Factors | | |
| Factor VIII (aka | Chain A: | GLN_A: 334(327)_LYS, ASN_A: 214(214)_LYS, |
| antihemophilic | ATRRYYLGAVELSWDYMQSDLG | ASP_A: 361(329)_LYS, ASP_A: 27(27)_LYS, |
| factor) (2r7e) | ELPVDARFPPRVPKSFPFNTSVVY | GLU_A: 211(211)_LYS, GLU_A: 331(324)_LYS, |
| | KKTLFVEFTDHLFNIAKPRPPWM | GLU_A: 332(325)_LYS, ASP_A: 363(331)_LYS, |
| | GLLGPTIQAEVYDTVVITLKNMAS | ASN_A: 714(682)_LYS, ASN_A: 41(4 O_LYS, |
| | HPVSLHAVGVSYWKASEGAEYD | ASP_A: 362(330)_LYS, ASN_A: 364(332)_LYS, |
| | DQTSQREKEDDKVFPGGSHTYVW | GLU_A: 720(688)_LYS, GLN_B: 1692(4)_LYS, |
| | QVLKENGPMASDPLCLTYSYLSH | ASP_A: 403(371)_LYS |
| | VDLVKDLNSGLIGALLVCREGSL | |
| | AKEKTQTLHKFILLFAVFDEGKS | |
| | WHSETKNAASARAWPKMHTVNG | |
| | YVNRSLPGLIGCHRKSVYWHVIG | |
| | MGTTPEVHSIFLEGHTFLVRNHRQ | |
| | ASLEISPITFLTAQTLLMDLGQFLL | |
| | FCHISSHQHDGMEAYVKVDSCPE | |
| | EPQFDDDNSPSFIQIRSVAKKHPKT | |
| | WVHYIAAEEEDWDYAPLVLAPD | |
| | DRSYKSQYLNNGPQRIGRKYKKV | |
| | RFMAYTDETFKTREAIQHESGILG | |
| | PLLYGEVGDTLLIIFKNQASRPYNI | |
| | YPHGITDVRPLYSRRLPKGVKHLK | |
| | DFPILPGEIFKYKWTVTVEDGPTK | |
| | SDPRCLTRYYSSFVNMERDLASG | |
| | LIGPLLICYKESVDQRGNQIMSDK | |
| | RNVILFSVFDENRSWYLTENIQRF | |
| | LPNPAGVQLEDPEFQASNIMHSIN | |
| | GYVFDSLQLSVCLHEVAYWYILSI | |
| | GAQTDFLSVFFSGYTFKHKMVYE | |
| | DTLTLFPFSGETVFMSMENPGLWI | |
| | LGCHNSDFRNRGMTALLKVSSCD | |
| | KNTGDYYEDSYED (SEQ ID NO: | |
| | XX) | |
| | Chain B: | |
| | RSFQKKTRHYFIAAVERLWDYGM | |
| | SSSPHVLRNRAQSGSVPQFKKVVF | |
| | QEFTDGSFTQPLYRGELNEHLGLL | |
| | GPYIRAEVEDNIMVTFRNQASRPY | |
| | SFYSSLISYEEDQRQGAEPRKNFV | |
| | KPNETKTYFWKVQHHMAPTKDE | |
| | FDCKAWAYSSDVDLEKDVHSGLI | |
| | GPLLVCHTNTLNPAHGRQVTVQE | |
| | FALFFTIFDETKSWYFTENMERNC | |
| | RAPCNIQMEDPTFKENYRFHAING | |
| | YIMDTLPGLVMAQDQRIRWYLLS | |
| | MGSNENIHSIHFSGHVFTVRKKEE | |
| | YKMALYNLYPGVFETVEMLPSKA | |
| | GIWRVECLIGEHLHAGMSTLFLV | |
| | YSNKCQTPLGMASGHIRDFQITAS | |
| | GQYGQWAPKLARLHYSGSINAW | |
| | STKEPFSWIKVDLLAPMIIHGIKTQ | |
| | GARQKFSSLYISQFIIMYSLDGKK | |
| | WQTYRGNSTGTLMVFFGNVDSSG | |
| | IKHNIFNPPIIARYIRLHPTHYSIRST | |
| | LRMELMGCDLNSCSMPLGMESK | |
| | AISDAQITASSYFTNMFATWSPSK | |
| | ARLHLQGRSNAWRPQVNNPKEW | |
| | LQVDFQKTMKVTGVTTQGVKSLL | |
| | TSMYVKEFLISSSQDGHQWTLEFQ | |
| | NGKVKVFQGNQDSFTPVVNSLDP | |
| | PLLTRYLRIHPQSWVHQIALRMEV | |
| | LGCEAQDLY (SEQ ID NO: XX) | |
| Other | | |
| Human serum | Chain A: | ASP_B: 301(297)_LYS, ASP_A: 301(297)_LYS, |
| albumin (1ao6) | SEVAHRFKDLGEENFKALVLIAFA | GLU_A: 505(501)_LYS, GLU_B: 505(501)_LYS, |
| | QYLQQCPFEDHVKLVNEVTEFAK | GLU_A: 82(78)_LYS, GLU_A: 542(538)_LYS, |
| | TCVADESAENCDKSLHTLFGDKL | GLU_B: 82(78)_LYS, GLU_B: 542(538)_LYS, |
| | CTVATLRETYGEMADCCAKQEPE | GLU_A: 17(13)_LYS, GLU_A: 37(33)_LYS, |
| | RNECFLQHKDDNPNLPRLVRPEV | ASP_A: 562(558)_LYS, GLU_B: 17(13)_LYS, |
| | DVMCTAFHDNEETFLKKYLYEIA | GLU_B: 37(33)_LYS, ASP_B: 375(371)_LYS, |
| | RRHPYFYAPELLFFAKRYKAAFTE | ASP_B: 562(558)_LYS |
| | CCQAADKAACLLPKLDELRDEGK | |
| | ASSAKQRLKCASLQKFGERAFKA | |
| | WAVARLSQRFPKAEFAEVSKLVT | |
| | DLTKVHTECCHGDLLECADDRAD | |
| | LAKYICENQDSISSKLKECCEKPLL | |
| | EKSHCIAEVENDEMPADLPSLAA | |
| | DFVESKDVCKNYAEAKDVFLGM | |
| | FLYEYARRHPDYSVVLLLRLAKT | |
| | YETTLEKCCAAADPHECYAKVFD | |
| | EFKPLVEEPQNLIKQNCELFEQLG | |
| | EYKFQNALLVRYTKKVPQVSTPT | |
| | LVEVSRNLGKVGSKCCKHPEAKR | |
| | MPCAEDYLSVVLNQLCVLHEKTP | |
| | VSDRVTKCCTESLVNRRPCFSALE | |
| | VDETYVPKEFNAETFTFHADICTL | |
| | SEKERQIKKQTALVELVKHKPKA | |
| | TKEQLKAVMDDFAAFVEKCCKA | |
| | DDKETCFAEEGKKLVAASQAA | |
| | (SEQ ID NO: XX) | |
| | Chain B: | |
| | SEVAHRFKDLGEENFKALVLIAFA | |
| | QYLQQCPFEDHVKLVNEVTEFAK | |
| | TCVADESAENCDKSLHTLFGDKL | |
| | CTVATLRETYGEMADCCAKQEPE | |
| | RNECFLQHKDDNPNLPRLVRPEV | |
| | DVMCTAFHDNEETFLKKYLYEIA | |
| | RRHPYFYAPELLFFAKRYKAAFTE | |
| | CCQAADKAACLLPKLDELRDEGK | |
| | ASSAKQRLKCASLQKFGERAFKA | |
| | WAVARLSQRFPKAEFAEVSKLVT | |
| | DLTKVHTECCHGDLLECADDRAD | |
| | LAKYICENQDSISSKLKECCEKPLL | |
| | EKSHCIAEVENDEMPADLPSLAA | |
| | DFVESKDVCKNYAEAKDVFLGM | |
| | FLYEYARRHPDYSVVLLLRLAKT | |
| | YETTLEKCCAAADPHECYAKVFD | |
| | EFKPLVEEPQNLIKQNCELFEQLG | |
| | EYKFQNALLVRYTKKVPQVSTPT | |
| | LVEVSRNLGKVGSKCCKHPEAKR | |
| | MPCAEDYLSVVLNQLCVLHEKTP | |
| | VSDRVTKCCTESLVNRRPCFSALE | |
| | VDETYVPKEFNAETFTFHADICTL | |
| | SEKERQIKKQTALVELVKHKPKA | |
| | TKEQLKAVMDDFAAFVEKCCKA | |
| | DDKETCFAEEGKKLVAASQAA | |
| | (SEQ ID NO: XX) | |
| Alpha 1-Antitrypsin | Chain A: | GLN_A: 212(193)_LYS, GLU_A: 86(67)_LYS, |
| | HPTFNKITPNLAEFAFSLYRQLAH | GLU_A: 175(156)_LYS, ASN_A: 278(259)_LYS, |
| | QSNSTNIFFSPVSIAAAFAMLSLGA | ASP_A: 280(261)_LYS, ASN_A: 46(27)_LYS, |
| | KGDTHDEILEGLNFNLTEIPEAQIH | GLU_A: 257(238)_LYS, GLU_A: 279(260)_LYS, |
| | EGFQELLRTLNQPDSQLQLTTGNG | GLN_A: 44(25)_LYS, ASP_A: 270(251)_LYS, |
| | LFLSEGLKLVDKFLEDVKKLYHSE | GLU_A: 277(258)_LYS, GLN_A: 305(286)_LYS, |
| | AFTVNFGDTEEAKKQINDYVEKG | ASN_A: 314(295)_LYS, GLU_A: 346(327)_LYS, |
| | TQGKIVDLVKELDRDTVFALVNYI | GLN_A: 91(72)_LYS |
| | FFKGKWERPFEVKDTEEEDFHVD | |
| | QVTTVKVPMMKRLGMFNIQHCK | |
| | KLSSWVLLMKYLGNATAIFFLPD | |
| | EGKLQHLENELTHDIITKFLENED | |
| | RRSASLHLPKLSITGTYDLKSVLG | |
| | QLGITKVFSNGADLSGVTEEAPLK | |
| | LSKAVHKAVLTIDEKGTEAAGAM | |
| | FLEAIPMSIPPEVKFNKPFVFLMIE | |
| | QNTKSPLFMGKVVNPTQK(SEQ | |
| | ID NO: XX) | |
| Hemoglobin (1bz0) | Chain A: | GLU_B: 43(43)_LYS, ASN_B: 19(19)_LYS, |
| | VLSPADKTNVKAAWGKVGAHAG | ASP_A: 75(75)_LYS, GLU_B: 6(6)_LYS, |
| | EYGAEALERMFLSFPTTKTYFPHF | ASP_B: 73(73)_LYS, ASP_A: 47(47)_LYS, |
| | DLSHGSAQVKGHGKKVADALTN | GLU_B: 101(101)_LYS, ASN_A: 68(68)_LYS, |
| | AVAHVDDMPNALSALSDLHAHK | ASP_A: 74(74)_LYS, ASN_A: 78(78)_LYS, |
| | LRVDPVNFKLLSHCLLVTLAAHLP | ASP_A: 94(94)_LYS, ASP_B: 79(79)_LYS, |
| | AEFTPAVHASLDKFLASVSTVLTS | ASP_B: 94(94)_LYS, ASP_B: 99(99)_LYS, |
| | KYR (SEQ ID NO: XX) | GLU_B: 121(121)_LYS |
| | Chain B: | |
| | VHLTPEEKSAVTALWGKVNVDE | |
| | VGGEALGRLLVVYPWTQRFFESF | |
| | GDLSTPDAVMGNPKVKAHGKKV | |
| | LGAFSDGLAHLDNLKGTFATLSEL | |
| | HCDKLHVDPENFRLLGNVLVCVL | |
| | AHHFGKEFTPPVQAAYQKVVAG | |
| | VANALAHKYH (SEQ ID NO: XX) | |

TABLE 2
Exemplary Transcription Factors that can be Supercharged
Classified according to their regulatory function:

| I. | constitutively-active - present in all cells at all times - general transcription factors, Sp1, | |
|---|---|---|
| | NF1, CCAAT | |
| II. | conditionally-active - requires activation | |
| | II.A | developmental (cell specific) - expression is tightly controlled, but, once expressed, |
| | | require no additional activation - GATA, HNF, PIT-1, MyoD, Myf5, Hox, Winged |
| | | Helix |
| | II.B | signal-dependent - requires external signal for activation |

| | |
|---|---|
| II.B.1 | extracellular ligand-dependent - nuclear receptors |
| II.B.2 | intracellular ligand-dependent - activated by small intracellular molecules - |
| | SREBP, p53, orphan nuclear receptors |
| II.B.3 | cell membrane receptor-dependent - second messenger signaling cascades |
| | resulting in the phosphorylation of the transcription factor |

| | |
|---|---|
| II.B.3.a | resident nuclear factors - reside in the nucleus regardless of |
| | activation state - CREB, AP-1, Mef2 |
| II.B.3.b | latent cytoplasmic factors - inactive form reside in the cytoplasm, |
| | but, when activated, are translocated into the nucleus - STAT, R- |
| | SMAD, NF-kB, Notch, TUBBY, NFAT |

Classified based on sequence similarity and hence the tertiary structure of their DNA binding
domains:

| 1 | Superclass: Basic Domains (Basic-helix-loop-helix) | |
|---|---|---|
| | 1.1 | Class: Leucine zipper factors (bZIP) |

| | |
|---|---|
| 1.1.1 | Family: AP-1(-like) components; includes (c-Fos/c-Jun) |
| 1.1.2 | Family: CREB |
| 1.1.3 | Family: C/EBP-like factors |
| 1.1.4 | Family: bZIP/PAR |
| 1.1.5 | Family: Plant G-box binding factors |
| 1.1.6 | Family: ZIP only |

| | |
|---|---|
| 1.2 | Class: Helix-loop-helix factors (bHLH) |

| | |
|---|---|
| 1.2.1 | Family: Ubiquitous (class A) factors |
| 1.2.2 | Family: Myogenic transcription factors (MyoD) |
| 1.2.3 | Family: Achaete-Scute |
| 1.2.4 | Family: Tal/Twist/Atonal/Hen |

| | |
|---|---|
| 1.3 | Class: Helix-loop-helix/leucine zipper factors (bHLH-ZIP) |

| | |
|---|---|
| 1.3.1 | Family: Ubiquitous bHLH-ZIP factors; includes USF (USF1, USF2); SREBP |
| | (SREBP) |
| 1.3.2 | Family: Cell-cycle controlling factors; includes c-Myc |

| | |
|---|---|
| 1.4 | Class: NF-1 |

| | |
|---|---|
| 1.4.1 | Family: NF-1 (A, B, C, X) |

| | |
|---|---|
| 1.5 | Class: RF-X |

| | |
|---|---|
| 1.5.1 | Family: RF-X (1, 2, 3, 4, 5, ANK) |

| | 1.6 | Class: bHSH |
|---|---|---|
| 2 | Superclass: Zinc-coordinating DNA-binding domains | |
| | 2.1 | Class: Cys4 zinc finger of nuclear receptor type |

| | |
|---|---|
| 2.1.1 | Family: Steroid hormone receptors |
| 2.1.2 | Family: Thyroid hormone receptor-like factors |

| | |
|---|---|
| 2.2 | Class: diverse Cys4 zinc fingers |

| | |
|---|---|
| 2.2.1 | Family: GATA-Factors |

| | |
|---|---|
| 2.3 | Class: Cys2His2 zinc finger domain |

| | |
|---|---|
| 2.3.1 | Family: Ubiquitous factors, includes TFIIIA, Sp1 |
| 2.3.2 | Family: Developmental/cell cycle regulators; includes Kruppel |
| 2.3.4 | Family: Large factors with NF-6B-like binding properties |

| | 2.4 | Class: Cys6 cysteine-zinc cluster |
|---|---|---|
| | 2.5 | Class: Zinc fingers of alternating composition |
| 3 | Superclass: Helix-turn-helix | |
| | 3.1 | Class: Homeo domain |

| | |
|---|---|
| 3.1.1 | Family: Homeo domain only; includes Ubx |
| 3.1.2 | Family: POU domain factors; includes Oct |
| 3.1.3 | Family: Homeo domain with LIM region |
| 3.1.4 | Family: homeo domain plus zinc finger motifs |

| | |
|---|---|
| 3.2 | Class: Paired box |

| | |
|---|---|
| 3.2.1 | Family: Paired plus homeo domain |
| 3.2.2 | Family: Paired domain only |

| | |
|---|---|
| 3.3 | Class: Fork head/winged helix |

| | |
|---|---|
| 3.3.1 | Family: Developmental regulators; includes forkhead |
| 3.3.2 | Family: Tissue-specific regulators |
| 3.3.3 | Family: Cell-cycle controlling factors |
| 3.3.0 | Family: Other regulators |

| | |
|---|---|
| 3.4 | Class: Heat Shock Factors |

| | |
|---|---|
| 3.4.1 | Family: HSF |

| | |
|---|---|
| 3.5 | Class: Tryptophan clusters |

| | |
|---|---|
| 3.5.1 | Family: Myb |
| 3.5.2 | Family: Ets-type |
| 3.5.3 | Family: Interferon regulatory factors |

| | |
|---|---|
| 3.6 | Class: TEA (transcriptional enhancer factor) domain |

| | |
|---|---|
| 3.6.1 | Family: TEA (TEAD1, TEAD2, TEAD3, TEAD4) |

| 4 | Superclass: beta-Scaffold Factors with Minor Groove Contacts | |
|---|---|---|
| | 4.1 | Class: RHR (Rel homology region) |

| | |
|---|---|
| 4.1.1 | Family: Rel/ankyrin; NF-kappaB |
| 4.1.2 | Family: ankyrin only |
| 4.1.3 | Family: NFAT (Nuclear Factor of Activated T-cells) (NFATC1, NFATC2, |
| | NFATC3) |

| | |
|---|---|
| 4.2 | Class: STAT |

| | |
|---|---|
| 4.2.1 | Family: STAT |

| | |
|---|---|
| 4.3 | Class: p53 |

| | |
|---|---|
| 4.3.1 | Family: p53 |

| | |
|---|---|
| 4.4 | Class: MADS box |

| | |
|---|---|
| 4.4.1 | Family: Regulators of differentiation; includes (Mef2) |
| 4.4.2 | Family: Responders to external signals, SRF (serum response factor) (SRF) |

| | |
|---|---|
| 4.5 | Class: beta-Barrel alpha-helix transcription factors |
| 4.6 | Class: TATA binding proteins |

| | |
|---|---|
| 4.6.1 | Family: TBP |
| 4.7.1 | Family: SOX genes, SRY |
| 4.7.2 | Family: TCF-1 (TCF1) |
| 4.7.3 | Family: HMG2-related, SSRP1 |
| 4.7.5 | Family: MATA |

| | |
|---|---|
| 4.8 | Class: Heteromeric CCAAT factors |

| | |
|---|---|
| 4.8.1 | Family: Heteromeric CCAAT factors |

| | |
|---|---|
| 4.9 | Class: Grainyhead |

| | |
|---|---|
| 4.9.1 | Family: Grainyhead |

| | |
|---|---|
| 4.10 | Class: Cold-shock domain factors |

| | |
|---|---|
| 4.10.1 | Family: csd |

| | |
|---|---|
| 4.11 | Class: Runt |

| | |
|---|---|
| 4.11.1 | Family: Runt |

| 0 | Superclass: Other Transcription Factors | |
|---|---|---|
| | 0.1 | Class: Copper fist proteins |
| | 0.2 | Class: HMGI(Y) (HMGA1) |

| | |
|---|---|
| 0.2.1 | Family: HMGI(Y) |

| | |
|---|---|
| 0.3 | Class: Pocket domain |
| 0.4 | Class: E1A-like factors |
| 0.5 | Class: AP2/EREBP-related factors |

| | |
|---|---|
| 0.5.1 | Family: AP2 |
| 0.5.2 | Family: EREBP |
| 0.5.3 | Superfamily: AP2/B3 |

| | |
|---|---|
| 0.5.3.1 | Family: ARF |
| 0.5.3.2 | Family: ABI |
| 0.5.3.3 | Family: RAV |

In certain embodiments, a subset of the mutation proposed in Table 1 for a particular protein are made to create the supercharged protein. In certain embodiments, at least two mutations are made. In certain embodiments, at least three mutations are made. In certain embodiments, at least four mutations are made. In certain embodiments, at least five mutations are made. In certain embodiments, at least ten mutations are made. In certain embodiments, at least fifteen mutations are made. In certain embodiments, at least twenty mutations are made. In certain embodiments, all the proposed mutations are made to create the superpositively charged protein. In certain embodiments, none of the proposed mutations are made but rather one or more charged moieties are added to the protein to create the superpositively charged protein.

In certain embodiments, the supercharged protein is a naturally occurring supercharged protein. In certain embodiments, the theoretical net charge on the naturally occurring supercharged protein is at least +1, at least +2, at least +3, at least +4, at least +5, at least +10, at least +15, at least +20, at least +25, at least +30, at least +35, or at least +40. In certain embodiments, the supercharged protein has a charge:molecular weight ratio of at least approximately 0.8. In certain embodiments, the supercharged protein has a charge:molecular weight ratio of at least approximately 1.0. In certain embodiments, the supercharged protein has a charge:molecular weight ratio of at least approximately 1.2. In certain embodiments, the supercharged protein has a charge:molecular weight ratio of at least approximately 1.4. In certain embodiments, the supercharged protein has a charge:molecular weight ratio of at least approximately 1.5. In certain embodiments, the supercharged protein has a charge:molecular weight ratio of at least approximately 1.6. In certain embodiments, the supercharged protein has a charge:molecular weight ratio of at least approximately 1.7. In certain embodiments, the supercharged protein has a charge:molecular weight ratio of at least approximately 1.8. In certain embodiments, the supercharged protein has a charge:molecular weight ratio of at least approximately 1.9. In certain embodiments, the supercharged protein has a charge:molecular weight ratio of at least approximately 2.0. In certain embodiments, the supercharged protein has a charge:molecular weight ratio of at least approximately 2.5. In certain embodiments, the supercharged protein has a charge:molecular weight ratio of at least approximately 3.0. In certain embodiments, the molecular weight of the protein ranges from approximately 4 kDa to approximately 100 kDa. In certain embodiments, the molecular weight of the protein ranges from approximately 10 kDa to approximately 45 kDa. In certain embodiments, the molecular weight of the protein ranges from approximately 5 kDa to approximately 50 kDa. In certain embodiments, the molecular weight of the protein ranges from approximately 10 kDa to approximately 60 kDa. In certain embodiments, the naturally occurring supercharged protein is histone related. In certain embodiments, the naturally occurring supercharged protein is ribosome related. Examples of naturally occurring supercharged proteins include, but are not limited to, cyclon (ID No.: Q9H6F5); PNRC1 (ID No.: Q12796); RNPS1 (ID No.: Q15287); SURF6 (ID No.: O75683); AR6P (ID No.: Q66PJ3); NKAP (ID No.: Q8N5F7); EBP2 (ID No.: Q99848); LSM11 (ID No.: P83369); RL4 (ID No.: P36578); KRR1 (ID No.: Q13601); RY-1 (ID No.: Q8WVK2); BriX (ID No.: Q8TDN6); MNDA (ID No.: P41218); H1b (ID No.: P16401); cyclin (ID No.: Q9UK58); MDK (ID No.: P21741); Midkine (ID No.: P21741); PROK (ID No.: Q9HC23); FGFS (ID No.: P12034); SFRS (ID No.: Q8N9Q2); AKIP (ID No.: Q9NWT8); CDK (ID No.: Q8N726); beta-defensin (ID No.: P81534); Defensin 3 (ID No.: P81534); PAVAC (ID No.: P18509); PACAP (ID No.: P18509); eotaxin-3 (ID No.: Q9Y258); histone H2A (ID No.: Q7L7L0); HMGB1 (ID No.: P09429); C-Jun (ID No.: P05412); TERF 1 (ID No.: P54274); N-DEK (ID No.: P35659); PIAS 1 (ID No.: O75925); Ku70 (ID No.: P12956); HBEGF (ID No.: Q99075); and HGF (ID No.: P14210). In certain embodiments, the supercharged protein utilized in the invention is U4/U6.U5 tri-snRNP-associated protein 3 (ID No.: Q8WVK2); beta-defensin (ID No.: P81534); Protein SFRS121P1 (ID No.: Q8N9Q2); midkine (ID No.: P21741); C—C motif chemokine 26 (ID No.: Q9Y258); surfeit locus protein 6 (ID No.: O75683); Aurora kinase A-interacting protein (ID No.: Q9NWT8); NF-kappa-B-activating protein (ID No.: Q8N5F7); histone H1.5 (ID No.: P16401); histone H2A type 3 (ID No.: Q7L7L0); 60S ribosomal protein L4 (ID No.: P36578); isoform 1 of RNA-binding protein with serine-rich domain 1 (ID No.: Q15287-1); isoform 4 of cyclin-dependent kinase inhibitor 2A (ID No.: Q8N726-1); isoform 1 of prokineticin-2 (ID No.: Q9HC23-1); isoform 1 of ADP-ribosylation factor-like protein 6-interacting protein 4 (ID No.: Q66PJ3-1); isoform long of fibroblast growth factor 5 (ID No.: P12034-1); or isoform 1 of cyclin-L1 (ID No.: Q9UK58-1). Other possible naturally occurring supercharged proteins from the human proteome that may be utilized in the present invention are included in the list below. The proteins listed have a charge:molecular weight ratio of greater than 0.8.

| | | | | |
|---|---|---|---|---|
| Ratio | Charge | Name | aa | MW |

Cationic Proteins
[‘3.49’, 23, ‘sp|P04553|HSP1_HUMAN Sperm protamine-P1 OS = Homo sapiens GN = PRM1’, 51, 6822]
[‘3.00’, 19, ‘sp|P09430|STP1_HUMAN Spermatid nuclear transition protein 1 OS = Homo sapiens GN = TNP1’, 55, 6424]
[‘2.19’, 23, ‘sp|Q9UNZ5|L10K_HUMAN Leydig cell tumor 10 kDa protein homolog OS = Homo sapiens GN = C19orf53’, 99, 10576]
[‘2.07’, 27, ‘sp|P04554|PRM2_HUMAN Protamine-2 OS = Homo sapiens GN = PRM2’, 102, 13050]
[‘1.80’, 18, ‘sp|Q5EE01|CUG2_HUMAN Cancer-up-regulated gene 2 protein OS = Homo sapiens GN = C6orf173’, 88, 10061]
[‘1.78’, 17, ‘sp|O00479|HMGN4_HUMAN High mobility group nucleosome-binding domain-containing protein 4 OS = Homo sapiens
GN = HMGN4’, 90, 9538]
[‘1.65’, 25, ‘sp|Q9BRT6|CL031_HUMAN UPF0446 protein C12orf31 OS = Homo sapiens GN = C12orf31’, 129, 15225]
[‘1.62’, 80, ‘sp|Q8IV32|CCD71_HUMAN Coiled-coil domain-containing protein 71 OS = Homo sapiens GN = CCDC71’, 467, 49618]
[‘1.59’, 24, ‘sp|Q05952|STP2_HUMAN Nuclear transition protein 2 OS = Homo sapiens GN = TNP2’, 138, 15640]
[‘1.57’, 22, ‘sp|Q07325|CXCL9_HUMAN C—X—C motif chemokine 9 OS = Homo sapiens GN = CXCL9’, 125, 14018]
[‘1.56’, 11, ‘sp|Q9Y2S6|CCD72_HUMAN Coiled-coil domain-containing protein 72 OS = Homo sapiens GN = CCDC72’, 64, 7066]
[‘1.55’, 29, ‘sp|Q8WVK2|SNUT3_HUMAN U4/U6.U5 tri-snRNP-associated protein 3 OS = Homo sapiens’, 155, 18860]
[‘1.55’, 11, ‘sp|P81534|D103A_HUMAN Beta-defensin 103 OS = Homo sapiens GN = DEFB103A’, 67, 7697]
[‘1.54’, 8, ‘sp|Q5VTU8|AT5EL_HUMAN ATP synthase subunit epsilon-like protein, mitochondrial OS = Homo sapiens GN = ATP5EP2’, 51,
5806]
[‘1.45’, 10, ‘sp|P84101|SERF2_HUMAN Small EDRK-rich factor 2 OS = Homo sapiens GN = SERF2’, 59, 6899]
[‘1.40’, 102, ‘sp|A6NNA2|SRR2L_HUMAN SRRM2-like protein OS = Homo sapiens’, 665, 72877]
[‘1.39’, 40, ‘sp|Q8N9E0|F133A_HUMAN Protein FAM133A OS = Homo sapiens GN = FAM133A’, 248, 28940]
[‘1.38’, 35, ‘sp|A6NF02|NPPL2_HUMAN NPIP-like protein ENSP00000346774 OS = Homo sapiens’, 221, 26005]
[‘1.37’, 11, ‘sp|Q7Z4L0|COX83_HUMAN Cytochrome c oxidase polypeptide 8C, mitochondrial OS = Homo sapiens GN = COX8C’, 72, 8128]
[‘1.35’, 34, ‘sp|O75200|NPPL1_HUMAN NPIP-like protein LOC440350 OS = Homo sapiens’, 221, 25868]
[‘1.32’, 18, ‘sp|Q6UXB2|VCC1_HUMAN VEGF co-regulated chemokine 1 OS = Homo sapiens GN = CXCL17’, 119, 13819]
[‘1.32’, 10, ‘sp|Q8N688|DB123_HUMAN Beta-defensin 123 OS = Homo sapiens GN = DEFB123’, 67, 8104]
[‘1.31’, 36, ‘sp|Q5U4N7|GDF5O_HUMAN Protein GDF5OS, mitochondrial OS = Homo sapiens GN = GDF5OS’, 250, 28153]
[‘1.31’, 12, ‘sp|O00198|HRK_HUMAN Activator of apoptosis harakiri OS = Homo sapiens GN = HRK’, 91, 9883]
[‘1.30’, 29, ‘sp|Q8WW32|HMGB4_HUMAN High mobility group protein B4 OS = Homo sapiens GN = HMGB4’, 186, 22404]
[‘1.28’, 23, ‘sp|Q8N9Q2|S12IP_HUMAN Protein SFRS12IP1 OS = Homo sapiens GN = SFRS12IP1’, 155, 18176]
[‘1.26’, 19, ‘sp|P21741|MK_HUMAN Midkine OS = Homo sapiens GN = MDK’, 143, 15585]
[‘1.26’, 16, ‘sp|Q08E93|F27E3_HUMAN Protein FAM27E3 OS = Homo sapiens GN = FAM27E3’, 113, 13507]
[‘1.23’, 44, ‘sp|Q96QD9|FYTD1_HUMAN Forty-two-three domain-containing protein 1 OS = Homo sapiens GN = FYTTD1’, 318, 35799]
[‘1.23’, 16, ‘sp|P62314|SMD1_HUMAN Small nuclear ribonucleoprotein Sm D1 OS = Homo sapiens GN = SNRPD1’, 119, 13281]
[‘1.23’, 13, ‘sp|Q9Y258|CCL26_HUMAN C-C motif chemokine 26 OS = Homo sapiens GN = CCL26’, 94, 10647]
[‘1.22’, 10, ‘sp|Q96PI1|SPRR4_HUMAN Small proline-rich protein 4 OS = Homo sapiens GN = SPRR4’, 79, 8793]
[‘1.21’, 24, ‘sp|B2CW77|KILIN_HUMAN Killin OS = Homo sapiens’, 178, 19957]
[‘1.20’, 10, ‘sp|Q9Y5V0|ZN706_HUMAN Zinc finger protein 706 OS = Homo sapiens GN = ZNF706’, 76, 8497]
[‘1.20’, 6, ‘sp|P56381|ATP5E_HUMAN ATP synthase subunit epsilon, mitochondrial OS = Homo sapiens GN = ATP5E’, 51, 5779]
[‘1.19’, 61, ‘sp|Q9HAH1|ZN556_HUMAN Zinc finger protein 556 OS = Homo sapiens GN = ZNF556’, 456, 51581]
[‘1.19’, 30, ‘sp|P17026|ZNF22_HUMAN Zinc finger protein 22 OS = Homo sapiens GN = ZNF22’, 224, 25915]
[‘1.18’, 16, ‘sp|Q9NRJ3|CCL28_HUMAN C-C motif chemokine 28 OS = Homo sapiens GN = CCL28’, 127, 14279]
[‘1.16’, 11, ‘sp|O43262|LEU2_HUMAN Leukemia-associated protein 2 OS = Homo sapiens GN = DLEU2’, 84, 10196]
[‘1.15’, 38, ‘sp|Q6PK04|CC137_HUMAN Coiled-coil domain-containing protein 137 OS = Homo sapiens GN = CCDC137’, 289, 33231]
[‘1.15’, 18, ‘sp|A8MYZ5|YC026_HUMAN IQ domain-containing protein ENSP00000381760 OS = Homo sapiens’, 130, 15797]
[‘1.15’, 16, ‘sp|Q5T7N7|F27E1_HUMAN Protein FAM27E1 OS = Homo sapiens GN = FAM27E1’, 126, 14751]
[‘1.15’, 16, ‘sp|Q5SNX5|F27E2_HUMAN Protein FAM27E2 OS = Homo sapiens GN = FAM27E2’, 125, 14710]
[‘1.15’, 16, ‘sp|O00585|CCL21_HUMAN C-C motif chemokine 21 OS = Homo sapiens GN = CCL21’, 134, 14646]
[‘1.15’, 6, ‘sp|Q13794|APR_HUMAN Phorbol-12-myristate-13-acetate-induced protein 1 OS = Homo sapiens GN = PMAIP1’, 54, 6030]
[‘1.14’, 13, ‘sp|P19875|MIP2A_HUMAN Macrophage inflammatory protein 2-alpha OS = Homo sapiens GN = CXCL2’, 107, 11388]
[‘1.14’, 12, ‘sp|Q9P021|CRIPT_HUMAN Cysteine-rich PDZ-binding protein OS = Homo sapiens GN = CRIPT’, 101, 11215]
[‘1.14’, 11, ‘sp|O14625|CXL11_HUMAN C—X—C motif chemokine 11 OS = Homo sapiens GN = CXCL11’, 94, 10364]
[‘1.13’, 10, ‘sp|P61580|NP10_HUMAN HERV-K_5q33.3 provirus Np9 protein OS = Homo sapiens’, 75, 8892]
[‘1.12’, 46, ‘sp|O75683|SURF6_HUMAN Surfeit locus protein 6 OS = Homo sapiens GN = SURF6’, 361, 41450]
[‘1.12’, 15, ‘sp|P0C7P0|CISD3_HUMAN CDGSH iron sulfur domain-containing protein 3, mitochondrial OS = Homo sapiens GN = CISD3’,
127, 14215]
[‘1.10’, 37, ‘sp|Q9Y2B4|T53G5_HUMAN TP53-target gene 5 protein OS = Homo sapiens GN = TP53TG5’, 290, 34019]
[‘1.10’, 33, ‘sp|Q9Y3A2|UTP11_HUMAN Probable U3 small nucleolar RNA-associated protein 11 OS = Homo sapiens GN = UTP11L’, 253,
30446]
[‘1.10’, 21, ‘sp|Q9HCT0|FGF22_HUMAN Fibroblast growth factor 22 OS = Homo sapiens GN = FGF22’, 170, 19662]
[‘1.10’, 11, ‘sp|P51671|CCL11_HUMAN Eotaxin OS = Homo sapiens GN = CCL11’, 97, 10731]
[‘1.09’, 14, ‘sp|Q9Y421|FA32A_HUMAN Protein FAM32A OS = Homo sapiens GN = FAM32A’, 112, 13178]
[‘1.09’, 12, ‘sp|Q2M2W7|CQ058_HUMAN UPF0450 protein C17orf58 OS = Homo sapiens GN = C17orf58’, 97, 11205]
[‘1.09’, 11, ‘sp|Q99616|CCL13_HUMAN C-C motif chemokine 13 OS = Homo sapiens GN = CCL13’, 98, 10986]
[‘1.09’, 11, ‘sp|P0C665|PRAC2_HUMAN Small nuclear protein PRAC2 OS = Homo sapiens GN = PRAC2’, 90, 10483]
[‘1.09’, 11, ‘sp|P0C0P6|NPS_HUMAN Neuropeptide S OS = Homo sapiens GN = NPS’, 89, 10103]
[‘1.08’, 21, ‘sp|Q8IXL9|IQCF2_HUMAN IQ domain-containing protein F2 OS = Homo sapiens GN = IQCF2’, 164, 19627]
[‘1.08’, 8, ‘sp|Q13891|BT3L2_HUMAN Transcription factor BTF3 homolog 2 OS = Homo sapiens GN = BTF3L2’, 67, 7605]
[‘1.08’, 7, ‘sp|P56378|68MP_HUMAN 6.8 kDa mitochondrial proteolipid OS = Homo sapiens GN = MP68’, 58, 6662]
[‘1.08’, 6, ‘sp|P15516|HIS3_HUMAN Histatin-3 OS = Homo sapiens GN = HTN3’, 51, 6149]
[‘1.07’, 26, ‘sp|Q5T7N8|F27D1_HUMAN Protein FAM27D1 OS = Homo sapiens GN = FAM27D1’, 215, 24905]
[‘1.07’, 24, ‘sp|Q9NWT8|AKIP_HUMAN Aurora kinase A-interacting protein OS = Homo sapiens GN = AURKAIP1’, 199, 22354]
[‘1.07’, 16, ‘sp|A8MQ11|PM2L5_HUMAN Postmeiotic segregation increased 2-like protein 5 OS = Homo sapiens GN = PMS2L5’, 134, 15169]
[‘1.07’, 15, ‘sp|Q6UXT8|F150A_HUMAN Protein FAM150A OS = Homo sapiens GN = FAM150A’, 129, 14268]
[‘1.06’, 61, ‘sp|Q14593|ZN273_HUMAN Zinc finger protein 273 OS = Homo sapiens GN = ZNF273’, 504, 58045]
[‘1.06’, 9, ‘sp|Q9ULZ1|APEL_HUMAN Apelin OS = Homo sapiens GN = APLN’, 77, 8569]
[‘1.05’, 10, ‘sp|Q9UGL9|CRCT1_HUMAN Cysteine-rich C-terminal protein 1 OS = Homo sapiens GN = CRCT1’, 99, 9735]
[‘1.05’, 10, ‘sp|P81277|PRRP_HUMAN Prolactin-releasing peptide OS = Homo sapiens GN = PRLH’, 87, 9639]
[‘1.04’, 31, ‘sp|P52744|ZN138_HUMAN Zinc finger protein 138 OS = Homo sapiens GN = ZNF138’, 262, 30591]
[‘1.04’, 11, ‘sp|Q6IPR1|LYRM5_HUMAN LYR motif-containing protein 5 OS = Homo sapiens GN = LYRM5’, 88, 10604]
[‘1.04’, 9, ‘sp|P09669|COX6C_HUMAN Cytochrome c oxidase polypeptide VIc OS = Homo sapiens GN = COX6C’, 75, 8781]
[‘1.04’, 7, ‘sp|Q9NRQ5|CK075_HUMAN UPF0443 protein C11orf75 OS = Homo sapiens GN = C11orf75’, 59, 6738]
[‘1.03’, 23, ‘sp|Q8NHZ7|MB3L2_HUMAN Methyl-CpG-binding domain protein 3-like 2 OS = Homo sapiens GN = MBD3L2’, 204, 22695]
[‘1.03’, 11, ‘sp|Q9HD34|LYRM4_HUMAN LYR motif-containing protein 4 OS = Homo sapiens GN = LYRM4’, 91, 10758]
[‘1.03’, 10, ‘sp|Q06250|WIT1_HUMAN Wilms tumor-associated protein OS = Homo sapiens GN = WIT1’, 92, 10038]
[‘1.02’, 40, ‘sp|Q9NP08|HMX1_HUMAN Homeobox protein HMX1 OS = Homo sapiens GN = HMX1’, 373, 39225]
[‘1.02’, 15, ‘sp|Q9H963|ZN702_HUMAN Zinc finger protein 702 OS = Homo sapiens GN = ZNF702’, 129, 15053]
[‘1.02’, 14, ‘sp|P37108|SRP14_HUMAN Signal recognition particle 14 kDa protein OS = Homo sapiens GN = SRP14’, 136, 14569]
[‘1.02’, 12, ‘sp|P52926|HMGA2_HUMAN High mobility group protein HMGI-C OS = Homo sapiens GN = HMGA2’, 109, 11832]
[‘1.02’, 7, ‘sp|P58511|F165B_HUMAN UPF0601 protein FAM165B OS = Homo sapiens GN = FAM165B’, 58, 6886]
[‘1.01’, 24, ‘sp|P52743|ZN137_HUMAN Zinc finger protein 137 OS = Homo sapiens GN = ZNF137’, 207, 24114]
[‘1.01’, 18, ‘sp|Q8N912|CN180_HUMAN Transmembrane protein C14orf180 OS = Homo sapiens GN = C14orf180’, 160, 18051]
[‘1.01’, 14, ‘sp|Q8N8V8|TM105_HUMAN Transmembrane protein 105 OS = Homo sapiens GN = TMEM105’, 129, 13990]
[‘1.01’, 14, ‘sp|Q5TZK3|F74A4_HUMAN Protein FAM74A4 OS = Homo sapiens GN = FAM74A4’, 123, 14772]
[‘1.01’, 14, ‘sp|P42127|ASIP_HUMAN Agouti-signaling protein OS = Homo sapiens GN = ASIP’, 132, 14515]
[‘1.01’, 10, ‘sp|P60468|SC61B_HUMAN Protein transport protein Sec61 subunit beta OS = Homo sapiens GN = SEC61B’, 96, 9974]
[‘1.01’, 9, ‘sp|P61581|NP11_HUMAN HERV-K_22q11.21 provirus Np9 protein OS = Homo sapiens’, 75, 8893]
[‘1.00’, 72, ‘sp|Q6ZQV5|ZN788_HUMAN Zinc finger protein 788 OS = Homo sapiens GN = ZNF788’, 615, 71992]
[‘1.00’, 70, ‘sp|Q5HYK9|ZN667_HUMAN Zinc finger protein 667 OS = Homo sapiens GN = ZNF667’, 610, 70157]
[‘1.00’, 26, ‘sp|Q9H0W7|THAP2_HUMAN THAP domain-containing protein 2 OS = Homo sapiens GN = THAP2’, 228, 26259]
[‘0.99’, 20, ‘sp|P35318|ADML_HUMAN ADM OS = Homo sapiens GN = ADM’, 185, 20420]
[‘0.99’, 18, ‘sp|P21246|PTN_HUMAN Pleiotrophin OS = Homo sapiens GN = PTN’, 168, 18942]
[‘0.99’, 13, ‘sp|P23582|ANFC_HUMAN C-type natriuretic peptide OS = Homo sapiens GN = NPPC’, 126, 13246]
[‘0.99’, 10, ‘sp|P02778|CXL10_HUMAN C—X—C motif chemokine 10 OS = Homo sapiens GN = CXCL10’, 98, 10881]
[‘0.98’, 15, ‘sp|P14555|PA2GA_HUMAN Phospholipase A2, membrane associated OS = Homo sapiens GN = PLA2G2A’, 144, 16082]
[‘0.98’, 12, ‘sp|Q8NDT4|ZN663_HUMAN Zinc finger protein 663 OS = Homo sapiens GN = ZNF663’, 106, 12434]
[‘0.98’, 12, ‘sp|O00175|CCL24_HUMAN C-C motif chemokine 24 OS = Homo sapiens GN = CCL24’, 119, 13133]
[‘0.97’, 17, ‘sp|Q5T6X4|F162B_HUMAN UPF0389 protein FAM162B OS = Homo sapiens GN = FAM162B’, 162, 17684]
[‘0.97’, 15, ‘sp|Q7Z4H4|ADM2_HUMAN ADM2 OS = Homo sapiens GN = ADM2’, 148, 15865]
[‘0.97’, 11, ‘sp|P09341|GROA_HUMAN Growth-regulated alpha protein OS = Homo sapiens GN = CXCL1’, 107, 11301]
[‘0.97’, 6, ‘sp|O15263|BD02_HUMAN Beta-defensin 2 OS = Homo sapiens GN = DEFB4’, 64, 7037]
[‘0.96’, 40, ‘sp|Q96N58|ZN578_HUMAN Zinc finger protein 578 OS = Homo sapiens GN = ZNF578’, 365, 42596]
[‘0.96’, 19, ‘sp|Q9NPH9|IL26_HUMAN Interleukin-26 OS = Homo sapiens GN = IL26’, 171, 19842]
[‘0.96’, 19, ‘sp|Q8NHX4|SPTA3_HUMAN Spermatogenesis-associated protein 3 OS = Homo sapiens GN = SPATA3’, 183, 19948]
[‘0.96’, 16, ‘sp|P59020|DSCR9_HUMAN Down syndrome critical region protein 9 OS = Homo sapiens GN = DSCR9’, 149, 16743]
[‘0.96’, 8, ‘sp|Q3LI70|KR196_HUMAN Keratin-associated protein 19-6 OS = Homo sapiens GN = KRTAP19-6’, 84, 9125]
[‘0.96’, 7, ‘sp|Q9Y6X1|SERP1_HUMAN Stress-associated endoplasmic reticulum protein 1 OS = Homo sapiens GN = SERP1’, 66, 7373]
[‘0.96’, 4, ‘sp|Q9P0U5|INGX_HUMAN Inhibitor of growth protein, X-linked OS = Homo sapiens GN = INGX’, 42, 5076]
[‘0.95’, 7, ‘sp|Q8N6R1|SERP2_HUMAN Stress-associated endoplasmic reticulum protein 2 OS = Homo sapiens GN = SERP2’, 65, 7430]
[‘0.94’, 33, ‘sp|Q9H7B2|BXDC1_HUMAN Brix domain-containing protein 1 OS = Homo sapiens GN = BXDC1’, 306, 35582]
[‘0.94’, 17, ‘sp|Q96MF4|CC140_HUMAN Coiled-coil domain-containing protein 140 OS = Homo sapiens GN = CCDC140’, 163, 18252]
[‘0.94’, 16, ‘sp|Q8WW36|ZCH13_HUMAN Zinc finger CCHC domain-containing protein 13 OS = Homo sapiens GN = ZCCHC13’, 166,
18005]
[‘0.94’, 12, ‘sp|O60519|CRBL2_HUMAN cAMP-responsive element-binding protein-like 2 OS = Homo sapiens GN = CREBL2’, 120, 13783]
[‘0.93’, 16, ‘sp|Q9H1E1|RNAS7_HUMAN Ribonuclease 7 OS = Homo sapiens GN = RNASE7’, 156, 17471]
[‘0.93’, 16, ‘sp|Q14236|EPAG_HUMAN Early lymphoid activation gene protein OS = Homo sapiens GN = EPAG’, 149, 17843]
[‘0.93’, 16, ‘sp|P0C7M6|IQCF3_HUMAN IQ domain-containing protein F3 OS = Homo sapiens GN = IQCF3’, 154, 18250]
[‘0.93’, 11, ‘sp|O43927|CXL13_HUMAN C—X—C motif chemokine 13 OS = Homo sapiens GN = CXCL13’, 109, 12664]
[‘0.93’, 9, ‘sp|Q9Y6G1|TM14A_HUMAN Transmembrane protein 14A OS = Homo sapiens GN = TMEM14A’, 99, 10712]
[‘0.93’, 9, ‘sp|Q7Z7B7|DB132_HUMAN Beta-defensin 132 OS = Homo sapiens GN = DEFB132’, 95, 10610]
[‘0.93’, 8, ‘sp|Q5T5B0|LCE3E_HUMAN Late cornified envelope protein 3E OS = Homo sapiens GN = LCE3E’, 92, 9506]
[‘0.93’, 7, ‘sp|Q9NPE3|NOLA3_HUMAN H/ACA ribonucleoprotein complex subunit 3 OS = Homo sapiens GN = NOLA3’, 64, 7705]
[‘0.92’, 23, ‘sp|O95707|RPP29_HUMAN Ribonuclease P protein subunit p29 OS = Homo sapiens GN = POP4’, 220, 25424]
[‘0.92’, 14, ‘sp|Q9NPJ4|PNRC2_HUMAN Proline-rich nuclear receptor coactivator 2 OS = Homo sapiens GN = PNRC2’, 139, 15590]
[‘0.92’, 11, ‘sp|O14599|VCY2_HUMAN Testis-specific basic protein Y 2 OS = Homo sapiens GN = BPY2’, 106, 12035]
[‘0.92’, 8, ‘sp|Q8WVI0|U640_HUMAN UPF0640 protein OS = Homo sapiens’, 70, 8696]
[‘0.92’, 5, ‘sp|Q96IX5|USMG5_HUMAN Up-regulated during skeletal muscle growth protein 5 OS = Homo sapiens GN = USMG5’, 58, 6457]
[‘0.91’, 8, ‘sp|P61582|NP12_HUMAN HERV-K_1q22 provirus Np9 protein OS = Homo sapiens’, 75, 8820]
[‘0.90’, 81, ‘sp|Q08AN1|ZN616_HUMAN Zinc finger protein 616 OS = Homo sapiens GN = ZNF616’, 781, 90263]
[‘0.90’, 42, ‘sp|Q8N5F7|NKAP_HUMAN NF-kappa-B-activating protein OS = Homo sapiens GN = NKAP’, 415, 47138]
[‘0.90’, 41, ‘sp|A6NM28|ZFP92_HUMAN Zinc finger protein 92 homolog OS = Homo sapiens GN = ZFP92’, 416, 45791]
[‘0.90’, 35, ‘sp|Q14093|CYLC2_HUMAN Cylicin-2 OS = Homo sapiens GN = CYLC2’, 348, 39078]
[‘0.90’, 18, ‘sp|Q6ZT77|ZN826_HUMAN Zinc finger protein 826 OS = Homo sapiens GN = ZNF826’, 177, 20579]
[‘0.90’, 10, ‘sp|Q5T751|LCE1C_HUMAN Late cornified envelope protein 1C OS = Homo sapiens GN = LCE1C’, 118, 11543]
[‘0.90’, 8, ‘sp|P61583|NP8_HUMAN HERV-K_3q12.3 provirus Np9 protein OS = Homo sapiens GN = ERVK5’, 75, 8907]
[‘0.90’, 7, ‘sp|Q30KQ2|DB130_HUMAN Beta-defensin 130 OS = Homo sapiens GN = DEFB130’, 79, 8735]
[‘0.89’, 35, ‘sp|O75698|HUG1_HUMAN Protein HUG-1 OS = Homo sapiens GN = HUG1’, 362, 39386]
[‘0.89’, 22, ‘sp|Q8N7Y1|PRR10_HUMAN Proline-rich protein 10 OS = Homo sapiens GN = PRR10’, 241, 25772]
[‘0.89’, 22, ‘sp|Q5TFG8|F164B_HUMAN UPF0418 protein FAM164B OS = Homo sapiens GN = FAM164B’, 222, 24665]
[‘0.89’, 18, ‘sp|Q7RTS1|BHLH8_HUMAN Class B basic helix-loop-helix protein 8 OS = Homo sapiens GN = BHLHB8’, 189, 20818]
[‘0.89’, 10, ‘sp|Q5T7P3|LCE1B_HUMAN Late cornified envelope protein 1B OS = Homo sapiens GN = LCE1B’, 118, 11626]
[‘0.89’, 10, ‘sp|Q5T754|LCE1F_HUMAN Late cornified envelope protein 1F OS = Homo sapiens GN = LCE1F’, 118, 11654]
[‘0.89’, 10, ‘sp|P19876|MIP2B_HUMAN Macrophage inflammatory protein 2-beta OS = Homo sapiens GN = CXCL3’, 107, 11342]
[‘0.89’, 9, ‘sp|P80098|CCL7_HUMAN C-C motif chemokine 7 OS = Homo sapiens GN = CCL7’, 99, 11200]
[‘0.89’, 7, ‘sp|Q969E1|LEAP2_HUMAN Liver-expressed antimicrobial peptide 2 OS = Homo sapiens GN = LEAP2’, 77, 8813]
[‘0.89’, 7, ‘sp|Q30KP9|DB135_HUMAN Beta-defensin 135 OS = Homo sapiens GN = DEFB135’, 77, 8753]
[‘0.88’, 50, ‘sp|Q96CS4|ZN689_HUMAN Zinc finger protein 689 OS = Homo sapiens GN = ZNF689’, 500, 56906]
[‘0.88’, 24, ‘sp|Q5EBM4|ZN542_HUMAN Zinc finger protein 542 OS = Homo sapiens GN = ZNF542’, 241, 27663]
[‘0.88’, 11, ‘sp|Q96BP2|CHCH1_HUMAN Coiled-coil-helix-coiled-coil-helix domain-containing protein 1 OS = Homo sapiens
GN = CHCHD1’, 118, 13474]
[‘0.88’, 9, ‘sp|Q6UX46|F150B_HUMAN Protein FAM150B OS = Homo sapiens GN = FAM150B’, 91, 10541]
[‘0.87’, 65, ‘sp|Q6ZR52|ZN493_HUMAN Zinc finger protein 493 OS = Homo sapiens GN = ZNF493’, 646, 75341]
[‘0.87’, 30, ‘sp|Q99848|EBP2_HUMAN Probable rRNA-processing protein EBP2 OS = Homo sapiens GN = EBNA1BP2’, 306, 34851]
[‘0.87’, 12, ‘sp|P62318|SMD3_HUMAN Small nuclear ribonucleoprotein Sm D3 OS = Homo sapiens GN = SNRPD3’, 126, 13916]
[‘0.87’, 10, ‘sp|A0PJW8|DAPL1_HUMAN Death-associated protein-like 1 OS = Homo sapiens GN = DAPL1’, 107, 11879]
[‘0.87’, 9, ‘sp|Q5T7P2|LCE1A_HUMAN Late cornified envelope protein 1A OS = Homo sapiens GN = LCE1A’, 110, 10982]
[‘0.87’, 5, ‘sp|Q96KF2|PRAC_HUMAN Small nuclear protein PRAC OS = Homo sapiens GN = PRAC’, 57, 5958]
[‘0.86’, 59, ‘sp|Q03923|ZNF85_HUMAN Zinc finger protein 85 OS = Homo sapiens GN = ZNF85’, 595, 68718]
[‘0.86’, 54, ‘sp|Q6N045|ZNP12_HUMAN Zinc finger protein ZnFP12 OS = Homo sapiens’, 540, 62759]
[‘0.86’, 43, ‘sp|Q8IZC7|ZN101_HUMAN Zinc finger protein 101 OS = Homo sapiens GN = ZNF101’, 436, 50339]
[‘0.86’, 41, ‘sp|P42696|RBM34_HUMAN RNA-binding protein 34 OS = Homo sapiens GN = RBM34’, 430, 48564]
[‘0.86’, 20, ‘sp|Q9Y324|FCF1_HUMAN rRNA-processing protein FCF1 homolog OS = Homo sapiens GN = FCF1’, 198, 23369]
[‘0.86’, 15, ‘sp|Q969E3|UCN3_HUMAN Urocortin-3 OS = Homo sapiens GN = UCN3’, 161, 17861]
[‘0.86’, 13, ‘sp|P09132|SRP19_HUMAN Signal recognition particle 19 kDa protein OS = Homo sapiens GN = SRP19’, 144, 16155]
[‘0.85’, 54, ‘sp|Q9BWE0|REPI1_HUMAN Replication initiator 1 OS = Homo sapiens GN = REPIN1’, 567, 63574]
[‘0.85’, 42, ‘sp|Q8NCK3|ZN485_HUMAN Zinc finger protein 485 OS = Homo sapiens GN = ZNF485’, 441, 50280]
[‘0.85’, 22, ‘sp|P11487|FGF3_HUMAN INT-2 proto-oncogene protein OS = Homo sapiens GN = FGF3’, 239, 26886]
[‘0.85’, 19, ‘sp|Q99748|NRTN_HUMAN Neurturin OS = Homo sapiens GN = NRTN’, 197, 22405]
[‘0.85’, 6, ‘sp|P15954|COX7C_HUMAN Cytochrome c oxidase subunit 7C, mitochondrial OS = Homo sapiens GN = COX7C’, 63, 7245]
[‘0.84’, 42, ‘sp|Q8N8L2|ZN491_HUMAN Zinc finger protein 491 OS = Homo sapiens GN = ZNF491’, 437, 50949]
[‘0.84’, 22, ‘sp|Q86XF7|ZN575_HUMAN Zinc finger protein 575 OS = Homo sapiens GN = ZNF575’, 245, 26763]
[‘0.84’, 9, ‘sp|Q5T752|LCE1D_HUMAN Late cornified envelope protein 1D OS = Homo sapiens GN = LCE1D’, 114, 11229]
[‘0.84’, 6, ‘sp|Q9NRX6|T167B_HUMAN Transmembrane protein 167B OS = Homo sapiens GN = TMEM167B’, 74, 8294]
[‘0.84’, 5, ‘sp|P80294|MT1H_HUMAN Metallothionein-1H OS = Homo sapiens GN = MT1H’, 61, 6039]
[‘0.83’, 50, ‘sp|Q9P255|ZN492_HUMAN Zinc finger protein 492 OS = Homo sapiens GN = ZNF492’, 531, 61158]
[‘0.83’, 50, ‘sp|A6NK75|ZNF98_HUMAN Zinc finger protein 98 OS = Homo sapiens GN = ZNF98’, 531, 61144]
[‘0.83’, 32, ‘sp|O15480|MAGB3_HUMAN Melanoma-associated antigen B3 OS = Homo sapiens GN = MAGEB3’, 346, 39179]
[‘0.83’, 29, ‘sp|Q96GY0|F164A_HUMAN UPF0418 protein FAM164A OS = Homo sapiens GN = FAM164A’, 325, 35062]
[‘0.83’, 26, ‘sp|Q96PP4|TSG13_HUMAN Testis-specific gene 13 protein OS = Homo sapiens GN = TSGA13’, 275, 31777]
[‘0.83’, 17, ‘sp|O15499|GSC2_HUMAN Homeobox protein goosecoid-2 OS = Homo sapiens GN = GSC2’, 205, 21544]
[‘0.83’, 10, ‘sp|P56847|TNG2_HUMAN Protein TNG2 OS = Homo sapiens GN = TNG2’, 110, 12856]
[‘0.83’, 7, ‘sp|Q9BYE3|LCE3D_HUMAN Late cornified envelope protein 3D OS = Homo sapiens GN = LCE3D’, 92, 9443]
[‘0.83’, 5, ‘sp|P07438|MT1B_HUMAN Metallothionein-1B OS = Homo sapiens GN = MT1B’, 61, 6115]
[‘0.82’, 31, ‘sp|Q6AZW8|ZN660_HUMAN Zinc finger protein 660 OS = Homo sapiens GN = ZNF660’, 331, 38270]
[‘0.82’, 11, ‘sp|O43612|OREX_HUMAN Orexin OS = Homo sapiens GN = HCRT’, 131, 13362]
[‘0.82’, 10, ‘sp|Q96DA6|TIM14_HUMAN Mitochondrial import inner membrane translocase subunit TIM14 OS = Homo sapiens
GN = DNAJC19’, 116, 12498]
[‘0.82’, 9, ‘sp|Q96A98|TIP39_HUMAN Tuberoinfundibular peptide of 39 residues OS = Homo sapiens GN = PTH2’, 100, 11202]
[‘0.82’, 9, ‘sp|P80162|CXCL6_HUMAN C—X—C motif chemokine 6 OS = Homo sapiens GN = CXCL6’, 114, 11897]
[‘0.81’, 23, ‘sp|Q9P031|TAP26_HUMAN Thyroid transcription factor 1-associated protein 26 OS = Homo sapiens GN = CCDC59’, 241, 28669]
[‘0.81’, 11, ‘sp|Q6ZST2|ZCH23_HUMAN Zinc finger CCHC domain-containing protein 23 OS = Homo sapiens GN = ZCCHC23’, 131, 14409]
[‘0.81’, 11, ‘sp|P62316|SMD2_HUMAN Small nuclear ribonucleoprotein Sm D2 OS = Homo sapiens GN = SNRPD2’, 118, 13526]
[‘0.81’, 10, ‘sp|O95182|NDUA7_HUMAN NADH dehydrogenase [ubiquinone] 1 alpha subcomplex subunit 7 OS = Homo sapiens
GN = NDUFA7’, 113, 12551]
[‘0.81’, 10, ‘sp|A6NFY7|LYRM8_HUMAN LYR motif-containing protein ENSP00000368165 OS = Homo sapiens’, 115, 12806]
[‘0.81’, 7, ‘sp|Q7Z3B0|CE043_HUMAN UPF0542 protein C5orf43 OS = Homo sapiens GN = C5orf43’, 74, 8625]
[‘0.80’, 72, ‘sp|Q9UII5|ZN107_HUMAN Zinc finger protein 107 OS = Homo sapiens GN = ZNF107’, 783, 90672]
[‘0.80’, 69, ‘sp|Q9Y3M9|ZN337_HUMAN Zinc finger protein 337 OS = Homo sapiens GN = ZNF337’, 751, 86874]
[‘0.80’, 49, ‘sp|Q5SXM1|ZN678_HUMAN Zinc finger protein 678 OS = Homo sapiens GN = ZNF678’, 525, 61411]
[‘0.80’, 47, ‘sp|Q96BV0|ZN775_HUMAN Zinc finger protein 775 OS = Homo sapiens GN = ZNF775’, 537, 59751]
[‘0.80’, 40, ‘sp|P51522|ZNF83_HUMAN Zinc finger protein 83 OS = Homo sapiens GN = ZNF83’, 428, 49778]
[‘0.80’, 19, ‘sp|Q9UGY1|NOL12_HUMAN Nucleolar protein 12 OS = Homo sapiens GN = NOL12’, 213, 24662]
[‘0.80’, 19, ‘sp|O76093|FGF18_HUMAN Fibroblast growth factor 18 OS = Homo sapiens GN = FGF18’, 207, 23988]
[‘0.80’, 16, ‘sp|P20800|EDN2_HUMAN Endothelin-2 OS = Homo sapiens GN = EDN2’, 178, 19959]
[‘0.80’, 8, ‘sp|Q9NRX3|NUA4L_HUMAN NADH dehydrogenase [ubiquinone] 1 alpha subcomplex subunit 4-like 2 OS = Homo sapiens
GN = NDUFA4L2’, 87, 9965]
[‘0.80’, 8, ‘sp|Q02221|CX6A2_HUMAN Cytochrome c oxidase polypeptide 6A2, mitochondrial OS = Homo sapiens GN = COX6A2’, 97,
10815]
[‘0.80’, 5, ‘sp|Q9P0U1|TOM7_HUMAN Mitochondrial import receptor subunit TOM7 homolog OS = Homo sapiens GN = TOMM7’, 55, 6248]
Histones
[‘2.70’, 59, ‘sp|P10412|H14_HUMAN Histone H1.4 OS = Homo sapiens GN = HIST1H1E’, 219, 21865]
[‘2.66’, 60, ‘sp|P16401|H15_HUMAN Histone H1.5 OS = Homo sapiens GN = HIST1H1B’, 226, 22580]
[‘2.60’, 58, ‘sp|P16402|H13_HUMAN Histone H1.3 OS = Homo sapiens GN = HIST1H1D’, 221, 22349]
[‘2.57’, 55, ‘sp|P16403|H12_HUMAN Histone H1.2 OS = Homo sapiens GN = HIST1H1C’, 213, 21364]
[‘2.55’, 53, ‘sp|P07305|H10_HUMAN Histone H1.0 OS = Homo sapiens GN = H1F0’, 194, 20862]
[‘2.47’, 54, ‘sp|Q02539|H11_HUMAN Histone H1.1 OS = Homo sapiens GN = HIST1H1A’, 215, 21842]
[‘2.10’, 46, ‘sp|P22492|H1T_HUMAN Histone H1t OS = Homo sapiens GN = HIST1H1T’, 207, 22018]
[‘1.79’, 40, ‘sp|Q92522|H1X_HUMAN Histone H1x OS = Homo sapiens GN = H1FX’, 213, 22487]
[‘1.63’, 42, ‘sp|Q75WM6|H1FNT_HUMAN Testis-specific H1 histone OS = Homo sapiens GN = H1FNT’, 234, 25888]
[‘1.60’, 18, ‘sp|P62805|H4_HUMAN Histone H4 OS = Homo sapiens GN = HIST1H4A’, 103, 11367]
[‘1.56’, 17, ‘sp|Q99525|H4G_HUMAN Histone H4-like protein type G OS = Homo sapiens GN = HIST1H4G’, 98, 11009]
[‘1.39’, 35, ‘sp|P60008|HILS1_HUMAN Spermatid-specific linker histone H1-like protein OS = Homo sapiens GN = HILS1’, 231, 25631]
[‘1.32’, 18, ‘sp|Q93079|H2B1H_HUMAN Histone H2B type 1-H OS = Homo sapiens GN = HIST1H2BH’, 126, 13892]
[‘1.32’, 18, ‘sp|O60814|H2B1K_HUMAN Histone H2B type 1-K OS = Homo sapiens GN = HIST1H2BK’, 126, 13890]
[‘1.31’, 20, ‘sp|Q71DI3|H32_HUMAN Histone H3.2 OS = Homo sapiens GN = HIST2H3A’, 136, 15388]
[‘1.31’, 20, ‘sp|P84243|H33_HUMAN Histone H3.3 OS = Homo sapiens GN = H3F3A’, 136, 15327]
[‘1.31’, 20, ‘sp|P68431|H31_HUMAN Histone H3.1 OS = Homo sapiens GN = HIST1H3A’, 136, 15404]
[‘1.31’, 18, ‘sp|Q99880|H2B1L_HUMAN Histone H2B type 1-L OS = Homo sapiens GN = HIST1H2BL’, 126, 13952]
[‘1.31’, 18, ‘sp|Q99879|H2B1M_HUMAN Histone H2B type 1-M OS = Homo sapiens GN = HIST1H2BM’, 126, 13989]
[‘1.31’, 18, ‘sp|Q99877|H2B1N_HUMAN Histone H2B type 1-N OS = Homo sapiens GN = HIST1H2BN’, 126, 13922]
[‘1.31’, 18, ‘sp|Q8N257|H2B3B_HUMAN Histone H2B type 3-B OS = Homo sapiens GN = HIST3H2BB’, 126, 13908]
[‘1.31’, 18, ‘sp|Q5QNW6|H2B2F_HUMAN Histone H2B type 2-F OS = Homo sapiens GN = HIST2H2BF’, 126, 13920]
[‘1.31’, 18, ‘sp|Q16778|H2B2E_HUMAN Histone H2B type 2-E OS = Homo sapiens GN = HIST2H2BE’, 126, 13920]
[‘1.31’, 18, ‘sp|P58876|H2B1D_HUMAN Histone H2B type 1-D OS = Homo sapiens GN = HIST1H2BD’, 126, 13936]
[‘1.31’, 18, ‘sp|P57053|H2BFS_HUMAN Histone H2B type F-S OS = Homo sapiens GN = H2BFS’, 126, 13944]
[‘1.31’, 18, ‘sp|P33778|H2B1B_HUMAN Histone H2B type 1-B OS = Homo sapiens GN = HIST1H2BB’, 126, 13950]
[‘1.31’, 18, ‘sp|P23527|H2B1O_HUMAN Histone H2B type 1-O OS = Homo sapiens GN = HIST1H2BO’, 126, 13906]
[‘1.31’, 18, ‘sp|P06899|H2B1J_HUMAN Histone H2B type 1-J OS = Homo sapiens GN = HIST1H2BJ’, 126, 13904]
[‘1.30’, 20, ‘sp|Q16695|H31T_HUMAN Histone H3.1t OS = Homo sapiens GN = HIST3H3’, 136, 15508]
[‘1.29’, 18, ‘sp|Q96A08|H2B1A_HUMAN Histone H2B type 1-A OS = Homo sapiens GN = HIST1H2BA’, 127, 14167]
[‘1.28’, 12, ‘sp|P05204|HMGN2_HUMAN Non-histone chromosomal protein HMG-17 OS = Homo sapiens GN = HMGN2’, 90, 9392]
[‘1.24’, 17, ‘sp|Q16777|H2A2C_HUMAN Histone H2A type 2-C OS = Homo sapiens GN = HIST2H2AC’, 129, 13988]
[‘1.23’, 17, ‘sp|Q93077|H2A1C_HUMAN Histone H2A type 1-C OS = Homo sapiens GN = HIST1H2AC’, 130, 14105]
[‘1.23’, 17, ‘sp|Q7L7L0|H2A3_HUMAN Histone H2A type 3 OS = Homo sapiens GN = HIST3H2A’, 130, 14121]
[‘1.23’, 17, ‘sp|Q6FI13|H2A2A_HUMAN Histone H2A type 2-A OS = Homo sapiens GN = HIST2H2AA3’, 130, 14095]
[‘1.23’, 17, ‘sp|P20671|H2A1D_HUMAN Histone H2A type 1-D OS = Homo sapiens GN = HIST1H2AD’, 130, 14107]
[‘1.23’, 17, ‘sp|P0C0S8|H2A1_HUMAN Histone H17/2A type 1 OS = Homo sapiens GN = HIST1H2AG’, 130, 14091]
[‘1.23’, 17, ‘sp|P04908|H2A1B_HUMAN Histone H2A type 1-B/E OS = Homo sapiens GN = HIST1H2AB’, 130, 14135]
[‘1.19’, 18, ‘sp|Q6NXT2|H3L_HUMAN Histone H3-like OS = Homo sapiens’, 135, 15213]
[‘1.18’, 16, ‘sp|Q96KK5|H2A1H_HUMAN Histone H2A type 1-H OS = Homo sapiens GN = HIST1H2AH’, 128, 13906]
[‘1.17’, 16, ‘sp|Q99878|H2A1J_HUMAN Histone H2A type 1-J OS = Homo sapiens GN = HIST1H2AJ’, 128, 13936]
[‘1.16’, 16, ‘sp|Q8IUE6|H2A2B_HUMAN Histone H2A type 2-B OS = Homo sapiens GN = HIST2H2AB’, 130, 13995]
[‘1.09’, 15, ‘sp|Q96QV6|H2A1A_HUMAN Histone H2A type 1-A OS = Homo sapiens GN = HIST1H2AA’, 131, 14233]
[‘1.08’, 16, ‘sp|P16104|H2AX_HUMAN Histone H2A.x OS = Homo sapiens GN = H2AFX’, 143, 15144]
[‘1.08’, 14, ‘sp|Q71UI9|H2AV_HUMAN Histone H2A.V OS = Homo sapiens GN = H2AFV’, 128, 13508]
[‘1.07’, 14, ‘sp|P0C0S5|H2AZ_HUMAN Histone H2A.Z OS = Homo sapiens GN = H2AFZ’, 128, 13552]
Ribosome
[‘2.87’, 19, ‘sp|P62861|RS30_HUMAN 40S ribosomal protein S30 OS = Homo sapiens GN = FAU’, 59, 6647]
[‘2.84’, 18, ‘sp|P62891|RL39_HUMAN 60S ribosomal protein L39 OS = Homo sapiens GN = RPL39’, 51, 6406]
[‘2.57’, 16, ‘sp|Q96EH5|RL39L_HUMAN 60S ribosomal protein L39-like OS = Homo sapiens GN = RPL39L’, 51, 6292]
[‘2.54’, 28, ‘sp|P61927|RL37_HUMAN 60S ribosomal protein L37 OS = Homo sapiens GN = RPL37’, 97, 11077]
[‘2.28’, 40, ‘sp|P47914|RL29_HUMAN 60S ribosomal protein L29 OS = Homo sapiens GN = RPL29’, 159, 17752]
[‘2.17’, 28, ‘sp|P49207|RL34_HUMAN 60S ribosomal protein L34 OS = Homo sapiens GN = RPL34’, 117, 13292]
[‘2.17’, 27, ‘sp|Q969Q0|RL36L_HUMAN 60S ribosomal protein L36a-like OS = Homo sapiens GN = RPL36AL’, 106, 12468]
[‘2.17’, 27, ‘sp|P83881|RL36A_HUMAN 60S ribosomal protein L36a OS = Homo sapiens GN = RPL36A’, 106, 12440]
[‘2.07’, 30, ‘sp|P42766|RL35_HUMAN 60S ribosomal protein L35 OS = Homo sapiens GN = RPL35’, 123, 14551]
[‘2.07’, 25, ‘sp|Q9Y3U8|RL36_HUMAN 60S ribosomal protein L36 OS = Homo sapiens GN = RPL36’, 105, 12253]
[‘1.97’, 35, ‘sp|P83731|RL24_HUMAN 60S ribosomal protein L24 OS = Homo sapiens GN = RPL24’, 157, 17778]
[‘1.92’, 30, ‘sp|P46779|RL28_HUMAN 60S ribosomal protein L28 OS = Homo sapiens GN = RPL28’, 137, 15747]
[‘1.90’, 44, ‘sp|P84098|RL19_HUMAN 60S ribosomal protein L19 OS = Homo sapiens GN = RPL19’, 196, 23465]
[‘1.85’, 19, ‘sp|P61513|RL37A_HUMAN 60S ribosomal protein L37a OS = Homo sapiens GN = RPL37A’, 92, 10275]
[‘1.72’, 37, ‘sp|Q07020|RL18_HUMAN 60S ribosomal protein L18 OS = Homo sapiens GN = RPL18’, 188, 21634]
[‘1.69’, 22, ‘sp|P62854|RS26_HUMAN 40S ribosomal protein S26 OS = Homo sapiens GN = RPS26’, 115, 13015]
[‘1.68’, 39, ‘sp|P50914|RL14_HUMAN 60S ribosomal protein L14 OS = Homo sapiens GN = RPL14’, 213, 23289]
[‘1.66’, 26, ‘sp|P62910|RL32_HUMAN 60S ribosomal protein L32 OS = Homo sapiens GN = RPL32’, 135, 15859]
[‘1.65’, 39, ‘sp|P61313|RL15_HUMAN 60S ribosomal protein L15 OS = Homo sapiens GN = RPL15’, 204, 24146]
[‘1.63’, 26, ‘sp|P46776|RL27A_HUMAN 60S ribosomal protein L27a OS = Homo sapiens GN = RPL27A’, 148, 16561]
[‘1.63’, 19, ‘sp|Q9P0J6|RM36_HUMAN 39S ribosomal protein L36, mitochondrial OS = Homo sapiens GN = MRPL36’, 103, 11784]
[‘1.62’, 39, ‘sp|P26373|RL13_HUMAN 60S ribosomal protein L13 OS = Homo sapiens GN = RPL13’, 211, 24261]
[‘1.61’, 52, ‘sp|Q02878|RL6_HUMAN 60S ribosomal protein L6 OS = Homo sapiens GN = RPL6’, 288, 32727]
[‘1.59’, 25, ‘sp|P61353|RL27_HUMAN 60S ribosomal protein L27 OS = Homo sapiens GN = RPL27’, 136, 15797]
[‘1.55’, 36, ‘sp|P40429|RL13A_HUMAN 60S ribosomal protein L13a OS = Homo sapiens GN = RPL13A’, 203, 23577]
[‘1.55’, 27, ‘sp|P62750|RL23A_HUMAN 60S ribosomal protein L23a OS = Homo sapiens GN = RPL23A’, 156, 17695]
[‘1.54’, 33, ‘sp|Q9NZE8|RM35_HUMAN 39S ribosomal protein L35, mitochondrial OS = Homo sapiens GN = MRPL35’, 188, 21514]
[‘1.53’, 19, ‘sp|P18077|RL35A_HUMAN 60S ribosomal protein L35a OS = Homo sapiens GN = RPL35A’, 110, 12537]
[‘1.50’, 71, ‘sp|P36578|RL4_HUMAN 60S ribosomal protein L4 OS = Homo sapiens GN = RPL4’, 427, 47697]
[‘1.49’, 15, ‘sp|Q9BQ48|RM34_HUMAN 39S ribosomal protein L34, mitochondrial OS = Homo sapiens GN = MRPL34’, 92, 10164]
[‘1.48’, 25, ‘sp|Q9UNX3|RL26L_HUMAN 60S ribosomal protein L26-like 1 OS = Homo sapiens GN = RPL26L1’, 145, 17256]
[‘1.48’, 25, ‘sp|P61254|RL26_HUMAN 60S ribosomal protein L26 OS = Homo sapiens GN = RPL26’, 145, 17258]
[‘1.47’, 42, ‘sp|P62753|RS6_HUMAN 40S ribosomal protein S6 OS = Homo sapiens GN = RPS6’, 249, 28680]
[‘1.46’, 11, ‘sp|P63173|RL38_HUMAN 60S ribosomal protein L38 OS = Homo sapiens GN = RPL38’, 70, 8217]
[‘1.45’, 11, ‘sp|O75394|RM33_HUMAN 39S ribosomal protein L33, mitochondrial OS = Homo sapiens GN = MRPL33’, 65, 7619]
[‘1.41’, 34, ‘sp|P62241|RS8_HUMAN 40S ribosomal protein S8 OS = Homo sapiens GN = RPS8’, 208, 24205]
[‘1.39’, 19, ‘sp|P62851|RS25_HUMAN 40S ribosomal protein S25 OS = Homo sapiens GN = RPS25’, 125, 13742]
[‘1.38’, 41, ‘sp|P62424|RL7A_HUMAN 60S ribosomal protein L7a OS = Homo sapiens GN = RPL7A’, 266, 29995]
[‘1.38’, 40, ‘sp|P18124|RL7_HUMAN 60S ribosomal protein L7 OS = Homo sapiens GN = RPL7’, 248, 29225]
[‘1.38’, 25, ‘sp|P46778|RL21_HUMAN 60S ribosomal protein L21 OS = Homo sapiens GN = RPL21’, 160, 18564]
[‘1.37’, 28, ‘sp|Q02543|RL18A_HUMAN 60S ribosomal protein L18a OS = Homo sapiens GN = RPL18A’, 176, 20762]
[‘1.36’, 9, ‘sp|P62273|RS29_HUMAN 40S ribosomal protein S29 OS = Homo sapiens GN = RPS29’, 56, 6676]
[‘1.35’, 37, ‘sp|P62917|RL8_HUMAN 60S ribosomal protein L8 OS = Homo sapiens GN = RPL8’, 257, 28024]
[‘1.35’, 21, ‘sp|P62266|RS23_HUMAN 40S ribosomal protein S23 OS = Homo sapiens GN = RPS23’, 143, 15807]
[‘1.32’, 39, ‘sp|O95478|NSA2_HUMAN Ribosome biogenesis protein NSA2 homolog OS = Homo sapiens GN = TINP1’, 260, 30065]
[‘1.30’, 20, ‘sp|Q86WX3|S19BP_HUMAN 40S ribosomal protein S19-binding protein 1 OS = Homo sapiens GN = RPS19BP1’, 136, 15433]
[‘1.28’, 22, ‘sp|Q9BYC9|RM20_HUMAN 39S ribosomal protein L20, mitochondrial OS = Homo sapiens GN = MRPL20’, 149, 17442]
[‘1.26’, 23, ‘sp|P62280|RS11_HUMAN 40S ribosomal protein S11 OS = Homo sapiens GN = RPS11’, 158, 18430]
[‘1.21’, 18, ‘sp|Q4U2R6|RM51_HUMAN 39S ribosomal protein L51, mitochondrial OS = Homo sapiens GN = MRPL51’, 128, 15094]
[‘1.19’, 20, ‘sp|P62277|RS13_HUMAN 40S ribosomal protein S13 OS = Homo sapiens GN = RPS13’, 151, 17222]
[‘1.19’, 17, ‘sp|P62899|RL31_HUMAN 60S ribosomal protein L31 OS = Homo sapiens GN = RPL31’, 125, 14462]
[‘1.16’, 20, ‘sp|P62269|RS18_HUMAN 40S ribosomal protein S18 OS = Homo sapiens GN = RPS18’, 152, 17718]
[‘1.14’, 17, ‘sp|P62829|RL23_HUMAN 60S ribosomal protein L23 OS = Homo sapiens GN = RPL23’, 140, 14865]
[‘1.12’, 33, ‘sp|P82914|RT15_HUMAN 28S ribosomal protein S15, mitochondrial OS = Homo sapiens GN = MRPS15’, 257, 29842]
[‘1.10’, 51, ‘sp|Q92901|RL3L_HUMAN 60S ribosomal protein L3-like OS = Homo sapiens GN = RPL3L’, 407, 46295]
[‘1.10’, 18, ‘sp|P62249|RS16_HUMAN 40S ribosomal protein S16 OS = Homo sapiens GN = RPS16’, 146, 16445]
[‘1.09’, 23, ‘sp|P18621|RL17_HUMAN 60S ribosomal protein L17 OS = Homo sapiens GN = RPL17’, 184, 21397]
[‘1.07’, 21, ‘sp|Q9UHA3|RLP24_HUMAN Probable ribosome biogenesis protein RLP24 OS = Homo sapiens GN = C15orf15’, 163, 19621]
[‘1.07’, 16, ‘sp|O60783|RT14_HUMAN 28S ribosomal protein S14, mitochondrial OS = Homo sapiens GN = MRPS14’, 128, 15138]
[‘1.06’, 16, ‘sp|O15235|RT12_HUMAN 28S ribosomal protein S12, mitochondrial OS = Homo sapiens GN = MRPS12’, 138, 15172]
[‘1.05’, 48, ‘sp|P39023|RL3_HUMAN 60S ribosomal protein L3 OS = Homo sapiens GN = RPL3’, 403, 46108]
[‘1.03’, 25, ‘sp|P27635|RL10_HUMAN 60S ribosomal protein L10 OS = Homo sapiens GN = RPL10’, 214, 24603]
[‘1.03’, 16, ‘sp|Q9P0M9|RM27_HUMAN 39S ribosomal protein L27, mitochondrial OS = Homo sapiens GN = MRPL27’, 148, 16072]
[‘1.03’, 11, ‘sp|P82921|RT21_HUMAN 28S ribosomal protein S21, mitochondrial OS = Homo sapiens GN = MRPS21’, 87, 10741]
[‘1.02’, 12, ‘sp|Q9BQC6|RT63_HUMAN Ribosomal protein 63, mitochondrial OS = Homo sapiens GN = MRP63’, 102, 12266]
[‘1.00’, 28, ‘sp|Q6DKI1|RL7L_HUMAN 60S ribosomal protein L7-like 1 OS = Homo sapiens GN = RPL7L1’, 246, 28660]
[‘0.99’, 22, ‘sp|P46781|RS9_HUMAN 40S ribosomal protein S9 OS = Homo sapiens GN = RPS9’, 194, 22591]
[‘0.98’, 53, ‘sp|O76021|RL1D1_HUMAN Ribosomal L1 domain-containing protein 1 OS = Homo sapiens GN = RSL1D1’, 490, 54972]
[‘0.97’, 32, ‘sp|Q5T653|RM02_HUMAN 39S ribosomal protein L2, mitochondrial OS = Homo sapiens GN = MRPL2’, 305, 33300]
[‘0.96’, 23, ‘sp|Q96L21|RL10L_HUMAN 60S ribosomal protein L10-like OS = Homo sapiens GN = RPL10L’, 214, 24518]
[‘0.96’, 21, ‘sp|Q9NVS2|RT18A_HUMAN 28S ribosomal protein S18a, mitochondrial OS = Homo sapiens GN = MRPS18A’, 196, 22183]
[‘0.96’, 9, ‘sp|Q71UM5|RS27L_HUMAN 40S ribosomal protein S27-like protein OS = Homo sapiens GN = RPS27L’, 84, 9477]
[‘0.96’, 9, ‘sp|P42677|RS27_HUMAN 40S ribosomal protein S27 OS = Homo sapiens GN = RPS27’, 84, 9461]
[‘0.93’, 38, ‘sp|Q15050|RRS1_HUMAN Ribosome biogenesis regulatory protein homolog OS = Homo sapiens GN = RRS1’, 365, 41193]
[‘0.90’, 14, ‘sp|Q6P1L8|RM14_HUMAN 39S ribosomal protein L14, mitochondrial OS = Homo sapiens GN = MRPL14’, 145, 15947]
[‘0.90’, 14, ‘sp|P39019|RS19_HUMAN 40S ribosomal protein S19 OS = Homo sapiens GN = RPS19’, 145, 16060]
[‘0.87’, 25, ‘sp|Q9HD33|RM47_HUMAN 39S ribosomal protein L47, mitochondrial OS = Homo sapiens GN = MRPL47’, 252, 29577]
[‘0.86’, 21, ‘sp|P62906|RL10A_HUMAN 60S ribosomal protein L10a OS = Homo sapiens GN = RPL10A’, 217, 24831]
[‘0.84’, 26, ‘sp|P15880|RS2_HUMAN 40S ribosomal protein S2 OS = Homo sapiens GN = RPS2’, 293, 31324]
[‘0.83’, 13, ‘sp|Q9Y3D5|RT18C_HUMAN 28S ribosomal protein S18c, mitochondrial OS = Homo sapiens GN = MRPS18C’, 142, 15849]
RS Domain
[‘1.74’, 44, ‘sp|Q01130|SFRS2_HUMAN Splicing factor, arginine/serine-rich 2 OS = Homo sapiens GN = SFRS2’, 221, 25476]
[‘1.66’, 93, ‘sp|Q08170|SFRS4_HUMAN Splicing factor, arginine/serine-rich 4 OS = Homo sapiens GN = SFRS4’, 494, 56678]
[‘1.35’, 26, ‘sp|P84103|SFRS3_HUMAN Splicing factor, arginine/serine-rich 3 OS = Homo sapiens GN = SFRS3’, 164, 19329]
[‘0.91’, 48, ‘sp|Q05519|SFR11_HUMAN Splicing factor arginine/serine-rich 11 OS = Homo sapiens GN = SFRS11’, 484, 53542]
Isoforms
[‘2.10’, 36, ‘sp|Q8N2M8-2|SFR16_HUMAN Isoform 2 of Splicing factor, arginine/serine-rich 16 OS = Homo sapiens GN = SFRS16’, 159,
17218]
[‘1.96’, 41, ‘sp|Q8IZA3-2|H1FOO_HUMAN Isoform 2 of Histone H1oo OS = Homo sapiens GN = H1FOO’, 207, 21010]
[‘1.93’, 51, ‘sp|Q9BUV0-3|CA063_HUMAN Isoform 3 of UPF0471 protein C1orf63 OS = Homo sapiens GN = C1orf63’, 226, 26604]
[‘1.93’, 10, ‘sp|Q9Y5P2-3|CSAG2_HUMAN Isoform 3 of Chondrosarcoma-associated gene 2/3A protein OS = Homo sapiens GN = CSAG2’,
48, 5216]
[‘1.87’, 28, ‘sp|Q8NAV1-2|PR38A_HUMAN Isoform 2 of Pre-mRNA-splicing factor 38A OS = Homo sapiens GN = PRPF38A’, 125, 15462]
[‘1.83’, 10, ‘sp|Q32NB8-4|PGPS1_HUMAN Isoform 4 of CDP-diacylglycerol--glycerol-3-phosphate 3-phosphatidyltransferase, mitochondrial
OS = Homo sapiens GN = PGS1’, 50, 5463]
[‘1.77’, 50, ‘sp|Q9BUV0-2|CA063_HUMAN Isoform 2 of UPF0471 protein C1orf63 OS = Homo sapiens GN = C1orf63’, 242, 28363]
[‘1.74’, 30, ‘sp|P49760-2|CLK2_HUMAN Isoform Short of Dual specificity protein kinase CLK2 OS = Homo sapiens GN = CLK2’, 139, 17569]
[‘1.68’, 46, ‘sp|Q16629-1|SFRS7_HUMAN Isoform 1 of Splicing factor, arginine/serine-rich 7 OS = Homo sapiens GN = SFRS7’, 238, 27366]
[‘1.68’, 25, ‘sp|P62847-2|RS24_HUMAN Isoform 2 of 40S ribosomal protein S24 OS = Homo sapiens GN = RPS24’, 130, 15068]
[‘1.66’, 59, ‘sp|Q8IZA3-1|H1FOO_HUMAN Isoform 1 of Histone H1oo OS = Homo sapiens GN = H1FOO’, 346, 35813]
[‘1.66’, 53, ‘sp|Q9BRL6-1|SFR2B_HUMAN Isoform 1 of Splicing factor, arginine/serine-rich 2B OS = Homo sapiens GN = SFRS2B’, 282,
32287]
[‘1.65’, 25, ‘sp|P62847-1|RS24_HUMAN Isoform 1 of 40S ribosomal protein S24 OS = Homo sapiens GN = RPS24’, 133, 15423]
[‘1.61’, 54, ‘sp|Q9BUV0-1|CA063_HUMAN Isoform 1 of UPF0471 protein C1orf63 OS = Homo sapiens GN = C1orf63’, 290, 33613]
[‘1.61’, 50, ‘sp|Q9BRL6-2|SFR2B_HUMAN Isoform 2 of Splicing factor, arginine/serine-rich 2B OS = Homo sapiens GN = SFRS2B’, 275,
31424]
[‘1.61’, 6, ‘sp|Q92876-3|KLK6_HUMAN Isoform 3 of Kallikrein-6 OS = Homo sapiens GN = KLK6’, 40, 4333]
[‘1.60’, 54, ‘sp|Q15287-1|RNPS1_HUMAN Isoform 1 of RNA-binding protein with serine-rich domain 1 OS = Homo sapiens GN = RNPS1’,
305, 34208]
[‘1.58’, 32, ‘sp|Q13875-2|MOBP_HUMAN Isoform 2 of Myelin-associated oligodendrocyte basic protein OS = Homo sapiens GN = MOBP’,
182, 20772]
[‘1.57’, 49, ‘sp|Q15287-2|RNPS1_HUMAN Isoform 2 of RNA-binding protein with serine-rich domain 1 OS = Homo sapiens GN = RNPS1’,
282, 31709]
[‘1.57’, 32, ‘sp|Q13875-1|MOBP_HUMAN Isoform 1 of Myelin-associated oligodendrocyte basic protein OS = Homo sapiens GN = MOBP’,
183, 20959]
[‘1.56’, 50, ‘sp|Q66PJ3-5|AR6P4_HUMAN Isoform 5 of ADP-ribosylation factor-like protein 6-interacting protein 4 OS = Homo sapiens
GN = ARL6IP4’, 304, 32178]
[‘1.55’, 44, ‘sp|Q9HB58-4|SP110_HUMAN Isoform 4 of Sp110 nuclear body protein OS = Homo sapiens GN = SP110’, 248, 28609]
[‘1.54’, 33, ‘sp|Q66PJ3-6|AR6P4_HUMAN Isoform 6 of ADP-ribosylation factor-like protein 6-interacting protein 4 OS = Homo sapiens
GN = ARL6IP4’, 215, 22007]
[‘1.51’, 28, ‘sp|P49761-2|CLK3_HUMAN Isoform 2 of Dual specificity protein kinase CLK3 OS = Homo sapiens GN = CLK3’, 152, 18971]
[‘1.44’, 18, ‘sp|Q14CB8-4|RHG19_HUMAN Isoform 4 of Rho GTPase-activating protein 19 OS = Homo sapiens GN = ARHGAP19’, 112,
12547]
[‘1.44’, 13, ‘sp|Q13875-3|MOBP_HUMAN Isoform 3 of Myelin-associated oligodendrocyte basic protein OS = Homo sapiens GN = MOBP’, 81,
9614]
[‘1.43’, 44, ‘sp|O75494-2|FUSIP_HUMAN Isoform 2 of FUS-interacting serine-arginine-rich protein 1 OS = Homo sapiens GN = FUSIP1’, 261,
31213]
[‘1.43’, 12, ‘sp|Q15651-2|HMGN3_HUMAN Isoform 2 of High mobility group nucleosome-binding domain-containing protein 3 OS = Homo
sapiens GN = HMGN3’, 77, 8377]
[‘1.42’, 56, ‘sp|Q13247-1|SFRS6_HUMAN Isoform SRP55-1 of Splicing factor, arginine/serine-rich 6 OS = Homo sapiens GN = SFRS6’, 344,
39586]
[‘1.42’, 44, ‘sp|O75494-1|FUSIP_HUMAN Isoform 1 of FUS-interacting serine-arginine-rich protein 1 OS = Homo sapiens GN = FUSIP1’, 262,
31300]
[‘1.42’, 8, ‘sp|Q70YC5-5|ZN365_HUMAN Isoform 6 of Protein ZNF365 OS = Homo sapiens GN = ZNF365’, 51, 5653]
[‘1.41’, 48, ‘sp|Q9UK58-3|CCNL1_HUMAN Isoform 3 of Cyclin-L1 OS = Homo sapiens GN = CCNL1’, 299, 34688]
[‘1.41’, 9, ‘sp|Q2NKX9-2|CB068_HUMAN Isoform 2 of UPF0561 protein C2orf68 OS = Homo sapiens GN = C2orf68’, 58, 6747]
[‘1.39’, 25, ‘sp|Q66K41-2|Z385C_HUMAN Isoform 2 of Zinc finger protein 385C OS = Homo sapiens GN = ZNF385C’, 174, 18242]
[‘1.38’, 10, ‘sp|Q9UQ07-3|MOK_HUMAN Isoform 3 of MAPK/MAK/MRK overlapping kinase OS = Homo sapiens GN = RAGE’, 73, 7879]
[‘1.37’, 42, ‘sp|Q13243-3|SFRS5_HUMAN Isoform SRP40-4 of Splicing factor, arginine/serine-rich 5 OS = Homo sapiens GN = SFRS5’, 269,
30858]
[‘1.36’, 23, ‘sp|Q6PGN9-4|PSRC1_HUMAN Isoform D of Proline/serine-rich coiled-coil protein 1 OS = Homo sapiens GN = PSRC1’, 163,
16980]
[‘1.36’, 15, ‘sp|Q6P1Q0-6|LTMD1_HUMAN Isoform 6 of LETM1 domain-containing protein 1 OS = Homo sapiens GN = LETMD1’, 99,
11221]
[‘1.36’, 10, ‘sp|O75920-2|SERF1_HUMAN Isoform Short of Small EDRK-rich factor 1 OS = Homo sapiens GN = SERF1A’, 62, 7336]
[‘1.35’, 68, ‘sp|Q7L4I2-1|RSRC2_HUMAN Isoform 1 of Arginine/serine-rich coiled-coil protein 2 OS = Homo sapiens GN = RSRC2’, 434,
50559]
[‘1.35’, 31, ‘sp|Q96HZ4-2|HES6_HUMAN Isoform 2 of Transcription cofactor HES-6 OS = Homo sapiens GN = HES6’, 214, 23483]
[‘1.35’, 24, ‘sp|Q8N726-1|CD2A2_HUMAN Isoform 4 of Cyclin-dependent kinase inhibitor 2A, isoform 4 OS = Homo sapiens
GN = CDKN2A’, 173, 18005]
[‘1.35’, 11, ‘sp|Q5JUX0-2|SPIN3_HUMAN Isoform 2 of Spindlin-3 OS = Homo sapiens GN = SPIN3’, 77, 8415]
[‘1.34’, 17, ‘sp|P49450-2|CENPA_HUMAN Isoform 2 of Histone H3-like centromeric protein A OS = Homo sapiens GN = CENPA’, 114,
13001]
[‘1.31’, 58, ‘sp|Q7L4I2-2|RSRC2_HUMAN Isoform 2 of Arginine/serine-rich coiled-coil protein 2 OS = Homo sapiens GN = RSRC2’, 386,
44878]
[‘1.29’, 40, ‘sp|Q13243-1|SFRS5_HUMAN Isoform SRP40-1 of Splicing factor, arginine/serine-rich 5 OS = Homo sapiens GN = SFRS5’, 272,
31263]
[‘1.28’, 47, ‘sp|Q9UK58-2|CCNL1_HUMAN Isoform 2 of Cyclin-L1 OS = Homo sapiens GN = CCNL1’, 320, 37273]
[‘1.28’, 15, ‘sp|Q66K41-3|Z385C_HUMAN Isoform 3 of Zinc finger protein 385C OS = Homo sapiens GN = ZNF385C’, 114, 11856]
[‘1.25’, 35, ‘sp|Q5BKY9-1|F133B_HUMAN Isoform 1 of Protein FAM133B OS = Homo sapiens GN = FAM133B’, 247, 28385]
[‘1.25’, 9, ‘sp|Q86SI9-3|CEI_HUMAN Isoform 3 of Protein CEI OS = Homo sapiens GN = C5orf38’, 70, 7333]
[‘1.24’, 47, ‘sp|Q96IZ7-1|RSRC1_HUMAN Isoform 1 of Arginine/serine-rich coiled-coil protein 1 OS = Homo sapiens GN = RSRC1’, 334,
38677]
[‘1.24’, 41, ‘sp|P62995-1|TRA2B_HUMAN Isoform 1 of Splicing factor, arginine/serine-rich 10 OS = Homo sapiens GN = SFRS10’, 288,
33665]
[‘1.24’, 30, ‘sp|Q86SI9-2|CEI_HUMAN Isoform 2 of Protein CEI OS = Homo sapiens GN = C5orf38’, 226, 24375]
[‘1.24’, 17, ‘sp|Q9HC23-1|PROK2_HUMAN Isoform 1 of Prokineticin-2 OS = Homo sapiens GN = PROK2’, 129, 14314]
[‘1.23’, 41, ‘sp|Q96S94-3|CCNL2_HUMAN Isoform 3 of Cyclin-L2 OS = Homo sapiens GN = CCNL2’, 298, 33839]
[‘1.23’, 33, ‘sp|Q5BKY9-2|F133B_HUMAN Isoform 2 of Protein FAM133B OS = Homo sapiens GN = FAM133B’, 237, 27193]
[‘1.23’, 17, ‘sp|Q9BTM1-1|H2AJ_HUMAN Isoform 1 of Histone H2A.J OS = Homo sapiens GN = H2AFJ’, 129, 14019]
[‘1.22’, 44, ‘sp|Q66PJ3-4|AR6P4_HUMAN Isoform 4 of ADP-ribosylation factor-like protein 6-interacting protein 4 OS = Homo sapiens
GN = ARL6IP4’, 338, 36210]
[‘1.22’, 11, ‘sp|Q8TEW8-4|PAR3L_HUMAN Isoform 4 of Partitioning-defective 3 homolog B OS = Homo sapiens GN = PARD3B’, 79, 9007]
[‘1.21’, 46, ‘sp|Q13247-3|SFRS6_HUMAN Isoform SRP55-3 of Splicing factor, arginine/serine-rich 6 OS = Homo sapiens GN = SFRS6’, 335,
38418]
[‘1.21’, 44, ‘sp|Q66PJ3-3|AR6P4_HUMAN Isoform 3 of ADP-ribosylation factor-like protein 6-interacting protein 4 OS = Homo sapiens
GN = ARL6IP4’, 341, 36612]
[‘1.20’, 45, ‘sp|Q66PJ3-2|AR6P4_HUMAN Isoform 2 of ADP-ribosylation factor-like protein 6-interacting protein 4 OS = Homo sapiens
GN = ARL6IP4’, 352, 37638]
[‘1.20’, 12, ‘sp|Q8N6C7-2|PGSF1_HUMAN Isoform 2 of Pituitary gland-specific factor 1 OS = Homo sapiens GN = PGSF1’, 91, 10048]
[‘1.19’, 38, ‘sp|Q13595-1|TRA2A_HUMAN Isoform Long of Transformer-2 protein homolog OS = Homo sapiens GN = TRA2A’, 282, 32688]
[‘1.17’, 45, ‘sp|Q66PJ3-1|AR6P4_HUMAN Isoform 1 of ADP-ribosylation factor-like protein 6-interacting protein 4 OS = Homo sapiens
GN = ARL6IP4’, 360, 38395]
[‘1.17’, 12, ‘sp|O75365-3|TP4A3_HUMAN Isoform 3 of Protein tyrosine phosphatase type IVA 3 OS = Homo sapiens GN = PTP4A3’, 87,
10494]
[‘1.16’, 24, ‘sp|P02686-3|MBP_HUMAN Isoform 3 of Myelin basic protein OS = Homo sapiens GN = MBP’, 197, 21493]
[‘1.15’, 22, ‘sp|P17096-3|HMGA1_HUMAN Isoform HMG-R of High mobility group protein HMG-I/HMG-Y OS = Homo sapiens
GN = HMGA1’, 179, 19694]
[‘1.15’, 7, ‘sp|Q8IU53-2|CASC2_HUMAN Isoform 2 of Protein CASC2, isoforms 1/2 OS = Homo sapiens GN = CASC2’, 55, 6154]
[‘1.14’, 13, ‘sp|P31260-2|HXA10_HUMAN Isoform 2 of Homeobox protein Hox-A10 OS = Homo sapiens GN = HOXA10’, 94, 11452]
[‘1.14’, 12, ‘sp|Q9NZQ0-2|RABJ_HUMAN Isoform 2 of Rab and DnaJ domain-containing protein OS = Homo sapiens GN = RBJ’, 90, 10621]
[‘1.14’, 10, ‘sp|Q8IVJ8-2|APRG1_HUMAN Isoform 2 of AP20 region protein 1 OS = Homo sapiens GN = APRG1’, 78, 8910]
[‘1.14’, 9, ‘sp|Q6QHF9-10|PAOX_HUMAN Isoform 12 of Peroxisomal N(1)-acetyl-spermine/spermidine oxidase OS = Homo sapiens
GN = PAOX’, 83, 8694]
[‘1.14’, 9, ‘sp|P02686-7|MBP_HUMAN Isoform 7 of Myelin basic protein OS = Homo sapiens GN = MBP’, 74, 8265]
[‘1.13’, 38, ‘sp|Q9UQ35-3|SRRM2_HUMAN Isoform 3 of Serine/arginine repetitive matrix protein 2 OS = Homo sapiens GN = SRRM2’, 311,
34212]
[‘1.13’, 22, ‘sp|P02686-4|MBP_HUMAN Isoform 4 of Myelin basic protein OS = Homo sapiens GN = MBP’, 186, 20245]
[‘1.13’, 20, ‘sp|P02686-5|MBP_HUMAN Isoform 5 of Myelin basic protein OS = Homo sapiens GN = MBP’, 171, 18590]
[‘1.13’, 12, ‘sp|P17096-2|HMGA1_HUMAN Isoform HMG-Y of High mobility group protein HMG-I/HMG-Y OS = Homo sapiens
GN = HMGA1’, 96, 10678]
[‘1.12’, 24, ‘sp|Q5HYI7-3|MTX3_HUMAN Isoform 3 of Metaxin-3 OS = Homo sapiens GN = MTX3’, 201, 22355]
[‘1.11’, 31, ‘sp|Q9GZR2-2|REXO4_HUMAN Isoform 2 of RNA exonuclease 4 OS = Homo sapiens GN = REXO4’, 250, 28390]
[‘1.11’, 8, ‘sp|Q6H9L7-4|TAIL1_HUMAN Isoform 4 of Thrombospondin and AMOP domain-containing isthmin-like protein 1 OS = Homo
sapiens GN = THSD3’, 76, 7995]
[‘1.10’, 20, ‘sp|Q15170-1|TCAL1_HUMAN Isoform 1 of Transcription elongation factor A protein-like 1 OS = Homo sapiens GN = TCEAL1’,
157, 18354]
[‘1.10’, 11, ‘sp|Q6ZUS6-3|CC149_HUMAN Isoform 3 of Coiled-coil domain-containing protein 149 OS = Homo sapiens GN = CCDC149’, 86,
10164]
[‘1.10’, 7, ‘sp|Q70UQ0-3|IKIP_HUMAN Isoform 3 of Inhibitor of nuclear factor kappa-B kinase-interacting protein OS = Homo sapiens
GN = IKIP’, 70, 7141]
[‘1.09’, 18, ‘sp|P02686-6|MBP_HUMAN Isoform 6 of Myelin basic protein OS = Homo sapiens GN = MBP’, 160, 17343]
[‘1.09’, 17, ‘sp|P49450-1|CENPA_HUMAN Isoform 1 of Histone H3-like centromeric protein A OS = Homo sapiens GN = CENPA’, 140,
15990]
[‘1.08’, 13, ‘sp|Q8WWL7-3|CCNB3_HUMAN Isoform 3 of G2/mitotic-specific cyclin-B3 OS = Homo sapiens GN = CCNB3’, 111, 12195]
[‘1.07’, 15, ‘sp|Q2NKX9-3|CB068_HUMAN Isoform 3 of UPF0561 protein C2orf68 OS = Homo sapiens GN = C2orf68’, 127, 14480]
[‘1.07’, 10, ‘sp|Q8IUX4-2|ABC3F_HUMAN Isoform 2 of DNA dC->dU-editing enzyme APOBEC-3F OS = Homo sapiens GN = APOBEC3F’,
79, 9444]
[‘1.06’, 9, ‘sp|Q8IU53-1|CASC2_HUMAN Isoform 1 of Protein CASC2, isoforms 1/2 OS = Homo sapiens GN = CASC2’, 76, 8607]
[‘1.06’, 8, ‘sp|Q9UBR5-3|CKLF_HUMAN Isoform CKLF3 of Chemokine-like factor OS = Homo sapiens GN = CKLF’, 67, 7652]
[‘1.05’, 20, ‘sp|Q2I0M5-2|RSPO4_HUMAN Isoform 2 of R-spondin-4 OS = Homo sapiens GN = RSPO4’, 172, 19606]
[‘1.05’, 8, ‘sp|Q9NPS7-2|F41CL_HUMAN Isoform 2 of Protein FAM41C-like OS = Homo sapiens’, 63, 7681]
[‘1.05’, 6, ‘sp|O75460-2|ERN1_HUMAN Isoform 2 of Serine/threonine-protein kinase/endoribonuclease IRE1 OS = Homo sapiens
GN = ERN1’, 70, 6648]
[‘1.04’, 46, ‘sp|Q5SSJ5-3|HP1B3_HUMAN Isoform 3 of Heterochromatin protein 1-binding protein 3 OS = Homo sapiens GN = HP1BP3’, 401,
44434]
[‘1.04’, 18, ‘sp|Q15973-2|ZN124_HUMAN Isoform 4 of Zinc finger protein 124 OS = Homo sapiens GN = ZNF124’, 156, 17830]
[‘1.04’, 8, ‘sp|Q9NPS7-1|F41CL_HUMAN Isoform 1 of Protein FAM41C-like OS = Homo sapiens’, 64, 7809]
[‘1.03’, 90, ‘sp|Q13427-1|PPIG_HUMAN Isoform 1 of Peptidyl-prolyl cis-trans isomerase G OS = Homo sapiens GN = PPIG’, 754, 88618]
[‘1.03’, 29, ‘sp|Q9BRU9-1|UTP23_HUMAN Isoform 1 of rRNA-processing protein UTP23 homolog OS = Homo sapiens GN = UTP23’, 249,
28430]
[‘1.03’, 18, ‘sp|Q6PH81-1|CP087_HUMAN Isoform 1 of UPF0547 protein C16orf87 OS = Homo sapiens GN = C16orf87’, 154, 17799]
[‘1.03’, 17, ‘sp|Q7Z6I8-2|CE024_HUMAN Isoform 2 of UPF0461 protein C5orf24 OS = Homo sapiens GN = C5orf24’, 155, 16724]
[‘1.03’, 17, ‘sp|P49759-2|CLK1_HUMAN Isoform Short of Dual specificity protein kinase CLK1 OS = Homo sapiens GN = CLK1’, 136, 16570]
[‘1.03’, 13, ‘sp|Q8NG50-4|RDM1_HUMAN Isoform 4 of RAD52 motif-containing protein 1 OS = Homo sapiens GN = RDM1’, 116, 13173]
[‘1.03’, 12, ‘sp|P17096-1|HMGA1_HUMAN Isoform HMG-I of High mobility group protein HMG-I/HMG-Y OS = Homo sapiens
GN = HMGA1’, 107, 11676]
[‘1.03’, 10, ‘sp|P48061-1|SDF1_HUMAN Isoform Beta of Stromal cell-derived factor 1 OS = Homo sapiens GN = CXCL12’, 93, 10665]
[‘1.02’, 17, ‘sp|P82912-3|RT11_HUMAN Isoform 3 of 28S ribosomal protein S11, mitochondrial OS = Homo sapiens GN = MRPS11’, 161,
16903]
[‘1.02’, 15, ‘sp|Q8N1T3-2|MYO1H_HUMAN Isoform 2 of Myosin-Ih OS = Homo sapiens GN = MYO1H’, 127, 14805]
[‘1.02’, 10, ‘sp|Q9NZ81-2|PRR13_HUMAN Isoform 2 of Proline-rich protein 13 OS = Homo sapiens GN = PRR13’, 98, 10531]
[‘1.02’, 7, ‘sp|Q9Y2A0-3|TPAP1_HUMAN Isoform 3 of p53-activated protein 1 OS = Homo sapiens GN = TP53AP1’, 60, 6937]
[‘1.01’, 32, ‘sp|Q9UBB5-3|MBD2_HUMAN Isoform 3 of Methyl-CpG-binding domain protein 2 OS = Homo sapiens GN = MBD2’, 302,
31744]
[‘1.01’, 19, ‘sp|Q9NWS8-4|RMND1_HUMAN Isoform 4 of Required for meiotic nuclear division protein 1 homolog OS = Homo sapiens
GN = RMND1’, 170, 19360]
[‘1.01’, 17, ‘sp|Q9H2U2-5|IPYR2_HUMAN Isoform 5 of Inorganic pyrophosphatase 2, mitochondrial OS = Homo sapiens GN = PPA2’, 157,
16961]
[‘1.01’, 13, ‘sp|P08949-1|NMB_HUMAN Isoform 1 of Neuromedin-B OS = Homo sapiens GN = NMB’, 121, 13255]
[‘1.00’, 37, ‘sp|Q09FC8-3|ZN415_HUMAN Isoform 3 of Zinc finger protein 415 OS = Homo sapiens GN = ZNF415’, 325, 37237]
[‘1.00’, 35, ‘sp|Q6ZN11-2|ZN793_HUMAN Isoform 2 of Zinc finger protein 793 OS = Homo sapiens GN = ZNF793’, 312, 35909]
[‘1.00’, 31, ‘sp|Q96IZ7-2|RSRC1_HUMAN Isoform 2 of Arginine/serine-rich coiled-coil protein 1 OS = Homo sapiens GN = RSRC1’, 276,
31528]
[‘1.00’, 8, ‘sp|Q7Z4H3-3|HDDC2_HUMAN Isoform 3 of HD domain-containing protein 2 OS = Homo sapiens GN = HDDC2’, 71, 8163]
[‘0.99’, 10, ‘sp|P56134-2|ATPK_HUMAN Isoform 2 of ATP synthase subunit f, mitochondrial OS = Homo sapiens GN = ATP5J2’, 88, 10363]
[‘0.98’, 50, ‘sp|Q3SXZ3-2|ZN718_HUMAN Isoform 2 of Zinc finger protein 718 OS = Homo sapiens GN = ZNF718’, 446, 51561]
[‘0.98’, 35, ‘sp|Q8IXZ2-2|ZC3H3_HUMAN Isoform 2 of Zinc finger CCCH domain-containing protein 3 OS = Homo sapiens GN = ZC3H3’,
335, 35929]
[‘0.98’, 24, ‘sp|Q9NP64-2|NO40_HUMAN Isoform 2 of Nucleolar protein of 40 kDa OS = Homo sapiens GN = ZCCHC17’, 217, 24918]
[‘0.97’, 48, ‘sp|Q499Z4-1|ZN672_HUMAN Isoform 1 of Zinc finger protein 672 OS = Homo sapiens GN = ZNF672’, 452, 50224]
[‘0.97’, 11, ‘sp|P10747-2|CD28_HUMAN Isoform 2 of T-cell-specific surface glycoprotein CD28 OS = Homo sapiens GN = CD28’, 101, 11527]
[‘0.97’, 9, ‘sp|Q9HC16-3|ABC3G_HUMAN Isoform 3 of DNA dC->dU-editing enzyme APOBEC-3G OS = Homo sapiens GN = APOBEC3G’,
79, 9385]
[‘0.97’, 5, ‘sp|Q16517-2|NNAT_HUMAN Isoform Beta of Neuronatin OS = Homo sapiens GN = NNAT’, 54, 6153]
[‘0.97’, 4, ‘sp|Q96T75-4|DSCR8_HUMAN Isoform 4 of Down syndrome critical region protein 8 OS = Homo sapiens GN = DSCR8’, 37, 4295]
[‘0.96’, 61, ‘sp|Q5VTL8-1|PR38B_HUMAN Isoform 1 of Pre-mRNA-splicing factor 38B OS = Homo sapiens GN = PRPF38B’, 546, 64467]
[‘0.96’, 14, ‘sp|Q8TCC3-3|RM30_HUMAN Isoform 3 of 39S ribosomal protein L30, mitochondrial OS = Homo sapiens GN = MRPL30’, 131,
15190]
[‘0.95’, 21, ‘sp|Q9NY12-1|NOLA1_HUMAN Isoform 1 of H/ACA ribonucleoprotein complex subunit 1 OS = Homo sapiens GN = NOLA1’,
217, 22347]
[‘0.95’, 14, ‘sp|Q7Z7F7-1|RM55_HUMAN Isoform 1 of 39S ribosomal protein L55, mitochondrial OS = Homo sapiens GN = MRPL55’, 128,
15128]
[‘0.95’, 14, ‘sp|Q7Z422-4|CA144_HUMAN Isoform 4 of UPF0485 protein C1orf144 OS = Homo sapiens GN = C1orf144’, 133, 14760]
[‘0.95’, 11, ‘sp|Q2T9K0-3|TMM44_HUMAN Isoform 3 of Transmembrane protein 44 OS = Homo sapiens GN = TMEM44’, 113, 12491]
[‘0.94’, 70, ‘sp|Q8NDQ6-4|ZN540_HUMAN Isoform 4 of Zinc finger protein 540 OS = Homo sapiens GN = ZNF540’, 637, 74992]
[‘0.94’, 56, ‘sp|Q8WXA9-1|SFR12_HUMAN Isoform 1 of Splicing factor, arginine/serine-rich 12 OS = Homo sapiens GN = SFRS12’, 508,
59380]
[‘0.94’, 43, ‘sp|Q3MIS6-2|ZN528_HUMAN Isoform 2 of Zinc finger protein 528 OS = Homo sapiens GN = ZNF528’, 395, 45715]
[‘0.94’, 22, ‘sp|O60258-2|FGF17_HUMAN Isoform 2 of Fibroblast growth factor 17 OS = Homo sapiens GN = FGF17’, 205, 23669]
[‘0.94’, 10, ‘sp|Q9BU19-4|ZN692_HUMAN Isoform 4 of Zinc finger protein 692 OS = Homo sapiens GN = ZNF692’, 96, 10818]
[‘0.93’, 27, ‘sp|Q6P1L5-2|AL2SC_HUMAN Isoform 2 of Amyotrophic lateral sclerosis 2 chromosomal region candidate gene 13 protein
OS = Homo sapiens GN = ALS2CR13’, 289, 29427]
[‘0.93’, 27, ‘sp|P12034-1|FGF5_HUMAN Isoform Long of Fibroblast growth factor 5 OS = Homo sapiens GN = FGF5’, 268, 29550]
[‘0.92’, 89, ‘sp|Q8N4W9-2|ZN808_HUMAN Isoform 2 of Zinc finger protein 808 OS = Homo sapiens GN = ZNF808’, 834, 96803]
[‘0.92’, 20, ‘sp|Q5T4W7-1|ARTN_HUMAN Isoform 1 of Artemin OS = Homo sapiens GN = ARTN’, 220, 22878]
[‘0.92’, 15, ‘sp|O15444-1|CCL25_HUMAN Isoform 1 of C-C motif chemokine 25 OS = Homo sapiens GN = CCL25’, 150, 16609]
[‘0.92’, 12, ‘sp|Q8IVJ8-3|APRG1_HUMAN Isoform 3 of AP20 region protein 1 OS = Homo sapiens GN = APRG1’, 119, 13172]
[‘0.91’, 67, ‘sp|Q8NDQ6-2|ZN540_HUMAN Isoform 2 of Zinc finger protein 540 OS = Homo sapiens GN = ZNF540’, 628, 73708]
[‘0.91’, 19, ‘sp|P05019-1|IGF1B_HUMAN Isoform IGF-IB of Insulin-like growth factor IB OS = Homo sapiens GN = IGF1’, 195, 21841]
[‘0.91’, 14, ‘sp|O60565-2|GREM1_HUMAN Isoform 2 of Gremlin-1 OS = Homo sapiens GN = GREM1’, 143, 16292]
[‘0.91’, 12, ‘sp|Q96A00-2|PP14A_HUMAN Isoform 2 of Protein phosphatase 1 regulatory subunit 14A OS = Homo sapiens GN = PPP1R14A’,
120, 13479]
[‘0.91’, 8, ‘sp|P08118-2|MSMB_HUMAN Isoform PSP57 of Beta-microseminoprotein OS = Homo sapiens GN = MSMB’, 77, 8778]
[‘0.90’, 53, ‘sp|Q9UK58-1|CCNL1_HUMAN Isoform 1 of Cyclin-L1 OS = Homo sapiens GN = CCNL1’, 526, 59633]
[‘0.90’, 40, ‘sp|Q03924-1|ZN117_HUMAN Isoform 1 of Zinc finger protein 117 OS = Homo sapiens GN = ZNF117’, 383, 45066]
[‘0.90’, 27, ‘sp|Q9BXY4-1|RSPO3_HUMAN Isoform 1 of R-spondin-3 OS = Homo sapiens GN = RSPO3’, 272, 30928]
[‘0.90’, 16, ‘sp|Q86SG4-3|DPCA2_HUMAN Isoform 3 of Dresden prostate carcinoma protein 2 OS = Homo sapiens GN = C15orf21’, 150,
17975]
[‘0.90’, 13, ‘sp|P47902-2|CDX1_HUMAN Isoform 2 of Homeobox protein CDX-1 OS = Homo sapiens GN = CDX1’, 130, 14660]
[‘0.89’, 44, ‘sp|Q9NXE8-1|CCD49_HUMAN Isoform 1 of Coiled-coil domain-containing protein 49 OS = Homo sapiens GN = CCDC49’, 425,
49647]
[‘0.89’, 44, ‘sp|Q03924-2|ZN117_HUMAN Isoform 2 of Zinc finger protein 117 OS = Homo sapiens GN = ZNF117’, 427, 50051]
[‘0.89’, 40, ‘sp|Q147U1-2|ZN846_HUMAN Isoform 2 of Zinc finger protein 846 OS = Homo sapiens GN = ZNF846’, 404, 45838]
[‘0.89’, 29, ‘sp|Q9BXY4-2|RSPO3_HUMAN Isoform 2 of R-spondin-3 OS = Homo sapiens GN = RSPO3’, 292, 33233]
[‘0.89’, 20, ‘sp|Q5T4W7-3|ARTN_HUMAN Isoform 3 of Artemin OS = Homo sapiens GN = ARTN’, 228, 23616]
[‘0.89’, 18, ‘sp|Q6UXX9-3|RSPO2_HUMAN Isoform 3 of R-spondin-2 OS = Homo sapiens GN = RSPO2’, 179, 20972]
[‘0.89’, 13, ‘sp|Q7Z422-2|CA144_HUMAN Isoform 2 of UPF0485 protein C1orf144 OS = Homo sapiens GN = C1orf144’, 132, 14604]
[‘0.89’, 9, ‘sp|Q8NFV4-3|ABHDB_HUMAN Isoform 3 of Abhydrolase domain-containing protein 11 OS = Homo sapiens GN = ABHD11’, 97,
10361]
[‘0.89’, 8, ‘sp|P48061-2|SDF1_HUMAN Isoform Alpha of Stromal cell-derived factor 1 OS = Homo sapiens GN = CXCL12’, 89, 10103]
[‘0.88’, 15, ‘sp|Q92466-3|DDB2_HUMAN Isoform D2 of DNA damage-binding protein 2 OS = Homo sapiens GN = DDB2’, 156, 17434]
[‘0.88’, 8, ‘sp|Q9HD64-2|GAGD2_HUMAN Isoform B of G antigen family D member 2 OS = Homo sapiens GN = XAGE1’, 81, 9077]
[‘0.88’, 7, ‘sp|Q9BZJ0-5|CRNL1_HUMAN Isoform 5 of Crooked neck-like protein 1 OS = Homo sapiens GN = CRNKL1’, 74, 7946]
[‘0.88’, 6, ‘sp|Q8TC05-3|MDM1_HUMAN Isoform 3 of Nuclear protein MDM1 OS = Homo sapiens GN = MDM1’, 69, 7926]
[‘0.87’, 74, ‘sp|Q9NYF8-4|BCLF1_HUMAN Isoform 4 of Bcl-2-associated transcription factor 1 OS = Homo sapiens GN = BCLAF1’, 747,
85937]
[‘0.87’, 67, ‘sp|Q8NDQ6-1|ZN540_HUMAN Isoform 1 of Zinc finger protein 540 OS = Homo sapiens GN = ZNF540’, 660, 77093]
[‘0.87’, 52, ‘sp|Q03936-2|ZNF92_HUMAN Isoform 2 of Zinc finger protein 92 OS = Homo sapiens GN = ZNF92’, 517, 60209]
[‘0.87’, 44, ‘sp|Q8NEP9-3|ZN555_HUMAN Isoform 3 of Zinc finger protein 555 OS = Homo sapiens GN = ZNF555’, 440, 51594]
[‘0.87’, 25, ‘sp|P22090|RS4Y1_HUMAN 40S ribosomal protein S4, Y isoform 1 OS = Homo sapiens GN = RPS4Y1’, 263, 29455]
[‘0.87’, 20, ‘sp|P55075-2|FGF8_HUMAN Isoform FGF-8A of Fibroblast growth factor 8 OS = Homo sapiens GN = FGF8’, 204, 23522]
[‘0.87’, 20, ‘sp|P12272-3|PTHR_HUMAN Isoform 3 of Parathyroid hormone-related protein OS = Homo sapiens GN = PTHLH’, 209, 23942]
[‘0.87’, 16, ‘sp|Q7Z7F7-2|RM55_HUMAN Isoform 2 of 39S ribosomal protein L55, mitochondrial OS = Homo sapiens GN = MRPL55’, 164,
18902]
[‘0.87’, 12, ‘sp|P10747-4|CD28_HUMAN Isoform 4 of T-cell-specific surface glycoprotein CD28 OS = Homo sapiens GN = CD28’, 123, 14013]
[‘0.86’, 33, ‘sp|Q8N8C0-2|ZN781_HUMAN Isoform 2 of Zinc finger protein 781 OS = Homo sapiens GN = ZNF781’, 327, 38274]
[‘0.86’, 29, ‘sp|Q15973-1|ZN124_HUMAN Isoform 3 of Zinc finger protein 124 OS = Homo sapiens GN = ZNF124’, 296, 33852]
[‘0.86’, 23, ‘sp|Q9H0A6-4|RNF32_HUMAN Isoform 4 of RING finger protein 32 OS = Homo sapiens GN = RNF32’, 235, 27130]
[‘0.86’, 21, ‘sp|Q8IWN7-2|RP1L1_HUMAN Isoform 2 of Retinitis pigmentosa 1-like 1 protein OS = Homo sapiens GN = RP1L1’, 222, 24854]
[‘0.86’, 20, ‘sp|Q6PI47-3|KCD18_HUMAN Isoform 3 of BTB/POZ domain-containing protein KCTD18 OS = Homo sapiens GN = KCTD18’,
221, 23414]
[‘0.86’, 18, ‘sp|O75494-4|FUSIP_HUMAN Isoform 4 of FUS-interacting serine-arginine-rich protein 1 OS = Homo sapiens GN = FUSIP1’, 173,
21000]
[‘0.86’, 13, ‘sp|P10747-3|CD28_HUMAN Isoform 3 of T-cell-specific surface glycoprotein CD28 OS = Homo sapiens GN = CD28’, 136, 15369]
[‘0.86’, 7, ‘sp|P16157-20|ANK1_HUMAN Isoform Mu20 of Ankyrin-1 OS = Homo sapiens GN = ANK1’, 74, 8374]
[‘0.85’, 45, ‘sp|Q68DY1-2|ZN626_HUMAN Isoform 2 of Zinc finger protein 626 OS = Homo sapiens GN = ZNF626’, 464, 53889]
[‘0.85’, 21, ‘sp|O60258-1|FGF17_HUMAN Isoform 1 of Fibroblast growth factor 17 OS = Homo sapiens GN = FGF17’, 216, 24891]
[‘0.85’, 17, ‘sp|P82912-1|RT11_HUMAN Isoform 1 of 28S ribosomal protein S11, mitochondrial OS = Homo sapiens GN = MRPS11’, 194,
20615]
[‘0.85’, 13, ‘sp|Q9BWV2-3|SPAT9_HUMAN Isoform 3 of Spermatogenesis-associated protein 9 OS = Homo sapiens GN = SPATA9’, 135,
15275]
[‘0.85’, 12, ‘sp|Q9Y5P2-1|CSAG2_HUMAN Isoform 1 of Chondrosarcoma-associated gene 2/3A protein OS = Homo sapiens GN = CSAG2’,
127, 14429]
[‘0.85’, 10, ‘sp|Q6RVD6-1|SPAT8_HUMAN Isoform 1 of Spermatogenesis-associated protein 8 OS = Homo sapiens GN = SPATA8’, 105,
11727]
[‘0.84’, 46, ‘sp|Q3SXZ3-1|ZN718_HUMAN Isoform 1 of Zinc finger protein 718 OS = Homo sapiens GN = ZNF718’, 478, 55404]
[‘0.84’, 36, ‘sp|Q3SY52-3|ZIK1_HUMAN Isoform 3 of Zinc finger protein interacting with ribonucleoprotein K OS = Homo sapiens
GN = ZIK1’, 384, 43717]
[‘0.84’, 24, ‘sp|Q9BU76-1|MMTA2_HUMAN Isoform 1 of Multiple myeloma tumor-associated protein 2 OS = Homo sapiens
GN = MMTAG2’, 263, 29411]
[‘0.84’, 24, ‘sp|Q8TD47|RS4Y2_HUMAN 40S ribosomal protein S4, Y isoform 2 OS = Homo sapiens GN = RPS4Y2’, 263, 29295]
[‘0.84’, 20, ‘sp|Q96CX3-2|ZN501_HUMAN Isoform 2 of Zinc finger protein 501 OS = Homo sapiens GN = ZNF501’, 215, 24880]
[‘0.84’, 20, ‘sp|Q147U1-3|ZN846_HUMAN Isoform 3 of Zinc finger protein 846 OS = Homo sapiens GN = ZNF846’, 210, 24075]
[‘0.84’, 9, ‘sp|P56134-1|ATPK_HUMAN Isoform 1 of ATP synthase subunit f, mitochondrial OS = Homo sapiens GN = ATP5J2’, 94, 10917]
[‘0.83’, 48, ‘sp|Q96S94-1|CCNL2_HUMAN Isoform 1 of Cyclin-L2 OS = Homo sapiens GN = CCNL2’, 520, 58147]
[‘0.83’, 27, ‘sp|Q9NWB6-2|ARGL1_HUMAN Isoform 2 of Arginine and glutamate-rich protein 1 OS = Homo sapiens GN = ARGLU1’, 273,
32885]
[‘0.83’, 24, ‘sp|P62701|RS4X_HUMAN 40S ribosomal protein S4, X isoform OS = Homo sapiens GN = RPS4X’, 263, 29597]
[‘0.83’, 23, ‘sp|Q6UXX9-1|RSPO2_HUMAN Isoform 1 of R-spondin-2 OS = Homo sapiens GN = RSPO2’, 243, 28314]
[‘0.83’, 20, ‘sp|P55075-3|FGF8_HUMAN Isoform FGF-8B of Fibroblast growth factor 8 OS = Homo sapiens GN = FGF8’, 215, 24711]
[‘0.83’, 12, ‘sp|Q8N3H0-1|F19A2_HUMAN Isoform 1 of Protein FAM19A2 OS = Homo sapiens GN = FAM19A2’, 131, 14620]
[‘0.83’, 12, ‘sp|Q6N063-3|OGFD2_HUMAN Isoform 3 of 2-oxoglutarate and iron-dependent oxygenase domain-containing protein 2
OS = Homo sapiens GN = OGFOD2’, 129, 14734]
[‘0.83’, 9, ‘sp|Q56VL3-2|OCAD2_HUMAN Isoform 2 of OCIA domain-containing protein 2 OS = Homo sapiens GN = OCIAD2’, 99, 11029]
[‘0.82’, 34, ‘sp|Q8N8C0-1|ZN781_HUMAN Isoform 1 of Zinc finger protein 781 OS = Homo sapiens GN = ZNF781’, 355, 41526]
[‘0.82’, 20, ‘sp|Q5T4W7-2|ARTN_HUMAN Isoform 2 of Artemin OS = Homo sapiens GN = ARTN’, 237, 24471]
[‘0.82’, 17, ‘sp|Q9NY12-2|NOLA1_HUMAN Isoform 2 of H/ACA ribonucleoprotein complex subunit 1 OS = Homo sapiens GN = NOLA1’,
199, 20834]
[‘0.81’, 37, ‘sp|Q96SQ7-2|ATOH8_HUMAN Isoform 2 of Protein atonal homolog 8 OS = Homo sapiens GN = ATOH8’, 416, 45785]
[‘0.81’, 22, ‘sp|Q9NP64-1|NO40_HUMAN Isoform 1 of Nucleolar protein of 40 kDa OS = Homo sapiens GN = ZCCHC17’, 241, 27569]
[‘0.81’, 22, ‘sp|Q92913-1|FGF13_HUMAN Isoform 1A of Fibroblast growth factor 13 OS = Homo sapiens GN = FGF13’, 245, 27563]
[‘0.81’, 21, ‘sp|P55075-1|FGF8_HUMAN Isoform FGF-8E of Fibroblast growth factor 8 OS = Homo sapiens GN = FGF8’, 233, 26525]
[‘0.81’, 18, ‘sp|O75494-3|FUSIP_HUMAN Isoform 3 of FUS-interacting serine-arginine-rich protein 1 OS = Homo sapiens GN = FUSIP1’, 183,
22222]
[‘0.81’, 9, ‘sp|Q7L592-3|CB056_HUMAN Isoform 3 of UPF0511 protein C2orf56, mitochondrial OS = Homo sapiens GN = C2orf56’, 99,
11289]
[‘0.81’, 7, ‘sp|Q6PDA7-3|SG11A_HUMAN Isoform 3 of Sperm-associated antigen 11A OS = Homo sapiens GN = SPAG11A’, 82, 9075]
[‘0.80’, 72, ‘sp|O14746-2|TERT_HUMAN Isoform 2 of Telomerase reverse transcriptase OS = Homo sapiens GN = TERT’, 807, 90225]
[‘0.80’, 54, ‘sp|Q86YE8-4|ZN573_HUMAN Isoform 4 of Zinc finger protein 573 OS = Homo sapiens GN = ZNF573’, 578, 67865]
[‘0.80’, 30, ‘sp|O95218-1|ZRAB2_HUMAN Isoform 1 of Zinc finger Ran-binding domain-containing protein 2 OS = Homo sapiens
GN = ZRANB2’, 330, 37404]
[‘0.80’, 24, ‘sp|Q96CX3-1|ZN501_HUMAN Isoform 1 of Zinc finger protein 501 OS = Homo sapiens GN = ZNF501’, 271, 31178]
[‘0.80’, 22, ‘sp|Q92915-1|FGF14_HUMAN Isoform 1 of Fibroblast growth factor 14 OS = Homo sapiens GN = FGF14’, 247, 27701]
[‘0.80’, 16, ‘sp|P82912-2|RT11_HUMAN Isoform 2 of 28S ribosomal protein S11, mitochondrial OS = Homo sapiens GN = MRPS11’, 193,
20459]

### Nucleic Acids

The present invention provides systems and methods for delivery of nucleic acids to cells in vivo or in vitro. Such systems and methods typically involve association of one or more nucleic acids with supercharged proteins to form a complex, and delivery of the complex to one or more cells. In some embodiments, the nucleic acid may have therapeutic activity. In some embodiments, delivery of the complex to cells involves administering a complex comprising supercharged proteins associated with a nucleic acid to a subject in need thereof. In some embodiments, a nucleic acid by itself may not be able to enter the interior of a cell, but is able to enter the interior of a cell when complexed with a supercharged protein. In some embodiments, a supercharged protein is utilized to allow a nucleic acid to enter a cell. Nucleic acids in accordance with the invention may themselves have therapeutic activity or may direct expression of an RNA and/or protein that has therapeutic activity. Therapeutic activities of nucleic acids are discussed in further detail below.

The term “nucleic acid,” in its broadest sense, includes any compound and/or substance that is or can be incorporated into an oligonucleotide chain. Exemplary nucleic acids for use in accordance with the present invention include, but are not limited to, one or more of DNA, RNA, hybrids thereof, RNAi-inducing agents, RNAi agents, siRNAs, shRNAs, miRNAs, antisense RNAs, ribozymes, catalytic DNA, RNAs that induce triple helix formation, aptamers, vectors, etc., described in further detail below.

Nucleic acids for use in accordance with the invention may be prepared according to any available technique including, but not limited to chemical synthesis, enzymatic synthesis, enzymatic or chemical cleavage of a longer precursor, etc. Methods of synthesizing RNAs are known in the art (see, e.g., Gait, M. J. (ed.) Oligonucleotide synthesis: a practical approach, Oxford [Oxfordshire], Washington, D.C.: IRL Press, 1984; and Herdewijn, P. (ed.) Oligonucleotide synthesis: methods and applications, Methods in Molecular Biology, v. 288 (Clifton, N.J.) Totowa, N.J.: Humana Press, 2005; both of which are incorporated herein by reference).

Nucleic acids may comprise naturally occurring nucleosides, modified nucleosides, naturally occurring nucleosides with hydrocarbon linkers (e.g., an alkylene) or a polyether linker (e.g., a PEG linker) inserted between one or more nucleosides, modified nucleosides with hydrocarbon or PEG linkers inserted between one or more nucleosides, or a combination of thereof. In some embodiments, nucleotides or modified nucleotides can be replaced with a hydrocarbon linker or a polyether linker provided that the function of the nucleic acid is not substantially reduced by the substitution.

It will be appreciated by those of ordinary skill in the art that nucleic acids in accordance with the present invention may comprise nucleotides entirely of the types found in naturally occurring nucleic acids, or may instead include one or more nucleotide analogs or have a structure that otherwise differs from that of a naturally occurring nucleic acid. U.S. Pat. Nos. 6,403,779; 6,399,754; 6,225,460; 6,127,533; 6,031,086; 6,005,087; 5,977,089 (each of which is incorporated herein by reference); and references therein disclose a wide variety of specific nucleotide analogs and modifications that may be used. See Crooke, S. (ed.) Antisense Drug Technology: Principles, Strategies, and Applications (1^{st} ed), Marcel Dekker; ISBN: 0824705661; 1st edition (2001; incorporated herein by reference) and references therein. For example, 2′-modifications include halo, alkoxy and allyloxy groups. In some embodiments, the 2′-OH group is replaced by a group selected from H, OR, R, halo, SH, SR, NH₂, NHR, NR₂ or CN, wherein R is C₁-C₆ alkyl, alkenyl, or alkynyl, and halo is F, Cl, Br, or I. Examples of modified linkages include phosphorothioate and 5′-N-phosphoramidite linkages.

Nucleic acids comprising a variety of different nucleotide analogs, modified backbones, or non-naturally occurring internucleoside linkages can be utilized in accordance with the present invention. Nucleic acids of the present invention may include natural nucleosides (i.e., adenosine, thymidine, guanosine, cytidine, uridine, deoxyadenosine, deoxythymidine, deoxyguanosine, and deoxycytidine) or modified nucleosides. Examples of modified nucleotides include base modified nucleoside (e.g., aracytidine, inosine, isoguanosine, nebularine, pseudouridine, 2,6-diaminopurine, 2-aminopurine, 2-thiothymidine, 3-deaza-5-azacytidine, 2′-deoxyuridine, 3-nitorpyrrole, 4-methylindole, 4-thiouridine, 4-thiothymidine, 2-aminoadenosine, 2-thiothymidine, 2-thiouridine, 5-bromocytidine, 5-iodouridine, inosine, 6-azauridine, 6-chloropurine, 7-deazaadenosine, 7-deazaguanosine, 8-azaadenosine, 8-azidoadenosine, benzimidazole, M1-methyladenosine, pyrrolo-pyrimidine, 2-amino-6-chloropurine, 3-methyl adenosine, 5-propynylcytidine, 5-propynyluridine, 5-bromouridine, 5-fluorouridine, 5-methylcytidine, 7-deazaadenosine, 7-deazaguanosine, 8-oxoadenosine, 8-oxoguanosine, O(6)-methylguanine, and 2-thiocytidine), chemically or biologically modified bases (e.g., methylated bases), modified sugars (e.g., 2′-fluororibose, 2′-aminoribose, 2′-azidoribose, 2′-O-methylribose, L-enantiomeric nucleosides arabinose, and hexose), modified phosphate groups (e.g., phosphorothioates and 5′-N-phosphoramidite linkages), and combinations thereof. Natural and modified nucleotide monomers for the chemical synthesis of nucleic acids are readily available. In some cases, nucleic acids comprising such modifications display improved properties relative to nucleic acids consisting only of naturally occurring nucleotides. In some embodiments, nucleic acid modifications described herein are utilized to reduce and/or prevent digestion by nucleases (e.g. exonucleases, endonucleases, etc.). For example, the structure of a nucleic acid may be stabilized by including nucleotide analogs at the 3′ end of one or both strands order to reduce digestion.

Modified nucleic acids need not be uniformly modified along the entire length of the molecule. Different nucleotide modifications and/or backbone structures may exist at various positions in the nucleic acid. One of ordinary skill in the art will appreciate that the nucleotide analogs or other modification(s) may be located at any position(s) of a nucleic acid such that the function of the nucleic acid is not substantially affected. To give but one example, modifications may be located at any position of a nucleic acid targeting moiety such that the ability of the nucleic acid targeting moiety to specifically bind to the target is not substantially affected. The modified region may be at the 5′-end and/or the 3′-end of one or both strands. For example, modified nucleic acid targeting moieties in which approximately 1 to approximately 5 residues at the 5′ and/or 3′ end of either of both strands are nucleotide analogs and/or have a backbone modification have been employed. A modification may be a 5′ or 3′ terminal modification. One or both nucleic acid strands may comprise at least 50% unmodified nucleotides, at least 80% unmodified nucleotides, at least 90% unmodified nucleotides, or 100% unmodified nucleotides.

Nucleic acids in accordance with the present invention may, for example, comprise a modification to a sugar, nucleoside, or internucleoside linkage such as those described in U.S. Patent Publications 2003/0175950, 2004/0192626, 2004/0092470, 2005/0020525, and 2005/0032733; each of which is incorporated herein by reference. The present invention encompasses the use of any nucleic acid having any one or more of the modification described therein. For example, a number of terminal conjugates, e.g., lipids such as cholesterol, lithocholic acid, aluric acid, or long alkyl branched chains have been reported to improve cellular uptake. Analogs and modifications may be tested using, e.g., using any appropriate assay known in the art, for example, to select those that result in improved target gene silencing by an RNAi agent, etc. In some embodiments, nucleic acids in accordance with the present invention may comprise one or more non-natural nucleoside linkages. In some embodiments, one or more internal nucleotides at the 3′-end, 5′-end, or both 3′- and 5′-ends of the nucleic acid targeting moiety are inverted to yield a linkage such as a 3′-3′ linkage or a 5′-5′ linkage.

In some embodiments, nucleic acids in accordance with the present invention are not synthetic, but are naturally-occurring entities that have been isolated from their natural environments.

### RNAi Agents

### RNA Interference

In some embodiments, nucleic acids that can be associated with supercharged proteins include agents that mediate RNA interference (RNAi). RNAi is a mechanism that inhibits expression of specific genes. RNAi typically inhibits gene expression at the level of translation, but can function by inhibiting gene expression at the level of transcription. RNAi targets include any RNA that might be present in cells, including but not limited to, cellular transcripts, pathogen transcripts (e.g., from viruses, bacteria, fungi, etc.), transposons, vectors, etc.

The RNAi pathway is initiated by the enzyme dicer, which cleaves long, double-stranded RNA (dsRNA) molecules into short fragments of 20-25 base pairs, optionally with a few unpaired overhang bases on one or both ends. One of the two strands of each fragment, known as the guide strand, is then incorporated into the RNA-induced silencing complex (RISC) and pairs with complementary sequences. The other strand is degraded during RISC activation. The most well-studied outcome of this recognition event is post-transcriptional gene silencing. This occurs when the guide strand specifically pairs with a target transcript and induces degradation of the target transcript by argonaute, the catalytic component of the RISC complex. Another outcome is epigenetic changes to a gene (e.g., histone modification and DNA methylation) affecting the degree to which the gene is transcribed.

Introduction of long double-stranded RNA (e.g., greater than 30 bp) into mammalian cells results in systemic, nonspecific inhibition of translation due to activation of the interferon response. A breakthrough occurred when it was found that this obstacle could be overcome by the use of synthetic short RNAs (e.g., 19-25 bp) that can be either delivered exogenously (Elbashir et al., 2001, Nature, 411:494; incorporated herein by reference) or expressed endogenously from RNA polymerase II or III promoters.

The phenomenon of RNAi is discussed in greater detail, for example, in the following references, each of which is incorporated herein by reference: Elbashir et al., 2001, Genes Dev., 15:188; Fire et al., 1998, Nature, 391:806; Tabara et al., 1999, Cell, 99:123; Hammond et al., Nature, 2000, 404:293; Zamore et al., 2000, Cell, 101:25; Chakraborty, 2007, Curr. Drug Targets, 8:469; and Morris and Rossi, 2006, Gene Ther., 13:553.

As used herein, the term “RNAi agent” refers to an RNA, optionally including one or more nucleotide analogs or modifications, having a structure characteristic of molecules that can mediate inhibition of gene expression through an RNAi mechanism. Generally, an RNAi agent includes a portion that is substantially complementary to a target RNA. In some embodiments, RNAi agents are at least partly double-stranded. In some embodiments, RNAi agents are single-stranded. In some embodiments, exemplary RNAi agents can include short interfering RNA (siRNA), short hairpin RNA (shRNA), and/or micro RNA (miRNA). In some embodiments, the term “RNAi agent” may refer to any RNA, RNA derivative, and/or nucleic acid encoding an RNA that induces an RNAi effect (e.g., degradation of target RNA and/or inhibition of translation).

As used herein, the term “RNAi-inducing agent” encompasses any entity that delivers, regulates, and/or modifies the activity of an RNAi agent. In some embodiments, RNAi-inducing agents may include vectors (other than naturally occurring molecules not modified by the hand of man) whose presence within a cell results in RNAi and leads to reduced expression of a transcript to which the RNAi-inducing agent is targeted. In some embodiments, an RNAi-inducing agent is an “RNAi-inducing vector,” which refers to a vector whose presence within a cell results in production of one or more RNAs that self-hybridize or hybridize to each other to form an RNAi agent (e.g. siRNA, shRNA, and/or miRNA). In various embodiments, this term encompasses plasmids, e.g., DNA vectors (whose sequence may comprise sequence elements derived from a virus), or viruses (other than naturally occurring viruses or plasmids that have not been modified by the hand of man), whose presence within a cell results in production of one or more RNAs that self-hybridize or hybridize to each other to form an RNAi agent. In general, the vector comprises a nucleic acid operably linked to expression signal(s) so that one or more RNAs that hybridize or self-hybridize to form an RNAi agent are transcribed when the vector is present within a cell. Thus the vector provides a template for intracellular synthesis of the RNA or RNAs or precursors thereof. In some embodiments, RNAi-inducing agents are compositions comprising RNAi agents and one or more pharmaceutically acceptable excipients and/or carriers. For the purposes of the present invention, any partly or fully double-stranded short RNA as described herein, one strand of which binds to a target transcript and reduces its expression (i.e., reduces the level of the transcript and/or reduces synthesis of the polypeptide encoded by the transcript) is considered to be an RNAi-inducing agent, regardless of whether it acts by triggering degradation, inhibiting translation, or by other means. In addition any precursor RNA structure that may be processed in vivo (i.e., within a cell or organism) to generate such an RNAi-inducing agent is useful in the present invention.

RNAi agents in accordance with the invention may target any portion of a transcript. In some embodiments, a target transcript is located within a coding sequence of a gene. In some embodiments, a target transcript is located within non-coding sequence. In some embodiments, a target transcript is located within an exon. In some embodiments, a target transcript is located within an intron. In some embodiments, a target transcript is located within a 5′ untranslated region (UTR) or 3′ UTR of a gene. In some embodiments, a target transcript is located within an enhancer region. In some embodiments, a target transcript is located within a promoter.

For any particular gene target, design of RNAi agents and/or RNAi-inducing agents typically follows certain guidelines. In general, it is desirable to avoid sections of target transcript that may be shared with other transcripts whose degradation is not desired. In some embodiments, RNAi agents and/or RNAi-inducing entities target transcripts and/or portions thereof that are highly conserved. In some embodiments, RNAi agents and/or RNAi-inducing entities target transcripts and/or portions thereof that are not highly conserved.

siRNAs and shRNAs

As used herein, an “siRNA” refers to an RNAi agent comprising an RNA duplex (referred to herein as a “duplex region”) that is approximately 19 base pairs (bp) in length and optionally further comprises one or two single-stranded overhangs. In some embodiments, an siRNA comprises a duplex region ranging from 15 bp to 29 bp in length and optionally further comprising one or two single-stranded overhangs. An siRNA is typically formed from two RNA molecules (i.e., two strands) that hybridize together. One strand of an siRNA includes a portion that hybridizes with a target transcript. In some embodiments, siRNAs mediate inhibition of gene expression by causing degradation of target transcripts.

As used herein, an “shRNA” refers to an RNAi agent comprising an RNA having at least two complementary portions hybridized or capable of hybridizing to form a double-stranded (duplex) structure sufficiently long to mediate RNAi (typically at least approximately 19 bp in length), and at least one single-stranded portion, typically ranging between approximately 1 nucleotide (nt) and approximately 10 nt in length that forms a loop. In some embodiments, an shRNA comprises a duplex portion ranging from 15 bp to 29 bp in length and at least one single-stranded portion, typically ranging between approximately 1 nt and approximately 10 nt in length that forms a loop. In some embodiments, the single-stranded portion is approximately 1 nt, approximately 2 nt, approximately 3 nt, approximately 4 nt, approximately 5 nt, approximately 6 nt, approximately 7 nt, approximately 8 nt, approximately 9 nt, or approximately 10 nt in length. In some embodiments, shRNAs are processed into siRNAs by cellular RNAi machinery (e.g., by Dicer). Thus, in some embodiments, shRNAs may be precursors of siRNAs. Regardless, siRNAs in general are capable of inhibiting expression of a target RNA, similar to siRNAs. As used herein, the term “short RNAi agent” is used to refer to siRNAs and shRNAs, collectively.

As mentioned above, short RNAi agents typically include a base-paired region (“duplex region”) between approximately 15 nt and approximately 29 nt long, e.g., approximately 19 nt long, and may optionally have one or more free or looped ends. In some embodiments, short RNAi agents have a duplex region of about 15 nt, about 16 nt, about 17 nt, about 18 nt, about 19 nt, about 20 nt, about 21 nt, about 22 nt, about 23 nt, about 24 nt, about 25 nt, about 26 nt, about 27 nt, about 28 nt, or about 29 nt in length. However, it is not required that the administered agent have this structure. For example, RNAi-inducing agents may comprise any structure capable of being processed in vivo to the structure of a short RNAi agent. In some embodiments, an RNAi-inducing agent is delivered to a cell, where it undergoes one or more processing steps before becoming a functional short RNAi agent. In such cases, those of ordinary skill in the art will appreciate that it is desirable for the RNAi-inducing agent to include sequences that may be necessary and/or helpful for its processing.

In describing RNAi-inducing agents and/or short RNAi agents, it is convenient to refer to an agent as having two strands. In general, the sequence of the duplex portion of one strand of an RNAi-inducing agent and/or short RNAi agent is substantially complementary to the target transcript in this region. The sequence of the duplex portion of the other strand of the RNAi-inducing agent and/or short RNAi agent is typically substantially identical to the targeted portion of the target transcript. The strand comprising the portion complementary to the target is referred to as the “antisense strand,” while the other strand is often referred to as the “sense strand.” The portion of the antisense strand that is complementary to the target may be referred to as the “inhibitory region.”

RNAi-inducing agents and/or short RNAi agents typically include a region (the “duplex region”), one strand of which contains an inhibitory region between 15 nt to 29 nt in length that is sufficiently complementary to a portion of the target transcript (the “target portion”), so that a hybrid (the “core region”) can form in vivo between this strand and the target transcript. The core region is understood not to include overhangs.

In some embodiments, short RNAi agents have an inhibitory region of about 15 nt, about 16 nt, about 17 nt, about 18 nt, about 19 nt, about 20 nt, about 21 nt, about 22 nt, about 23 nt, about 24 nt, about 25 nt, about 26 nt, about 27 nt, about 28 nt, or about 29 nt in length. In some embodiments, short RNAi agents have an inhibitory region of about 19 nt in length. In some embodiments, hybridization of one strand of a short RNAi agent to its target transcript yields a core region of about 15 nt, about 16 nt, about 17 nt, about 18 nt, about 19 nt, about 20 nt, about 21 nt, about 22 nt, about 23 nt, about 24 nt, about 25 nt, about 26 nt, about 27 nt, about 28 nt, or about 29 nt in length. In some embodiments, hybridization of one strand of a short RNAi agent to its target transcript yields a core region of about 19 nt in length.

Target transcripts are often cleaved near the center of the duplex region. In some embodiments, target transcripts are cleaved at 11 nt or 12 nt downstream of the first base pair of the duplex that forms between the siRNA and target transcript (see, e.g., Elbashir et al., 2001, Genes Dev., 15:188; incorporated herein by reference).

In some embodiments, siRNAs comprise 3′-overhangs at one or both ends of the duplex region. In some embodiments, an shRNA comprises a 3′ overhang at its free end. In some embodiments, siRNAs comprise a single nucleotide 3′-overhang. In some embodiments, siRNAs comprise a 3′-overhang of 2 nt. In some embodiments, siRNAs comprise a 3′-overhang of 1 nt. Overhangs, if present, may, but need not be, complementary to the target transcript. siRNAs with 2 nt-3 nt overhangs on their 3′-ends are frequently efficient in reducing target transcript levels than siRNAs with blunt ends.

Any desired sequence (e.g., UU) may simply be appended to the 3′ ends of antisense and/or sense core regions to generate 3′-overhangs. In general, overhangs containing one or more pyrimidines, usually U, T, or dT, are employed. When synthesizing RNAi-inducing agents, it may be more convenient to use T rather than U in the overhang(s). Use of dT rather than T may confer increased stability.

In some embodiments, the inhibitory region of a short RNAi agent is 100% complementary to a region of a target transcript. However, in some embodiments, the inhibitory region of a short RNAi agent is less than 100% complementary to a region of a target transcript. The inhibitory region need only be sufficiently complementary to a target transcript such that hybridization can occur, e.g., under physiological conditions in a cell and/or in an in vitro system that supports RNAi (e.g., a Drosophila extract system).

One of ordinary skill in the art will appreciate that short RNAi agent duplexes may tolerate mismatches and/or bulges, particularly mismatches within the central region of the duplex, while still leading to effective silencing. One of skill in the art will also recognize that it may be desirable to avoid mismatches in the central portion of the short RNAi agent/target transcript core region (see, e.g., Elbashir et al., EMBO J. 20:6877, 2001). For example, the 3′ nucleotides of the antisense strand of the siRNA often do not contribute significantly to specificity of the target recognition and may be less critical for target cleavage.

In some embodiments, short RNAi agents having duplex regions that exhibit one or more mismatches typically have no more than 6 total mismatches. In some embodiments, short RNAi agents have 1, 2, 3, 4, 5, or 6 total mismatches in their duplex regions. In some embodiments, the duplex regions have stretches of perfect complementarity that are at least 5 nt in length (e.g., 6, 7, or more nt). In some embodiments, no more than 20% of the nucleotides within a duplex region are mismatched. In some embodiments, no more than 15% of the nucleotides within a duplex region are mismatched. In some embodiments, no more than 10% of the nucleotides within a duplex region are mismatched. In some embodiments, no more than 5% of the nucleotides within a duplex region are mismatched. In some embodiments, none of the nucleotides within a duplex region are mismatched. Duplex regions may include two stretches of perfect complementarity separated by a region of mismatch. In some embodiments, there are multiple areas of mismatch.

In some embodiments, core regions (e.g., formed by hybridization of one strand of a short RNAi agent with a target transcript), which exhibit one or more mismatches typically, have no more than 6 total mismatches. In some embodiments, core regions have 1, 2, 3, 4, 5, or 6 total mismatches. In some embodiments, core regions comprise stretches of perfect complementarity that are at least 5 nt in length (e.g., 6, 7, or more nt). In some embodiments, no more than 20% of the nucleotides within a core region are mismatched. In some embodiments, no more than 15% of the nucleotides within a core region are mismatched. In some embodiments, no more than 10% of the nucleotides within a core region are mismatched. In some embodiments, no more than 5% of the nucleotides within a core region are mismatched. In some embodiments, none of the nucleotides within a core region are mismatched. Core regions may include two stretches of perfect complementarity separated by a region of mismatch. In some embodiments, there are multiple areas of mismatch.

In some embodiments, one or both strands of a short RNAi agent may include one or more “extra” nucleotides that form a “bulge.” One or more bulges (e.g., 5 nt-10 nt long) may be present.

In some embodiments, short RNAi agents can be designed and/or predicted using one or more of a large number of available algorithms. To give but a few examples, the following resources can be utilized to design and/or predict RNAi agents: algorithms found at Alnylum Online, Dharmacon Online, OligoEngine Online, Molecula Online, Ambion Online, BioPredsi Online, RNAi Web Online, Chang Bioscience Online, Invitrogen Online, LentiWeb Online GenScript Online, Protocol Online; Reynolds et al., 2004, Nat. Biotechnol., 22:326; Naito et al., 2006, Nucleic Acids Res., 34:W448; Li et al., 2007, RNA, 13:1765; Yiu et al., 2005, Bioinformatics, 21:144; and Jia et al., 2006, BMC Bioinformatics, 7: 271; each of which is incorporated herein by reference).

micro RNAs

micro RNAs (miRNAs) are genomically encoded non-coding RNAs of about 21-23 nucleotides in length that help regulate gene expression, particularly during development (see, e.g., Bartel, 2004, Cell, 116:281; Novina and Sharp, 2004, Nature, 430:161; and U.S. Patent Publication 2005/0059005; also reviewed in Wang and Li, 2007, Front. Biosci., 12:3975; and Zhao, 2007, Trends Biochem. Sci., 32:189; each of which are incorporated herein by reference). The phenomenon of RNA interference, broadly defined, includes the endogenously induced gene silencing effects of miRNAs as well as silencing triggered by foreign dsRNA. Mature miRNAs are structurally similar to siRNAs produced from exogenous dsRNA, but before reaching maturity, miRNAs first undergo extensive post-transcriptional modification. An miRNA is typically expressed from a much longer RNA-coding gene as a primary transcript known as a pri-miRNA, which is processed in the cell nucleus to a 70-nucleotide stem-loop structure called a pre-miRNA by the microprocessor complex. This complex consists of an RNase III enzyme called Drosha and a dsRNA-binding protein Pasha. The dsRNA portion of this pre-miRNA is bound and cleaved by dicer to produce the mature miRNA molecule that can be integrated into the RISC complex; thus, miRNA and siRNA share the same cellular machinery downstream of their initial processing (Gregory et al., 2006, Meth. Mol. Biol., 342:33; incorporated herein by reference). In general, miRNAs are not perfectly complementary to their target transcripts.

In some embodiments, miRNAs can range between 18 nt-26 nt in length. Typically, miRNAs are single-stranded. However, in some embodiments, miRNAs may be at least partially double-stranded. In certain embodiments, miRNAs may comprise an RNA duplex (referred to herein as a “duplex region”) and may optionally further comprises one or two single-stranded overhangs. In some embodiments, an RNAi agent comprises a duplex region ranging from 15 bp to 29 bp in length and optionally further comprising one to three single-stranded overhangs. An miRNA may be formed from two RNA molecules that hybridize together, or may alternatively be generated from a single RNA molecule that includes a self-hybridizing portion. The duplex portion of an miRNA usually, but does not necessarily, comprise one or more bulges consisting of one or more unpaired nucleotides. One strand of an miRNA includes a portion that hybridizes with a target RNA. In certain embodiments, one strand of the miRNA is not precisely complementary with a region of the target RNA, meaning that the miRNA hybridizes to the target RNA with one or more mismatches. In some embodiments, one strand of the miRNA is precisely complementary with a region of the target RNA, meaning that the miRNA hybridizes to the target RNA with no mismatches. Typically, miRNAs are thought to mediate inhibition of gene expression by inhibiting translation of target transcripts. However, in some embodiments, miRNAs may mediate inhibition of gene expression by causing degradation of target transcripts.

In some embodiments, miRNAs have a duplex region of about 15 nt, about 16 nt, about 17 nt, about 18 nt, about 19 nt, about 20 nt, about 21 nt, about 22 nt, about 23 nt, about 24 nt, about 25 nt, about 26 nt, about 27 nt, about 28 nt, or about 29 nt in length. In some embodiments, miRNAs have an inhibitory region of about 15 nt, about 16 nt, about 17 nt, about 18 nt, about 19 nt, about 20 nt, about 21 nt, about 22 nt, about 23 nt, about 24 nt, about 25 nt, about 26 nt, about 27 nt, about 28 nt, or about 29 nt in length.

In some embodiments, miRNAs have duplex regions that exhibit one or more mismatches in their duplex regions. In some embodiments, miRNAs have duplex regions that exhibit 1, 2, 3, 4, 5, 6, 7, 8, or 9 total mismatches in their duplex regions. In some embodiments, the duplex regions have stretches of perfect complementarity that are 1, 2, 3, 4, 5, 6, 7, 8, or 9 nt in length. Duplex regions may include two stretches of perfect complementarity separated by a region of mismatch. In some embodiments, there are multiple areas of mismatch. In some embodiments, about 50% of the nucleotides within a duplex region are mismatched. In some embodiments, about 40% of the nucleotides within a duplex region are mismatched. In some embodiments, about 30% of the nucleotides within a duplex region are mismatched. In some embodiments, about 20% of the nucleotides within a duplex region are mismatched. In some embodiments, about 10% of the nucleotides within a duplex region are mismatched. In some embodiments, about 5% of the nucleotides within a duplex region are mismatched.

In some embodiments, core regions (e.g., formed by hybridization of one strand of an miRNA with a target transcript) have 1, 2, 3, 4, 5, 6, 7, 8, or 9 total mismatches. In some embodiments, core regions comprise stretches of perfect complementarity that are 1, 2, 3, 4, 5, 6, 7, 8, or 9 nt in length. Core regions may include two stretches of perfect complementarity separated by a region of mismatch. In some embodiments, there are multiple areas of mismatch. In some embodiments, there are multiple areas of mismatch. In some embodiments, about 50% of the nucleotides within a core region are mismatched. In some embodiments, about 40% of the nucleotides within a core region are mismatched. In some embodiments, about 30% of the nucleotides within a core region are mismatched. In some embodiments, about 20% of the nucleotides within a core region are mismatched. In some embodiments, about 10% of the nucleotides within a core region are mismatched. In some embodiments, about 5% of the nucleotides within a core region are mismatched.

In some embodiments, one or both strands of an miRNA may include one or more “extra” nucleotides that form a “bulge.” One or more bulges (e.g., 5 nt-10 nt long) may be present.

In some embodiments, short RNAi agents can be designed and/or predicted using one or more of a large number of available algorithms. To give but a few examples, the following resources can be utilized to design and/or predict RNAi agents: algorithms at PicTar Online, Protocol Online, EMBL Online; Rehmsmeier et al., 2004, RNA, 10:1507; Kim et al., 2006, BMC Bioinformatics, 7:411; Lewis et al., 2003, Cell, 115:787; and Krek et al., 2005, Nat. Genet., 37:495; each of which is incorporated herein by reference.

### Antisense RNAs

In some embodiments, nucleic acids that can be associated with supercharged proteins include antisense RNAs. Antisense RNAs are typically RNA strands of various lengths that bind to target transcripts and block their translation (e.g., either through degradation of mRNA and/or by sterically blocking critical steps of the translation process).

Antisense RNAs exhibit many of the same characteristics of RNAi agents described above. For example, antisense RNAs exhibit sufficient complementarity to a target transcript to allow hybridization of the antisense RNA to the target transcript. Mismatches are tolerated, as described above for RNAi agents, as long as hybridization to the target can still occur. In general, antisense RNAs are longer than short RNAi agents, and can be of any length, as long as hybridization can still occur. In some embodiments, antisense RNAs are about 20 nt, about 30 nt, about 40 nt, about 50 nt, about 75 nt, about 100 nt, about 150 nt, about 200 nt, about 250 nt, about 500 nt, or longer. In some embodiments, antisense RNAs comprise an inhibitory region that hybridizes with a target transcript of about 20 nt, about 30 nt, about 40 nt, about 50 nt, about 75 nt, about 100 nt, about 150 nt, about 200 nt, about 250 nt, about 500 nt, or longer.

### Ribozymes

In some embodiments, nucleic acids that can be associated with supercharged proteins include ribozymes. A ribozyme (from ribonucleic acid enzyme; also called RNA enzyme or catalytic RNA) is an RNA molecule that catalyzes a chemical reaction. Many natural ribozymes catalyze either the hydrolysis of one of their own phosphodiester bonds, or the hydrolysis of bonds in other RNAs, but they have also been found to catalyze the aminotransferase activity of the ribosome.

In some embodiments, ribozymes used for gene-knockdown applications have a catalytic domain that is flanked by sequences complementary to a target transcript. The mechanism of gene silencing generally involves binding of a ribozyme to a target transcript via Watson-Crick base pairing, followed by cleavage of the phosphodiester backbone of the target transcript by transesterification (Kurreck, 2003, Eur. J. Biochem., 270:1628; Sun et al., 2000, Pharmacol. Rev., 52:325; Doudna and Cech, 2002, Nature, 418:222; Goodchild, 2000, Curr. Opin. Mol. Ther., 2:272; Michienzi and Rossi, 2001, Methods Enzymol., 341:581; each of which is incorporated herein by reference). Once the target transcript is destroyed, ribozymes dissociate and subsequently can repeat cleavage on additional substrates. In some embodiments, a ribozyme to be associated with a supercharged protein is a hammerhead ribozyme. Hammerhead ribozymes were first isolated from viroid RNAs that undergo site-specific self-cleavage as part of their replication process.

In some embodiments, ribozymes are naturally-occurring ribozymes, including but not limited to, peptidyl transferase 23S rRNA, RNase P, Group I and Group II introns, GIR1 branching ribozyme, leadzyme, hairpin ribozyme, hammerhead ribozyme, HDV ribozyme, mammalian CPEB3 ribozyme, VS ribozyme, glmS ribozyme, and CoTC ribozyme.

In some embodiments, ribozymes are artificial ribozymes. For example, artificially-produced self-cleaving RNAs that have good enzymatic activity have been produced. Tang and Breaker (1997, Proc. Natl. Acad. Sci., 97:5784; incorporated herein by reference) isolated self-cleaving RNAs by in vitro selection of RNAs originating from random-sequence RNAs. Some of the synthetic ribozymes that were produced had novel structures, while some were similar to the naturally occurring hammerhead ribozyme.

In some embodiments, techniques used to discover artificial ribozymes involve Darwinian evolution. This approach takes advantage of RNA's dual nature as both a catalyst and an informational polymer, thereby allowing an investigator to produce vast populations of RNA catalysts using polymerase enzymes. Ribozymes are mutated by reverse transcribing them with reverse transcriptase into various cDNA and amplified with mutagenic PCR. The selection parameters in these experiments often differ. To give but one example, an approach for selecting a ligase ribozyme might involve using biotin tags, which are covalently linked to a substrate. If a candidate ribozyme possesses the desired ligase activity, a streptavidin matrix can be used to recover the active molecules.

### Deoxyribozymes

In some embodiments, nucleic acids that can be associated with supercharged proteins include catalytic DNAs (“deoxyribozymes”). Deoxyribozymes bind to RNA substrates, typically via Watson-Crick base pairing, and site-specifically cleave target transcripts, similarly to ribozymes. Deoxyribozymes molecules have been produced by in vitro evolution since no natural examples of DNA enzymes are known. Two different catalytic motifs, with different cleavage site specificities, have been identified. Deoxyribozymes have been produced with different cleavage specificities, allowing researchers to target all possible dinucleotide sequences.

### Aptamers

In some embodiments, nucleic acids that can be associated with supercharged proteins include aptamers. Aptamers are oligonucleic acid molecules that bind specific target molecules. Aptamers may be engineered through repeated rounds of in vitro selection (e.g., via systematic evolution of ligands by exponential enrichment, “SELEX”) to bind to various molecular targets such as small molecules, proteins, nucleic acids, cells, tissues, and/or organisms. Aptamers typically bind to their targets due to the three-dimensional structure of the aptamer. Aptamers generally do not bind to their targets via traditional Watson-Crick base pairing.

The first aptamer-based drug approved by the U.S. Food and Drug Administration (FDA) in treatment for age-related macular degeneration (AMD), called MACUGEN® (OSI Pharmaceuticals). In addition, ARC 1779 (Archemix, Cambridge, Mass.) is a potent, selective, first-in-class antagonist of von Willebrand Factor (vWF) and is being evaluated in patients diagnosed with acute coronary syndrome (ACS) who are undergoing percutaneous coronary intervention (PCI).

In general, unmodified aptamers are usually cleared rapidly from the bloodstream, with a half-life of minutes to hours. This is presumably due to nuclease degradation and clearance from the body by the kidneys, which occur because aptamers tend to have low molecular weights. Unmodified aptamers may be particularly suited for treating transient conditions (e.g., blood clotting), and/or for treating organs where local delivery is possible (e.g., the eye, skin, etc.). Rapid clearance can be desirable in applications such as in vivo diagnostic imaging. For example, a tenascin-binding aptamer (Schering A G) can be utilized for cancer imaging. In some embodiments, aptamers with increased half-lives are desirable. Certain modifications (e.g., 2′-fluorine-substituted pyrimidines, polyethylene glycol (PEG) linkage, etc.) may increase the half-life of aptamers.

RNA that Induce Triple Helix Formation

In some embodiments, nucleic acids that can be associated with supercharged proteins include RNAs that induce triple helix formation. In some embodiments, endogenous target gene expression may be reduced by targeting deoxyribonucleotide sequences complementary to the regulatory region of the target gene (i.e., the target gene's promoter and/or enhancers) to form triple helical structures that prevent transcription of the target gene in target muscle cells in the body (see generally, Helene, 1991, Anticancer Drug Des. 6:569; Helene et al., 1992, Ann, N.Y. Acad. Sci. 660:27; and Maher, 1992, Bioassays 14:807).

### Vectors

In some embodiments, nucleic acids that can be associated with supercharged proteins include vectors. As used herein, “vector” refers to a nucleic acid molecule which can transport another nucleic acid to which it has been linked. In some embodiment, vectors can achieve extra-chromosomal replication and/or expression of nucleic acids to which they are linked in a host cell such as a eukaryotic and/or prokaryotic cell. Exemplary vectors include plasmids, cosmids, viruses, viral genomes, artificial chromosomes, bacterial artificial chromosomes, and/or yeast artificial chromosomes. In certain embodiments, vectors include elements such as promoters, enhancers, ribosomal binding sites, etc.

In some embodiments, vectors are capable of directing the expression of operatively linked genes (“expression vectors”). In some embodiments, expression of the operatively linked gene may result in production of a functional nucleic acid (e.g., RNAi agent, antisense RNA, aptamer, ribozyme, etc.). In some embodiments, expression of the operatively linked gene may result in production of a protein (e.g., a therapeutic, diagnostic, and/or prophylactic protein). In some embodiments, a therapeutic protein is a protein-based drug (e.g., an antibody-based drug, a peptide-based drug, etc.). In some embodiments, a prophylactic protein may be a protein antigen and/or antibody. In some embodiments, a diagnostic protein may be one that exhibits certain characteristics before delivery to a cell by a supercharged protein, but exhibits detectably different characteristics after delivery.

In some embodiments, a vector is a viral vector. In some embodiments, a vector is of bacterial origin. In some embodiments, a vector is of fungal origin. In some embodiments, a vector is of eukaryotic origin. In some embodiments, a vector is of prokaryotic origin. In some embodiments, a vector may be delivered to a cell via a supercharged protein, where it subsequently replicates in vivo. In some embodiments, a vector may be delivered to a cell via a supercharged protein, where it is subsequently transcribed in vivo.

### Labeled Nucleic Acids

In some embodiments, nucleic acids in accordance with the invention are tagged with a detectable label. Suitable labels that can be used in accordance with the invention include, but are not limited to, fluorescent, chemiluminescent, phosphorescent, and/or radioactive labels. In some embodiments, nucleic acids comprise at least one nucleotide that is attached to at least one fluorescent moiety (e.g., fluorescein, rhodamine, coumarin, cyanine-3, cyanine-5, Alexa Fluor, and DyLight Fluor, etc.). Any fluorescent moiety that can be associated with a nucleic acid can be utilized in accordance with the invention. In some embodiments, nucleic acids comprise at least one radioactive nucleotide (e.g., a nucleotide containing ³²P or ³⁵S). In some embodiments, nucleic acids comprise at least one nucleotide that is attached to at least one radioactive moiety.

### Cellular Nucleic Acids Targeted by Delivered Nucleic Acids

In some embodiments, nucleic acids (e.g., siRNAs, shRNAs, miRNAs, antisense RNAs, ribozymes, etc.) to be delivered to cells using supercharged proteins are useful for targeting cellular nucleic acids for degradation. Any cellular nucleic acid can be targeted for degradation. Exemplary cellular nucleic acids that can be targeted for degradation include, but are not limited to, GAPDH, β-actin, β-tubulin, and c-myc.

### Peptides and Proteins

The present invention provides systems and methods for delivery of proteins or peptides to cells in vivo or in vitro. Such systems and methods typically involve association of one or more peptides or proteins with supercharged proteins to form a complex, and delivery of the complex to one or more cells. In some embodiments, the protein or peptide may have therapeutic activity. In some embodiments, delivery of the complex to cells involves administering a complex comprising supercharged proteins associated with a peptide or protein to a subject in need thereof. In some embodiments, a peptide or protein by itself may not be able to enter the interior of a cell, but is able to enter the interior of a cell when complexed with a supercharged protein. In some embodiments, a supercharged protein is utilized to allow a peptide or protein to enter a cell. Peptides or proteins in accordance with the invention may themselves have therapeutic activity.

### Small Molecules

The present invention provides systems and methods for delivery of small molecules to cells in vivo or in vitro. Such systems and methods typically involve association of one or more small molecules with supercharged proteins to form a complex, and delivery of the complex to one or more cells. In some embodiments, the small molecule may have therapeutic activity. Preferably, though not necessarily, the drug is one that has already been deemed safe and effective for use in humans or animals by the appropriate governmental agency or regulatory body. In certain embodiments, the small molecule is a drug approved by the U.S. Food and Drug Administration for use in humans or other animals. For example, drugs approved for human use are listed by the FDA under 21 C.F.R. §§330.5, 331 through 361, and 440 through 460, incorporated herein by reference; drugs for veterinary use are listed by the FDA under 21 C.F.R. §§500 through 589, incorporated herein by reference. All listed drugs are considered acceptable for use in accordance with the present invention. In some embodiments, delivery of the complex to cells involves administering a complex comprising supercharged proteins associated with a small molecule to a subject in need thereof. In some embodiments, a small molecule by itself may not be able to enter the interior of a cell, but is able to enter the interior of a cell when complexed with a supercharged protein. In some embodiments, a supercharged protein is utilized to allow a small molecule to enter a cell.

### Formation of Complexes

The present invention provides complexes comprising supercharged proteins associated with one or more agents to be delivered. In some embodiments, supercharged proteins are associated with one or more agents to be delivered by non-covalent interactions. In some embodiments, supercharged proteins are associated with one or more nucleic acids by electrostatic interactions. In certain embodiments, supercharged proteins have an overall net positive charge, and the agent to be delivered such as nucleic acids have an overall net negative charge.

In certain embodiments, supercharged proteins are associated with one or more agents to be delivered by covalent interactions. For example, a supercharged protein may be fused to a peptide or protein to be delivered. Covalent interaction may be direct or indirect. In some embodiments, such covalent interactions are mediated by one or more linkers. In some embodiments, the linker is a cleavable linker. In certain embodiments, the cleavable linker comprises an amide, ester, or disulfide bond. For example, the linker may be an amino acid sequence that is cleavable by a cellular enzyme. In certain embodiments, the enzyme is a protease. In other embodiments, the enzyme is an esterase. In some embodiments, the enzyme is one that is more highly expressed in certain cell types than in other cell types. For example, the enzyme may be one that is more highly expressed in tumor cells than in non-tumor cells. Exemplary linkers and enzymes that cleave those linkers are presented in Table 3.

TABLE 3
Cleavable Linkers

| | |
|---|---|
| Linker Sequence | Enzyme(s) Targeting Linker |

| | |
|---|---|
| X¹-AGVF-X (SEQ | lysosomal thiol proteinases (see, e.g., Duncan et al., 1982, Biosci. Rep., |
| ID NO: XX) | 2: 1041-46; incorporated herein by reference) |
| X-GFLG-X (SEQ | lysosomal cysteine proteinases (see, e.g., Vasey et al., Clin. Canc. Res., |
| ID NO: XX) | 1999, 5: 83-94; incorporated herein by reference) |
| X-FK-X (SEQ ID | Cathepsin B - ubiquitous, overexpressed in many solid tumors, such as |
| NO: XX) | breast cancer (see, e.g., Dubowchik et al., 2002, Bioconjugate Chem., |
| | 13: 855-69; incorporated herein by reference) |
| X-A*L-X (SEQ ID | Cathepsin B - ubiquitous, overexpressed in many solid tumors, such as |
| NO: XX) | breast cancer (see, e.g., Trouet et al., 1982, Proc. Natl. Acad. Sci., USA, |
| | 79: 626-29; incorporated herein by reference) |
| X-A*LA*L-X | Cathepsin B - ubiquitous, overexpressed in many solid tumors (see, e.g., |
| (SEQ ID NO: XX) | Schmid et al., 2007, Bioconjugate Chem, 18: 702-16; incorporated herein |
| | by reference) |
| X-AL*AL*A-X | Cathepsin D - ubiquitous (see, e.g., Czerwinski et al., 1998, Proc. Natl. |
| (SEQ ID NO: XX) | Acad. Sci., USA, 95: 11520-25; incorporated herein by reference) |

¹X denotes a supercharged protein and/or agent to be delivered
*refers to observed cleavage site

To give but one particular example, a +36 GFP may be associated with an agent to be delivered by a cleavable linker, such as ALAL (SEQ ID NO: XX), to generate +36 GFP-(GGS)₄-ALAL-(GGS)₄-X (where X is the agent to be delivered).

In certain embodiments, the agent to be delivered is a nucleic acid. In some embodiments, complexes are formed by incubating supercharged proteins with nucleic acids. In some embodiments, formation of complexes is carried out in a buffered solution. In some embodiments, formation of complexes is carried out at or around pH 7. In some embodiments, formation of complexes is carried out at about pH 5, about pH 6, about pH 7, about pH 8, or about pH 9. Formation of complexes is typically carried out at a pH that does not negatively affect the function of the supercharged protein and/or nucleic acid.

In some embodiments, formation of complexes is carried out at room temperature. In some embodiments, formation of complexes is carried out at or around 37° C. In some embodiments, formation of complexes is carried out below 4° C., at about 4° C., at about 10° C., at about 15° C., at about 20° C., at about 25° C., at about 30° C., at about 35° C., at about 37° C., at about 40° C., or higher than 40° C. Formation of complexes is typically carried out at a temperature that does not negatively affect the function of the supercharged protein and/or nucleic acid.

In some embodiments, formation of complexes is carried out in serum-free medium. In some embodiments, formation of complexes is carried out in the presence of CO₂ (e.g., about 1%, about 2%, about 3%, about 4%, about 5%, about 6%, or more).

In some embodiments, formation of complexes is carried out using concentrations of nucleic acid of about 100 nm. In some embodiments, formation of complexes is carried out using concentrations of nucleic acid of about 25 nM, about 50 nM, about 75 nM, about 90 nM, about 100 nM, about 110 nM, about 125 nM, about 150 nM, about 175 nM, or about 200 nM. In some embodiments, formation of complexes is carried out using concentrations of supercharged protein of about 40 nM. In some embodiments, formation of complexes is carried out using concentrations of supercharged protein of about 10 nM, about 20 nM, about 30 nM, about 40 nM, about 50 nM, about 60 nM, about 70 nM, about 80 nM, about 90 nM, or about 100 nM.

In some embodiments, formation of complexes is carried out under conditions of excess nucleic acid. In some embodiments, formation of complexes is carried out with ratios of nucleic acid:supercharged protein of about 20:1, about 10:1, about 9:1, about 8:1, about 7:1, about 6:1, about 5:1, about 4:1, about 3:1, about 2:1, or about 1:1. In some embodiments, formation of complexes is carried out with ratios of nucleic acid:supercharged protein of about 3:1. In some embodiments, formation of complexes is carried out with ratios of supercharged protein:nucleic acid of about 20:1, about 10:1, about 9:1, about 8:1, about 7:1, about 6:1, about 5:1, about 4:1, about 3:1, about 2:1, or about 1:1.

In some embodiments, formation of complexes is carried out by mixing supercharged protein with nucleic acid, and agitating the mixture (e.g., by inversion). In some embodiments, formation of complexes is carried out by mixing supercharged protein with nucleic acid, and allowing the mixture to sit still. In some embodiments, the formation of the complex is carried out in the presence of a pharmaceutically acceptable carrier or excipient. In some embodiments, the complex is further combined with a pharmaceutically acceptable carrier or excipient. Exemplary excipients or carriers include water, solvents, lipids, proteins, peptides, endosomolytic agents (e.g., chloroquine, pyrene butyric acid), small molecules, carbohydrates, buffers, natural polymers, synthetic polymers (e.g., PLGA, polyurethane, polyesters, polycaprolactone, polyphosphazenes), pharmaceutical agents, etc.

In some embodiments, complexes comprising supercharged protein and nucleic may migrate more slowly in gel electrophoresis assays than either the supercharged protein alone or the nucleic acid alone.

### Applications

The present invention provides supercharged proteins or complexes comprising supercharged proteins, naturally occurring or engineered, associated with agents to be delivered, as well as methods for using such complexes. Any agent may be delivered using the inventive system. In the case of delivering nucleic acids, since nucleic acids generally have net negative charges, supercharged proteins that associate with nucleic acids are typically superpositively charged proteins. The inventive supercharged proteins or complexes may be used to treat or prevent any disease that can benefit, e.g., from the delivery of an agent to a cell. The inventive supercharged proteins or complexes may also be used to transfect or treat cells for research purposes.

In some embodiments, supercharged proteins or complexes in accordance with the invention may be used for research purposes, e.g., to efficiently deliver nucleic acids to cells in a research context. In some embodiments, supercharged proteins may be used as research tools to efficiently transform cells with nucleic acids. In some embodiments, supercharged proteins may be used as research tools to efficiently introduce RNAi agents into cells for purposes of studying RNAi mechanisms. In some embodiments, supercharged proteins may be used as research tools to silence genes in a cell. In certain embodiments, supercharged proteins may be used to deliver a peptide or protein into a cell for the purpose of studying the biological activity of the peptide or protein. In certain embodiments, supercharged proteins may be introduced into a cell for the purpose of studying the biological activity of the peptide or protein. In certain embodiments, supercharged proteins may be used to deliver a small molecule into a cell for the purpose of studying the biological activity of the small molecule.

In some embodiments, supercharged proteins or complexes in accordance with the present invention may be used for therapeutic purposes. In some embodiments, supercharged proteins or complexes in accordance with the present invention may be used for treatment of any of a variety of diseases, disorders, and/or conditions, including but not limited to one or more of the following: autoimmune disorders (e.g. diabetes, lupus, multiple sclerosis, psoriasis, rheumatoid arthritis); inflammatory disorders (e.g. arthritis, pelvic inflammatory disease); infectious diseases (e.g. viral infections (e.g., HIV, HCV, RSV), bacterial infections, fungal infections, sepsis); neurological disorders (e.g. Alzheimer's disease, Huntington's disease; autism; Duchenne muscular dystrophy); cardiovascular disorders (e.g. atherosclerosis, hypercholesterolemia, thrombosis, clotting disorders, angiogenic disorders such as macular degeneration); proliferative disorders (e.g. cancer, benign neoplasms); respiratory disorders (e.g. chronic obstructive pulmonary disease); digestive disorders (e.g. inflammatory bowel disease, ulcers); musculoskeletal disorders (e.g. fibromyalgia, arthritis); endocrine, metabolic, and nutritional disorders (e.g. diabetes, osteoporosis); urological disorders (e.g. renal disease); psychological disorders (e.g. depression, schizophrenia); skin disorders (e.g. wounds, eczema); blood and lymphatic disorders (e.g. anemia, hemophilia); etc.

Supercharged proteins or complexes of the invention may be used in a clinical setting. For example, a supercharged protein may be associated with a nucleic acid that can be used for therapeutic applications. Such nucleic acids may include functional RNAs that are used to reduce levels of one or more target transcripts (e.g., siRNAs, shRNAs, microRNAs, antisense RNAs, ribozymes, etc.). In some embodiments, a disease, disorder, and/or condition may be associated with abnormally high levels of one or more particular mRNAs and/or proteins. To give but one particular example, many forms of breast cancer are associated with increased expression of the epidermal growth factor receptor (EGFR). Supercharged proteins may be utilized to deliver an RNAi agent that targets EGFR mRNA to cells (e.g., breast cancer tumor cells). Supercharged proteins may be efficiently taken up by tumor cells, resulting in delivery of the RNAi agent. Upon delivery, the RNAi agent may be effective to reduce levels of EGFR mRNA, thereby reducing levels of EGFR protein. Such a method may be an effective treatment for breast cancers (e.g., breast cancers associated with elevated levels of EGFR). One of ordinary skill in the art will recognize that similar methods may be used to treat any disease, disorder, and/or condition that is associated with elevated levels of one or more particular mRNAs and/or proteins.

In some embodiments, a disease, disorder, and/or condition may be associated with abnormally low levels of one or more particular mRNAs and/or proteins. To give but one particular example, tyrosinemia is a disorder in which the body cannot effectively break down the amino acid tyrosine. There are three types of tyrosinemia, each caused by a deficiency in a different enzyme. Supercharged proteins may be used to treat tyrosinemia by delivering a vector that drives expression of the deficient enzyme. Upon delivery of the vector to cells, cellular machinery can direct expression of the deficient enzyme, thereby treating a patient's tyrosinemia. One of ordinary skill in the art will recognize that similar methods may be used to treat any disease, disorder, and/or condition that is associated with abnormally low levels of one or more particular mRNAs and/or proteins.

As demonstrated in Examples 2 and 3, supercharged protein-based nucleic acid delivery to cells is successful, even using cell lines that are resistant to nucleic acid transfection using conventional cationic lipid-based transfection methods. Thus, in some embodiments, supercharged proteins are utilized to deliver nucleic acids to cells which are resistant to other methods of nucleic acid delivery (e.g., cationic lipid-based transformation methods, such as use of lipofectamine). Furthermore, the present inventors have demonstrated that, surprisingly, superpositively charged proteins can be used at low nanomolar (nM) concentrations (e.g., 1 nm to 100 nm) to effectively deliver nucleic acids to cells. In some embodiments, supercharged proteins can be used at about 1 nm, about 5 nm, about 10 nm, about 25 nm, about 50 nm, about 75 nm, about 100 nm, or higher than about 100 nm to effectively deliver nucleic acids to cells.

In some embodiments, a supercharged protein may be a therapeutic agent. For example, a supercharged protein may be a supercharged variant of a protein drug (e.g., abatacept, adalimumab, alefacept, erythropoietin, etanercept, human growth hormone, infliximab, insulin, trastuzumab, interferons, etc.). In some embodiments, a supercharged protein may be a therapeutic agent, and an associated nucleic acid may be useful for targeting delivery of the therapeutic protein to a target site. For example, a supercharged protein may be a supercharged variant of a protein drug (e.g., abatacept, adalimumab, alefacept, erythropoietin, etanercept, human growth hormone, infliximab, insulin, trastuzumab, interferons, etc.), and an associated nucleic acid may be an aptamer that efficiently targets the therapeutic protein to a target organ, tissue, and/or cell. The supercharged protein can also be an imaging, diagnostic, or other detection agent.

In some embodiments, one or both of the supercharged protein and an agent to be delivered (if present) may have detectable qualities. For example, one or both of the supercharged protein and the agent may comprise at least one fluorescent moiety. In some embodiments, the supercharged protein has inherent fluorescent qualities (e.g., GFP). In some embodiments, one or both of the supercharged protein and the agent to be delivered may be associated with at least one fluorescent moiety (e.g., conjugated to a fluorophore, fluorescent dye, etc.). Alternatively or additionally, one or both of the supercharged protein and the agent to be delivered may comprise at least one radioactive moiety (e.g., protein may comprise ³⁵S; nucleic acid may comprise ³²P; etc.). Such detectable moieties may be useful for detecting and/or monitoring delivery of the supercharged proteins or complexes to target sites.

In some embodiments, the supercharged protein or an agent associated with a supercharged protein includes a detectable label. These molecules can be used in detection, imaging, disease staging, diagnosis, or patient selection. Suitable labels include fluorescent, chemiluminescent, enzymatic labels, colorimetric, phosphorescent, density-based labels, e.g., labels based on electron density, and in general contrast agents, and/or radioactive labels.

### Pharmaceutical Compositions

The present invention provides supercharged proteins and complexes comprising supercharged proteins associated with at least one agent to be delivered. Thus, the present invention provides pharmaceutical compositions comprising one or more supercharged proteins or one or more such complexes, and one or more pharmaceutically acceptable excipients. Pharmaceutical compositions may optionally comprise one or more additional therapeutically active substances. In accordance with some embodiments, a method of administering pharmaceutical compositions comprising one or more supercharged proteins or one or more complexes comprising supercharged proteins associated with at least one agent to be delivered to a subject in need thereof is provided. In some embodiments, compositions are administered to humans. For the purposes of the present disclosure, the phrase “active ingredient” generally refers to a supercharged protein or complex comprising a supercharged protein and at least one agent to be delivered as described herein.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, shaping and/or packaging the product into a desired single- or multi-dose unit.

A pharmaceutical composition in accordance with the invention may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the invention will vary, depending upon the identity, size, and/or condition of the subject treated and further depending upon the route by which the composition is to be administered. By way of example, the composition may comprise between 0.1% and 100% (w/w) active ingredient.

Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21^{st} Edition, A. R. Gennaro (Lippincott, Williams & Wilkins, Baltimore, Md., 2006; incorporated herein by reference) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention.

In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical formulations. Excipients such as cocoa butter and suppository waxes, coloring agents, coating agents, sweetening, flavoring, and/or perfuming agents can be present in the composition, according to the judgment of the formulator.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and/or combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween®20], polyoxyethylene sorbitan [Tween®60], polyoxyethylene sorbitan monooleate [Tween®80], sorbitan monopalmitate [Span®40], sorbitan monostearate [Span®60], sorbitan tristearate [Span®65], glyceryl monooleate, sorbitan monooleate [Span®80]), polyoxyethylene esters (e.g. polyoxyethylene monostearate [Myrj®45], polyoxyethylene hydrogenated castor oil, polyethoxylated castor oil, polyoxymethylene stearate, and Solutol®), sucrose fatty acid esters, polyethylene glycol fatty acid esters (e.g. Cremophor®), polyoxyethylene ethers, (e.g. polyoxyethylene lauryl ether [Brij° 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic®F 68, Poloxamer®188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan); alginates; polyethylene oxide; polyethylene glycol; inorganic calcium salts; silicic acid; polymethacrylates; waxes; water; alcohol; etc.; and combinations thereof.

Exemplary preservatives may include, but are not limited to, antioxidants, chelating agents, antimicrobial preservatives, antifungal preservatives, alcohol preservatives, acidic preservatives, and/or other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybenzoate, and/or phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus®, Phenonip®, methylparaben, Germall° 115, Germaben®II, Neolone™, Kathon™, and/or Euxyl®.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and/or combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof.

Liquid dosage forms for oral and parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as Cremophor®, alcohols, oils, modified oils, glycols, polysorbates, cyclodextrins, polymers, and/or combinations thereof.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it is often desirable to slow the absorption of the active ingredient from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

Compositions for rectal or vaginal administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, an active ingredient is mixed with at least one inert, pharmaceutically acceptable excipient such as sodium citrate or dicalcium phosphate and/or fillers or extenders (e.g. starches, lactose, sucrose, glucose, mannitol, and silicic acid), binders (e.g. carboxymethylcellulose, alginates, gelatin, polyvinylpyrrolidinone, sucrose, and acacia), humectants (e.g. glycerol), disintegrating agents (e.g. agar, calcium carbonate, potato or tapioca starch, alginic acid, certain silicates, and sodium carbonate), solution retarding agents (e.g. paraffin), absorption accelerators (e.g. quaternary ammonium compounds), wetting agents (e.g. cetyl alcohol and glycerol monostearate), absorbents (e.g. kaolin and bentonite clay), and lubricants (e.g. talc, calcium stearate, magnesium stearate, solid polyethylene glycols, sodium lauryl sulfate), and mixtures thereof. In the case of capsules, tablets and pills, the dosage form may comprise buffering agents.

Solid compositions of a similar type may be employed as fillers in soft and hard-filled gelatin capsules using such excipients as lactose or milk sugar as well as high molecular weight polyethylene glycols and the like. Solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

Dosage forms for topical and/or transdermal administration of a composition may include ointments, pastes, creams, lotions, gels, powders, solutions, sprays, inhalants and/or patches. Generally, an active ingredient is admixed under sterile conditions with a pharmaceutically acceptable excipient and/or any needed preservatives and/or buffers as may be required. Additionally, the present invention contemplates the use of transdermal patches, which often have the added advantage of providing controlled delivery of a compound to the body. Such dosage forms may be prepared, for example, by dissolving and/or dispensing the compound in the proper medium. Alternatively or additionally, rate may be controlled by either providing a rate controlling membrane and/or by dispersing the compound in a polymer matrix and/or gel.

Suitable devices for use in delivering intradermal pharmaceutical compositions described herein include short needle devices such as those described in U.S. Pat. Nos. 4,886,499; 5,190,521; 5,328,483; 5,527,288; 4,270,537; 5,015,235; 5,141,496; and 5,417,662. Intradermal compositions may be administered by devices which limit the effective penetration length of a needle into the skin, such as those described in PCT publication WO 99/34850 and functional equivalents thereof. Jet injection devices which deliver liquid compositions to the dermis via a liquid jet injector and/or via a needle which pierces the stratum corneum and produces a jet which reaches the dermis are suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/37705 and WO 97/13537. Ballistic powder/particle delivery devices which use compressed gas to accelerate vaccine in powder form through the outer layers of the skin to the dermis are suitable. Alternatively or additionally, conventional syringes may be used in the classical mantoux method of intradermal administration.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Topically-administrable formulations may, for example, comprise from about 1% to about 10% (w/w) active ingredient, although the concentration of active ingredient may be as high as the solubility limit of the active ingredient in the solvent. Formulations for topical administration may further comprise one or more of the additional ingredients described herein.

A pharmaceutical composition may be prepared, packaged, and/or sold in a formulation suitable for pulmonary administration via the buccal cavity. Such a formulation may comprise dry particles which comprise the active ingredient and which have a diameter in the range from about 0.5 nm to about 7 nm or from about 1 nm to about 6 nm. Such compositions are conveniently in the form of dry powders for administration using a device comprising a dry powder reservoir to which a stream of propellant may be directed to disperse the powder and/or using a self propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nm and at least 95% of the particles by number have a diameter less than 7 nm. Alternatively, at least 95% of the particles by weight have a diameter greater than 1 nm and at least 90% of the particles by number have a diameter less than 6 nm. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50% to 99.9% (w/w) of the composition, and active ingredient may constitute 0.1% to 20% (w/w) of the composition. A propellant may further comprise additional ingredients such as a liquid non-ionic and/or solid anionic surfactant and/or a solid diluent (which may have a particle size of the same order as particles comprising the active ingredient).

Pharmaceutical compositions formulated for pulmonary delivery may provide an active ingredient in the form of droplets of a solution and/or suspension. Such formulations may be prepared, packaged, and/or sold as aqueous and/or dilute alcoholic solutions and/or suspensions, optionally sterile, comprising active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 0.1 nm to about 200 nm.

Formulations described herein as being useful for pulmonary delivery are useful for intranasal delivery of a pharmaceutical composition. Another formulation suitable for intranasal administration is a coarse powder comprising the active ingredient and having an average particle from about 0.2 μm to 500 μm. Such a formulation is administered in the manner in which snuff is taken, i.e. by rapid inhalation through the nasal passage from a container of the powder held close to the nose.

Formulations suitable for nasal administration may, for example, comprise from about as little as 0.1% (w/w) and as much as 100% (w/w) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, 0.1% to 20% (w/w) active ingredient, the balance comprising an orally dissolvable and/or degradable composition and, optionally, one or more of the additional ingredients described herein. Alternately, formulations suitable for buccal administration may comprise a powder and/or an aerosolized and/or atomized solution and/or suspension comprising active ingredient. Such powdered, aerosolized, and/or aerosolized formulations, when dispersed, may have an average particle and/or droplet size in the range from about 0.1 nm to about 200 nm, and may further comprise one or more of any additional ingredients described herein.

A pharmaceutical composition may be prepared, packaged, and/or sold in a formulation suitable for ophthalmic administration. Such formulations may, for example, be in the form of eye drops including, for example, a 0.1/1.0% (w/w) solution and/or suspension of the active ingredient in an aqueous or oily liquid excipient. Such drops may further comprise buffering agents, salts, and/or one or more other of any additional ingredients described herein. Other opthalmically-administrable formulations which are useful include those which comprise the active ingredient in microcrystalline form and/or in a liposomal preparation. Ear drops and/or eye drops are contemplated as being within the scope of this invention.

General considerations in the formulation and/or manufacture of pharmaceutical agents may be found, for example, in Remington: The Science and Practice of Pharmacy 21^{st} ed., Lippincott Williams & Wilkins, 2005 (incorporated herein by reference).

### Administration

The present invention provides methods comprising administering supercharged proteins or complexes in accordance with the invention to a subject in need thereof. Supercharged proteins or complexes, or pharmaceutical, imaging, diagnostic, or prophylactic compositions thereof, may be administered to a subject using any amount and any route of administration effective for preventing, treating, diagnosing, or imaging a disease, disorder, and/or condition (e.g., a disease, disorder, and/or condition relating to working memory deficits). The exact amount required will vary from subject to subject, depending on the species, age, and general condition of the subject, the severity of the disease, the particular composition, its mode of administration, its mode of activity, and the like. Compositions in accordance with the invention are typically formulated in dosage unit form for ease of administration and uniformity of dosage. It will be understood, however, that the total daily usage of the compositions of the present invention will be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

Supercharged proteins or complexes comprising supercharged proteins associated with at least one agent to be delivered and/or pharmaceutical, prophylactic, diagnostic, or imaging compositions thereof may be administered to animals, such as mammals (e.g., humans, domesticated animals, cats, dogs, mice, rats, etc.). In some embodiments, supercharged proteins or complexes and/or pharmaceutical, prophylactic, diagnostic, or imaging compositions thereof are administered to humans.

Supercharged proteins or complexes comprising supercharged proteins associated with at least one agent to be delivered and/or pharmaceutical, prophylactic, diagnostic, or imaging compositions thereof in accordance with the present invention may be administered by any route. In some embodiments, supercharged proteins or complexes, and/or pharmaceutical, prophylactic, diagnostic, or imaging compositions thereof, are administered by one or more of a variety of routes, including oral, intravenous, intramuscular, intra-arterial, intramedullary, intrathecal, subcutaneous, intraventricular, transdermal, interdermal, rectal, intravaginal, intraperitoneal, topical (e.g. by powders, ointments, creams, gels, lotions, and/or drops), mucosal, nasal, buccal, enteral, vitreal, intratumoral, sublingual; by intratracheal instillation, bronchial instillation, and/or inhalation; as an oral spray, nasal spray, and/or aerosol, and/or through a portal vein catheter. In some embodiments, supercharged proteins or complexes, and/or pharmaceutical, prophylactic, diagnostic, or imaging compositions thereof, are administered by systemic intravenous injection. In specific embodiments, supercharged proteins or complexes and/or pharmaceutical, prophylactic, diagnostic, or imaging compositions thereof may be administered intravenously and/or orally. In specific embodiments, supercharged proteins or complexes, and/or pharmaceutical, prophylactic, diagnostic, or imaging compositions thereof, may be administered in a way which allows the supercharged protein or complex to cross the blood-brain barrier, vascular barrier, or other epithelial barrier.

However, the invention encompasses the delivery of supercharged proteins or complexes, and/or pharmaceutical, prophylactic, diagnostic, or imaging compositions thereof, by any appropriate route taking into consideration likely advances in the sciences of drug delivery.

In general the most appropriate route of administration will depend upon a variety of factors including the nature of the supercharged protein or complex comprising supercharged proteins associated with at least one agent to be delivered (e.g., its stability in the environment of the gastrointestinal tract, bloodstream, etc.), the condition of the patient (e.g., whether the patient is able to tolerate particular routes of administration), etc. The invention encompasses the delivery of the pharmaceutical, prophylactic, diagnostic, or imaging compositions by any appropriate route taking into consideration likely advances in the sciences of drug delivery.

In certain embodiments, compositions in accordance with the invention may be administered at dosage levels sufficient to deliver from about 0.0001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, prophylactic, or imaging effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations).

Supercharged proteins or complexes comprising supercharged proteins associated with at least one agent to be delivered may be used in combination with one or more other therapeutic, prophylactic, diagnostic, or imaging agents. By “in combination with,” it is not intended to imply that the agents must be administered at the same time and/or formulated for delivery together, although these methods of delivery are within the scope of the invention. Compositions can be administered concurrently with, prior to, or subsequent to, one or more other desired therapeutics or medical procedures. In general, each agent will be administered at a dose and/or on a time schedule determined for that agent. In some embodiments, the invention encompasses the delivery of pharmaceutical, prophylactic, diagnostic, or imaging compositions in combination with agents that may improve their bioavailability, reduce and/or modify their metabolism, inhibit their excretion, and/or modify their distribution within the body.

In will further be appreciated that therapeutically, prophylactically, diagnostically, or imaging active agents utilized in combination may be administered together in a single composition or administered separately in different compositions. In general, it is expected that agents utilized in combination with be utilized at levels that do not exceed the levels at which they are utilized individually. In some embodiments, the levels utilized in combination will be lower than those utilized individually.

The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a composition useful for treating cancer in accordance with the invention may be administered concurrently with a chemotherapeutic agent), or they may achieve different effects (e.g., control of any adverse effects).

### Kits

The invention provides a variety of kits for conveniently and/or effectively carrying out methods of the present invention. Typically kits will comprise sufficient amounts and/or numbers of components to allow a user to perform multiple treatments of a subject(s) and/or to perform multiple experiments.

In some embodiments, kits comprise one or more of (i) a supercharged protein, as described herein; (ii) an agent to be delivered; (iii) instructions for forming complexes comprising supercharged proteins associated with at least one agent.

In some embodiments, kits comprise one or more of (i) a supercharged protein, as described herein; (ii) a nucleic acid; (iii) instructions for forming complexes comprising supercharged proteins associated with at least one nucleic acid.

In some embodiments, kits comprise one or more of (i) a supercharged protein, as described herein; (ii) a peptide or protein; (iii) instructions for forming complexes comprising supercharged proteins associated with at least one peptide or protein to be delivered.

In some embodiments, kits comprise one or more of (i) a supercharged protein, as described herein; (ii) a small molecule; (iii) instructions for forming complexes comprising supercharged proteins associated with at least one small molecule.

In some embodiments, kits comprise one or more of (i) a supercharged protein or complex comprising supercharged proteins associated with at least one agent to be delivered, as described herein; (ii) at least one pharmaceutically acceptable excipient; (iii) a syringe, needle, applicator, etc. for administration of a pharmaceutical, prophylactic, diagnostic, or imaging composition to a subject; and (iv) instructions for preparing pharmaceutical composition and for administration of the composition to the subject.

In some embodiments, kits comprise one or more of (i) a pharmaceutical composition comprising a supercharged protein or complex comprising supercharged proteins associated with at least one agent to be delivered, as described herein; (ii) a syringe, needle, applicator, etc. for administration of the pharmaceutical, prophylactic, diagnostic, or imaging composition to a subject; and (iii) instructions for administration of the pharmaceutical, prophylactic, diagnostic, or imaging composition to the subject.

In some embodiments, kits comprise one or more components useful for modifying proteins of interest to produce supercharged proteins. These kits typically include all or most of the reagents needed create supercharged proteins. In certain embodiments, such a kit includes computer software to aid a researcher in designing a supercharged protein in accordance with the invention. In certain embodiments, such a kit includes reagents necessary for performing site-directed mutagenesis.

In some embodiments, kits may include additional components or reagents. For example, kits may comprise buffers, reagents, primers, oligonucleotides, nucleotides, enzymes, buffers, cells, media, plates, tubes, instructions, vectors, etc. In some embodiments, kits may comprise instructions for use.

In some embodiments, kits include a number of unit dosages of a pharmaceutical, prophylactic, diagnostic, or imaging composition comprising supercharged proteins or complexes comprising supercharged proteins and at least one agent to be delivered. A memory aid may be provided, for example in the form of numbers, letters, and/or other markings and/or with a calendar insert, designating the days/times in the treatment schedule in which dosages can be administered. Placebo dosages, and/or calcium dietary supplements, either in a form similar to or distinct from the dosages of the pharmaceutical, prophylactic, diagnostic, or imaging compositions, may be included to provide a kit in which a dosage is taken every day.

Kits may comprise one or more vessels or containers so that certain of the individual components or reagents may be separately housed. Kits may comprise a means for enclosing individual containers in relatively close confinement for commercial sale (e.g., a plastic box in which instructions, packaging materials such as styrofoam, etc., may be enclosed). Kit contents are typically packaged for convenience use in a laboratory.

These and other aspects of the present invention will be further appreciated upon consideration of the following Examples, which are intended to illustrate certain particular embodiments of the invention but are not intended to limit its scope, as defined by the claims.

### EXAMPLES

### Example 1

### Supercharging Proteins can Impart Extraordinary Resilience

### Materials and Methods

### Design Procedure and Supercharged Protein Sequences

Solvent-exposed residues (shown in grey below) were identified from published structural data (Weber et al., 1989, Science, 243:85; Dirr et al., 1994, J. Mol. Biol., 243:72; Pedelacq et al., 2006, Nat. Biotechnol., 24:79; each of which is incorporated herein by reference) as those having AvNAPSA <150, where AvNAPSA is average neighbor atoms (within 10 Å) per sidechain atom. Charged or highly polar solvent-exposed residues (DERKNQ) were mutated either to Asp or Glu, for negative-supercharging; or to Lys or Arg, for positive-supercharging. Additional surface-exposed positions to mutate in green fluorescent protein (GFP) variants were chosen on the basis of sequence variability at these positions among GFP homologues.

### Protein Expression and Purification

Synthetic genes optimized for E. coli codon usage were purchased from DNA 2.0, cloned into a pET expression vector (Novagen), and overexpressed in E. coli BL21(DE3) pLysS for 5-10 hours at 15° C. Cells were harvested by centrifugation and lysed by sonication. Proteins were purified by Ni-NTA agarose chromotography (Qiagen), buffer-exchanged into 100 mM NaCl, 50 mM potassium phosphate pH 7.5, and concentrated by ultrafiltration (Millipore). All GFP variants were purified under native conditions.

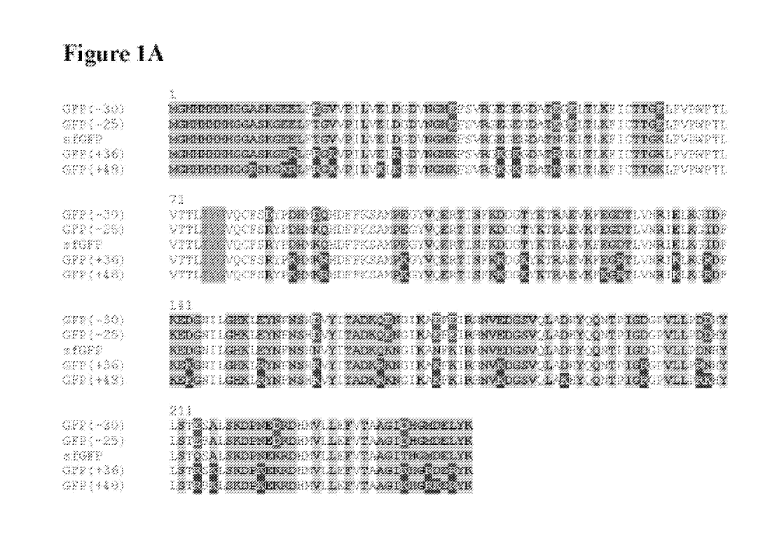

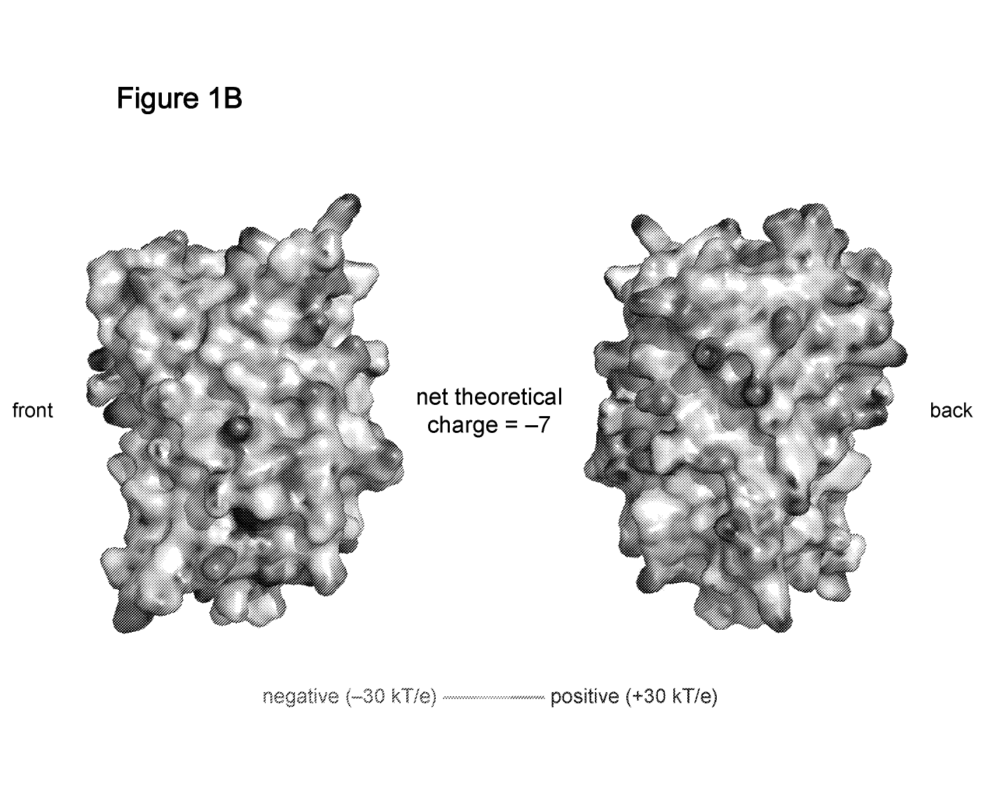

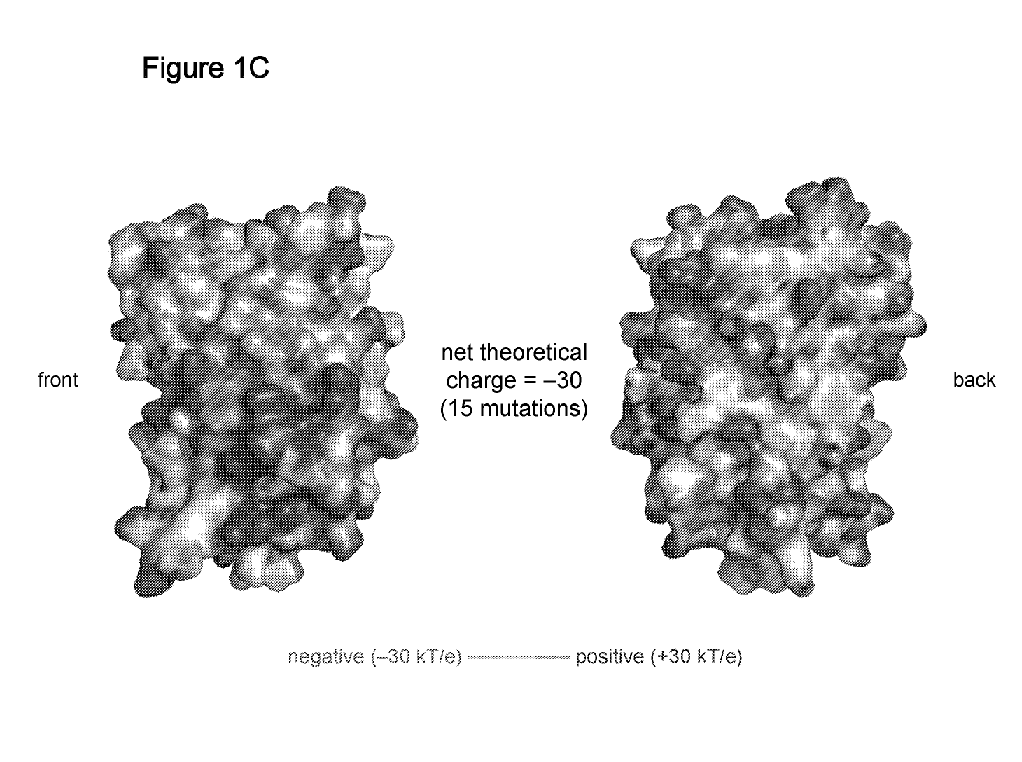

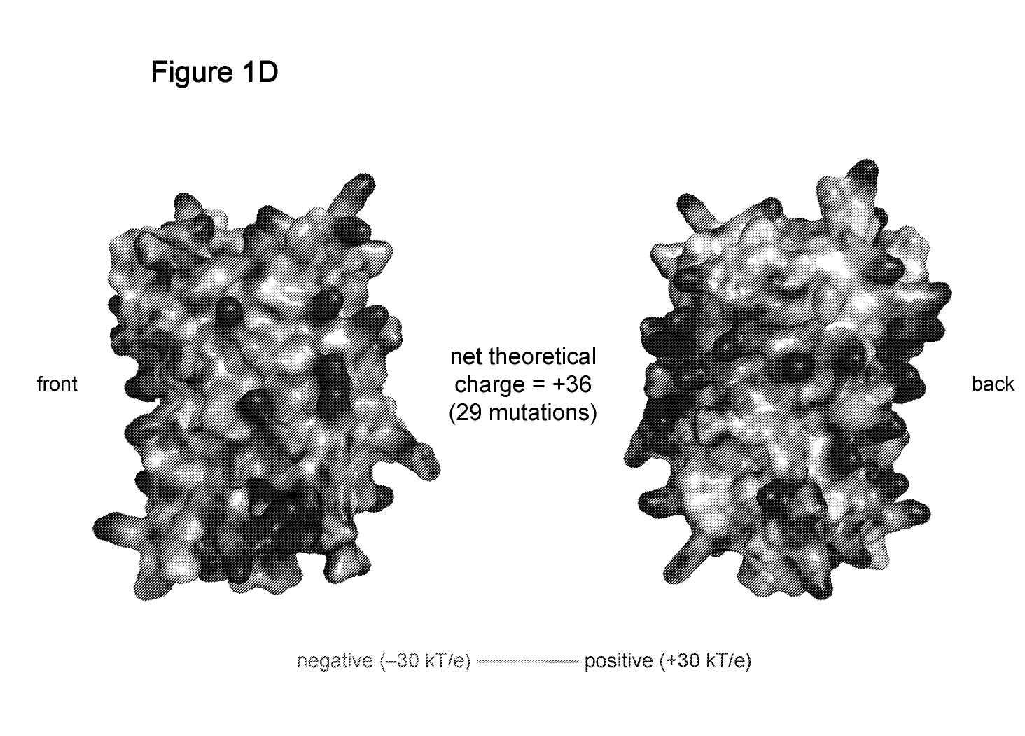

### Electrostatic Surface Potential Calculations (FIG. 1B-D)

Models of −30 and +48 supercharged GFP variants were based on the crystal structure of superfolder GFP (Pedelacq et al., 2006, Nat. Biotechnol., 24:79; incorporated herein by reference). Electrostatic potentials were calculated using APBS (Baker et al., 2001, Proc. Natl. Acad. Sci., USA, 98:10037; incorporated herein by reference) and rendered with PyMol (Delano, 2002, The PyMOL Molecular Graphics System, www.pymol.org; incorporated herein by reference) using a scale of −25 kT/e (red) to +25 kT/e (blue).

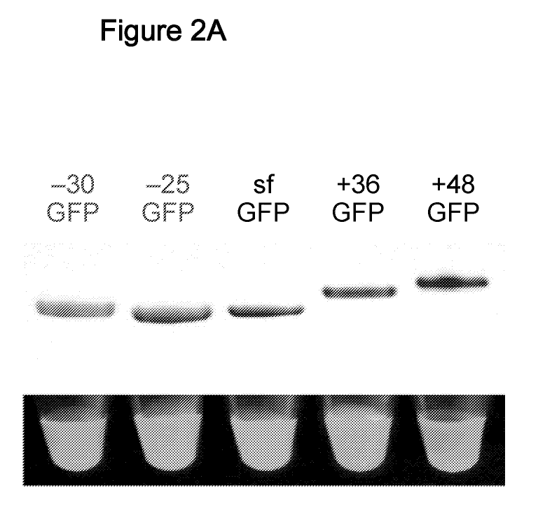

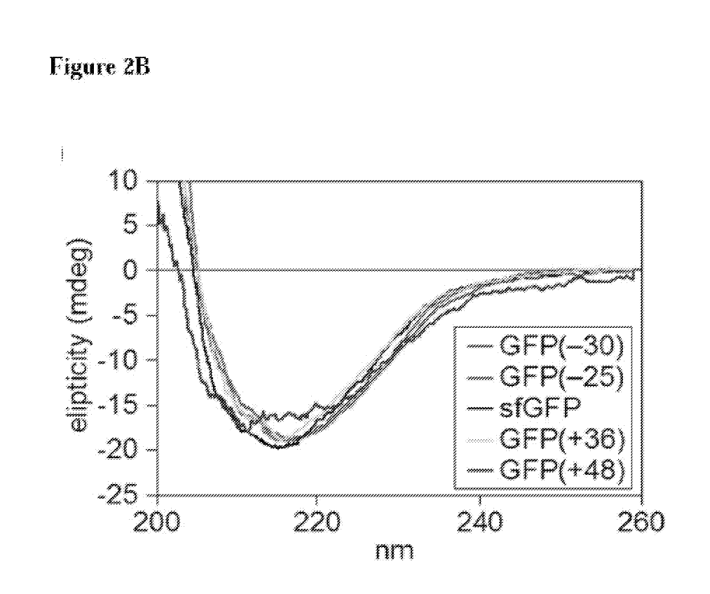

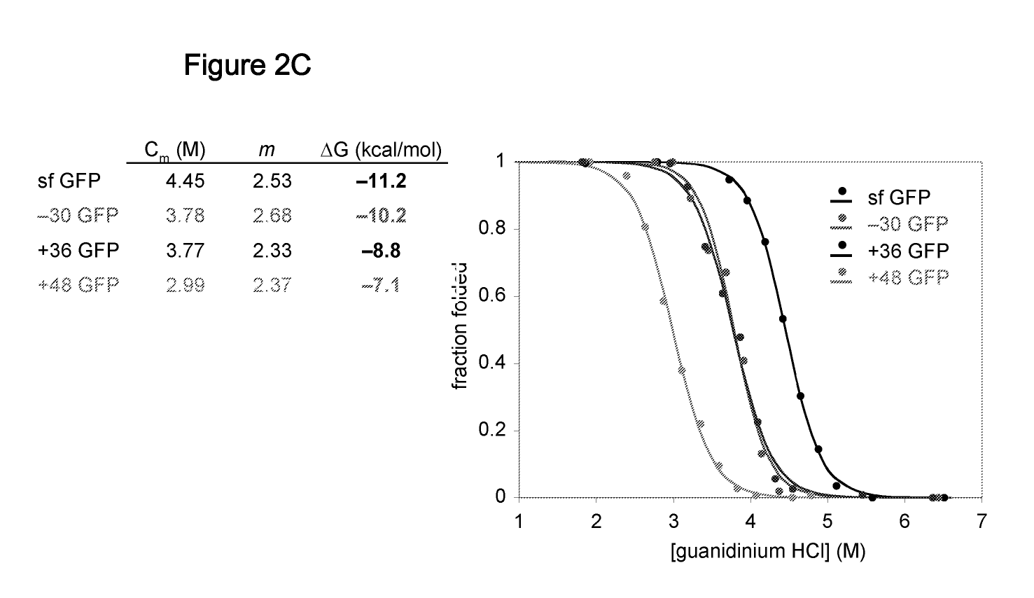

### Protein Staining and UV-Induced Fluorescence (FIG. 2A)

0.2 μg of each GFP variant was analyzed by electrophoresis in a 10% denaturing polyacrylamide gel and stained with Coomassie brilliant blue dye. 0.2 μg of the same protein samples in 25 mM Tris pH 8.0 with 100 mM NaCl was placed in a 0.2 mL Eppendorf tube and photographed under UV light (360 nm).

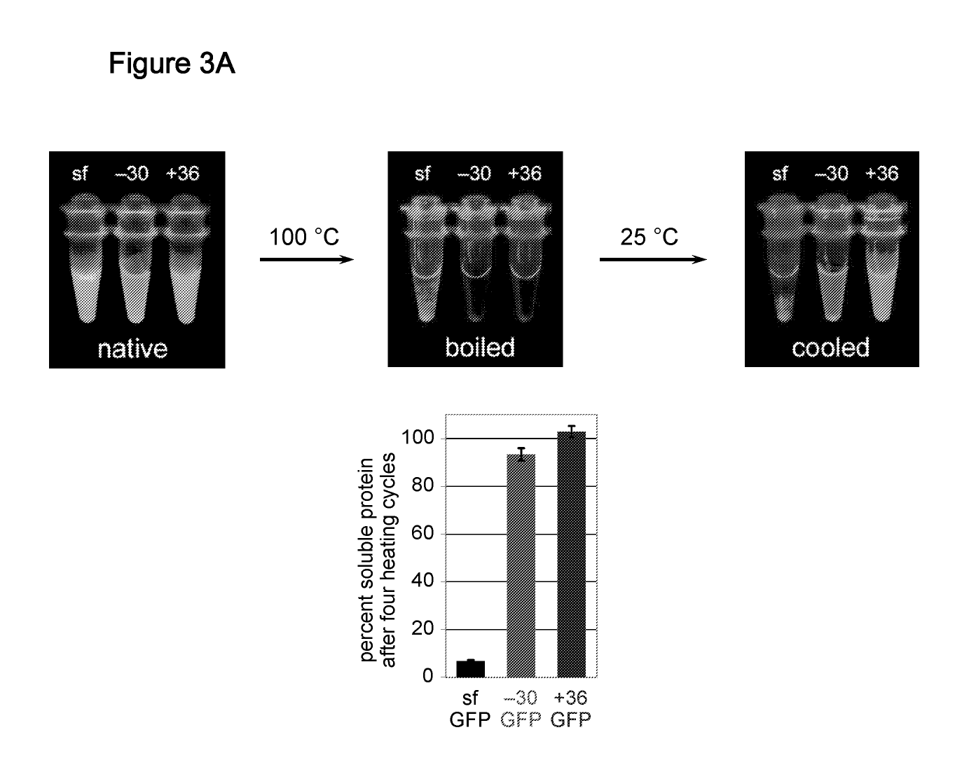

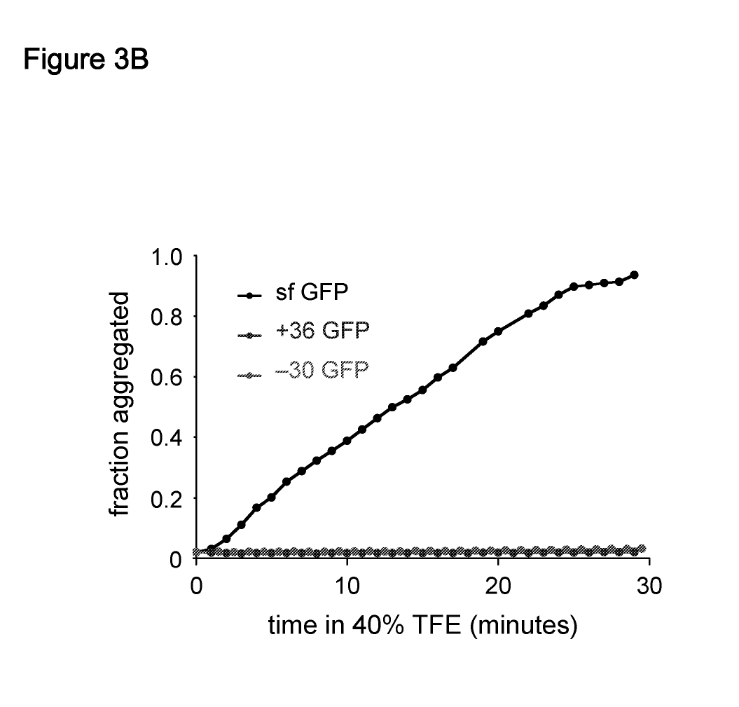

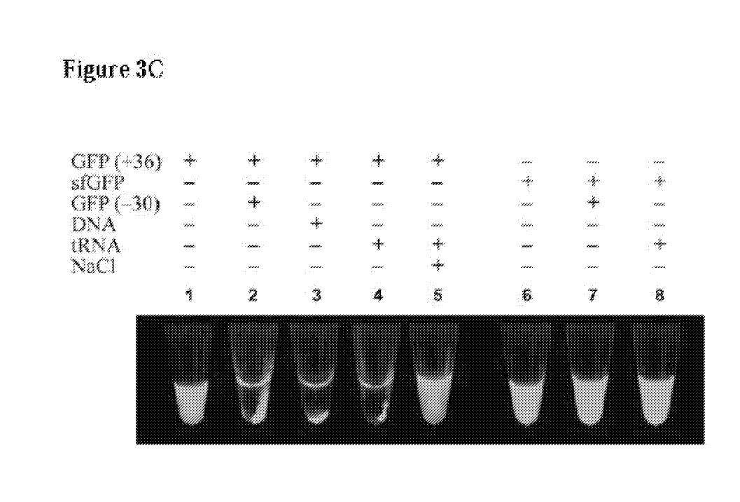

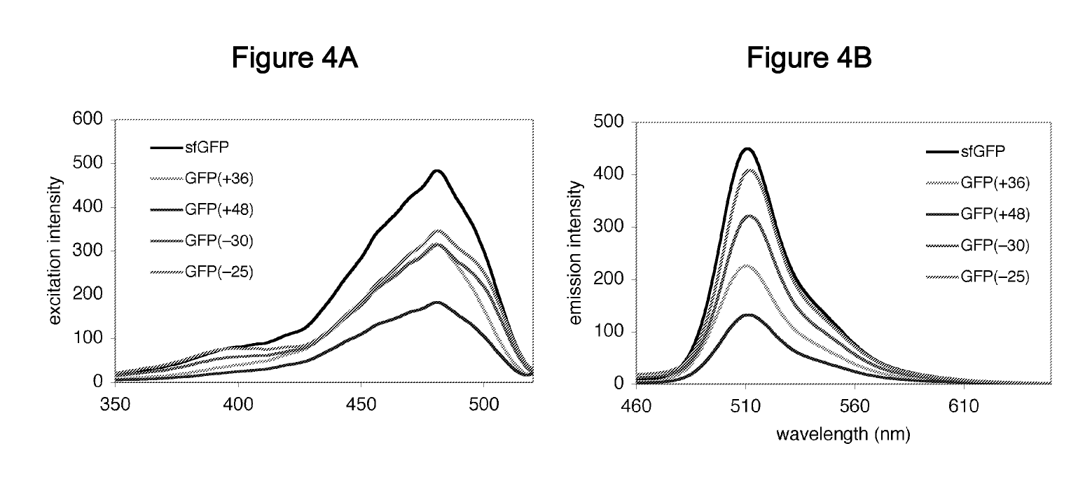

Thermal Denaturation and Aggregation (FIG. 3A)

Purified GFP variants were diluted to 2 mg/mL in 25 mM Tris pH 8.0, 100 mM NaCl, and 10 mM beta-mercaptoethanol (BME), then photographed under UV illumination (“native”). The samples were heated to 100° C. for 1 minute, then photographed again under UV illumination (“boiled”). Finally, the samples were cooled 2 hours at room temperature and photographed again under UV illumination (“cooled”).

### Chemically Induced Aggregation (FIG. 3B)

2,2,2-trifluoroethanol (TFE) was added to produce solutions with 1.5 mg/mL protein, 25 mM Tris pH 7.0, 10 mM BME, and 40% TFE. Aggregation at 25° C. was monitored by right-angle light scattering.

### Size-Exclusion Chromotography (Table 4)

The multimeric state of GFP variants was determined by analyzing 20-50 μg of protein on a Superdex 75 gel-filtration column. Buffer was 100 mM NaCl, 50 mM potassium phosphate pH 7.5. Molecular weights were determined by comparison with a set of monomeric protein standards of known molecular weights analyzed separately under identical conditions.

TABLE 4
Calculated and experimentally determined protein properties.

| | MW | length | | | | | | ΔG | native | % soluble |
|---|---|---|---|---|---|---|---|---|---|---|
| name | (kD) | (aa) | nₚₒₛ | n_{neg} | n_{charged} | Qₙₑₜ | pI | (kcal/mol)^{a} | MW (kD)^{b} | after boiling^{c} |
| GFP (−30) | 27.8 | 248 | 19 | 49 | 68 | −30 | 4.8 | 10.2 | n.d. | 98 |
| GFP (−25) | 27.8 | 248 | 21 | 46 | 67 | −25 | 5.0 | n.d. | n.d. | n.d. |
| sfGFP | 27.8 | 248 | 27 | 34 | 61 | −7 | 6.6 | 11.2 | n.d. | 4 |
| GFP (+36) | 28.5 | 248 | 56 | 20 | 76 | +36 | 10.4 | 8.8 | n.d. | 97 |
| GFP (+48) | 28.6 | 248 | 63 | 15 | 78 | +48 | 10.8 | 7.1 | n.d. | n.d. |

nₚₒₛ, number of positively charged amino acids (per monomer)
n_{neg}, number of negatively charged amino acids
n_{charged}, total number of charged amino acids
Qₙₑₜ, theoretical net charge at neutral pH
pI, calculated isoelectric point
n.d., not determined
^{a}measured by guanidinium denaturation (FIG. 2C).
^{b}measured by size-exclusion chromatography.
^{c}percent protein remaining in supernatant after 5 min at 100° C., cooling to 25° C., and brief centrifugation.

### Supercharged GFP

A variant of green fluorescent protein (GFP) called “superfolder GFP” (sfGFP) has been highly optimized for folding efficiency and resistance to denaturants (Pedelacq et al., 2006, Nat. Biotechnol., 24:79; incorporated herein by reference). Superfolder GFP has a net charge of −7, similar to that of wild-type GFP. Guided by a simple algorithm to calculate solvent exposure of amino acids (see Materials and Methods), a supercharged variant of GFP was designed. Supercharged GFP has a theoretical net charge of +36 and was created by mutating 29 of its most solvent-exposed residues to positively charged amino acids (FIG. 1). The expression of genes encoding either sfGFP or supercharged GFP (“GFP(+36)”) yielded intensely green-fluorescent bacteria. Following protein purification, the fluorescence properties of GFP(+36) were measured and found to be very similar to those of sfGFP.

Additional supercharged GFPs having net charges of +48, −25, and −30 were designed and purified, all of which were also found to exhibit sfGFP-like fluorescence (FIG. 2A). All supercharged GFP variants showed circular dichroism spectra similar to that of sfGFP, indicating that the proteins have similar secondary structure content (FIG. 2B). The thermodynamic stabilities of the supercharged GFP variants were only modestly lower than that of sfGFP (1.0-4.1 kcal/mol, FIG. 2C and Table 4) despite the presence of as many as 36 mutations.

Although sfGFP is the product of a long history of GFP optimization (Giepmans et al., 2006, Science, 312:217; incorporated herein by reference), it remains susceptible to aggregation induced by thermal or chemical unfolding. Heating sfGFP to 100° C. induced its quantitative precipitation and the irreversible loss of fluorescence (FIG. 3A). In contrast, supercharged GFP(+36) and GFP(−30) remained soluble when heated to 100° C., and recovered significant fluorescence upon cooling (FIG. 3A). While 40% 2,2,2-trifluoroethanol (TFE) induced the complete aggregation of sfGFP at 25° C. within minutes, the +36 and −30 supercharged GFP variants suffered no significant aggregation or loss of fluorescence under the same conditions for hours (FIG. 3B).

Supercharged GFP variants show a strong, reversible avidity for highly charged macromolecules of the opposite charge (FIG. 3C). When mixed together in 1:1 stoichiometry, GFP(+36) and GFP(−30) immediately formed a green fluorescent co-precipitate, indicating the association of folded proteins. GFP(+36) similarly co-precipitated with high concentrations of RNA or DNA. Addition of NaCl was sufficient to dissolve these complexes, consistent with the electrostatic basis of their formation. In contrast, sfGFP was unaffected by the addition of GFP(−30), RNA, or DNA (FIG. 3C).

### CONCLUSION

In summary, monomeric and multimeric proteins of varying structures and functions can be “supercharged” by simply replacing their most solvent-exposed residues with like-charged amino acids. Supercharging profoundly alters the intermolecular properties of proteins, imparting remarkable aggregation resistance and the ability to associate in folded form with oppositely charged macromolecules like “molecular Velcro.”

In contrast to these dramatic intermolecular effects, the intramolecular properties of the seven supercharged proteins studied here, including folding, fluorescence, ligand binding, and enzymatic catalysis, remained largely intact. Supercharging therefore may represent a useful approach for reducing the aggregation tendency and improving the solubility of proteins without abolishing their function. These principles may be particularly useful in de novo protein design efforts, where unpredictable protein handling properties including aggregation remain a significant challenge.

These observations may also illuminate the modest net-charge distribution of natural proteins (Knight et al., 2004, Proc. Natl. Acad. Sci., USA, 101:8390; Gitlin et al., 2006, Angew Chem Int Ed Engl, 45:3022; each of which is incorporated herein by reference): the net charge of 84% of Protein Data Bank (PDB) polypeptides, for example, falls within ±10. The results above argue against the hypothesis that high net charge creates sufficient electrostatic repulsion to force unfolding. Indeed, GFP(+48) has a higher positive net charge than any polypeptide currently in the PDB, yet retains the ability to fold and fluoresce. Instead, these findings suggest that nonspecific intermolecular adhesions may have disfavored the evolution of too many highly charged natural proteins. Almost all natural proteins with very high net charge, such as ribosomal proteins L3 (+36) and L15 (+44), which bind RNA, or calsequestrin (−80), which binds calcium cations, associate with oppositely charged species as part of their essential cellular functions.

### Example 2

### Supercharged Proteins can be Used to Efficiently Deliver Nucleic Acids to Cells

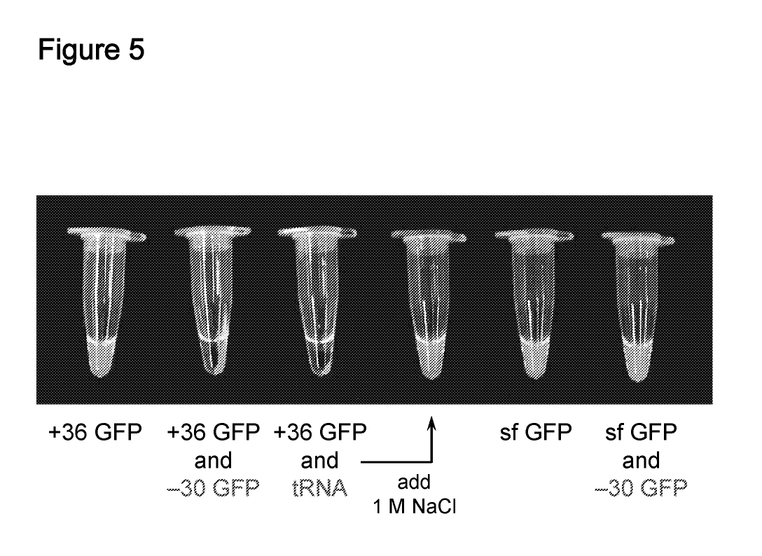

FIG. 5 demonstrates that supercharged GFPs associate non-specifically and reversibly with oppositely charged macromolecules (“protein Velcro”). Such interactions can result in the formation of precipitates. Unlike aggregates of denatured proteins, these precipitates contain folded, fluorescent GFP and dissolve in 1 M salt. Shown here are: +36 GFP alone; +36 GFP mixed with −30 GFP; +36 GFP mixed with tRNA; +36 GFP mixed with tRNA in 1 M NaCl; superfolder GFP (“sf GFP”; −7 GFP); and sfGFP mixed with −30 GFP.

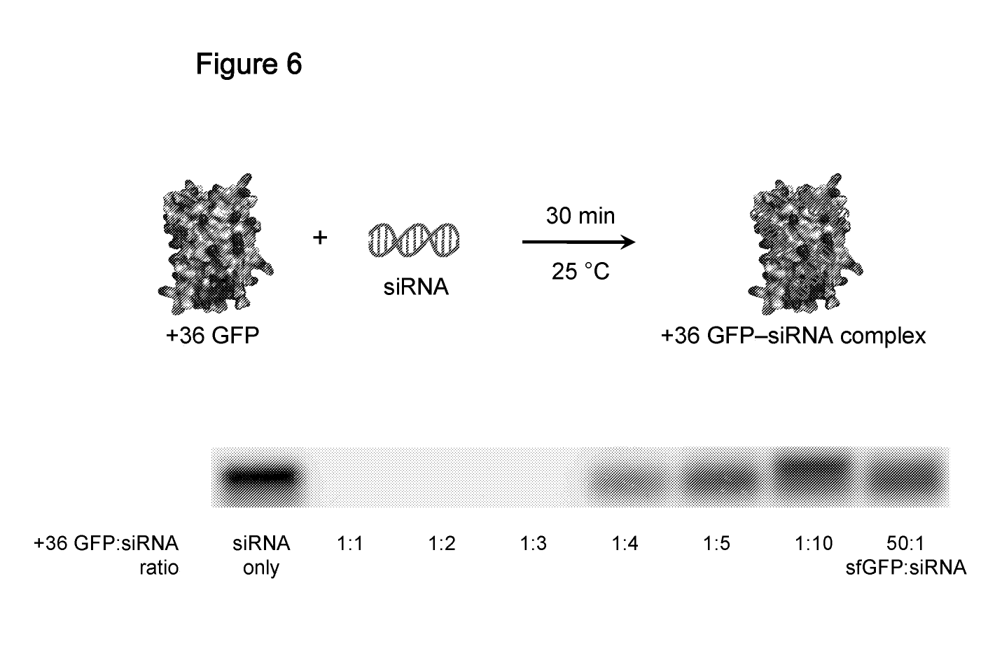

FIG. 6 demonstrates that superpositively charged GFP binds siRNA. The binding stoichiometry between +36 GFP and siRNA was determined by mixing various ratios of the two components (30 minutes at 25° C.) and running the mixture on a 3% agarose gel (Kumar et al., 2007, Nature, 449:39; incorporated herein by reference). Ratios of +36 GFP:siRNA tested were 0:1, 1:1, 1:2, 1:3, 1:4, 1:5, and 1:10. +36 GFP/siRNA complexes did not co-migrate with siRNA in an agarose gel. +36 GFP was shown to form a stable complex with siRNA in a ˜1:3 stoichiometry, indicating that one supercharged GFP binds approximately three siRNA molecules. This property allows the application of low quantities of superpositively charged GFP to deliver siRNA effectively to cells. Moreover, because the delivery reagent is fluorescent, and therefore observable by fluorescence microscopy, siRNA delivery can be assessed using this spectroscopic technique. In contrast, non-superpositive proteins did not bind siRNA. A 50:1 ratio of sfGFP:siRNA was also tested, but, even at such high levels of excess, sfGFP did not associate with siRNA.

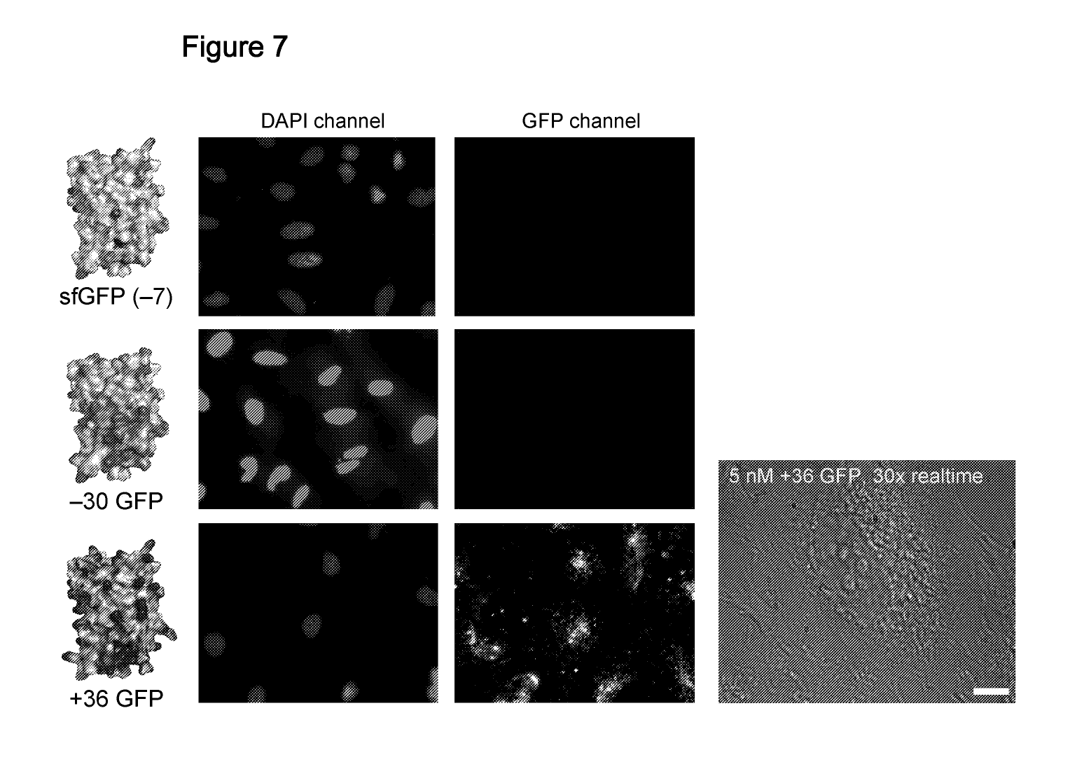

FIG. 7 demonstrates that superpositively charged GFP penetrates cells. HeLa cells were incubated with 1 nM GFP for 3 hours, washed, fixed, and stained. Three GFP variants were tested in this experiment: sf GFP (−7), −30 GFP, and +36 GFP. +36 GFP, but not sfGFP or −30 GFP, was shown to potently penetrate HeLa cells within minutes. Localization was shown to begin at the cell membrane, becoming punctate and intracellular thereafter. +36 GFP was shown to be stable in HeLa cells for ≧5 days. Results are shown in FIG. 7. On the left is DAPI staining of DNA to mark the position of cells. In the middle is GFP staining to show where cellular uptake of GFP occurred. On the right is a movie showing localization as it occurs.

In order to demonstrate the utility of superpositively charged GFP for siRNA delivery, we compared siRNA transfection efficiency using Lipofectamine 2000™ (Invitrogen), a commonly used and commercially available cationic lipid transfection reagent, to superpositively charged GFP-based siRNA transfection in HeLa cells.

Generally, for a cell culture condition with a total volume of 1 mL, cells are plated to ˜80% confluency in 10% serum/media. The serum/media solution is removed, and cells are washed twice with PBS and 500 μL of serum-free media. In a separate vessel, 500 μL of serum free media is added, to which 1 μL of 50 μM siRNA solution (total concentration 100 nM) and 1.66 μL of 15 μM sc(+36)GFP (total concentration 40 nM) are added. The contents are mixed by inversion and allowed to incubate for 5 minutes. After such time, the mixture is added to the well containing 500 μL of serum-free media to give a final concentration of 50 nM siRNA and 20 nM scGFP. This solution is placed in a 37° C. incubator (5% CO₂) for 4 hours, removed, and washed twice with PBS. Cells are then treated with 1 mL 10% FBS/media. Cells were allowed to incubate for 4 days before being harvested to determine gene knockdown.

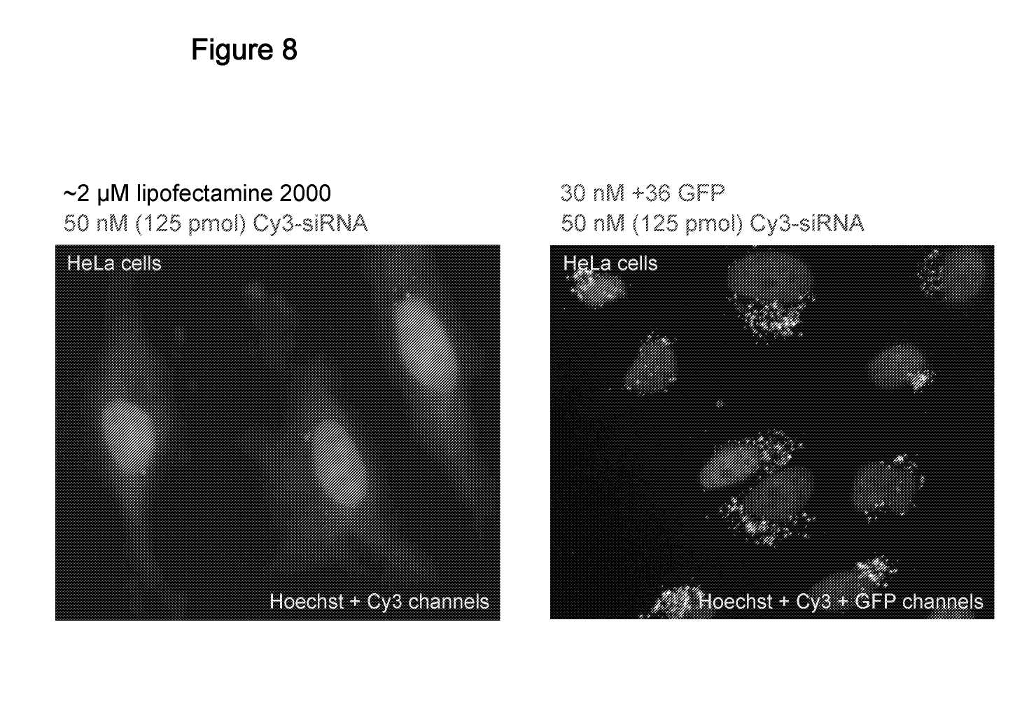

FIG. 8 demonstrates that superpositively charged GFP is able to deliver siRNA into human cells. In particular, +36 GFP was shown to deliver siRNA into HeLa cells. +36 GFP delivered higher quantities of siRNA at a much higher transfection efficiency than Lipofectamine. HeLa cells were treated with either: ˜2 μM lipofectamine 2000 and 50 nM (125 pmol) Cy3-siRNA (left); or 30 nM of +36 GFP and 50 nM (125 pmol) Cy3-siRNA (right). Unlike Lipofectamine, +36 GFP did not induce cytotoxicity, particularly upon addition of antibiotics such as penicillin and streptomycin.

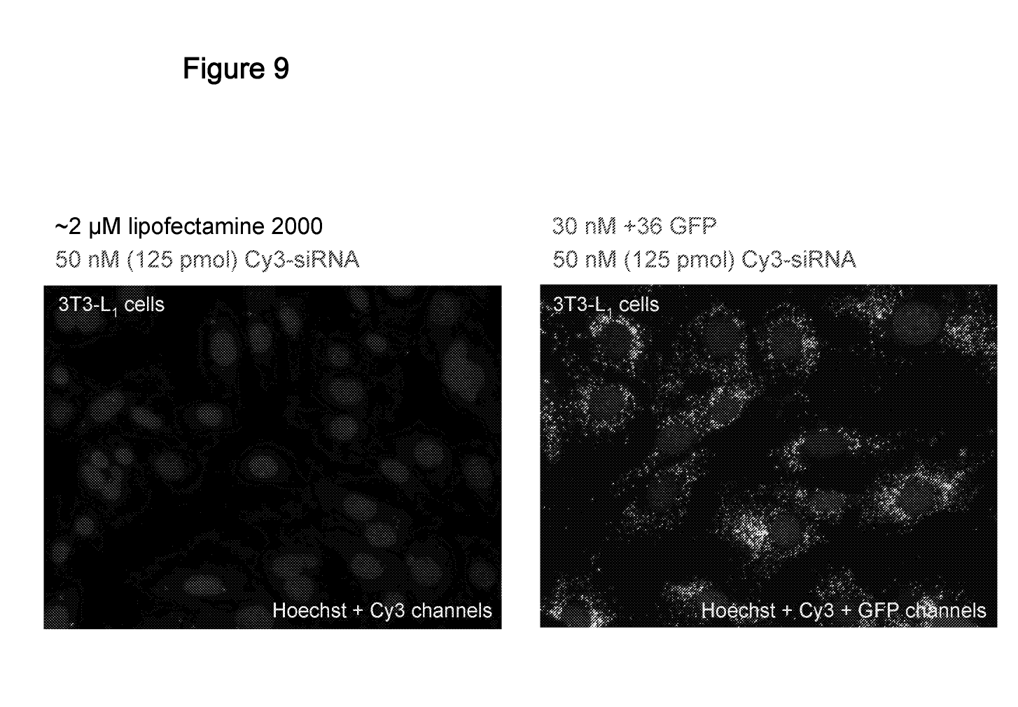

In order to demonstrate the broad utility of supercharged proteins for nucleic acid delivery, this experiment has been repeated in a variety of cells, including cells that are resistant to cationic lipid-based siRNA transfection. FIGS. 9-11 demonstrate that superpositively charged GFP is able to deliver siRNA into cell lines that are resistant to traditional transfection methods. FIG. 9 demonstrates that superpositively charged GFP is able to deliver siRNA into 3T3-L₁ pre-adipocyte cells (“3T3L cells”). 3T3L cells were treated with either: ˜2 μM Lipofectamine 2000 and 50 nM (125 pmol) Cy3-siRNA (left); or 30 nM +36 GFP and 50 nM (125 pmol) Cy3-siRNA (right). Murine 3T3-L₁ pre-adipocyte cells were poorly transfected by Lipofectamine but were efficiently transfected by +36 GFP. Hoescht channel, blue, was used to visualize DNA, thereby marking the position of cells; Cy3 channel, red, was used to visualize Cy3-tagged siRNA; GFP channel, green, was used to visualize GFP. Yellow indicates sites of co-localization between siRNA and GFP. Unlike Lipofectamine, +36 GFP did not induce cytotoxicity, particularly upon addition of antibiotics such as penicillin and streptomycin.

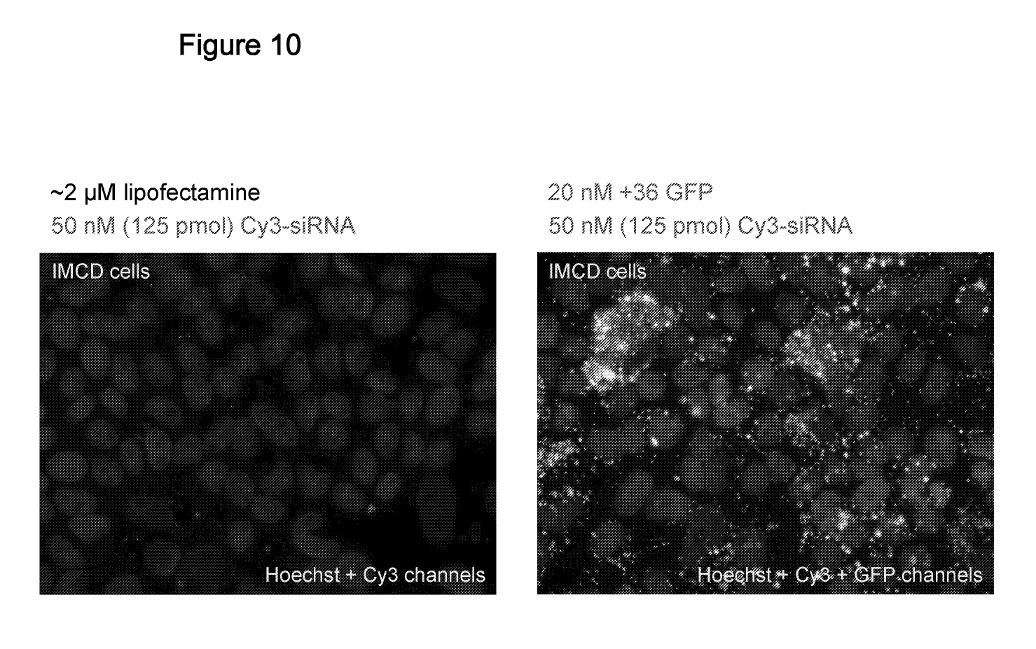

FIG. 10 demonstrates that superpositively charged GFP is able to deliver siRNA into rat IMCD cells. Rat IMCD cells were treated with either ˜2 μM Lipofectamine 2000 and 50 nM (125 pmol) Cy3-siRNA (left); or 20 nM +36 GFP and 50 nM (125 pmol) Cy3-siRNA (right). Rat IMCD cells were poorly transfected by Lipofectamine but were efficiently transfected with +36 GFP. Hoescht channel, blue, was used to visualize DNA, thereby marking the position of cells; Cy3 channel, red, was used to visualize Cy3-tagged siRNA; GFP channel, green, was used to visualize GFP. Yellow indicates sites of co-localization between siRNA and GFP. Unlike Lipofectamine, +36 GFP did not induce cytotoxicity, particularly upon addition of antibiotics such as penicillin and streptomycin.

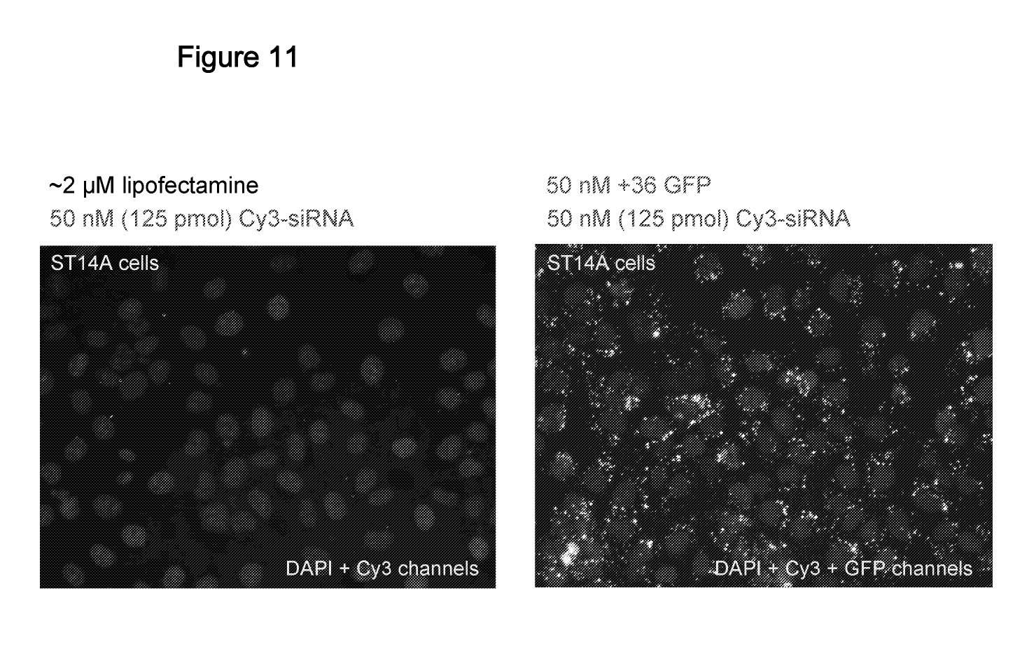

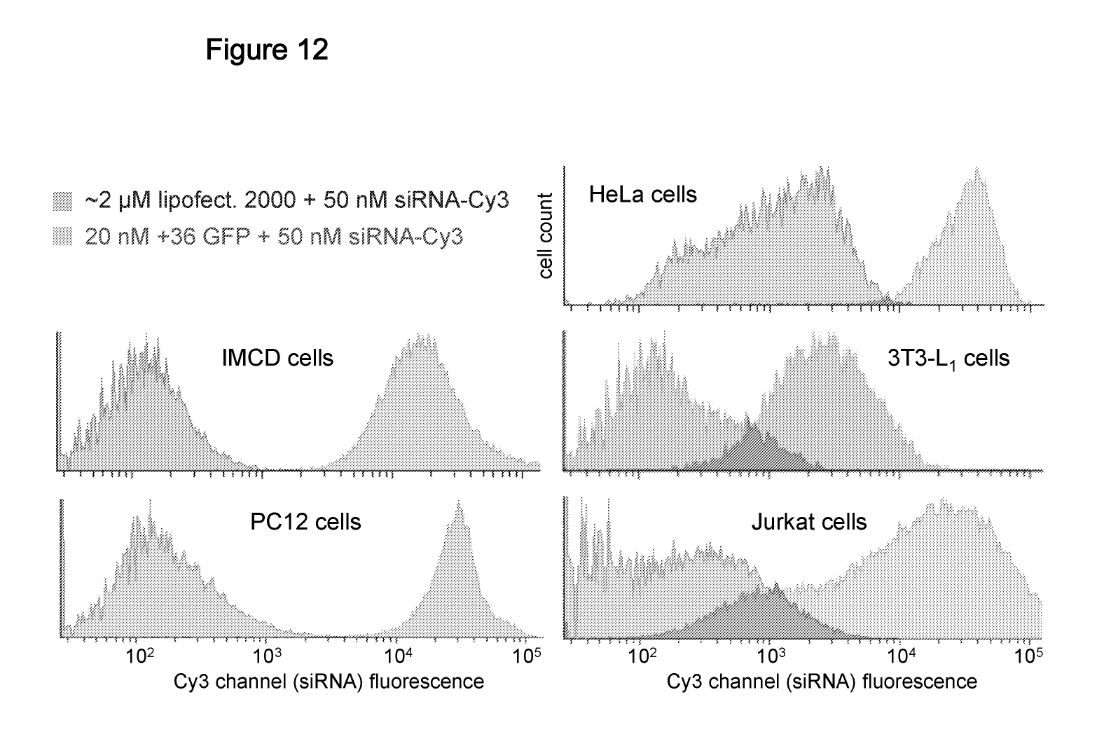

FIG. 11 demonstrates that superpositively charged GFP is able to deliver siRNA into human ST14A neurons. Human ST14A neurons were treated with either ˜2 μM Lipofectamine 2000 and 50 nM (125 pmol) Cy3-siRNA; or 50 nM +36 GFP and 50 nM (125 pmol) Cy3-siRNA. Human ST14A neurons were weakly transfected by Lipofectamine but were efficiently transfected by +36 GFP. DAPI channel, blue, was used to visualize DNA, thereby marking the position of cells; Cy3 channel, red, was used to visualize Cy3-tagged siRNA; GFP channel, green, was used to visualize GFP. Yellow indicates sites of co-localization between siRNA and GFP. Results similar to those presented in FIGS. 9-11 were observed in two other cell types that are resistant to traditional transfection methods (i.e., Jurkat cells and PC12 cells). Unlike Lipofectamine, +36 GFP did not induce cytotoxicity, particularly upon addition of antibiotics such as penicillin and streptomycin.

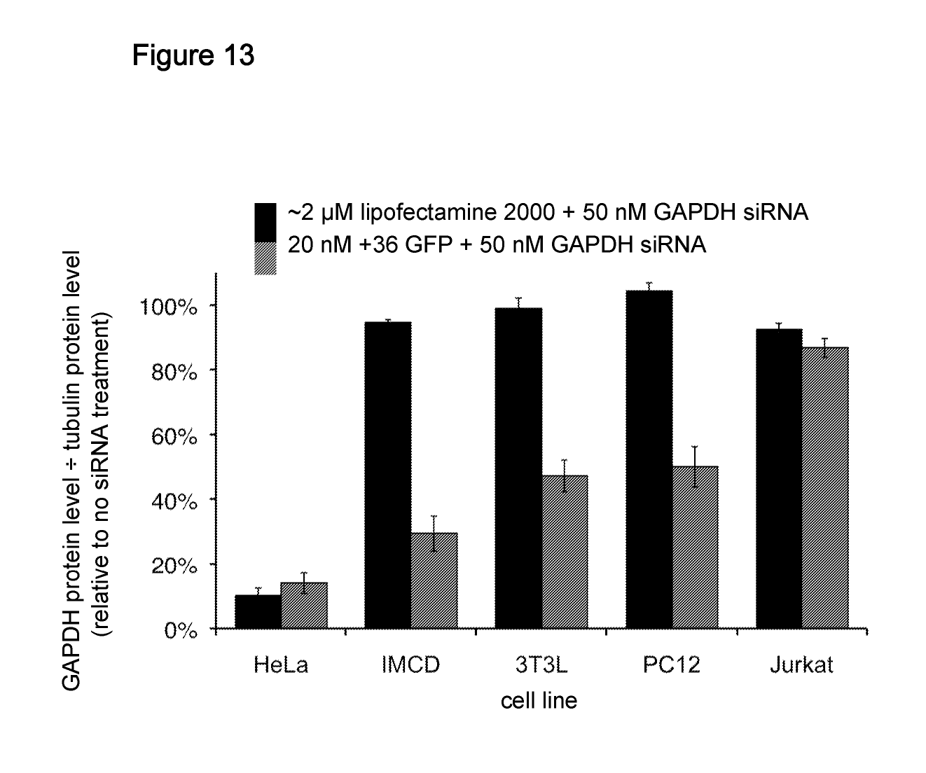

FIG. 13 presents flow cytometry analysis of siRNA transfection experiments. Each column corresponds to experiments performed with different transfection methods: Lipofectamine (blue); and 20 nM +36 GFP (red). Each chart corresponds to experiments performed with different cell types: IMCD cells, PC12 cells, HeLa cells, 3T3L cells, and Jurkat cells. The X-axis represents measurements obtained from the Cy3 channel, which is a readout of siRNA fluorescence. The Y-axis represents cell count in flow cytometry experiments. Flow cytometry data indicate that cells were more efficiently transfected with siRNA using +36 GFP than Lipofectamine.

In order to demonstrate the effectiveness of +36 GFP-delivered siRNA to suppress gene expression, cellular levels of GAPDH were examined by western blot. As shown in FIG. 13, +36 GFP effectively delivered siRNA to cells and suppressed GAPDH at levels comparable to that of lipofectamine. 50 nM GAPDH siRNA was transfected into five different cell types (HeLa, IMCD, 3T3L, PC12, and Jurkat cell lines) using either ˜2 μM lipofectamine 2000 (black bars) or 20 nM +36 GFP (green bars). The Y-axis represents GAPDH protein levels as a fraction of tubulin protein levels.

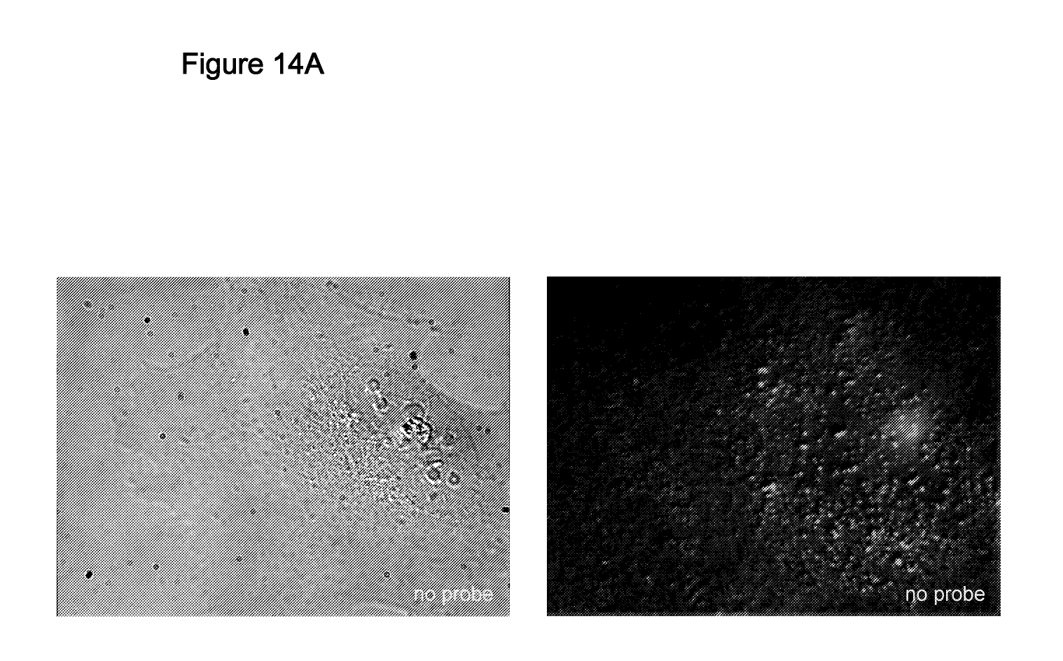

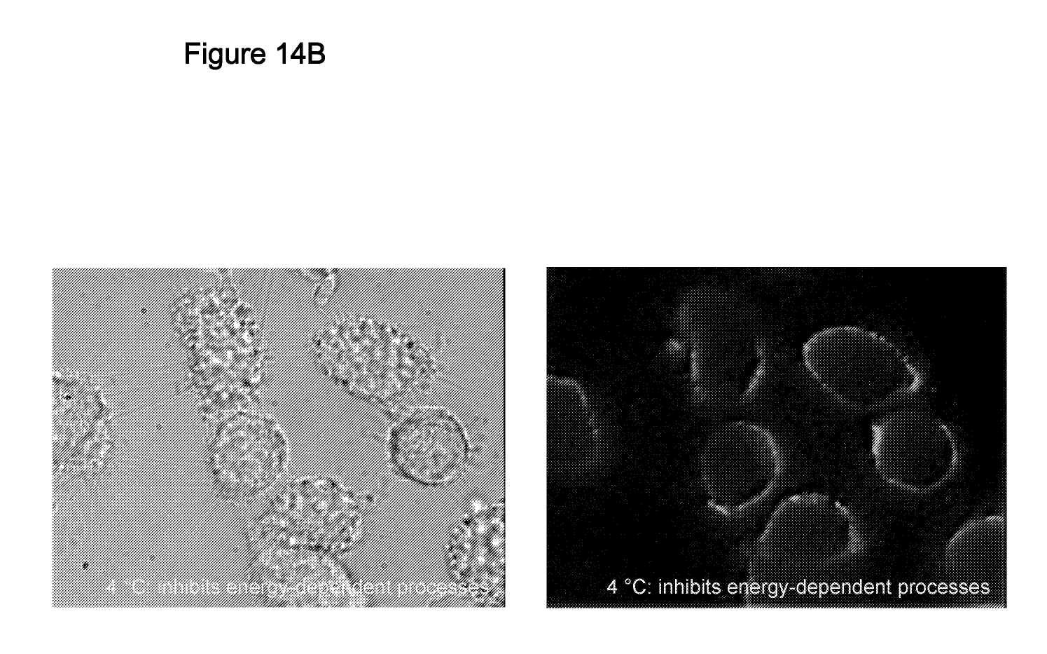

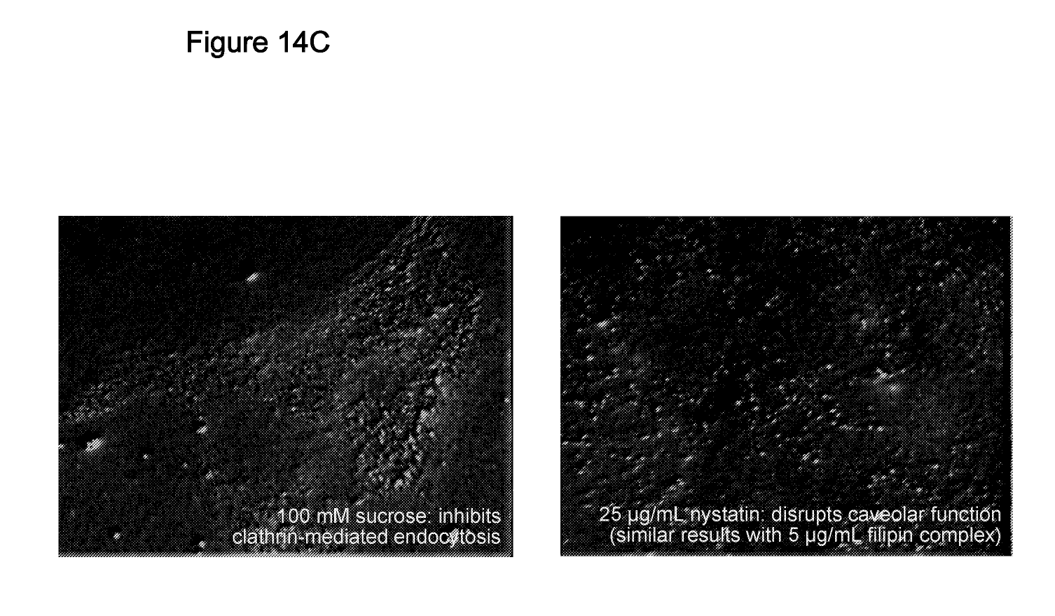

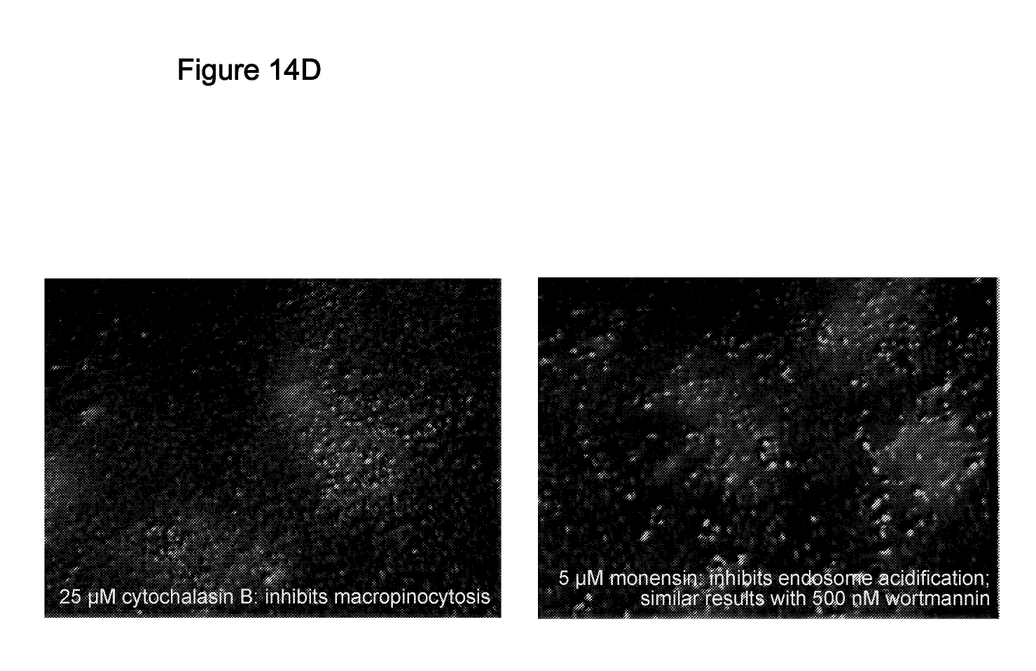

FIG. 14 demonstrates the effects of a variety of mechanistic probes of cell penetration on superpositively charged GFP-mediated siRNA transfection. HeLa cells were treated with one of a variety of probes for 30 minutes and were then treated with 5 nM +36 GFP. Cells were then washed with heparin+probe and imaged in PBS+probe. Samples included: no probe; 4° C. preincubation (inhibits energy-dependent processes); 100 mM sucrose (inhibits clathrin-mediated endocytosis); 25 μg/ml nystatin (disrupts caveolar function); 25 μM cytochalisin B (inhibits macropinocytosis); and 5 μM monensin (inhibits endosome receptor recycling). Experiments at 4° C. demonstrated that cell penetration of +36 GFP involves energy consumption. Experiments with sucrose and nystatin demonstrate that cellular uptake of +36 GFP does not involve clathrin-mediated endocytosis or caveolar endocytosis. Experiments with cytochalasin B and monensin demonstrate that cellular uptake of +36 GFP does not involve macropinocytosis, but is likely to involve early endosomes.

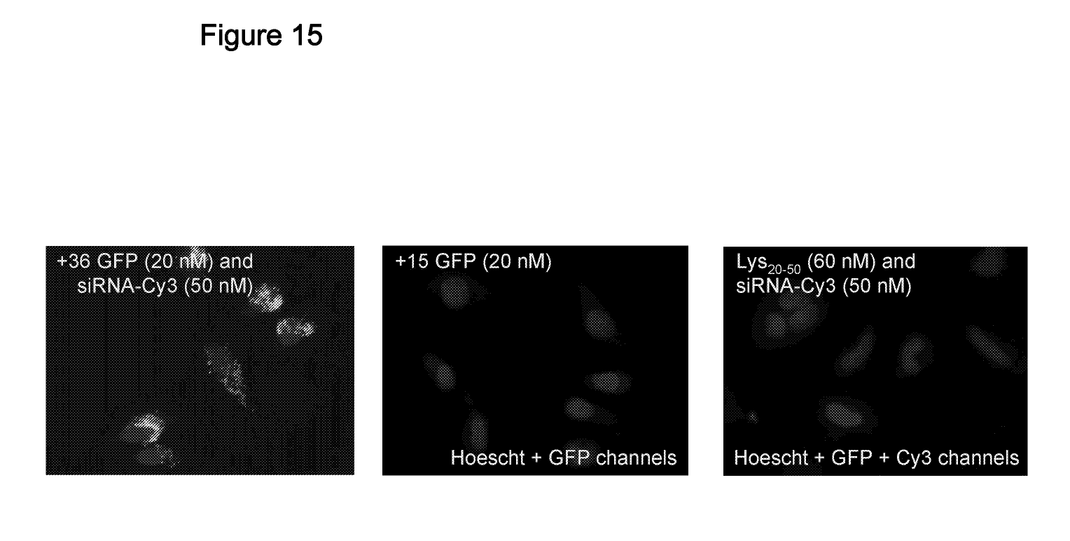

FIG. 15 demonstrates various factors contributing to cell-penetrating activity. Charge density was shown to contribute to cell-penetrating activity. For example, 60 nM Arg₆ was shown not to transfect siRNA. Charge magnitude was shown to contribute to cell-penetrating activity. For example, +15 GFP was shown not to penetrate cells or transfect siRNA. “Protein-like” character was also shown to contribute to cell-penetrating activity. For example, 60 nM Lys₂₀₋₅₀ was shown not to transfect siRNA. The present invention demonstrates that, in some embodiments, charge density is not sufficient to allow a protein to penetrate into cells. The present invention demonstrates that, in some situations, charge magnitude may necessary but not sufficient to allow a protein to penetrate into cells. The present invention further shows that some protein-like features may contribute to cell penetration.

### Example 3

### Mammalian Cell Penetration, siRNA Transfection, and DNA Transfection by Supercharged Green Fluorescent Proteins

We recently described resurfacing proteins without abolishing their structure or function through the extensive mutagenesis of non-conserved, solvent-exposed residues (Lawrence M S, Phillips K J, Liu D R (2007) Supercharging proteins can impart unusual resilience. J. Am. Chem. Soc. 129:10110-10112; International PCT patent application, PCT/US07/70254, filed Jun. 1, 2007, published as WO 2007/143574 on Dec. 13, 2007; U.S. provisional patent applications, U.S. Ser. No. 60/810,364, filed Jun. 2, 2006, and U.S. Ser. No. 60/836,607, filed Aug. 9, 2006; each of which is incorporated herein by reference). When the replacement residues are all positively or all negatively charged, the resulting “supercharged” proteins can retain their activity while gaining unusual properties such as robust resistance to aggregation and the ability to bind oppositely charged macromolecules. For example, we reported that a green fluorescent protein with a +36 net theoretical charge (+36 GFP) was highly aggregation-resistant, could retain fluorescence even after being boiled and cooled, and reversibly complexed DNA and RNA through electrostatic interactions.

A variety of cationic peptides with the ability to penetrate mammalian cells including peptides derived from HIV Tat (Frankel A D, Pabo C O (1988) Cellular uptake of the tat protein from human immunodeficiency virus. Cell 55: 1189-1193; Green M, Loewenstein P M (1988) Automonous functional domains of chemically synthesized human immunodeficiency virus tat trans-activator protein. Cell 55: 1179-1188; each of which is incorporated herein by reference) and penetratin from the Antennapedia homeodomain (Thoren P E, Persson D, Karlsson M, Norden B (2000) The antennapedia peptide penetratin translocates across lipid bilayers—the first direct observation. FEBS Lett 482: 265-268; incorporated herein by reference) have been previously described. Schepartz and coworkers have recently shown that small, folded proteins containing a minimal cationic motif embedded within a type II polyproline helix efficiently penetrate eukaryotic cells (Daniels D S, Schepartz A (2007) Intrinsically cell-permeable miniature proteins based on a minimal cationic PPII motif. J Am Chem Soc 129: 14578-14579; Smith B A, Daniels D S, Coplin A E, Jordan G E, McGregor L M, et al. (2008) Minimally cationic cell-permeable miniature proteins via alpha-helical arginine display. J Am Chem Soc 130: 2948-2949; each of which is incorporated herein by reference). Raines and coworkers recently engineered proteins with a surface-exposed poly-arginine patch that confers the ability to penetrate cells (Fuchs S M, Raines R T (2007) Arginine grafting to endow cell permeability. ACS Chem Biol 2: 167-170; Fuchs S M, Rutkoski T J, Kung V M, Groeschl R T, Raines R T (2007) Increasing the potency of a cytotoxin with an arginine graft. Protein Eng Des Sel 20: 505-509; each of which is incorporated herein by reference). In light of these studies, we hypothesized that superpositively charged proteins such as +36 GFP might associate with negatively charged components of the cell membrane in a manner that results in cell penetration.

In the present Example, we describe the cell-penetrating characteristics of superpositively charged GFP variants with net charges of +15, +25, and +36. We found that +36 GFP potently enters cells through sulfated peptidoglycan-mediated, actin-dependent endocytosis. When pre-mixed with siRNA, +36 GFP delivers siRNA effectively and without cytotoxicity into a variety of cell lines, including several known to be resistant to cationic lipid-mediated transfection. The siRNA delivered into cells using +36 GFP was able to effect gene silencing in four out of five mammalian cell lines tested. Comparison of the siRNA transfection ability of +36 GFP with that of several synthetic peptides of comparable or greater charge magnitude and charge density suggests that the observed mode of siRNA delivery may require protein-like features of +36 GFP that are not present among cationic peptides. When fused to an endosomolytic peptide derived from hemagglutinin, +36 GFP is also able to transfect plasmid DNA into several cell lines that resist cationic lipid-mediated transfection in a manner that enables plasmid-based gene expression.

### Results

### Mammalian Cell Penetration by Supercharged GFPs.

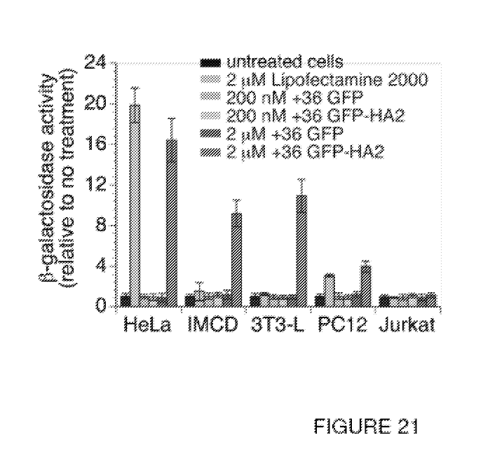

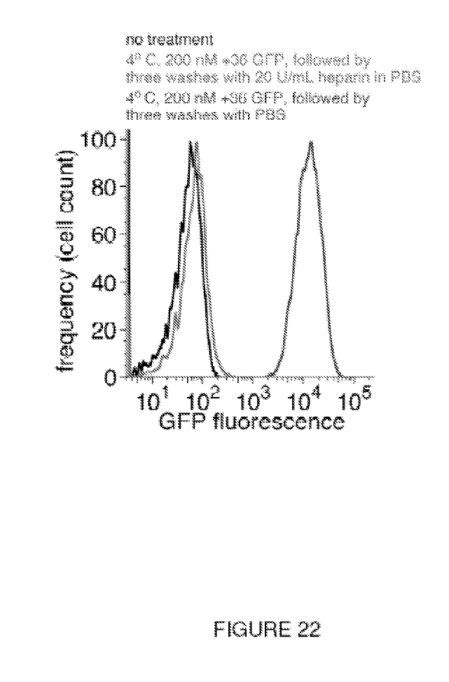

We previously generated and characterized a series of resurfaced variants of “superfolder GFP” (sfGFP) (Pedelacq J D, Cabantous S, Tran T, Terwilliger T C, Waldo G S (2006) Engineering and characterization of a superfolder green fluorescent protein. Nat Biotechnol 24: 79-88; incorporated herein by reference) with theoretical net charges ranging from −30 to +48 that retain fluorescence (Lawrence M S, Phillips K J, Liu D R (2007) Supercharging proteins can impart unusual resilience. J Am Chem Soc 129: 10110-10112; incorporated herein by reference). The evaluation of the ability of these supercharged GFPs to penetrate mammalian cells requires a method to remove surface-bound, non-internalized GFP. We therefore confirmed that washing conditions known to remove surface-bound cationic proteins from cells (Pedelacq J D, Cabantous S, Tran T, Terwilliger T C, Waldo G S (2006) Engineering and characterization of a superfolder green fluorescent protein. Nat Biotechnol 24: 79-88) also effectively remove cell surface-bound superpositively charged GFP. We treated HeLa cells with +36 GFP at 4° C., a temperature that allows +36 GFP to bind to the outside of cells but blocks internalization (vide infra). Cells were washed three times at 4° C. with either PBS or with PBS containing heparin and analyzed by flow cytometry for GFP fluorescence. Cells washed with PBS were found to have significant levels of GFP (presumably surface-bound), while cells washed with PBS containing heparin exhibited GFP fluorescence intensity very similar to that of untreated cells (FIG. 22). These observations confirmed the effectiveness of three washes with heparin at removing surface-bound superpositively charged GFP.

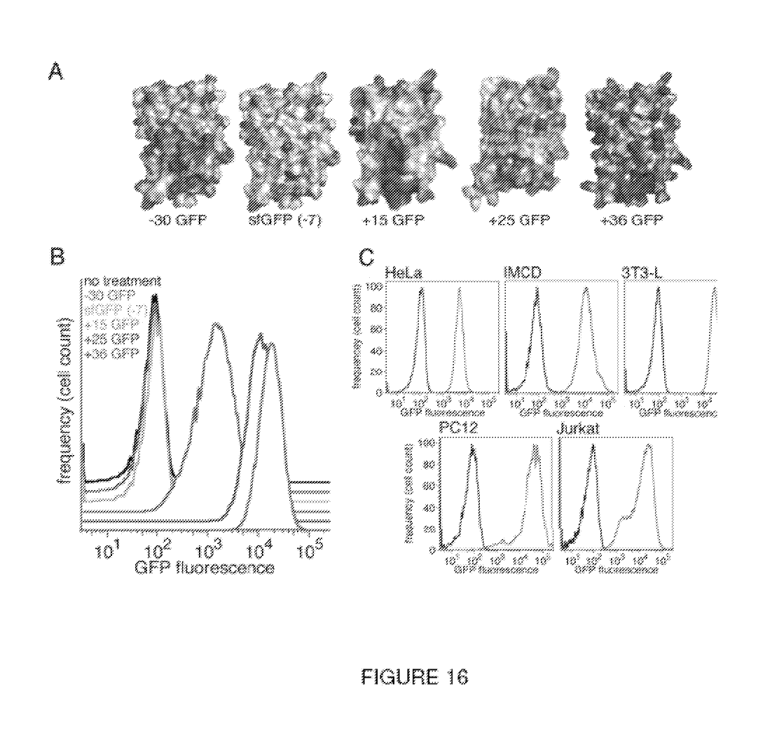

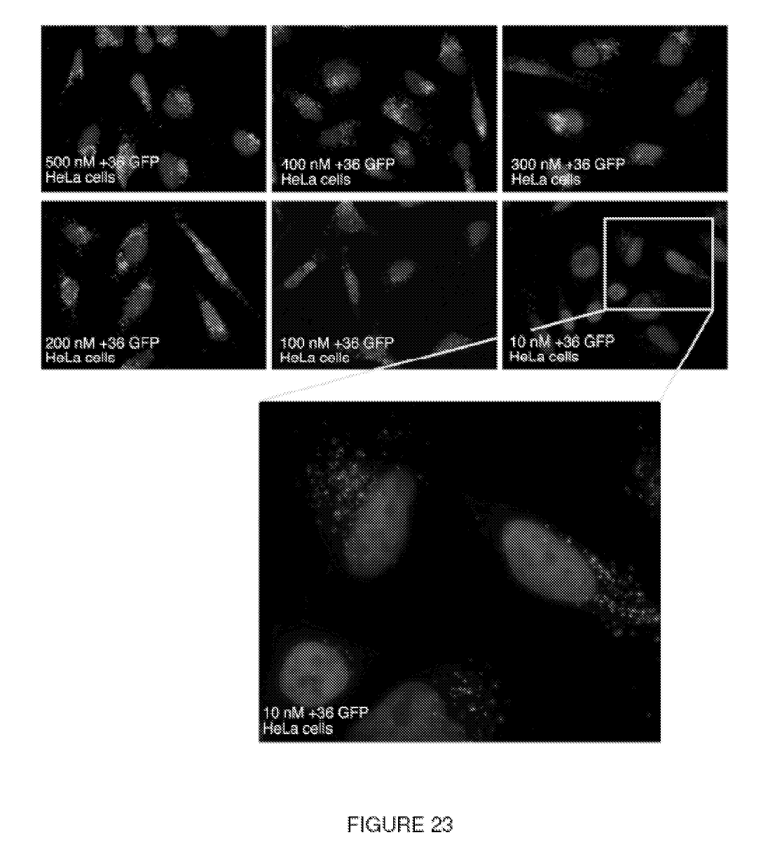

Next we incubated HeLa cells with 10-500 nM sfGFP (theoretical net charge of −7), −30 GFP, +15 GFP, +25 GFP, or +36 GFP for 4 hours at 37° C. (FIG. 16A). After incubation, cells were washed three times with PBS containing heparin and analyzed by flow cytometry. No detectable internalized protein was observed in cells treated with sfGFP or −30 GFP. HeLa cells treated with +25 GFP or +36 GFP, however, were found to contain high levels of internalized GFP. In contrast, cells treated with +15 GFP contained 10-fold less internalized GFP, indicating that positive charge magnitude is an important determinant of effective cell penetration (FIG. 16B). We found that +36 GFP readily penetrates HeLa cells even at concentrations as low as 10 nM (FIG. 23).

In order to test the generality of cell penetration by +36 GFP, we repeated these experiments using four additional mammalian cell types: inner medullary collecting duct (IMCD) cells, 3T3-L pre-adipocytes, rat pheochromocytoma PC12 cells, and Jurkat T-cells. Flow cytometry analysis revealed that 200 nM +36 GFP effectively penetrates all five types of cells tested (FIG. 16C). Internalization of +36 GFP in stably adherent HeLa, IMCD, and 3T3-L cell lines was confirmed by fluorescence microscopy (vide infra). Real-time imaging showed +36 GFP bound rapidly to the cell membrane of HeLa cells and was internalized within minutes as punctate foci that migrated towards the interior of the cell and consolidated into larger foci, consistent with uptake via endocytosis.

### Mechanistic Probes of +36 GFP Cell Penetration

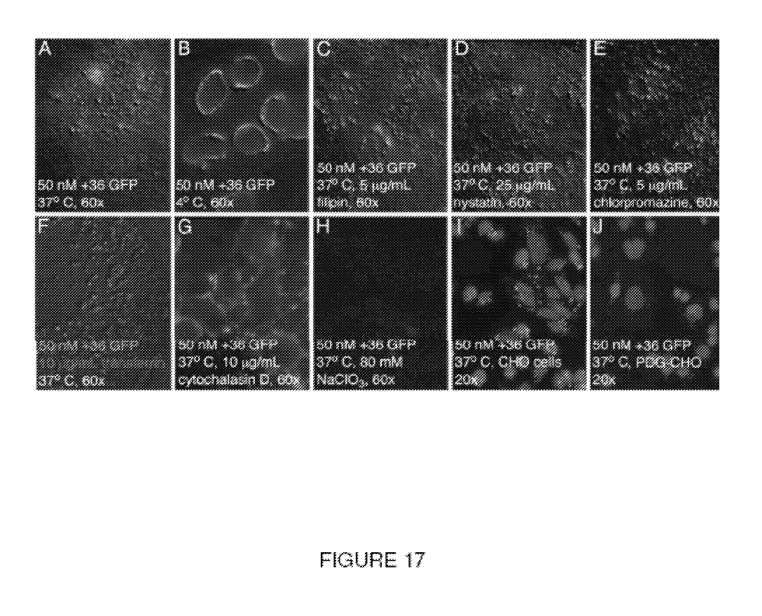

To illuminate the mechanism by which +36 GFP enters cells, we repeated the cell penetration experiments in HeLa cells under a variety of conditions that each blocks a different component of an endocytosis pathway (Payne C K, Jones S A, Chen C, Zhuang X (2007) Internalization and trafficking of cell surface proteoglycans and proteoglycan-binding ligands. Traffic 8: 389-401; Veldhoen S, Laufer S D, Trampe A, Restle T (2006) Cellular delivery of small interfering RNA by a non-covalently attached cell-penetrating peptide: quantitative analysis of uptake and biological effect. Nucleic Acids Res 34: 6561-6573; each of which is incorporated herein by reference). Cell penetration of +36 GFP was not observed when HeLa cells were cooled to 4° C. prior to and during +36 GFP treatment (FIG. 17B). This result suggests that uptake of +36 GFP requires an energy-dependent process, consistent with endocytosis (Deshayes S, Morris M C, Divita G, Heitz F (2005) Cell-penetrating peptides: tools for intracellular delivery of therapeutics. Cell Mol Life Sci 62: 1839-1849; incorporated herein by reference). We next evaluated the effects of 5 μg/mL filipin or 25 μg/mL nystatin, small molecules known to inhibit caveolin-dependent endocytosis. Neither inhibitor significantly altered +36 GFP internalization (FIGS. 17C and 17D, respectively). Treatment with chlorpromazine, a known inhibitor of clathrin-mediated endocytosis, similarly had little effect on +36 GFP cell penetration (FIG. 17E). In addition, simultaneous treatment of HeLa cells with 50 nM +36 GFP and 10 μg/mL of fluorescently labeled transferrin, a protein known to be internalized in a clathrin-dependent manner (Hopkins C R, Trowbridge I S (1983) Internalization and processing of transferrin and the transferrin receptor in human carcinoma A431 cells. J Cell Biol 97: 508-521; incorporated herein by reference), resulted in little GFP/transferrin co-localization (FIG. 17F). Treatment with cytochalasin D, an actin polymerization inhibitor, however, significantly decreased +36 GFP cell penetration (FIG. 17G). Taken together, these results are consistent with a model in which +36 GFP uptake proceeds through an endocytotic pathway that is energy-dependent, requires actin polymerization, and does not require clathrin or caveolin.

Based on previous studies on the mechanism of cellular uptake of cationic peptides (Payne C K, Jones S A, Chen C, Zhuang X (2007) Internalization and trafficking of cell surface proteoglycans and proteoglycan-binding ligands. Traffic 8: 389-401; Fuchs S M, Raines R T (2004) Pathway for polyarginine entry into mammalian cells. Biochemistry 43: 2438-2444; each of which is incorporated herein by reference), we hypothesized that anionic cell-surface proteoglycans might serve as receptors to mediate +36 GFP internalization. To probe this hypothesis we pre-treated HeLa cells with 80 mM sodium chlorate, an inhibitor of ATP sulphurylase, an enzyme required for the biosynthesis of sulfated proteoglycans (Baeuerle P A, Huttner W B (1986) Chlorate—a potent inhibitor of protein sulfation in intact cells. Biochem Biophys Res Commun 141: 870-877; incorporated herein by reference). These conditions completely blocked +36 GFP penetration (FIG. 17H). As a further probe of the role proteoglycans play in +36 GFP uptake, we compared internalization in wild-type Chinese hamster ovary (CHO) cells with proteoglycan-deficient CHO cells (PGD-CHO) that lack xylosyltransferase, an enzyme required for glycosaminoglycan synthesis. Wild-type CHO cells (FIG. 17I), but not PGD-CHO cells (FIG. 17J), efficiently internalized +36 GFP. These findings suggest that +36 GFP penetration of mammalian cells requires binding to sulfated cell-surface peptidoglycans.

+36 GFP Binds siRNA and Delivers siRNA into a Variety of Mammalian Cell Lines

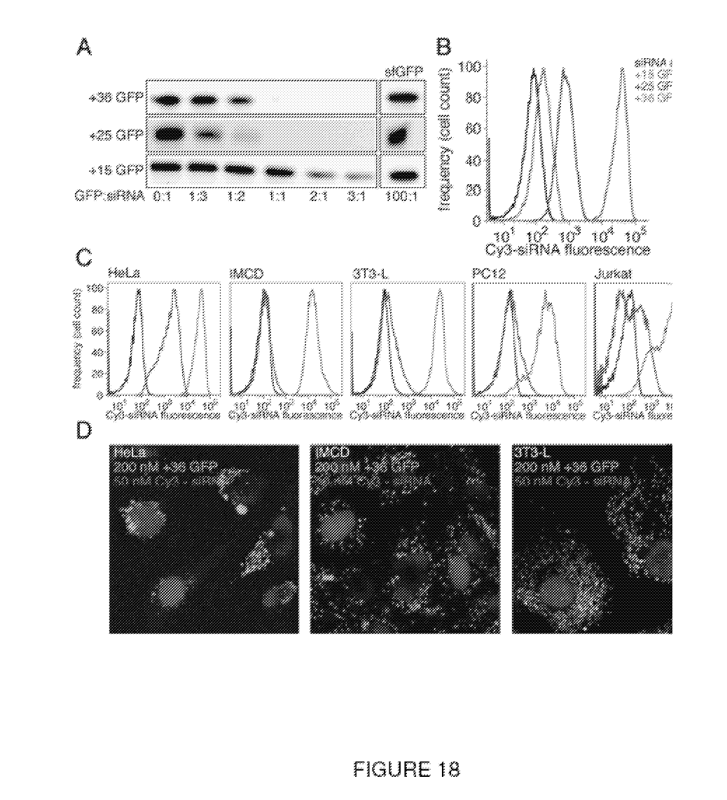

We have observed the ability of superpositively charged proteins to form complexes with DNA and tRNA (Lawrence et al. (2007) Supercharging proteins can impart unusual resilience. J Am Chem Soc 129: 10110-10112; incorporated herein by reference). In light of these results, we evaluated the ability of +15, +25, and +36 GFP to bind siRNA in vitro in a variety of stoichiometric ratios. Using a gel-shift assay (Kumar P, Wu H, McBride J L, Jung K E, Kim M H, et al. (2007) Transvascular delivery of small interfering RNA to the central nervous system. Nature 448: 39-43; incorporated herein by reference), we observed binding of +25 and +36 GFP to siRNA with a stoichiometry of ˜2:1, while greater than five +15 GFP proteins on average were required to complex a single siRNA molecule (FIG. 18A). In contrast, 100 equivalents of sfGFP did not detectably bind siRNA under the assay conditions.

Next we examined the ability of +15, +25, and +36 GFP to deliver bound siRNA into HeLa cells. A Cy3-conjugated GAPDH siRNA (Ambion) was briefly mixed with 200 nM +36 GFP and the resulting mixture was added to cells in serum-free media for 4 hours. The cells were washed three times with PBS containing heparin and analyzed by flow cytometry for Cy3-siRNA uptake. We observed that +25 and +36 GFP delivered 100- and 1000-fold more siRNA into HeLa cells, respectively, than treatment with siRNA alone (FIG. 3B), and ˜20-fold more siRNA than was delivered with the common cationic lipid transfection reagent Lipofectamine 2000 (FIG. 18C). In contrast, +15 GFP did not efficiently transfect siRNA into HeLa cells (FIG. 18B).

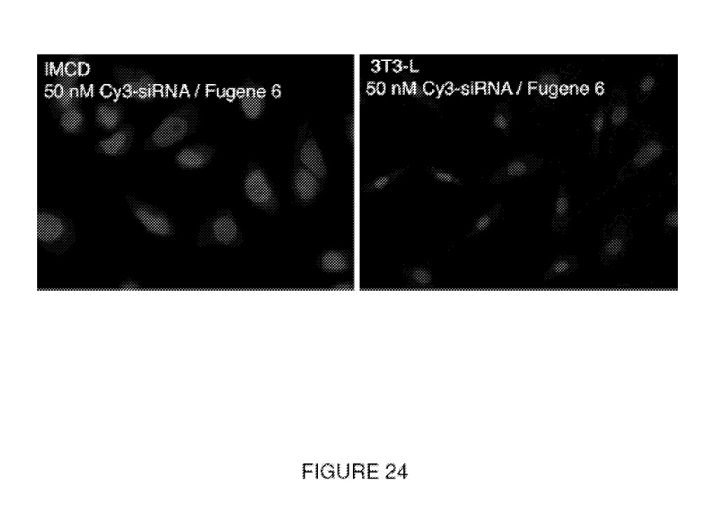

In addition to HeLa cells, +36 GFP was able to efficiently deliver siRNA in IMCD cells, 3T3-L preadipocytes, rat pheochromocytoma PC12 cells, and Jurkat T-cells, four cell lines that are resistant to siRNA transfection using Lipofectamine 2000 (Carlotti F, Bazuine M, Kekarainen T, Seppen J, Pognonec et al. (2004) Lentiviral vectors efficiently transduce quiescent mature 3TL-L1 adipocytes. Mol Ther 9: 209-217; Ma H, Zhu J, Maronski M, Kotzbauer P T, Lee V M, Dichter M A, et al. (2002) Non-classical nuclear localization signal peptides for high efficiency lipofection of primary neurons and neuronal cell lines. Neuroscience 112: 1-5; McManus M T, Haines B B, Dillon C P, Whitehurst C E, van Parijs L, et al. (2002) Small interfering RNA-mediated gene silencing in T lymphocytes. J Immunol 169: 5754-5760; Strait K A, Stricklett P K, Kohan J L, Miller M B, Kohan D E (2007) Calcium regulation of endothelin-1 synthesis in rat inner medullary collecting duct. Am J Physiol Renal Physiol 293: F601-606; each of which is incorporated herein by reference). Treatment with Lipofectamine 2000 and Cy3-siRNA resulted in efficient siRNA delivery in HeLa cells, but no significant delivery of siRNA into IMCD, 3T3-L, PC 12, or Jurkat cells (FIG. 18C). Treatment of IMCD or 3T3-L cells with Fugene 6 (Roche), a different cationic lipid transfection agent, and Cy3-siRNA also did not result in significant siRNA delivery these cells (FIG. 24). In contrast, treatment with +36 GFP and Cy3-siRNA resulted in significant siRNA levels in all five cell lines tested (FIG. 18C). Compared with Lipofectamine 2000, +36 GFP resulted in 20- to 200-fold higher levels of Cy3 signal in all cases. Based on the effectiveness of three heparin washes at removing non-internalized +36 GFP, (FIG. 22) we attribute these higher Cy3 levels to higher levels of internalized Cy3-siRNA rather than to cell surface-bound +36 GFP/Cy3-siRNA complexes. Consistent with this interpretation, fluorescence microscopy of the adherent cell lines used in this study (HeLa, IMCD, and 3T3-L) reveal internalized Cy3-siRNA and +36 GFP in punctate foci that we presume to be endosomes (FIG. 18D). These results collectively indicate that +36 GFP can effectively deliver siRNA into a variety of mammalian cell lines, including several that are poorly transfected by commonly used cationic lipid transfection reagents.

When HeLa cells were treated with the a premixed solution containing 200 nM +36 GFP and 50 nM Cy3-siRNA in the presence of cytochalasin D or at 4° C., no internalized GFP or Cy3 siRNA was observed (FIG. 30). These data support a mechanism of siRNA delivery that is dependent on endocytosis and actin polymerization, consistent with the present inventors' mechanistic studies of +36 GFP in the absence of siRNA.

### Size and Cytotoxicity of +36 GFP-siRNA Complexes.

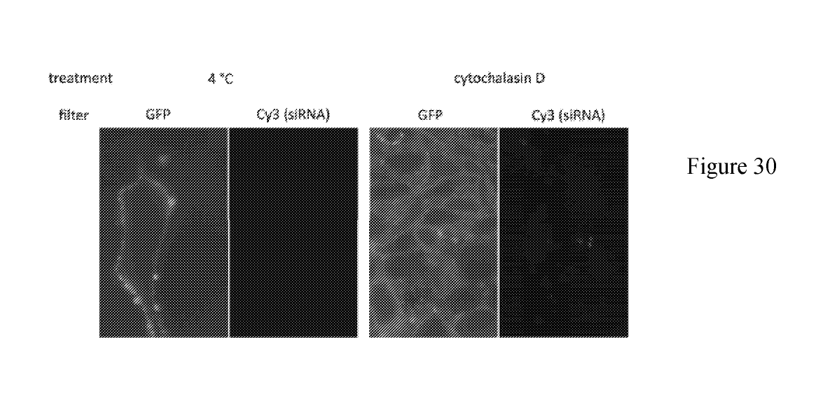

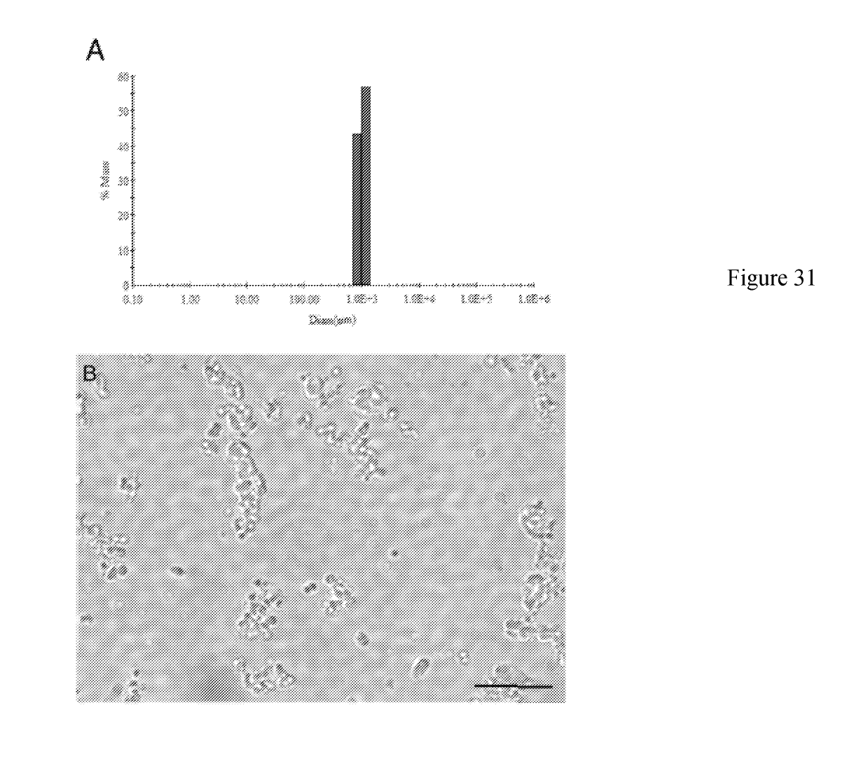

+36 GFP-siRNA complexes were analyzed by dynamic light scattering (DLS) using stoichiometric ratios identical to those used for transfection. From a mixture containing 20 μM +36 GFP and 5 μM siRNA, we observed a fairly monodisperse population of particles with a hydrodynamic radius (Hr) of 880.6±62.2 nm (FIG. 31A), consistent with microscopy data (FIG. 31B). These observations demonstrate the potential for +36 GFP to form large particles when mixed with siRNA, a phenomena observed by previous researchers using cationic delivery reagents (Deshayes et al., 2005, Cell Mol. Life. Sci., 62:1839-49; and Meade and Dowdy, 2008, Adv. Drug Deliv. Rev., 60:530-36; both of which are incorporated herein by reference).

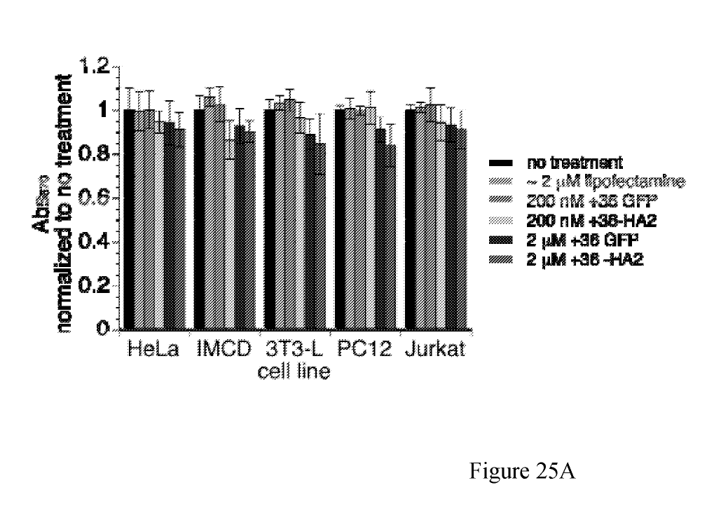

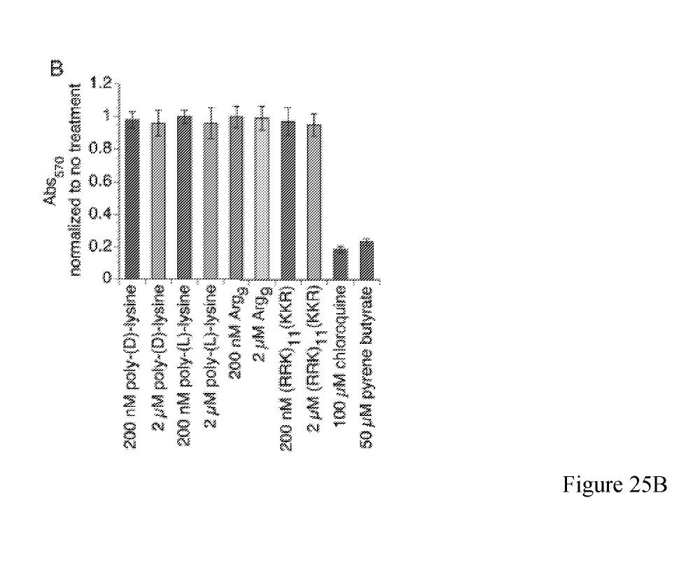

To assess the cytotoxicity of +36 GFP-siRNA complexes, we performed MTT assays on all five cell lines 24 hours after treatment with 0.2 to 2 μM +36 GFP and 50 nM siRNA. These assays revealed no significant apparent cytotoxicity to HeLa, IMCD, 3T3-L, PC12, or Jurkat cells (FIG. 25A).

Gene Silencing with +36 GFP-Delivered siRNA

While the above results demonstrate the ability of +36 GFP to deliver siRNA into a variety of mammalian cells, they do not establish the availability of this siRNA for gene silencing. Based on the punctate localization of intracellular +36 GFP (FIG. 18D), we anticipated that gene silencing would require at least partial escape of +36 GFP-transfected siRNA from endosomes. To evaluate the gene suppression activity of siRNA delivered with +36 GFP, we treated HeLa, IMCD, 3T3-L, PC12, and Jurkat cells with a solution containing 50 nM of GAPDH-targeting siRNA and either ˜2 μM Lipofectamine 2000 or 200 nM +36 GFP. Cells were exposed to the siRNA transfection solution for 4 hours, then grown for up to 4 days.

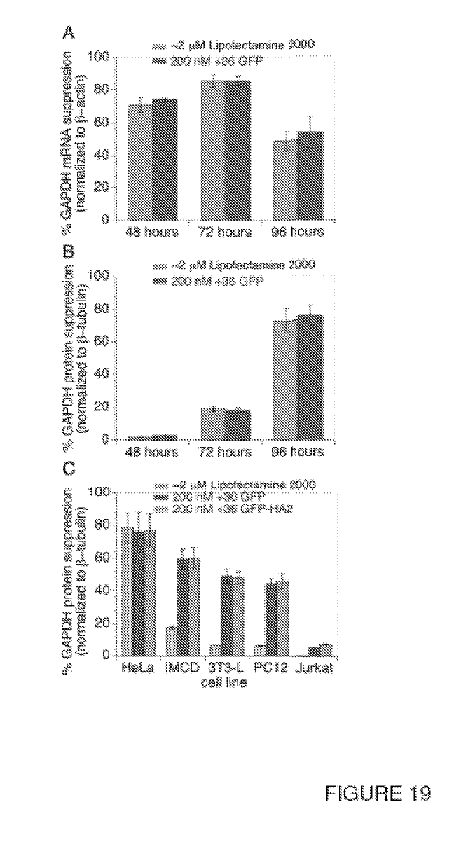

In HeLa cells, observed decreases in GAPDH mRNA and protein levels indicate that both Lipofectamine 2000 and +36 GFP mediate efficient siRNA-induced suppression of GAPDH expression with similar kinetics. GAPDH-targeting siRNA delivered with Lipofectamine 2000 or +36 GFP resulted in a ˜85% decrease in GAPDH mRNA level after 72 hours (FIG. 19A). Similarly, a decrease in GAPDH protein levels of ˜75% was observed in HeLa cells 96 hours after delivery of siRNA with Lipofectamine 2000 or with +36 GFP (FIG. 19B). Similarly, delivery of β-actin targeting siRNA with either ˜2 μM Lipofectamine 2000 or 200 nM +36 GFP resulted in a decrease in β-actin protein levels in HeLa cells of 70-78% for both transfection agents (FIG. 19B).

In contrast to the efficiency of gene suppression in HeLa cells, treatment with Lipofectamine 2000 and 50 nM siRNA in IMCD, 3T3-L, PC12, and Jurkat cells effected no significant decrease in GAPDH protein levels (FIG. 19C), consistent with the resistance of these cell lines to cationic lipid-mediated transfection (FIG. 18C). However, treatment with 200 nM +36 GFP and 50 nM siRNA resulted in 44-60% suppression of GAPDH protein levels in IMCD, 3T3-L, and PC12 cells (FIG. 19C). Despite efficient siRNA delivery by +36 GFP (FIG. 18C), we observed no significant siRNA-mediated suppression of GAPDH expression in Jurkat cells (FIG. 19C).

We speculated that enhancing the escape of +36 GFP-delivered siRNA from endosomes may increase the effectiveness of gene silencing. In an attempt to chemically disrupt endocytotic vesicles, cells were treated with 200 nM +36 GFP and 50 nM siRNA together with either chloroquine, a small molecule known to have endosomolytic activity (Erbacher P, Roche A C, Monsigny M, Midoux P (1996) Putative role of chloroquine in gene transfer into a human hepatoma cell line by DNA/lactosylated polylysine complexes. Exp Cell Res 225, 186-194; incorporated herein by reference), or pyrene butyric acid, which has been shown to increase cytosolic distribution of internalized poly-arginine (Takeuchi T, Kosuge M, Tadokoro A, Sugiura Y, Nishi M, et al. (2006) Direct and rapid cytosolic delivery using cell-penetrating peptides mediated by pyrenebutyrate. ACS Chem Biol 1: 299-303; incorporated herein by reference). Addition of these reagents to mixtures containing +36 GFP and siRNA proved cytotoxic in the cell lines tested. In addition, we generated and purified a C-terminal fusion of +36 GFP and the hemagglutinin 2 (HA2) peptide, which has been reported to enhance endosome degradation (Lundberg P, El-Andaloussi S, Sutlu T, Johansson H, Langel U (2007) Delivery of short interfering RNA using endosomolytic cell-penetrating peptides. FASEB J 21: 2664-2671; incorporated herein by reference). As was the case with +36 GFP, the HA2-fused variant exhibited low cytotoxicity in the five cell lines tested (FIG. 25A). While the delivery of siRNA with +36 GFP-HA2 fusion resulted in decreased GAPDH protein levels in HeLa, IMCD, 3T3-L, and PC12 cells, the degree of suppression was comparable to that arising from the use of +36 GFP (FIG. 19C).

Together, these results indicate that +36 GFP and +36 GFP-HA2 are capable of delivering siRNA and effecting gene silencing in a variety of mammalian cells, including some cell lines that do not exhibit gene silencing when treated with siRNA and cationic lipid-based transfection agents.

Stability of +36 GFP and Stability of RNA and DNA Complexed with +36 GFP

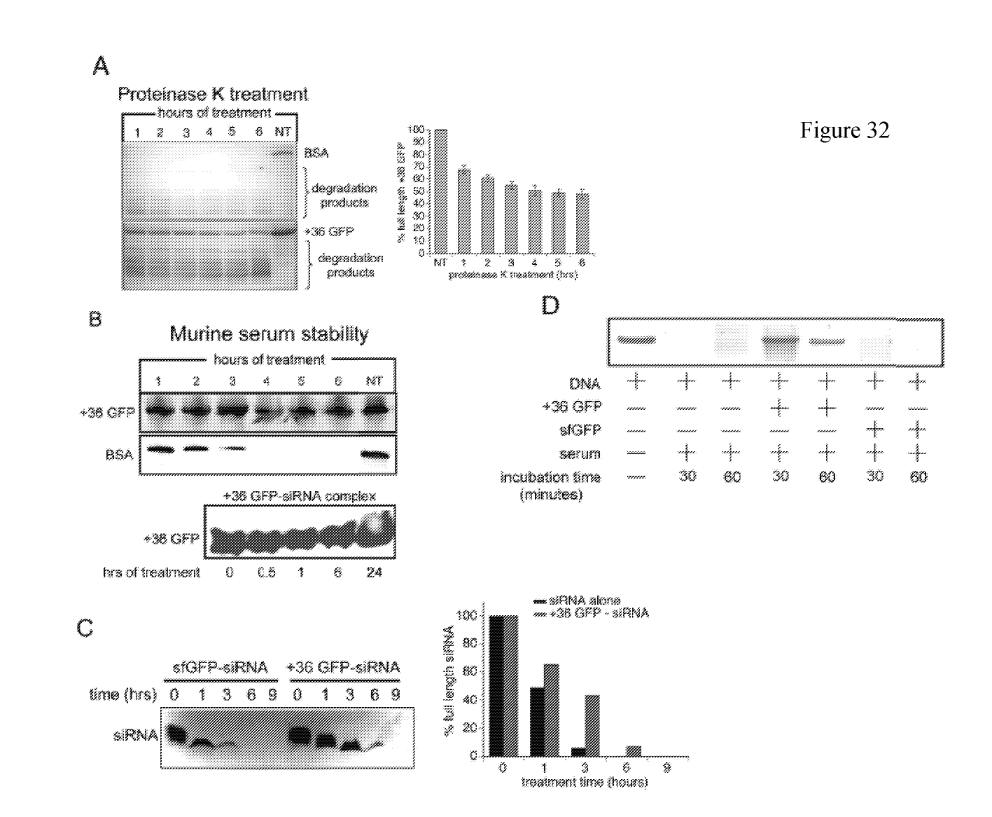

In addition to generality across different mammalian cell types and low cytotoxicity, siRNA delivery agents may be resistant to rapid degradation. Treatment of +36 GFP with proteinase K (a robust, broad-spectrum protease) revealed that +36 GFP exhibits significant protease resistance compared with bovine serum albumin. While no uncleaved BSA remained one hour after proteinase K digestion, 68% of +36 GFP remained uncleaved after one hour, and 48% remained uncleaved after six hours (FIG. 32A). We also treated +36 GFP with murine serum at 37° C. (FIG. 32B). After six hours, no significant degradation was observed, suggesting its potential in vivo serum stability. In comparison, when bovine serum albumin was incubated in mouse serum for the same period of time, 71% degradation was observed after three hours, and complete degradation by four hours.

The ability of +36 GFP to protect siRNA and plasmid DNA from degradation was assessed. siRNA or siRNA pre-complexed with +36 GFP was treated with murine serum at 37° C. After three hours, only 5.9% of the siRNA remained intact in the sample lacking +36 GFP, while 34% of the siRNA remained intact in the sample pre-complexed with +36 GFP (FIG. 32C). Similarly, while plasmid DNA was nearly completely degraded by murine serum after 30 minutes at 37° C., virtually all plasmid DNA pre-complexed with +36 GFP remained intact after 30 minutes, and 84% of plasmid DNA was intact after one hour (FIG. 32D). These results together indicate that +36 GFP is capable of significantly inhibiting serum-mediated siRNA and plasmid DNA degradation.

Comparison of +36 GFP with Synthetic Cationic Peptides

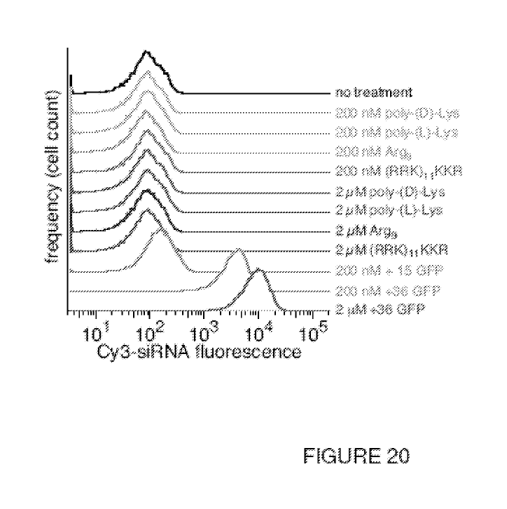

To probe the features of superpositively charged GFPs that impart their ability to deliver siRNA into cells, we compared the siRNA transfection ability of +36 GFP at 200 nM with that of a panel of synthetic cationic peptides at 200 nM or 2 μM. This panel consisted of poly-(L)-Lys (a mixture containing an average of ˜30 Lys residues per polypeptide), poly-(D)-Lys, Arg₉, and a synthetic +36 peptide ((KKR)₁₁RRK) that contains the same theoretical net charge and Lys:Arg ratio as +36 GFP. MTT assays on HeLa cells treated with these synthetic polycations indicated low cytoxicity at the concentrations used, consistent with that of superpositively charged GFPs (FIG. 25B). None of the four synthetic peptides tested delivered a detectable amount of Cy3-siRNA into HeLa cells as assayed by flow cytometry, even when used at concentrations 10-fold higher than those needed for +36 GFP to effect efficient siRNA delivery or for +15 GFP to effect detectable siRNA delivery (FIG. 20).

Coupled with our observation that +15 GFP exhibits low cell penetration and siRNA binding activity in comparison to +25 and +36 GFP (FIGS. 18A and 18B), these results indicate that while GFP must be sufficiently positively charged to acquire the ability to enter cells and transfect siRNA efficiently, positive charge magnitude and charge density are not sufficient to confer transfection activity. Instead, our findings suggest that protein-like features of +36 GFP such as size, globular shape, or stability may be required to achieve the full set of cell penetration and siRNA transfection activities that we observed.

### +36 GFP-Mediated Transfection of Plasmid DNA

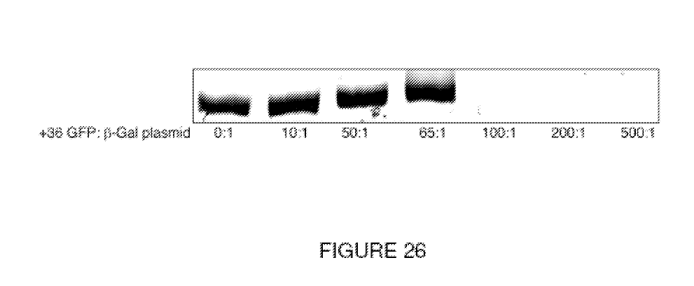

Similar to the case with siRNA, we observed by gel-shift assay that +36 GFP forms a complex with plasmid DNA (FIG. 26). To test if +36 GFP can deliver plasmid DNA to cells in a manner that supports plasmid-based gene expression, we treated HeLa, IMCD, 3T3-L, PC12, and Jurkat cells with a β-galactosidase expression plasmid premixed with Lipofectamine 2000, +36 GFP, or a C-terminal fusion of +36 GFP and the hemagglutinin 2 (HA2) peptide, which has been reported to enhance endosome degradation (Lundberg et al., 2007, Faseb J., 21:2664-71; incorporated herein by reference). After 24 hours, cells were analyzed for β-galactosidase activity using a fluorogenic substrate-based assay.

Consistent with our previous results (FIGS. 18 and 19), Lipofectamine 2000 treatment resulted in significant β-galactosidase activity in HeLa cells, but only modest β-galactosidase activity in PC12 cells, and no detectable activity in any of the other three cell lines tested (FIG. 21). In contrast, plasmid transfection mediated by 2 μM +36 GFP-HA2 resulted in significant β-galactosidase activity in HeLa, IMCD, and 3T3-L cells, and modest activity in PC12 cells (FIG. 21). Interestingly, treatment with plasmid DNA and 2 μM +36 GFP did not result in detectable β-galactosidase activity (FIG. 21), suggesting that the hemagglutinin-derived peptide enhances DNA transfection or plasmid-based expression efficiency despite its lack of effect on siRNA-mediated gene silencing (FIG. 19C).

These results collectively indicate that +36 GFP-HA2 is able to deliver plasmid DNA into mammalian cells, including several cell lines resistant to cationic lipid-mediated transfection, in a manner that enables plasmid-based gene expression. Higher concentrations of +36 GFP-HA2 are required to mediate plasmid DNA transfection than the amount of +36 GFP or +36 GFP-HA2 needed to induce efficient siRNA transfection.

### CONCLUSION

The present inventors have characterized the cell penetration, siRNA delivery, siRNA-mediated gene silencing, and plasmid DNA transfection properties of three superpositively charged GFP variants with net charges of +15, +25, and +36. The present inventors discovered that +36 GFP is highly cell permeable and capable of efficiently delivering siRNA into a variety of mammalian cell lines, including those resistant to cationic lipid-based transfection, with low cytotoxicity.

Mechanistic studies revealed that +36 GFP enters cells through a clathrin- and caveolin-independent endocytosis pathway that requires sulfated cell-surface proteoglycans and actin polymerization. This delivery pathway differs from previously described strategies for nucleic acid delivery to eukaryotic cells that rely on cell-specific targeting to localize their nucleic acid cargo (Song et al., 2005, Nat. Biotechnol., 23:709-17; Kumar et al., 2007, Nature, 448:39-43; and Cardoso et al., 2007, J. Gene Med., 9:170-83; all of which are incorporated herein by reference). For use in cell culture and even in certain in vivo applications, a general, noncell type-specific approach to nucleic acid delivery may be desirable.

In four of the five cell lines tested, +36 GFP-mediated siRNA delivery induces significant suppression of gene expression. Moreover, a +36 GFP-hemagglutinin peptide fusion can mediate plasmid DNA transfection in a manner that enables plasmid-based gene expression in the same four cell lines. The presently demonstrated ability to transfect RNA 21 base pairs in length as well as plasmid DNA over 5,000 bp in length suggests that +36 GFP and its derivatives may serve as general nucleic acid delivery vectors.

Many traditional delivery methods rely on the synthesis of covalently linked transfection agent-nucleic acid conjugates such as, carbon nanotube-siRNA (Liu et al., 2007, Agnew Chem. Int. Ed. Engl., 46:2023-27; incorporated herein by reference), nanoparticle-siRNA (Rosi et al., 2006, Science, 312:1027-30; incorporated herein by reference), TAT peptide-siRNA (Fisher et al., 2002, J. Biol. Chem., 277:22980-84; incorporated herein by reference), cholesterol-siRNA (Soutschek et al., 2004, Nature, 432:173-78; incorporated herein by reference), and dynamic polyconjugate-siRNA (Rozema et al., 2007, Proc. Natl. Acad. Sci., USA, 104:12982-87; incorporated herein by reference). Use of +36 GFP simply requires mixing the protein and nucleic acid together. Moreover, the reagent described here is purified directly from bacterial cells and used without chemical co-transfectants such as exogenous calcium or chloroquine.

The present inventors previously reported that +36 GFP is thermodynamically almost as stable as sfGFP but unlike the latter is able to refold after boiling and cooling (Lawrence et al., 2007, J. Am. Chem. Soc., 129:10110-12; incorporated herein by reference). The present inventors have now demonstrated that +36 GFP exhibits resistance to proteolysis, stability in murine serum, and significant protection of complexed siRNA in murine serum. Thus, the present invention encompasses the recognition that these systems may be useful for in vivo nucleic acid delivery (e.g., to human, mammalian, non-human, or non-mammalian cells).

Thus, the present invention describes for the first time use of protein resurfacing methods for the potent delivery of nucleic acids into mammalian cells. This surprising and significant potency (Deshayes et al., 2007, Meth. Mol. Biol., 386:299-308; and Lundberg et al., 2007, Faseb J., 21:2664-71; both of which are incorporated herein by reference) is complemented by low cytotoxicity, stability in mammalian serum, generality across various mammalian cell types including several that resist traditional transfection methods, the ability to transfect both small RNAs and large DNA plasmids, straightforward preparation from E. coli cells, and simple use by mixing with an unmodified nucleic acid of interest. Thus the present invention encompasses the recognition that supercharged proteins represent a new class of solutions to general nucleic acid delivery problems in mammalian cells.

### Materials and Methods

### Cell Culture

HeLa, IMCD, PC12, and 3T3-L cells were cultured in Dulbecco's modification of Eagle's medium (DMEM, purchased from Sigma) with 10% fetal bovine serum (FBS, purchased from Sigma), 2 mM glutamine, 5 I.U. penicillin, and 5 μg/mL streptamycin. Jurkat cells were cultured in RPMI 1640 medium (Sigma) with 10% FBS, 2 mM glutamine, 5 I.U. penicillin, and 5 μg/mL streptamycin. All cells were cultured at 37° C. with 5% CO₂. PC12 cells were purchased from ATCC.

### Expression and Purification of Supercharged GFP Proteins

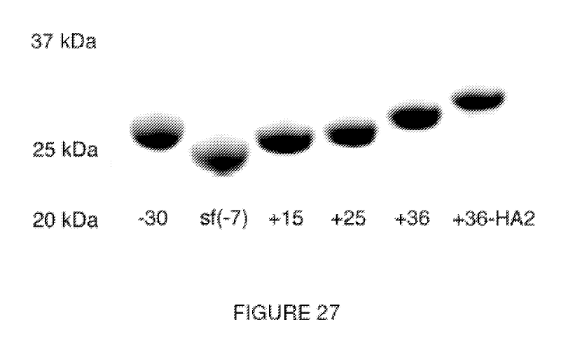

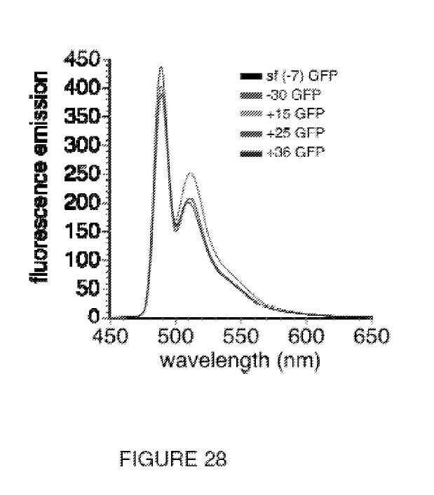

Supercharged GFP variants (protein sequences are listed below) were purified using a variation on our previously reported method. Briefly, GFP was overexpressed in BL21(DE3) E. coli. Cells were lysed by sonication in 2 M NaCl in PBS which was found to increase overall yield of isolated GFP, and purified as previously described (Lawrence M S, Phillips K J, Liu D R (2007) Supercharging proteins can impart unusual resilience. J Am Chem Soc 129: 10110-10112; incorporated herein by reference). Purified GFPs were quantitated by absorbance at 488 nm assuming an extinction coefficient of 8.33×10⁴ M⁻¹cm⁻¹ (Pedelacq J D, Cabantous S, Tran T, Terwilliger T C, Waldo G S (2006) Engineering and characterization of a superfolder green fluorescent protein. Nat Biotechnol 24: 79-88; incorporated herein by reference). Protein purity was evaluated by SDS PAGE and Coomassie Blue staining (FIG. 27). Fluorescence emission spectra of the GFP variants used in this work are similar (FIG. 28).

### Protein Sequences of Supercharged GFP Variants

−30 GFP:

(SEQ ID NO: XX)

MGHHHHHHGGASKGEELFDGVVPILVELDGDVNGHEFSVRGEGEGDATEG
ELTLKFICTTGELPVPWPTLVTTLTYGVQCFSDYPDHMDQHDFFKSAMPE
GYVQERTISFKDDGTYKTRAEVKFEGDTLVNRIELKGIDFKEDGNILGHK
LEYNFNSHDVYITADKQENGIKAEFEIRHNVEDGSVQLADHYQQNTPIG
DGPVLLPDDHYLSTESALSKDPNEDRDHMVLLEFVTAAGIDHGMDELYK
+15 GFP:

(SEQ ID NO: XX)

MGHHHHHHGGASKGERLFTGVVPILVELDGDVNGHKFSVRGEGEGDATRG
KLTLKFICTTGKLPVPWPTLVTTLTYGVQCFSRYPKHMKRHDFFKSAMPE
GYVQERTISFKKDGTYKTRAEVKFEGRTLVNRIELKGRDFKEKGNILGHK
LEYNFNSHNVYITADKRKNGIKANFKIRHNVKDGSVQLADHYQQNTPIGR
GPVLLPRNHYLSTRSALSKDPKEKRDHMVLLEFVTAAGITHGMDELYK
+25 GFP:

(SEQ ID NO: XX)

MGHHHHHHGGASKGERLFTGVVPILVELDGDVNGHKFSVRGKGKGDATRG
KLTLKFICTTGKLPVPWPTLVTTLTYGVQCFSRYPKHMKRHDFFKSAMPK
GYVQERTISFKKDGTYKTRAEVKFEGRTLVNRIKLKGRDFKEKGNILGH
KLRYNFNSHNVYITADKRKNGIKANFKIRHNVKDGSVQLADHYQQNTPIG
RGPVLLPRNHYLSTRSALSKDPKEKRDHMVLLEFVTAAGITHGMDELYK
+36 GFP:

(SEQ ID NO: XX)

MGHHHHHHGGASKGERLFRGKVPILVELKGDVNGHKFSVRGKGKGDATRG
KLTLKFICTTGKLPVPWPTLVTTLTYGVQCFSRYPKHMKRHDFFKSAMPK
GYVQERTISFKKDGKYKTRAEVKFEGRTLVNRIKLKGRDFKEKGNILGH
KLRYNFNSHKVYITADKRKNGIKAKFKIRHNVKDGSVQLADHYQQNTPIG
RGPVLLPRNHYLSTRSKLSKDPKEKRDHMVLLEFVTAAGIKHGRDERYK
+36 GFP-HA2:

(SEQ ID NO: XX)

MGHHHHHHGGASKGERLFRGKVPILVELKGDVNGHKFSVRGKGKGDATRG
KLTLKFICTTGKLPVPWPTLVTTLTYGVQCFSRYPKHMKRHDFFKSAMPK
GYVQERTISFKKDGKYKTRAEVKFEGRTLVNRIKLKGRDFKEKGNILGH
KLRYNFNSHKVYITADKRKNGIKAKFKIRHNVKDGSVQLADHYQQNTPIG
RGPVLLPRNHYLSTRSKLSKDPKEKRDHMVLLEFVTAAGIKHGRDERYK
GSAGSAAGSGEFGLFGAIAGFIENGWEGMIDG

### Gel-Shift Assay

Gel-shift assays were based on the method of Kumar et al. (Kumar P, Wu H, McBride J L, Jung K E, Kim M H, et al. (2007) Transvascular delivery of small interfering RNA to the central nervous system. Nature 448: 39-43; incorporated herein by reference). siRNA (10 pmol) or plasmid DNA (22 fmol) was mixed with the specified quantity of a GFP variant in phosphate buffered saline (PBS) for 10 minutes at 25° C. The resulting solution was analyzed by non-denaturing electrophoresis using a 15% acrylamide gel for siRNA or a 1% agarose gel for plasmid DNA, stained with ethidium bromide, and visualized with UV light.

### Cationic Lipid-Based and GFP-Based Transfection

Transfections using Lipofectamine 2000 (Invitrogen) and Fugene 6 (Roche) were performed following the manufacturer's protocol. Although the molecular weight of these reagents are not provided by the manufacturer, the working concentration of Lipofectamine 2000 during transfection is 2 μg/mL and based on an assumption that the molecular weight of this cationic lipid is ≦1,000 Da we estimate that this concentration corresponds to ≧˜2 μM.

Cells were plated in a 12-well tissue culture plate at a density of 80,000 cells per well. After 12 hours at 37° C., the cells were washed with 4° C. (PBS) and for HeLa, IMCD, 3T3-L, and PC12 cells the media were replaced with 500 μL of serum-free DMEM at 4° C.

Jurkat cells were transferred from the culture plate wells into individual 1.5 mL tubes, pelleted by centrifugation, and resuspended in 500 μL of serum-free RPMI 1640 at 4° C.

A solution of GFP and either siRNA or plasmid DNA was mixed in 500 μL of either 4° C. DMEM (for HeLa, IMCD, 3T3-L, and PC12 cells) or 4° C. RPMI 1640 (for Jurkat cells). After 5 min at 25° C., this solution was added to the cells and slightly agitated to mix. After 4 hours at 37° C., the solution was removed from the cells and replaced with 37° C. media containing 10% FBS. GAPDH-targeting Cy3-labeled siRNA and unlabeled siRNA were purchased from Ambion. Plasmid transfections were performed using pSV-β-galactosidase (Promega). β-galactosidase activity was measured using the β-fluor assay kit (Novagen) following the manufacturer's protocol.

### Fixed-Cell Imaging

Four hours after treatment with GFP and Cy3-siRNA, cells were trypsinized and replated in medium containing 10% FBS on glass slides coated with Matrigel (BD Biosciences). After 24 hours at 37° C., cells were fixed with 4% formaldehyde in PBS, stained with DAPI where indicated, and imaged with a Leica DMRB inverted microscope equipped with filters for GFP and Cy3 emission. Images were prepared using OpenLab software (Improvision). Exposure times for GFP and Cy3 were fixed at 350 msec and 500 msec, respectively.

### Live-Cell Imaging

For experiments using small-molecule inhibitors, cells were plated on a glass-bottomed tissue culture plate (MatTek, 50 mm uncoated plastic dishes with #1.5 glass thickness and a 14 mm glass diameter) and incubated with inhibitor for 1 hour at 37° C., followed by treatment with 50 nM +36 GFP and inhibitor for an additional 1 hour at 37° C. The resulting cells were washed three times with PBS containing the inhibitor and 20 U/mL heparin to remove surface-associated GFP, with the exception that cells treated with 50 nM +36 GFP at 4° C. were washed only one time with PBS containing 20 U/mL heparin to remove GFP bound to the glass slide but to still allow a perimeter of some cell surface-bound GFP to be visible.

Cells were imaged using an inverted microscope (Olympus IX70) in an epi-fluorescent configuration with an oil-immersion objective (numerical aperture 1.45, 60×, Olympus). GFP was excited with the 488 nm line an argon ion laser (Melles-Griot), and Alexa Fluor 647 was excited with a 633 nm helium-neon laser (Melles-Griot). Long- and short-wavelength emissions were spectrally separated by a 650 nm long-pass dichroic mirror (Chroma) and imaged onto a CCD camera (CoolSnap HQ). A 665 nm long-pass filter was used for Alexa Fluor 647 detection, and a 535/20 nm bandpass filter for GFP. Imaging was conducted at 37° C.

### RT-QPCR

Cells were washed with PBS 48, 72, or 96 hours after transfection and total RNA was extracted using the Ribopure kit (Ambion) following the manufacturer's protocol. Samples were treated with 1 uL DNase I (Ambion) and incubated for 30 minutes at 37° C. DNase I was inactived with DNase I Inactivation Reagent (Ambion) following the manufacturer's protocol. Complementary DNA was generated from 800 ng of RNA using the Retroscript kit (Ambion) following the manufacturer's protocol. QPCR reactions contained 1× IQ SYBR green Master Mix (BioRad), 3 nM ROX reference dye (Stratagene), 2.5 μL of reverse transcription reaction mixture, and 200 nM of both forward and reverse primers:

(SEQ ID NO: XX)

Forward GAPDH 5′-CAACTCACTCAAGATTGTCAGCAA-3′

(SEQ ID NO: XX)

Reverse GAPDH 5′-GGGATGGACTGTGGTCATGA-3′

(SEQ ID NO: XX)

Forward β-actin 5′-ATAGCACAGCCTGGATAGCAACGTAC-3′

(SEQ ID NO: XX)

Reverse β-actin 5′-CACCTTCTACAATGAGCTGCGTGTG-3′

QPCR reactions were subjected to the following program on a Stratagene MX3000p QPCR system: 15 minutes at 95° C., then 40 cycles of (30 seconds at 95° C., 1 minute at 55° C., and 30 seconds at 72° C.). Amplification was quantified during the 72° C. step. Dissociation curves were obtained by subjecting samples to 1 minute at 95° C., 30 seconds at 55° C., and 30 seconds at 95° C. and monitoring fluorescence during heating from 55° C. to 95° C. Threshold cycle values were determined using MxPro v3.0 software (Stratagene) and analyzed by the ΔΔCt method.

### Western Blotting

Cells were washed once with 4° C. PBS 96 hours after transfection. Cells were lysed with 200 μL RIPA buffer (Boston Bioproducts) containing a protease inhibitor cocktail (Roche) for 5 minutes. The resulting cell lysate was analyzed by SDS-PAGE on a 4-12% acrylamide gel (Invitrogen).

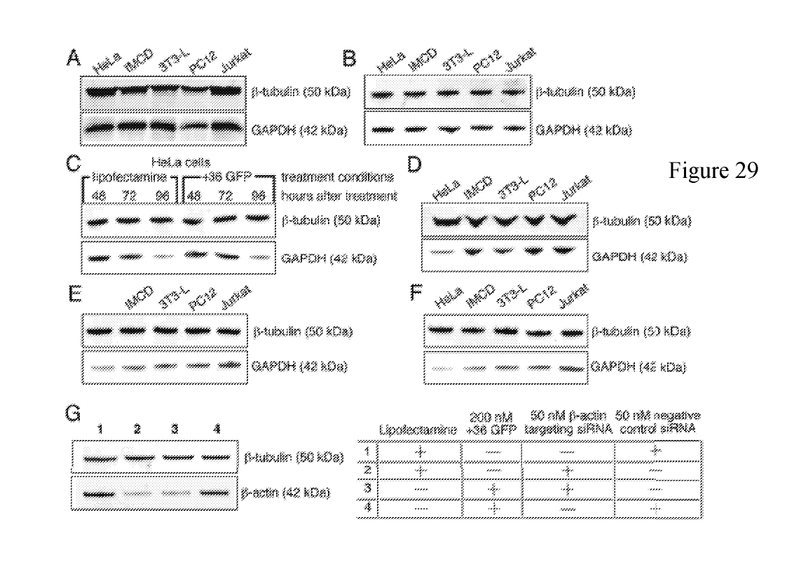

The proteins on the gel were transferred by electroblotting onto a PVDF membrane (Millipore) pre-soaked in methanol. Membranes were blocked in 5% milk for 1 hour, and incubated in primary antibody in 5% milk overnight at 4° C. All antibodies were purchased from Abcam. The membrane was washed three times with PBS and treated with secondary antibody (Alexa Fluor 680 goat anti-rabbit IgG (Invitrogen) or Alexa Fluor 800 rabbit anti-mouse IgG (Rockland)) in blocking buffer (Li-COR Biosciences) for 30 minutes. The membrane was washed three times with 50 mM Tris, pH 7.4 containing 150 mM NaCl and 0.05% Tween-20 and imaged using an Odyssey infrared imaging system (Li-COR Biosciences). Images were analyzed using Odyssey imaging software version 2.0. Representative data are shown in FIG. 29. GAPDH suppression levels shown are normalized to β-tubulin protein levels; 0% suppression is defined as the protein level in cells treated with ˜2 μM Lipofectamine 2000 and 50 nM negative control siRNA.

### Flow Cytometry

Cells were washed three times with 20 U/mL heparin (Sigma) in PBS to remove non-internalized GFP. Adherent cells were trypsinized, resuspended in 1 mL PBS with 1% FBS and 75 U/mL DNase (New England Biolabs). Flow cytometry was performed on a BD LSRII instrument at 25° C. Cells were analyzed in PBS using filters for GFP (FITC) and Cy3 emission. At least 10⁴ cells were analyzed for each sample.

### Synthetic Cationic Peptides

(Arg)₉ and (KKR)₁₁(RRK) were purchased from Chi Scientific and used at a purity of ≧95%. Poly-(L)-Lys and poly-(D)-Lys were purchased from Sigma. Poly-(L)-Lys is a mixture with a molecular weight window of 1,000-5,000 Da, and a median molecular weight of 3,000 Da. Poly-(D)-Lys is a mixture with a molecular weight window of 1,000-5,000 Da, and a median molecular weight of 2,500 Da. Stock solutions of all synthetic peptides were prepared at a concentration of 20 μM in PBS.

### +36 GFP-siRNA Particle Size Characterization

Dynamic light scattering was performed using a Protein Solution DynaPro instrument at 25° C. using 20 μM +36 GFP and 5 μM siRNA in PBS. A purified 20-bp RNA duplex (5′ GCAUGCCAUUACCUGGCCAU 3′, from IDT; SEQ ID NO: XX) was used in these experiments. Data were modeled to fit an isotrophic sphere. 5 μL of solution analyzed by DLS (20 μM +36 GFP and 5 μM siRNA in PBS) was imaged using a Leica DMRB inverted microscope.

### Stability Assays

To assess siRNA stability in murine serum, siRNA (10 pmol) was mixed with sfGFP (40 pmol), mixed with +36 GFP (40 pmol), or incubated alone in PBS for 10 minutes at 25° C. The resulting solution was added to four volumes of mouse serum (20 μL total) and incubated at 37° C. for the indicated times. 15 μL of the resulting solution was diluted in water to a total volume of 100 μL. 100 μL of TRI reagent (Ambion) and 30 μL of chloroform was added. After vigorous mixing and centrifugation at 1,000 G for 15 minutes, the aqueous layer was recovered. siRNA was precipitated by the addition of 15 μL of 3 M sodium acetate, pH 5.5, and two volumes of 95% ethanol. siRNA was resuspended in 10 mM Tris pH 7.5 and analyzed by gel electrophoresis on a 15% acrylamide gel. Serum stability of +36 GFP when complexed with siRNA was simultaneously measured by anti-GFP Western blot with 5 μL of the incubation.

To assess the stability of plasmid DNA complexed with +36 GFP in murine serum, plasmid DNA (0.0257 pmol) was mixed with either 2.57 pmol, 100 eq. or 12.84 pmol, 500 eq. of either sfGFP or +36 GFP in 4 μL of PBS for 10 minutes. To this solution was added 16 μL of mouse serum (20 μL total) and incubated at 37° C. for the indicated times. DNA was isolated by phenol chloroform extraction and analyzed by gel electrophoresis on a 1% agarose gel, stained with ethidium bromide, and visualized with UV light.

To assess the stability of proteins in murine serum, 100 pmol of each protein in 2 μL of PBS was mixed with 8 μL of murine serum (Sigma) and incubated at 37° C. The samples were mixed with SDS protein loading buffer and heated to 90° C. for 10 minutes. The resulting mixture was analyzed by SDS-PAGE on a 4-12% acrylamide gel (Invitrogen) and imaged by Western blot.

To assess stability in the presence of proteinase K, 100 pmol of +36 GFP or BSA was treated with 0.6 units of proteinase K (New England Biosciences) at 37° C. The samples were mixed with SDS protein loading buffer, heated to 90° C. for 10 minutes, and analyzed by SDS-PAGE on a 4-12% acrylamide gel (Invitrogen).

### Example 4

### Supercharged Proteins are Effective Protein Delivery Reagents

mCherry, a fluorescent protein, was fused to each of +36 GFP (via a cleavable linker having amino acid sequence ALAL, SEQ ID NO: XX), TAT, and Arg₉ to generate three mCherry fusion proteins. These fusions were tested for their ability to deliver mCherry to HeLa, IMCD, and PC12 cells.

In order to assess how well +36 GFP delivers proteins to cells HeLa, PC12 and 3T3-L cells were treated with either (1) mCherry-TAT, (2) mCherry-R₉, or (3) mCherry-+36 GFP. Cells were treated with 50 nM, 500 nM, 1 μM, or 2 μM material for 4 hours in DMEM, followed by heparin wash and FACS.

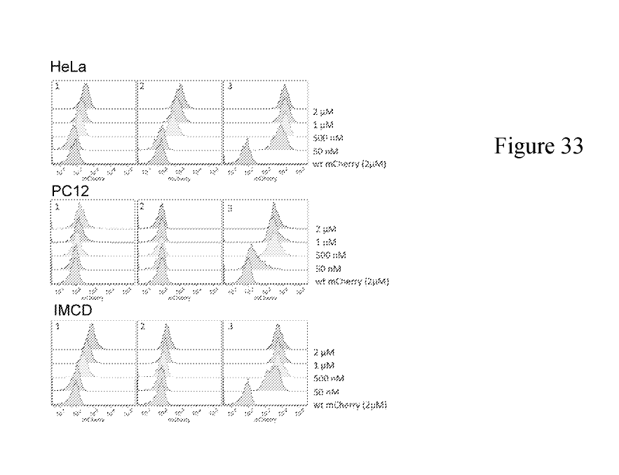

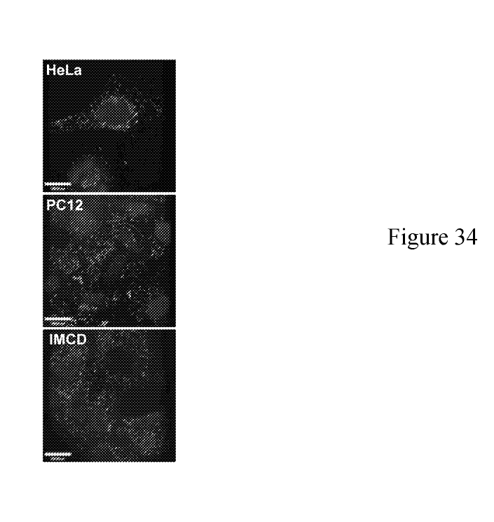

mCherry-ALAL-+36 GFP penetrated cells much more potently than mCherry-TAT or mCherry Arg₉ (FIG. 33). FIG. 34 shows internalization of these three fusions via fluorescence microscopy. Data show that +36 GFP is a highly potent and general protein delivery reagent (FIG. 34).

### Example 5

### Mining Genomes for Natural Supercharged Proteins

The present invention encompasses the recognition that genomes (e.g., the human genome) can be mined to identify natural supercharged proteins that might be useful for delivery of agents (e.g., nucleic acids, proteins, etc.). Ten human proteins were expressed and purified (i.e., C-Jun (Protein Accession No.: P05412); TERF 1 (P54274); Defensin 3 (P81534); Eotaxin (Q9Y258); N-DEK (P35659); PIAS 1 (O75925); Ku70 (P12956); Midkine (P21741); HBEGF (Q99075); HGF (P14210); SFRS12-IP1 (Q8N9Q2); Cyclon (Q9H6F5)), and four of these (i.e., HBEGF, N-DEK, C-jun, and 2HGF) displayed the ability to bind to siRNA and deliver siRNA to cells (i.e., cultured HeLa cells).

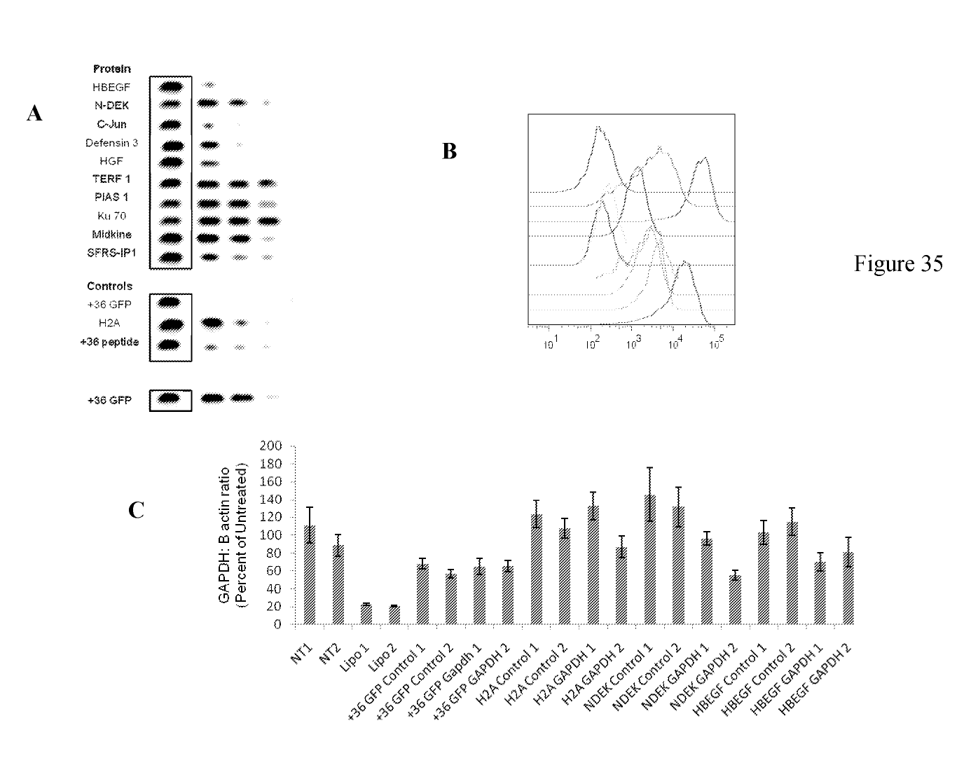

Human proteins were assayed for binding to siRNA by gel shift assay. Gel-shift assays were based on the method of Kumar et al. (Kumar P, Wu H, McBride J L, Jung K E, Kim M H, et al. (2007) Transvascular delivery of small interfering RNA to the central nervous system. Nature 448: 39-43; incorporated herein by reference). Ambion negative control siRNA (˜150 ng) was mixed with the specified quantity of human protein in phosphate buffered saline (PBS) for 10 minutes at 25° C. The resulting solution was analyzed for unbound siRNA by non-denaturing electrophoresis using a 15% acrylamide gel for siRNA, stained with ethidium bromide, and visualized with UV light (FIG. 35A).

Human proteins were assayed for delivery of siRNA to Hela cells. Cells were plated in a 12-well tissue culture plate at a density of 80,000 cells per well. After 12 hours at 37° C., the cells were washed with 4° C. (PBS) and replaced with 500 μL of serum-free DMEM at 4° C. A solution of human protein and Ambion negative control Cy3-labeled siRNA was mixed in 500 μL of 4° C. DMEM. After 5 min at 25° C., this solution was added to the cells and slightly agitated to mix. Final concentration of human proteins was 1 micromolar and siRNA was 50 micromolar. After 4 hours at 37° C., the solution was removed from the cells and replaced with 37° C. media containing 10% FBS. Cells were then analyzed for siRNA delivery by fixed cell imaging and flow cytometry. Internalization of protein-siRNA complexes is shown in FIG. 35B.

HeLa cells were transfected with Ambion Cy3-labeled siRNA using human proteins, incubated for three days, and then assayed for degradation of a targeted mRNA (FIG. 35C). Targeted GAPDH mRNA levels were compared to β-actin mRNA levels. “Control” indicates use of a non-targeting siRNA. Lipofectamine 2000 was used as a positive control.

### Example 6

### Pyrene Butyric Acid Improves Consistency of Gene Silencing

The present inventors have discovered that pyrene butyrate, an endosomolytic agent (Futaki et al., 2006, ACS Chem. Biol., 1:299; incorporated herein by reference), can increase gene silencing effects and decrease batch-to-batch variability. Without wishing to be bound by any one particular theory, such variability may be caused by variable ion endosome escape efficiency). Thus, the present inventors have developed a method for improving the efficiency, consistency, and reproducibility of gene silencing.

The protocol below utilizes +36 GFP and pyrene butyric acid (PBA), but can readily be generalized to any supercharged protein and any endosomolytic agent (e.g., chloroquine, HA2, melittin).

HeLa cells were grown to ˜80% confluency in a 12-well plate. DMEM/10% FBS was removed and the cells were washed 3 times with PBS. To each well was added 1 mL of a solution containing 50 μM PBA in PBS. Cells were incubated in this solution for 5 minutes at 37° C. In a small plastic tube, 200 fmol of GAPDH-suppressing siRNA (2 μL of a 100 μM siRNA solution) and 800 fmol +36 GFP were pre-mixed and allowed to incubate for 5 minutes at 25° C. One quarter (¼) of the total volume of the siRNA/+36 GFP complex was added to each well containing 1 mL 50 μM PBA in PBS. The tissue culture tray was agitated slightly to homogenize the solution in each well, resulting in a solution containing 50 μM siRNA and 200 μM +36 GFP. Cells were incubated under these conditions for 3 hours at 37° C. The 50 μM PBA/PBS solution was removed and cells were washed three times with PBS, followed by the addition of 1 mL DMEM in 10% FBS. Cells were incubated under these conditions for 4 days, and knockdown of GAPDH expression was quantitated by Western blot.

About 20% cytotoxicity was observed after 3 hour incubation in 50 μM PBA/PBS. Much higher cytotoxicity (˜80%) was observed when HeLa cells were incubated in 50 μM PBA/PBS for ≧4 hours. Cytotoxicity of PBA may vary by cell type.

### EQUIVALENTS AND SCOPE

Those skilled in the art will recognize, or be able to ascertain using no more than routine experimentation, many equivalents to the specific embodiments, described herein. The scope of the present invention is not intended to be limited to the above Description, but rather is as set forth in the appended claims.

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not been specifically set forth in haec verba herein. It is also noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any supercharged protein; any nucleic acid; any method of production; any method of use; etc.) can be excluded from any one or more claims, for any reason, whether or not related to the existence of prior art.

1. (canceled)

2. (canceled)

3. A method of introducing a supercharged protein, or an agent associated with a supercharged protein, or both, into a cell, comprising:
contacting said supercharged protein, or a supercharged protein and an agent associated with the supercharged protein with said cell under conditions sufficient to allow penetration of said supercharged protein, or said agent associated with a supercharged protein, into the cell, thereby introducing a supercharged protein, or an agent associated with a supercharged protein, or both, into a cell.

4. The method of claim 3, further confirming that said supercharged protein or agent associated with said supercharged protein has penetrated said cell by one or more of detecting a label, detecting a biological change in said cell, or detecting a response in a subject to which the supercharged protein, or an agent associated with a supercharged protein was administered.

5. A complex comprising:
a supercharged protein, wherein the supercharged protein has an overall net charge greater than its corresponding unmodified protein; and
one or more nucleic agents.

6. The complex of claim 5, wherein the supercharged protein has an overall net positive charge.

7. The complex of claim 6, wherein the overall net positive charge is about +5, about +10, about +15, about +20, about +25, about +30, about +35, or about +40.

8-15. (canceled)

15. The complex of claim 6, wherein the supercharged protein of interest is more positively charged at physiological pH than its corresponding unmodified protein.

16. The complex of claim 6, wherein the supercharged protein of interest is at least +5 or at least +10 more positively charged at physiological pH than its corresponding unmodified protein.

17-20. (canceled)

21. The complex of claim 5, wherein the supercharged protein is green fluorescent protein (GFP).

22. (canceled)

23. The complex of claim 5, wherein the supercharged protein is a superpositively charged GFP (+36 GFP) of the sequence:
(SEQ ID NO: 7)
MGHHHHHHGGASKGERLFRGKVPILVELKGDVNGHKFSVRGKGKGDATRG
KLTLKFICTTGKLPVPWPTLVTTLTYGVQCFSRYPKHMKRHDFFKSAMPK
GYVQERTISFKKDGKYKTRAEVKFEGRTLVNRIKLKGRDFKEKGNILGHK
LRYNFNSHKVYITADKRKNGIKAKFKIRHNVKDGSVQLADHYQQNTPIGR
GPVLLPRNHYLSTRSKLSKDPKEKRDHMVLLEFVTAAGIKHGRDERYK.

24-27. (canceled)

28. The complex of claim 5, wherein the supercharged protein comprises a stretch of about 50 amino acids of the amino acid sequence as set forth in SEQ ID NO: 7.

29-33. (canceled)

34. The complex of claim 5, wherein the supercharged protein comprises an amino acid sequence that is about 80% identical, about 90% identical, or about 95% identical to the amino acid sequence set forth in SEQ ID NO: 7.

35-36. (canceled)

37. The complex of claim 5, wherein the supercharged protein is a fusion protein of green fluorescent protein and hemagglutinin 2 (HA2) peptide.

38. The complex of claim 5, wherein the supercharged protein is a fusion protein of green fluorescent protein and hemagglutinin 2 (HA2) peptide of the sequence:
(SEQ ID NO: 94)
MGHHHHHHGGASKGERLFRGKVPILVELKGDVNGHKFSVRGKGKGDATRG
KLTLKFICTTGKLPVPWPTLVTTLTYGVQCFSRYPKHMKRHDFFKSAMPK
GYVQERTISFKKDGKYKTRAEVKFEGRTLVNRIKLKGRDFKEKGNILGHK
LRYNFNSHKVYITADKRKNGIKAKFKIRHNVKDGSVQLADHYQQNTPIGR
GPVLLPRNHYLSTRSKLSKDPKEKRDHMVLLEFVTAAGIKHGRDERYKGS
AGSAAGSGEFGLFGAIAGFIENGWEGMIDG.

39. The complex of claim 5, wherein the nucleic acid comprises RNA or DNA.

40. (canceled)

41. The complex of claim 5, wherein the nucleic acid comprises an RNAi agent.

42-47. (canceled)

48. The complex of claim 5, wherein the nucleic acid comprises a vector.

49-50. (canceled)

51. The complex of claim 5, wherein the ratio of supercharged protein to nucleic acid is about 1:1, about 1:2, about 1:3, about 1:4, or about 1:5.

52-57. (canceled)

58. A complex comprising:
a protein selected from the group consisting of cyclon (ID No.: Q9H6F5), PNRC1 (ID No.: Q12796), RNPS1 (ID No.: Q15287), SURF6 (ID No.: O75683), AR6P (ID No.: Q66PJ3), NKAP (ID No.: Q8N5F7), EBP2 (ID No.: Q99848), LSM11 (ID No.: P83369), RL4 (ID No.: P36578), KRR1 (ID No.: Q13601), RY-1 (ID No.: Q8WVK2), BriX (ID No.: Q8TDN6), MNDA (ID No.: P41218), H1b (ID No.: P16401), cyclin (ID No.: Q9UK58), MDK (ID No.: P21741), PROK (ID No.: Q9HC23), FGF5 (ID No.: P12034), SFRS (ID No.: Q8N9Q2), AKIP (ID No.: Q9NWT8), CDK (ID No.: Q8N726), beta-defensin (ID No.: P81534), PAVAC (ID No.: P18509), eotaxin-3 (ID No.: Q9Y258), histone H2A (ID No.: Q7L7L0), and HMGB1 (ID No.: P09429); and
one or more polynucleotides, peptides, proteins, or small molecules.

59-77. (canceled)

78. A pharmaceutical composition comprising
a complex of claim 5; and
a pharmaceutically acceptable excipient.

79. A method comprising steps of:
providing a subject susceptible to, suffering from, or displaying one or more symptoms of a disease, disorder, or condition;
administering the complex of claim 5 to the subject, such that at least one symptom is improved.

80-93. (canceled)
